(19) United States
(12) Patent Application Publication
(10) Pub. No.: US 2026/0167938 A1
(43) Pub. Date: Jun. 18, 2026

(54) DNA ORIGAMI TRAPS FOR LARGE VIRUSES

(71) Applicant: Technische Universität München, München (DE)

(72) Inventors: Alba Monferrer i Sureda, München (DE); Christian Sigl, München (DE); Hendrik Dietz, Haar (DE)

(21) Appl. No.: 19/124,197

(22) PCT Filed: Oct. 31, 2023
(86) PCT No.: PCT/EP2023/080373

§ 371 Date: Apr. 24, 2025

(30) Foreign Application Priority Data
Oct. 31, 2022 (EP) 22204809.2

(51) Int. Cl. C12N 7/00

(52) U.S. Cl. CPC C12N 7/00

(57) ABSTRACT

The present invention relates to a three-dimensional polynucleotide-based open shells for encapsulating a virus or viral particle, to a composition comprising a mixture of such three-dimensional polynucleotide-based open shells, to a composition comprising a virus or viral particle encapsulated by such three-dimensional polynucleotide-based open shells, and to a method for encapsulating a virus, a viral particle or a subviral particle by using such a three-dimensional polynucleotide-based open shells.

### FIELD OF THE INVENTION

The present invention relates to a three-dimensional polynucleotide-based open shells for encapsulating a virus, a viral particle or a subviral particle, to a composition comprising a mixture of such three-dimensional polynucleotide-based open shells, to a composition comprising a virus, a viral particle or a subviral particle encapsulated by such three-dimensional polynucleotide-based open shells, and to a method for encapsulating a virus, a viral particle or a subviral particle by using such a three-dimensional polynucleotide-based open shells.

### BACKGROUND OF THE INVENTION

Viral infections cause millions of deaths per year globally, enormous suffering and morbidity, and impose huge drains on societies and economies in health care costs, lost work time, and other less easily measured burdens such as mental health issues associated with loss of parents, children, and care givers or stigmatization. Climate change and global migration are projected to increase the threat of viral outbreaks because vectors spread to regions that so far were too cold for them to survive. The burden of virus infections will further increase due to habitat encroachment by humans, urbanization and megacities with increasing population density, increasing travel not only locally but also far distance, and numerous other drivers of disease emergence⁴¹. Viruses are the pathogen class most likely to adapt to new environmental conditions because of their short generation time and genetic variability allowing rapid evolution⁴². For the majority of viral diseases (˜70% of current WHO-listed viruses), no effective treatment is available. The few existing antiviral therapies are almost exclusively targeted to a specific virus and do not allow application against a newly emerging pathogen. In addition, antiviral therapy typically faces the challenge that it must be started very soon after infection to be effective, before the viral load gets too high and caused disease symptoms. Emerging virus threats require a rapid response, but broadly applicable ready-to-use antivirals do not exist.

In this context, it is useful to first consider how current antiviral therapies work. Existing antiviral drugs target either virus-specific proteins, mostly polymerases, or essential virus or cellular structures that enable virus replication and spread. The major targetable steps in a virus replication cycle are (1) virus particles docking to the cell membrane of host cells; (2) uptake into the host cell; (3) release of the virus capsid into the cytoplasm and transport of the viral genome to the replication spot; (4) synthesis of viral nucleic acids and proteins and posttranslational processing of viral proteins; (5) assembly of virus components into new viral particles; (6) release of the newly formed viruses from the infected cell. Most clinically available antivirals are polymerase-inhibitors that are specific for a given viral enzyme. Examples include acyclovir⁴³, active against herpes simplex and varizella zoster virus; tenofovir, active against hepatitis B virus (HBV) and HIV and sofosbuvir, active against hepatitis C virus (HCV). Examples for drugs targeting different stages of the virus life cycle are: enfuvirtide⁴⁴, which inhibits HIV fusion (stage 2); amantadine⁴⁵, which inhibits influenza A virus uncoating (stage 3); or the neuraminidase inhibitor oseltamivir⁴⁶, which interferes with influenza virus release from host cells (stage 6)⁴⁶. These drugs, however, can only act when a virus is replicating or spreading but cannot kill or neutralize it. None of these antivirals is broadly applicable.

Viruses come in many shapes and sizes. Their dimensions range from the 10 to the 1000 nm scale. For example, adeno-associated virus (AAV) is a rather small icosahedral, non-enveloped virus with an approximate and reproducible diameter of 20 nm per particle. Influenza viruses are enveloped and medium-size viruses with dimensions on the 80 to 150 nm scale. Influenza viruses are also pleomorphic, meaning that the particles may adopt a variety of shapes and dimensions including spherical, peanut-shaped or even filamentous. Mimivirus is a representative of a rather large virus with its ˜700 nm diameter.

For all viruses, attachment to the host cell membrane is a prerequisite for cell penetration, infection, and replication.

Preventing viruses from entering cells is increasingly being considered for the development of antiviral treatments. Examples of virus entry inhibitors include peptides,¹ antibodies,² dendrimers,³⁻⁵ nanoparticles and polymers coated with virus-binding moieties.^{6,7} The majority of these entry inhibitors function on a molecule-to-molecule basis, meaning that one copy of the antiviral agent targets one viral surface protein. More recently, multivalent antiviral concepts have been put forward that display multiple virus-binding molecules in complex geometries intended to match more mesoscale structural aspects of the target pathogen, as exemplified with virus-binding two-dimensional,⁸⁻¹⁰ and three-dimensional DNA architectures.^{11,12} Multivalent virus-covering nanoarchitectures offer additional options to leverage avidity effects associated with multivalent interactions between antiviral and virus. Multivalent binding leads to exponential amplification of binding strength with valency and can enable achieving virtually irreversible target binding with individually weak and reversible virus binders. Virus surface alterations that reduce the binding strength of individual binders as for example caused by mutational drift may thus be less problematic in the context of the multivalent antiviral relative to a monovalent binder. It is also conceivable that the virus-binding moieties used in the multivalent nanoarchitectures themselves do not necessarily need to have neutralizing activity, since the entry-inhibitory effect will at least in part be accomplished by the virus-surface occluding material of the DNA nanoarchitecture.

It has previously been found that icosahedral DNA origami half-shells¹¹ can engulf and neutralize viruses up to 85 nm in diameter by mechanically blocking binding interactions with cell surfaces and therefore preventing the infection of host cells. Since there are many larger human viral pathogens of high relevance such as e.g., Influenza, Corona or Herpes viruses, it was sought to expand that approach to also be able to target such pathogens. Influenza viruses are enveloped viruses with dimensions on the 80 to 200 nm scale that occur in a variety of shapes including spherical, peanut-shaped, and filamentous.¹³ However, the previously developed virus-engulfing shell prototypes were either too restricted in size and shape to accommodate such virus particles or too cumbersome to produce to be of use in a real-world application.

The genomes of viruses frequently present mutations, which may lead to a diminished, or even potentially abolished, success of treatment options, such as vaccinations. Thus, there is a great need for therapeutic interventions that permit the fast adaptation to new emerging developments with respect to, for example, the infectivity of a given virus. None of the approaches mentioned above are modular and flexible enough to enable a fast adaptation of the structures to mutational changes of the viruses.

Thus, while different strategies for the treatment of viral infections have been developed or suggested up to date, there is still a need for the development of a concept of a generic antiviral drug platform for targeting a variety of viral pathogens. In particular, a concept would be desirable that does not rely on prior detailed knowledge about genetics and properties of the target virus. Additionally, it is of particular importance to develop an antiviral drug platform that is amenable for mass production.

### SUMMARY OF THE INVENTION

It is an object of the present invention to provide constructs that enable the encapsulation of a virus, a viral particle or a subviral particle. The solution to that problem, i.e., the use of simple macromolecular building blocks, such as DNA-based nanostructures, has not yet been taught or suggested by the prior art.

Therefore, in one aspect, the disclosure provides a three-dimensional polynucleotide-based open shell [1] (FIG. 26) encasing a cavity [2] and comprising an opening [3] for accessing said cavity, comprising an n-gonal pyramid [4] formed by n identical copies of a first type of an acute isosceles triangular prismoid t1 [5], wherein n is an integer selected from 7, 8, 9, 10, 11, 12, 13, 14 and 15, wherein the base plane [6] of each prismoid points to the outside of said open shell, and the upper plane [7] points to said cavity, wherein the two large side planes [8,9] of each prismoid contain a first pattern [10] and a second pattern [11] of one or more protrusions and/or one or more receptacles, wherein said first and said second patterns are complementary to each other, and wherein the small side plane [12] comprises a third pattern [13] of one or more protrusions and/or one or more receptacles; wherein said first type of an acute isosceles triangular prismoid is a self-assembling DNA-based building block comprising between 7,500 and 10,500 base pairs.

In another aspect, the present invention relates to the three-dimensional polynucleotide-based open shell according to the present invention for use in the treatment of a patient infected by, suspected to be infected by, or bearing the risk of becoming infected by, a virus, a viral particle or a subviral particle.

In another aspect, the present invention relates to a composition comprising a mixture of a three-dimensional polynucleotide-based open shells according to the present invention, wherein said mixture comprises three-dimensional polynucleotide-based open shells having values of n ranging from 7 to 15.

In another aspect, the present invention relates to the composition according to the present invention for use in the treatment of a patient infected by, suspected to be infected by, or bearing the risk of becoming infected by, a virus, a viral particle or a subviral particle.

In another aspect, the present invention relates to a method for encapsulating a virus, a viral particle or a subviral particle, comprising the steps of: providing a three-dimensional polynucleotide-based open shell according to the present invention, and contacting said macromolecule-based nanostructure with a medium comprising, or suspected to comprise, said virus, said viral particle or said subviral particle.

In another aspect, the present invention relates to a method for the treatment of a patient infected by, suspected to be infected by, or bearing the risk of becoming infected by, a virus, a viral particle or a subviral particle, comprising the step of: administering the three-dimensional polynucleotide-based open shell according to the present invention, or the composition according to the present invention to said patient.

In another aspect, the present invention relates to a method for the treatment of a patient infected by, or suspected to be infected by, a virus, a viral particle or a subviral particle, comprising the step of: contacting said patient, or a bodily fluid of said patient, with the three-dimensional polynucleotide-based open shell according to the present invention, or the composition according to the present invention.

In another aspect, the disclosure provides a composition comprising a virus, a viral particle or a subviral particle encapsulated by a three-dimensional polynucleotide-based open shell according to the present invention or by a three-dimensional polynucleotide-based open shell from the composition according to the present invention.

The disclosure contemplates all combinations of any one or more of the foregoing aspects and/or embodiments, as well as combinations with any one or more of the embodiments set forth in the detailed description and examples.

Other features, objects, and advantages of the compositions and methods herein will be apparent from the description and drawings, and from the claims.

### BRIEF DESCRIPTION OF THE DRAWINGS

FIG. 1 shows the C10 cone DNA origami design (n-gonal pyramid as defined in claim 1.a) with n=10). (A) Left: Schematical model of the C10 conical shell assembly. Cylinders indicate single DNA double helices. Each cone is designed to contain ten isosceles triangular subunits. Right: Schematics of C10 cones covering virus particles. (B) Schematical model of the subunit design, as implemented with multi-layer DNA origami in square-lattice packing. Arrows indicate shape-complementary docking sides located on sides 1 and 2 (S1 and S2). (C) 3D electron density map determined by single particle cryo electron microscopy revealing close agreement between designed and actual overall shape of the wedge subunit (see FIG. 9 for cryo-EM 3D class averages and field of view micrograph)

FIG. 2 shows the characterization of cone assembly. (A) Laser-scanned fluorescence image of a 1% agarose gel on which cone assembly reaction mixtures were electrophoresed, with samples taken at the indicated time points. The wedge subunit concentration was 5 nM, incubation temperature was 40° C., and the solution contained 25 mM MgCl₂. M: marker lane. Sc: M13-8064 scaffold as reference. (B) Exemplary negative stain TEM micrograph showing a field of view with cone assembly products. Inset: schematics of typical orientations in which cones adhere on TEM support grid. Scale bar: 100 nm. (C) Two-dimensional TEM class averages of distinct cone assembly species with base-adhered orientations (1). Scale bar: 50 nm. (D) Inner diameter measurements of (1) 2D class averages for each cone, as well as their frequency of occurrence. (E) Cryo-EM field of view micrograph showing different orientations of cones. Scale bar: 100 nM. (F) Cryo-EM 3D reconstructions of the C9 and C10 cones, with inner diameters and depth measurements.

FIG. 3 shows the stabilization of cone assembly for future in vivo applications. (A) Schematic illustration of the stabilization workflow: UV-point welding, oligolysine-PEG coating, and glutaraldehyde cross-linking of coating. (B) Design schematics showing details of the wedge subunit's strand diagram to indicate the positioning of additional thymidines (yellow dots) for the UV-point welding of t1 subunits. Diagram was prepared using caDNAno v0.2.4.³⁸ Blue: scaffold strand, grey: staple strands. (C) Laser-scanned fluorescence image of a 1% agarose gel on which cone assembly reaction mixtures were electrophoresed that had been exposed to irradiation with 310 nm light for the indicated times. The gel was run in the 3 mM MgCl₂, which are conditions in which non-crosslinked cones immediately disassemble into wedge subunits (see ctrl or 0 min lane for example). Inset: zoom into the high-molecular weight circular cone assembly products, with each band attributed to a closed cone with the indicated wedge subunit numbers. (D) Exemplary negative stain TEM images taken of non-irradiated (and thus not stabilized) versus irradiated cones in the presence of the indicated MgCl₂ concentrations. Scale bar: 100 nm. (E) Exemplary negative stain TEM images taken of solely UV-point welded cone assemblies treated with DNase I (0.001 U/μL) compared to samples that were additionally coated with oligolysine-PEG (1:0.6, P: N ratio) and chemically cross-linked with glutaraldehyde. Scale bar: 100 nm.

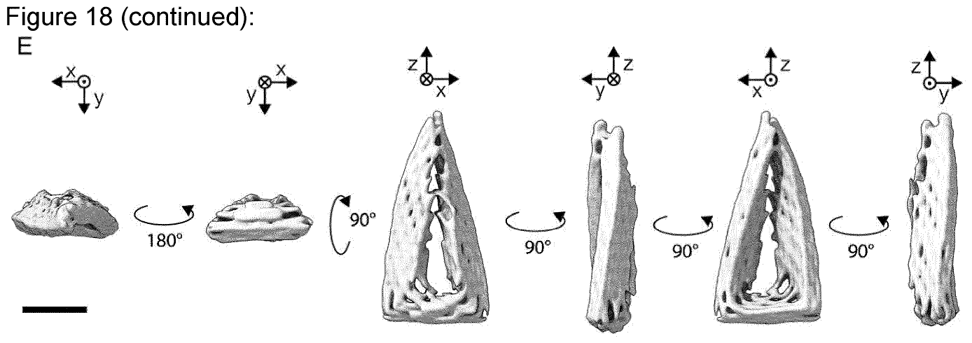

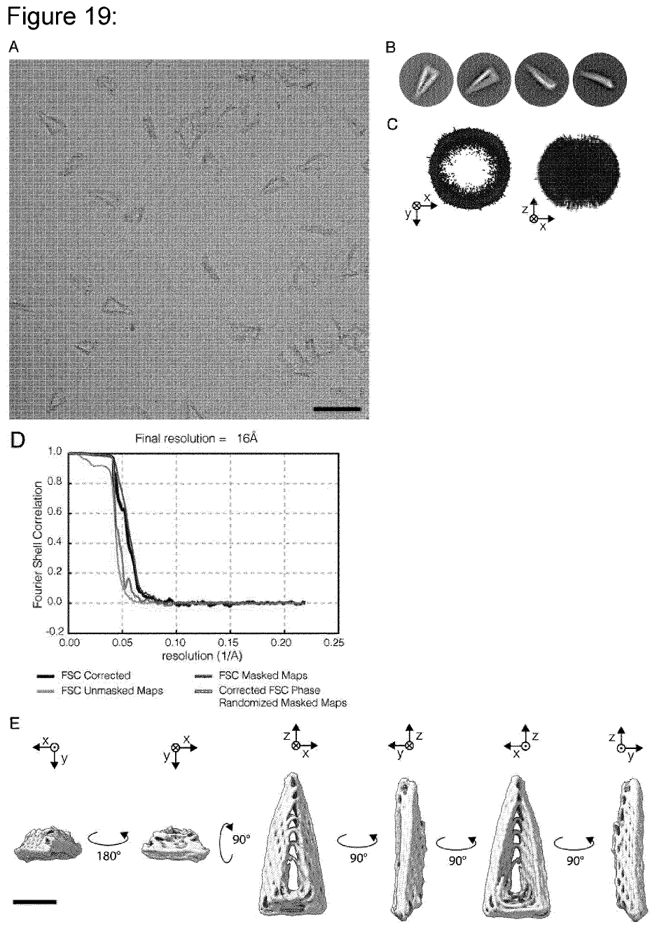

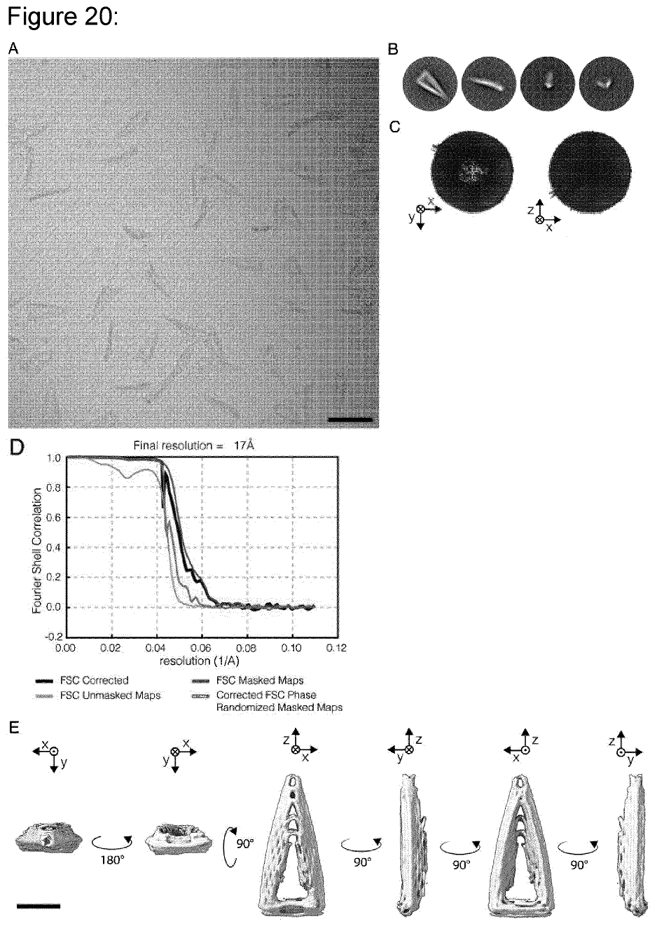

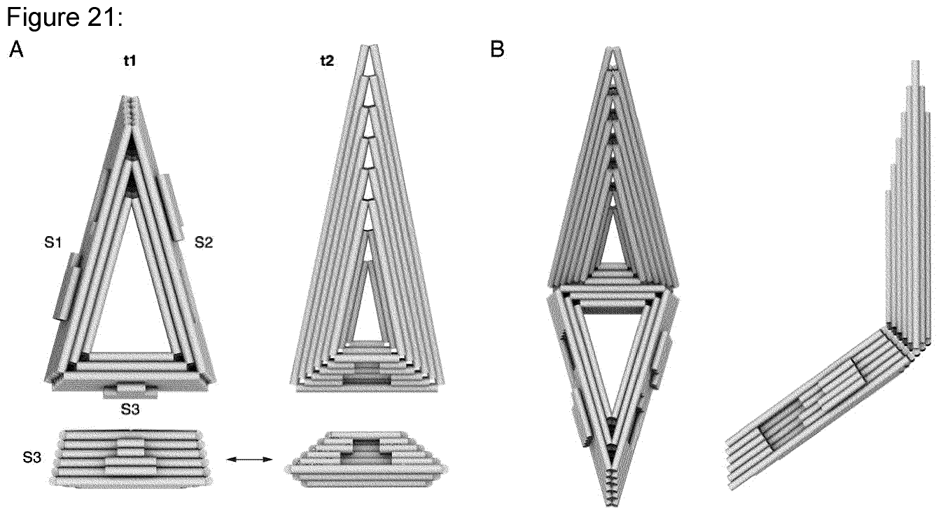

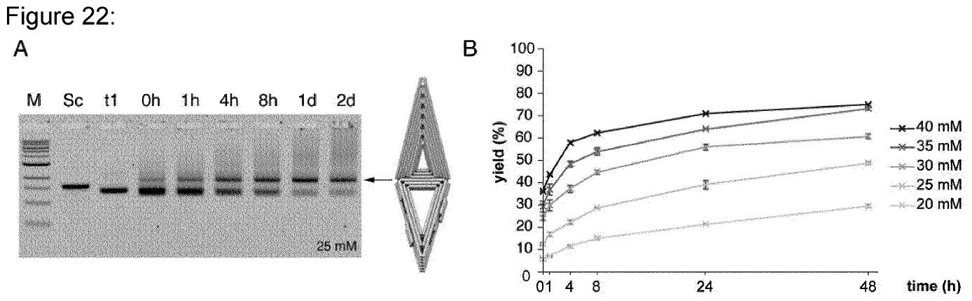

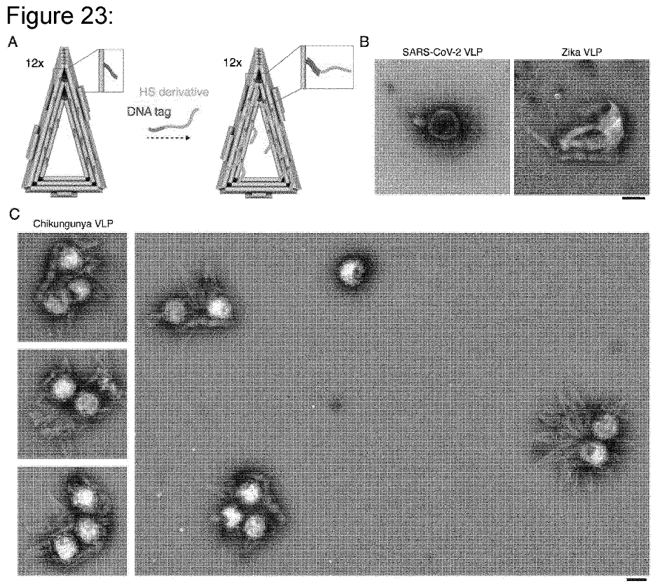

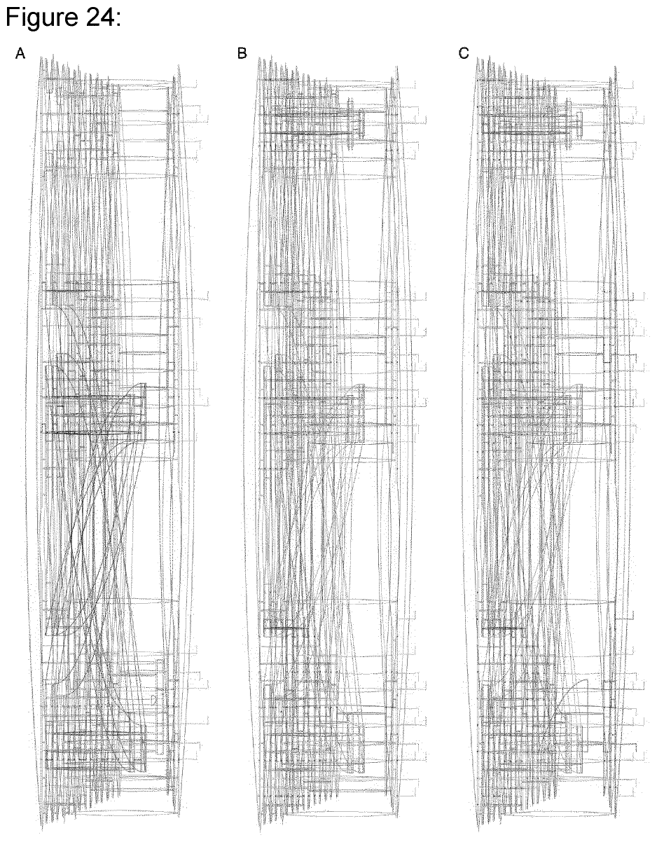

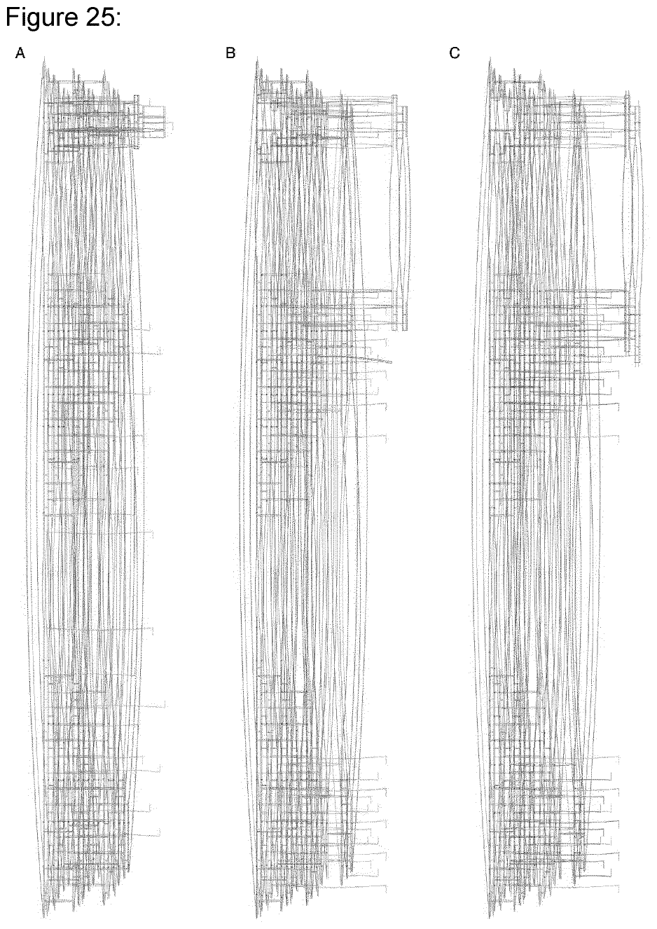

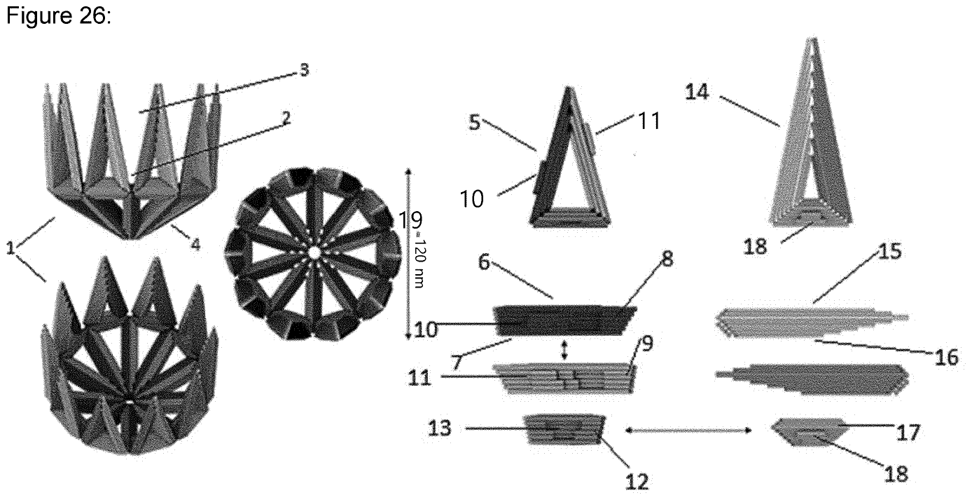

In this context, it should be noted that with respect to FIGS. 3B, 24 and 25 (see below), those figures show a schematic view of part of the complex arrangements of the different oligonucleotides forming the polynucleotide-based open shells of the present invention. All oligonucleotides used in forming these polynucleotide-based open shells are listed in Tables 1 to 3 and are included in the Sequence Listing. Thus, Tables 1 to 3 contain all sequence information needed in order to generate the nanostructures schematically shown in FIGS. 3B, 24 and 25, which are included for illustration purposes only. No additional sequence information is included in those figures.

FIG. 4 shows the engulfing of Influenza virus particles with cones. (A) Schematics of how cones may be functionalized with virus binding moieties. Red: single stranded DNA extensions called ‘handles’. Blue: DNA-tagged antibodies. (B) Influenza A/PR/8/34 virus trapping with cone assemblies featuring six copies of CR9114 antibodies per wedge subunit. Negative stain TEM images of single virus particles covered with different number of cones. Depending on the size and overall shape of the virus particles, up to three cones coordinated to cover the entirety of spherical/peanut shaped viruses, and even more copies of cones adapted to cover a filamentous Influenza particle. Scale bar: 50 nm. (C) Negative stain TEM images of cones coordinating to trap more than one virus particle at a time. Scale bar: 50 nm. (D) Slices through a single particle 3D tomogram of an Influenza virus fully engulfed by two cones in a sandwich-like assembly, acquired with a negative-staining TEM tilt series. Scale bar: 25 nm.

FIG. 5 shows spiked cone assemblies with enhanced surface coverage. (A, B) Schematical model of the spiked cone design that utilizes a second wedge block (t2) designed to assemble onto the cone's base. (C) Exemplary negative stain TEM micrographs of spiked cone assemblies in different distinct views. (D) Exemplary TEM micrographs showing Influenza A/PR/8/34 virus particles engulfed in spiked cone assemblies functionalized with 6×CR9114 antibodies per wedge subunit. (E) Slices of a negative stain 3D TEM tomogram of a single Influenza virus particle fully engulfed by a single spiked cone, achieving a better surface coverage than non-spiked cones. All scale bars: 50 nm.

FIG. 6 shows the schematic representations of design parameters for t1 and t2. (A) Cross-section of 3×6 DNA helices in a square lattice array, in both straight and tilted configurations. (B) Representation of corner angles (a and B) and lengths of the reference helices (aₓ and bₓ). (C) Representation of single-stranded DNA loops bridging a corner design. (D) Representation of a beveled angle corner design.

FIG. 7 shows the Cryo-EM determination of t1 version 1. (A) Exemplary micrograph. Scale bar: 100 nm. (B) Representative 2D class averages. (C) 3D histograms representing the orientational distribution of particles. (D) FSC plot. (E) Six different views of the electron density map. Scale bar: 25 nm.

FIG. 8 shows the Cryo-EM determination of t1 version 2. (A) Exemplary micrograph. Scale bar: 100 nm. (B) Representative 2D class averages. (C) 3D histograms representing the orientational distribution of particles. (D) FSC plot. (E) Six different views of the electron density map. Scale bar: 25 nm.

FIG. 9 shows the Cryo-EM determination of t1 version 3. (A) Exemplary micrograph. Scale bar: 100 nm. (B) Representative 2D class averages. (C) 3D histograms representing the orientational distribution of particles. (D) FSC plot. (E) Six different views of the electron density map. Scale bar: 25 nm.

FIG. 10 shows Cryo-EM electron density maps of t1 and t2 triangles. Cryo-EM was used to validate the DNA origami designs in an iterative process. It allowed to correct the twist of first versions into nearly twist-free objects (last versions).

FIG. 11 shows negative stain TEM of t1's folding reaction crude. This micrograph shows how t1 triangles start to assemble into cones during the folding reaction. Extra staples from the folding can be seen in the background. Scale bar: 100 nm.

FIG. 12 shows negative stain TEM of unspecific stacking of cones induced by high ionic strength. Lateral and top views of unspecific cone stacking. Scale bar: 100 nm.

FIG. 13 shows 2D class averages of cones extracted from negative stain TEM. Vertex-adhered cones have larger diameters and frayed circumference compared to base-adhered cones containing the same number of wedge building blocks. Scale bar: 100 nm.

FIG. 14 shows Cryo-EM of cones. (A) Different views of the electron density map of the C9 cone. Scale bar: 50 nm. (B) Different views of the electron density map of the C10 cone. Scale bar: 50 nm. (C) 3D histograms representing the orientational distribution of C9 cones. (D) Like in C but for C10 cones.

FIG. 15 shows 3D measurement of dimensions of cryo-EM reconstructions. (A) C9 cone. (B) C10 cone.

FIG. 16 shows a Multibody Analysis of the C9 object. (A) Nine masks (colored, semi-transparent) enclosing the reconstruction of the C9 object used for Multibody Refinement. (B) Principal Component Analysis of refined orientations of individual rigid bodies from a 9-body Multibody Refinement. (C) Distribution of particle weights along the 1st principal component (PC). (D) Reconstructions of two subsets of the particle ensemble. Subset 1 (orange) contains particles with weight value-999 to 0 along PC1, subset 2 (blue) contains particles with values 0 to 999.

FIG. 17 shows negative stain TEM of a negative control for Influenza A/PR/8/34 trapping with cones. Field of view demonstrating no binding of Influenza virus particles without the antibody coating. Scale bar: 100 nm.

FIG. 18 shows the Cryo-EM determination of t2 version 1. (A) Exemplary micrograph. Scale bar: 100 nm. (B) Representative 2D class averages. (C) Histogram representing the orientational distribution of particles. (D) FSC plot. (E) Six different views of the electron density map. Scale bar: 25 nm.

FIG. 19 shows the Cryo-EM determination of t2 version 2. (A) Exemplary micrograph. Scale bar: 100 nm. (B) Representative 2D class averages. (C) 3D histograms representing the orientational distribution of particles. (D) FSC plot. (E) Six different views of the electron density map. Scale bar: 25 nm.

FIG. 20 shows the Cryo-EM determination of t2 version 3. (A) Exemplary micrograph. Scale bar: 100 nm. (B) Representative 2D class averages. (C) 3D histograms representing the orientational distribution of particles. (D) FSC plot. (E) Six different views of the electron density map. Scale bar: 25 nm.

FIG. 21 shows a cylindrical representation of triangles 1 and 2 assembly features. (A) t1's side 3 can be functionalized with a protrusion orthogonal to sides 1 and 2 for the assembly of t2, which has a complementary feature in the form of a recess. (B) Dimer representation in two different views.

FIG. 22 shows t1-t2 dimer assembly characterization. (A) Exemplary laser-scanned fluorescent image of a 1.5% agarose gel showing the assembly of t1 with t2 in a 1:1 ratio over the course of 2 days, with a triangle monomer concentration of 5 nM incubated at 40° C. in presence of 25 mM MgCl₂. Sc: M13-8064 scaffold as reference. Sides 1 and 2 of t1 were passivated to avoid the cone assembly. (B) % of completely assembled dimers at different time points and different MgCl₂ concentrations. The % were extracted from agarose gels like the one shown in (A). Error bars show standard deviations of triplicates.

FIG. 23 shows broadband virus trapping with heparan sulfate-modified spiked cones. (A) Schematics of how cones may be functionalized with virus binding moieties. Red: single stranded DNA extensions called ‘handles. Orange: HS polymers. Trapping was performed with spiked cones featuring 12 heparan sulfate moieties per wedge subunit. (B) Exemplary negative stain TEM micrographs showing trapped SARS-COV-2 and Zika virus-like particles (VLPs). (C) Negative stain TEM micrograph showing trapped Chikungunya VLPs. Due to the smaller size of the CHIK-VLPs, up to three virus particles fit into the large cavity of the spiked cone, which significantly deformed themselves to maximize their contact with the viruses. All scale bars: 50 nm.

FIG. 24 shows the caDNAno design diagram for triangle 1 (A) version 1, (B) version 2, and (C) version 3. Blue: scaffold strand, colorful: staple strands. Designs prepared with caDNAno v0.2.4.

FIG. 25 shows the caDNAno design diagram for triangle 2 (A) version 1, (B) version 2, and (C) version 3. Blue: scaffold strand, colorful: staple strands. Designs prepared with caDNAno v0.2.4.

FIG. 26 shows the schematic representation of the three-dimensional polynucleotide-based open shells of the present invention including the reference numbers used in the claims.

### DETAILED DESCRIPTION OF THE INVENTION

The present disclosure provides constructs that enable the encapsulation of a virus, a viral particle or a subviral particle.

Unless defined otherwise, all technical and scientific terms used herein have the same meaning as commonly understood by those of ordinary skill in the art to which this invention pertains.

The terms “comprising” and “including” are used herein in their open-ended and non-limiting sense unless otherwise noted. With respect to such latter embodiments, the term “comprising” thus includes the narrower term “consisting of”.

The terms “a” and “an” and “the” and similar references in the context of describing the invention (especially in the context of the following claims) are to be construed to cover both the singular and the plural, unless otherwise indicated herein or clearly contradicted by context. For example, the term “a cell” includes a plurality of cells, including mixtures thereof. Where the plural form is used for compounds, salts, and the like, this is taken to mean also a single compound, salt, or the like.

Therefore, in one aspect, the disclosure provides a three-dimensional polynucleotide-based open shell [1] (the reference numbers refer to FIG. 26) encasing a cavity [2] and comprising an n-gonal pyramid [4] formed by n identical copies of a first type of an acute isosceles triangular prismoid t1 [5], wherein n is an integer selected from 7, 8, 9, 10, 11, 12, 13, 14 and 15, wherein the base plane [6] of each prismoid points to the outside of said open shell, and the upper plane [7] points to said cavity, wherein the two large side planes [8,9] of each prismoid contain a first pattern [10] and a second pattern [11] of one or more protrusions and/or one or more receptacles, wherein said first and said second patterns are complementary to each other, and wherein the small side plane [12] comprises a third pattern [13] of one or more protrusions and/or one or more receptacles; wherein said first type of an acute isosceles triangular prismoid is a self-assembling DNA-based building block

In a particular embodiment, the self-assembling DNA-based building block comprises between 7,500 and 10,500 base pairs.

In a particular embodiment, the molecular weight of each self-assembling DNA-based building block is between 4.5 and 7 MDa.

In a particular embodiment, the disclosure provides a three-dimensional polynucleotide-based open shell, which is DNA-based.

In the context of the present disclosure, the term “polynucleotide-based open shell, which is DNA-based” refers to a DNA-based nanostructure that is formed by a set of DNA-based macromolecules. DNA-based nanostructures similar to the ones used in accordance with the present invention are described in detail in of WO 2021/165528 and in Sigl et al., loc. cit.

In the context of the present disclosure, the term “DNA” refers to deoxyribonucleic acid composed of a single-strand of monomeric units called nucleotides, wherein each nucleotide is composed of a nitrogen-containing nucleobase, a 2-deoxyribose sugar moiety, and a phosphate group, wherein the individual nucleotides are linked in the single-strand by a phosphate group linking the OH group in position 5′ of a 2-deoxyribose sugar moiety to the OH group in 3′ of a neighboring 2-deoxyribose sugar moiety. In particular embodiments, the nitrogen-containing nucleobases are independently selected from cytosine [C], guanine [G], adenine [A] and thymine [T]. In particular embodiments, one or more of the nucleobases are non-canonical bases, in particular a non-canonical base selected from the list of: a modified adenosine, in particular N6-carbamoyl-methyladenine or N6-methyadenine; a modified guanine, in particular 7-deazaguanine or 7-methylguanine; a modified cytosine, N4-methylcytosine, 5-carboxylcytosine, 5-formylcytosine, 5-glycosylhydroxymethylcytosine, 5-hydroxycytosine, or 5-methylcytosine; a modified thymidine, in particular α-glutamyl thymidine or α-putrescinyl thymine; a uracil or a modification thereof, in particular uracil, base J, 5-dihydroxypentauracil; or 5-hydroxymethyldeoxyuracil; deoxyarchaeosine and 2,6-diaminopurine. A stretch of a single-strand of DNA may interact with a complementary stretch of DNA by interaction of complementary nucleobases, wherein cytosine and guanine, and adenine and thymine, are complementary to each other, respectively by forming two (A/T) and three (G/C) hydrogen bonds between the nucleobases. Two single-strands of DNA may be fully complementary to each other, as in the case of genomic DNA, or may be partially complementary to each other, including situations, where one single-strand of DNA is partially complementary to two or more other single-stranded DNA strands. The interaction of two complementary single-stranded DNA sequences results in the formation of a double-stranded DNA double helix.

As is well known, DNA has evolved in nature as carrier of the genetic information encoding proteins. DNA further includes non-coding regions that include regions having regulatory functions. Thus, any DNA-based application usually critically depends on the specific DNA sequence and is almost always only enabled by naming the specific DNA sequence. In contrast, in the context of the present invention, such coding and/or regulatory functions do not play any role and may or may not be present, since the underlying DNA sequences are solely designed and selected in a way that the desired arrangement of double-helical subunits is formed. Thus, in one embodiment any form of a long single-stranded DNA sequence, whether naturally occurring DNA (such as the DNA of a bacteriophage) or synthetically produced DNA may be selected as template, and a set of short single-stranded DNA sequences may be designed, wherein each sequence is complementary to one or more different parts of the template and thus forms one or more double-helical sections. Collectively, all such double-helical sections created by interaction of the full set of short single-stranded DNA sequences with the template, then form the desired three-dimensional arrangement. Starting from a given single-stranded template sequence, the design of a set of complementary can be set up using known techniques, such as, for example, the methods described for the synthesis of megadalton-scale discrete objects with structurally well-defined 3D shapes^{15, 40, 49-60}. In particular, iterative design with caDNAno³⁸ paired with elastic-network-guided molecular dynamics simulations⁶¹ can be used.

In addition to the interaction of complementary nucleobases of different stretches of single-stranded DNA via hydrogen bonds, additional interactions between different DNA strands are possible, including the interactions between the ends of two double-stranded DNA helices by protrusion and recess features using either blunt ends or sticky ends for increased stability and specificity⁶², thus enabling the design and the formation of complex DNA-based nanostructures via the shape-complementarity of double-helical subunits. Thus, two three-dimensional arrangements formed in accordance with the previous paragraph, may interact with each other by interactions between double-helical subunits present on the two three-dimensional arrangements, including specific interactions between two three-dimensional arrangements having complementary protrusions and recessions (or knobs and holes).

In a particular embodiment, the DNA-based nanostructure is formed by self-assembling DNA-based building blocks.

In a particular embodiment, each of said self-assembling DNA-based building blocks is formed by a single-stranded DNA template strand and a set of oligonucleotides complementary to said single-stranded DNA template, wherein each of said oligonucleotides is either complementary to one contiguous DNA sequence stretch or to at least two non-contiguous DNA sequence stretches on said single-stranded DNA template.

In a particular embodiment, the DNA-based nanostructure consists of between 4 and 180 of such self-assembling DNA-based building blocks.

In particular embodiments, said single-stranded DNA template is single-stranded DNA of filamentous bacteriophage, or is derived from single-stranded DNA of filamentous bacteriophage.

In the context of the present invention, the term “filamentous bacteriophage” refers to a type of bacteriophage, or virus of bacteria, which is characterized by its filament-like shape that usually contains a genome of circular single-stranded DNA and infects Gram-negative bacteria. Filamentous phage includes Ff phage, such as M13, f1 and fd1 phage, and Pf1 phage.

In particular embodiments, said single-stranded DNA template has a sequence according to SEQ ID NO: 1 (M13 8064) (see Table 1). In particular other embodiments, said single-stranded DNA template has the sequence M13 7249 (see SEQ ID NO: 2 of WO 2021/165528).

In particular embodiments, said single-stranded DNA is circular.

In the context of the present invention, a single-stranded DNA template that is “derived from single-stranded DNA of filamentous bacteriophage” refers to a DNA construct that is derived from a naturally occurring of published DNA sequence of a filamentous bacteriophage by one or more of: (i) opening of the circular structure to a linear sequence; (ii) deletion of one or more nucleotides; (iii) insertion of one or more nucleotides; (iii) substitution of one or more nucleotides; (iv) addition of one or more nucleotides; and (v) modification of one or more nucleotides. While any such variation might have detrimental, or at least rather unpredictable, effects on bacteriophage biology, its infectivity and its ability to propagate, such effects do not play any role in the context of the present invention, since, as already mentioned above, said single-stranded DNA template is only used as naked template without any requirement for having any functional property, and all structural aspects, such as the correct formation the three-dimensional shape of said self-assembling DNA-based building blocks, are implemented by the proper choice of said set of complementary oligonucleotides.

In particular embodiments, said single-stranded DNA template has at least 80%, particularly at least 90%, more particularly at least 95%, sequence identity to the sequence of a naturally occurring or published sequence of a filamentous bacteriophage, in particular to a M13, f1 or fd1 phage, in particular to a sequence selected from SEQ ID NO: 1 (M13 8064) and M13 7249 (see SEQ ID NO: 2 of WO 2021/165528). In this context, it should be mentioned that the single-stranded DNA template is used in the present invention as template only, so that the exact sequence does not have any biological role and/or function. Instead, any sequence of similar length could be used, since the setup of the three-dimensional structure of the polynucleotide-based open shell is essentially achieved by synthesizing a set of oligonucleotides having complementarity with two or more sequence stretches on said single-stranded DNA template. That set of complementary oligonucleotides can be designed manually, but is easier by using computer programs such as caDNAno³⁷ Thus, bacteriophage sequences listed above are given as examples only.

In the context of the present invention, the term “acute isosceles triangular prismoid” refers to a polyhedron, wherein all vertices lie in two parallel planes, which is a triangular prismoid having two planes in the form of acute isosceles triangles.

In a particular embodiment, the present invention relates to a DNA-based nanostructure, wherein each said triangular prismoid, is formed by m triangular planes, wherein m is an integer independently selected from 4, 5, 6, 7 and 8, in particular independently selected from 5, 6 and 7, more particularly wherein said integer is 6, wherein the three, or four, respectively, edges of each of said m planes are formed by n parallel stretches of DNA double helices, wherein n is an integer independently selected from 1, 2, 3, 4, 5 and 6 in particular independently selected from 2, 3, 4 and 5, more particularly independently selected from 3 and 4,
wherein each plane is connected to a plane above and/or a plane beyond said plane (i) by stacking interactions between the DNA double helices forming said planes, and (ii) partially by DNA stretches within said single-stranded DNA template and/or said oligonucleotides forming said DNA-based building block bridging at least two of said planes, and
wherein at least two of the three, or four, respectively, side trapezoids comprise a specific pattern of recesses and/or extrusions formed by missing or additional DNA double helical stretches for specific interaction with a complementary pattern on the side trapezoid of another one of said self-assembling DNA-based building blocks.

In a particular embodiment, the average length of each of the n stretches of DNA double helices in the m planes of a triangular, or rectangular, respectively, prismoid is between 80 and 200 base pairs.

In particular embodiments, said triangular prismoid is a triangular frustum.

In the context of the present invention, the term “triangular frustum” refers to a three-dimensional geometric shape in the form of a triangular pyramid, where the tip of the pyramid has been removed resulting in a plane on the top parallel to the basis of the pyramid.

In a particular embodiment, for at least part of said self-assembling DNA-based building blocks the length of at least one edge of each of said m planes is decreasing from the first to the m^{th} plane, so that a bevel angle θ results between planes perpendicular to said first plane and the trapezoid plane formed by said m edges (see FIG. 6). In particular embodiments, all three trapezoid planes exhibit a bevel angle.

In a particular embodiment, a bevel angle is between 16° and 26°, particularly between 18° and 24°, more particularly between 20° and 22°, most particularly about 20.9°

In a particular embodiment, said DNA-based nanostructure comprises at least one set of self-assembling DNA-based building blocks, wherein all three, or four, respectively, side trapezoids comprise a specific pattern of recesses and/or extrusions formed by missing or additional DNA double helical stretches for specific interaction with a complementary pattern on the side trapezoid of another one of said self-assembling DNA-based building blocks.

In a particular embodiment, said three-dimensional polynucleotide-based open shell further comprises n copies of a second type of an acute isosceles triangular prismoid t2 [14], wherein a first side [15] of each prismoid points to the outside of said open shell, and the opposite side [16] points to said cavity and/or to said opening for accessing said cavity, wherein one plane [17] of said second type of prismoid structure [14] comprises a fourth pattern [18] of one or more protrusions and/or one or more receptacles which is complementary to said third pattern [13].

In a particular embodiment, said DNA-based nanostructure comprises two sets of self-assembling DNA-based building blocks, in particular the self-assembling DNA-based building blocks t1 and t2.

In an alternative aspect of the present invention, the invention relates to a macromolecule-based nanostructure, which is an RNA-based nanostructure.

In the context of the present disclosure, the term “RNA” refers to ribonucleic acid composed of a single-strand of monomeric units called nucleotides, wherein each nucleotide is composed of a nitrogen-containing nucleobase, a ribose sugar moiety, and a phosphate group, wherein the individual nucleotides are linked in the single-strand by a phosphate group linking the OH group in position 5′ of a ribose sugar moiety to the OH group in 3′ of a neighboring ribose sugar moiety. In particular embodiments, the nitrogen-containing nucleobases are independently selected from cytosine [C], guanine [G], adenine [A] and uracil [U]. In particular embodiments, one or more of the nucleobases are non-canonical bases, in particular a non-canonical base selected from the list of: pseudouridine, ribothymidine, and inosine. Unlike DNA, RNA is most often in a single-stranded form, but the formation of double-stranded forms is possible by interaction of complementary nucleobases, wherein cytosine and guanine, and adenine and uracil, are complementary to each other, respectively by forming two (A/U) and three (G/C) hydrogen bonds between the nucleobases. In a particular embodiment, the disclosure provides a macromolecule-based nanostructure, which is an RNA-based nanostructure.

In the context of the present invention, the term “cavity” relates to the space enclosed by said DNA-based nanostructure. In particular embodiments, said cavity resembles a sphere, where a spherical segment has been cut off, with the cutting plane being formed by the self-assembling DNA-based building blocks at the borders of said DNA-based nanostructure. In particular embodiments, the cutting plane is a great circle so that the DNA-based nanostructure is a half-shell.

In a particular embodiment, said upper plane [7] and/or, when present, said opposite side [16] comprise one or more attachment sites for the attachment of one or more binding molecules, which are specifically or non-specifically interacting with a virus, a viral particle or a subviral particle.

In particular embodiments, said one or more binding molecules are specifically interacting with said virus, said viral particle or said subviral particle by being able to bind and to inactivate, said viral particle or said subviral particle.

In a particular embodiment, said binding molecules are specifically interacting with a virus, a viral particle or a subviral particle. In particular, said binding molecules are selected from antibodies and antigen-binding fragments thereof comprising at least an antigen-binding site of an antibody, in particular at least a VH domain of an antibody, or at least a combination of a VH and a VL domain of an antibody particularly scFv fragments.

In a particular other embodiment, said binding molecules are non-specifically interacting with a virus, a viral particle or a subviral particle, in particular constructs comprising at least one sulfonated or sulfated polysaccharide group, particularly a construct comprising one or two sulfonated or sulfated polysaccharide groups, more particularly wherein said sulfonated or sulfated polysaccharide is independently selected from the list of heparin, heparan sulfate, hybrid heparan sulfates, carrageenans, cellulose sulfate, dextrin 2-sulfate, aptamers, peptides, host-receptor domains, sialic acid.

In the context of the present application, the term “viral particle” relates to a virus-like particle that resembles the three-dimensional structure of an intact virus without being biologically active, and the term “subviral particle” relates to a smaller virus-like particle smaller particles with less or smaller subunits, which can be produced for some viruses by expressing not all and/or only portions of one or more major viral capsid proteins. These artificial viral particles or subviral particles retain the structures and antigenic properties of their native viruses, including the virus-specific molecular patterns and high density of B-cell and T-cell epitopes to induce potent innate, humoral, and cellular immune responses, respectively, in animals and humans⁶⁸.

Importantly, in addition to targeting specific receptors, many viruses also weakly interact with different biological substances, including sulfated of sulfonated polysaccharides (63; see Table 4).

In the context of the present application, the term “sulfonated or sulfated polysaccharide group” relates to a group comprising a polysaccharide comprising at least one sulfated hydroxy group or at least one sulfonated glycosylamino group.

Importantly, in addition to targeting specific receptors, many viruses also weakly interact with different biological substances, including sulfated of sulfonated polysaccharides (⁶³; see Table 4).

In particular embodiments, said polysaccharide comprising at least one sulfated hydroxy group or at least one sulfonated glycosylamino group is independently selected from the list of heparin, heparan sulfate, hybrid heparan sulfates, carrageenans, cellulose sulfate, and dextrin 2-sulfate.

In particular embodiments, said polysaccharide comprising at least one sulfated hydroxy group or at least one sulfonated glycosylamino group consists of between 3 and 15 disaccharide units, in particular 4, 5, 6, 7, 8 of 9 units, particularly 4 or 9 monosaccharide units.

In particular embodiments, said disaccharide units comprise two or three O- and/or N-sulfonate groups per disaccharide unit, in particular three O- and/or N-sulfonate groups.

In particular embodiments, said polysaccharide comprising at least one sulfated hydroxy group or at least one sulfonated glycosylamino group is independently selected from heparin, heparan sulfate, and hybrid heparan sulfates.

In the context of the present invention, the terms “heparin” and “heparan sulfate” both relate to a family of linear sulfated, heterogeneous polysaccharides found on the cell membrane and in the extracellular matrix as part of heparan sulfate proteoglycans (HSPGs). They are composed of repeating 1→4 linked disaccharide units, in which one monosaccharide is an α-D-glucosamine residue and the other an uronic acid (or, in a salt form, an uronate). Heparin is a structurally similar polysaccharide found within mast cells as a component of serglycin proteoglycans. Heparan sulfate and heparin can be defined as follows: first, in heparin, the uronates are predominantly α-L-iduronate, whereas in heparan sulfate, the uronates are mainly, β-D-glucuronates, the C-5 epimers of α-L-iduronate. Second, in heparan sulfate, the D-glucosamine residues are predominantly N-acetylated, whereas in heparin, they are N-sulfonated. Finally, whereas at least 70-80% of heparin is composed of the disaccharide L-iduronate 2-O-sulfate α(1→4) D-glucosamine N,6-sulfate, in heparan sulfate around 40-60% of the disaccharides consist of (1→4) D-glucuronate β (1→4) D-glucosamine, that can be either N-acetylated or N-sulfonated. Together, these structural characteristics make heparin more sulfated and, hence, more charged than heparan sulfate. It has become apparent, however, that the designations heparin or heparan sulfate are less clear-cut than this description implies, and that polysaccharides isolated from some organisms appear to be hybrid constructs. In the context of the present invention, the term “hybrid heparan sulfate” is used to refer to such hybrids having structures being a mixture of the “typical” heparin structural elements (L-iduronates; high degree of sulfonation) and the “typical” heparan sulfate structural elements (D-glucuronate; N-acetylation and 6-O-sulfonation).

Heparan sulfate proteoglycans (HSPG)^{63; 64} are commonly found on the surface of mammalian cells. The weak interactions of viruses with HSPG are conserved across virus families and thus appear generically beneficial for the virus lifecycle. For example, HSPG-virus interactions may enable an infection-enhancing diffusive search of virus particles for their specific host cell receptors on the surface of cells. The interactions of heparan sulfate (HS) with viruses have already been exploited for medical purposes, for example in virus-sequestering coatings of condoms that are based on HS-decorated dendrimers³⁻⁵. Other investigations have frequently involved the surface functionalization of nanoparticles and polymers with HS derivatives to create virus-binding complexes with antiviral activity^{6; 7; 65; 66}. Commonly, a high level of multivalency is required to increase the strength of binding between the HS-nanoparticles and viruses. The reversible nature of the binding can lead to undesirable unbinding and release of infectious viruses from the virus-sequestering coatings, or the requirement for high concentrations of the therapeutically active agent to be maintained⁵.

In particular embodiments, said macromolecule-based nanostructure comprises, on average, between one and 10 binding molecules attached to the interior site of the cavity formed by said macromolecule-based nanostructure, in particular between 4 and 10, in particular four, five, six, seven, eight, nine or ten binding molecules.

In particular embodiments, one or more of said self-assembling DNA-based building blocks is linked to a construct comprising at least one sulfonated or sulfated polysaccharide group pointing to the interior of said cavity, particularly a construct comprising one or two sulfonated or sulfated polysaccharide groups.

In particular embodiments, said three-dimensional polynucleotide-based open shell is a DNA-based nanostructure in accordance with the present invention, wherein said at least one binding molecule is linked to one of said triangular prismoids forming the DNA-based nanostructure in a way that said at least one binding molecule is located on the inside of said DNA-based nanostructure and is pointing into the cavity formed by said DNA-based nanostructure.

In a particular embodiment, each prismoid comprises between 1 and 45, in particular between 1 and 32 of said attachment sites, particular between 3 and 10 attachment sites. In particular embodiments, all prismoids comprise said attachments sites. In other embodiments, only the t1 prismoids comprise said attachments sites, or only the t2 prismoids comprise said attachments sites.

In a particular embodiment, said attachment sites are first single-stranded oligonucleotides.

In a particular embodiment, said binding molecules are attached to said attachment sites by second single-stranded oligonucleotides, which are linked to one or more binding molecules and are complementary to, or otherwise able to enter site-specific interactions with, said first single-stranded oligonucleotides. In particular embodiments, each of said single-stranded oligonucleotides is linked to one binding molecule. In other embodiments, each of said single-stranded oligonucleotides is linked to two binding molecules.

In a particular embodiment, each of said first and of said optional second types of said acute isosceles triangular prismoids is a DNA-based nanostructure formed by self-assembling DNA-based building blocks, in particular wherein said DNA-based nanostructure is formed by a single-stranded DNA template strand and a set of oligonucleotides complementary to said single-stranded DNA template, wherein each of said oligonucleotides is either complementary to one contiguous DNA sequence stretch or to at least two non-contiguous DNA sequence stretches on said single-stranded DNA template.

In particular embodiments, the apex angle of the acute isosceles triangles forming the opposing planes of said acute isosceles triangular prismoids is between 15° and 60°, in particular between 20° to 30°.

In a particular embodiment, n is an integer selected from 9, 10, 11, 12 and 13.

In a particular embodiment, said three-dimensional polynucleotide-based open shell further comprises chemical crosslinks between different prismoids further comprises one or more cross-linkages within one of said triangular prismoids, and/or between two of said triangular prismoids.

In the context of the present invention, the term “cross-linkage” refers to any permanent or intermittent linkage within one of said triangular prismoids, and/or between two of said triangular prismoids. Any such linkage may be achieved a priori by linking two of the oligonucleotides being used for forming the self-assembling DNA-based building blocks prior to the assembly, or a posteriori, e. g. by chemically or photochemically adding linkages between different parts of the three-dimensional nanostructure. Permanent linkages may, for example, be created by photochemically cross-linking T residues appropriately positioned in the structure under formation of covalent cyclobutane pyrimidine dimer (CPD) bonds¹⁹, and intermittent linkages may, for example, be created by photochemically cross-linking the blunt ends of two double-helical subunits between a 3-cyanovinylcarbazole (cnvK) moiety positioned at a first blunt end and a thymine residue (T) positioned at the other blunt end⁶⁷.

In a particular embodiment, said three-dimensional polynucleotide-based open shell further comprises chemical crosslinks between different triangular prismoids. In a particular embodiment, said chemical crosslinks are obtained by UV irradiation.

In a particular embodiment, said three-dimensional polynucleotide-based open shell further comprises a coating of the outer surface of said open shell with a polycationic molecule.

In a particular embodiment, said polycationic molecule is a polylysine, particularly polylysine-PEG.

In a particular embodiment, said three-dimensional polynucleotide-based open shell further comprises cross-links of free amino groups of said polylysine, particularly with an alkane dialdehyde, in particular with glutaraldehyde.

In a particular embodiment, said opening [3] has a diameter [19] between 100 and 200 nm.

In the context of the present invention, the term “diameter” refers to the diameter [19] as shown in FIG. 26.

In particular embodiments, three-dimensional polynucleotide-based open shell has a molecular weight between 30 MDa and 80 MDa (t1 only), particularly between 40 MDa and 70 MDa, and between 60 MDa and 160 MDa (t1 plus t2), particularly between 80 MDa and 140 MDa.

In particular embodiments, the volume of the cavity encased by said three-dimensional polynucleotide-based open shell (in nm³) is between 80,000 and 200,000, particularly between 100,000 and 140,000.

In another aspect, the present invention relates to a composition comprising a mixture of a three-dimensional polynucleotide-based open shells according to the present invention, wherein said mixture comprises three-dimensional polynucleotide-based open shells having values of n ranging from 7 to 15.

particularly ranging from 9 to 13, with a maximum in the range of 9 to 11.

In another aspect, the present invention relates to the composition according to the present invention for use in the treatment of a patient infected by, suspected to be infected by, or bearing the risk of becoming infected by, a virus, a viral particle or a subviral particle.

In another aspect, the present invention relates to a method for encapsulating a virus, a viral particle or a subviral particle, comprising the steps of: providing a three-dimensional polynucleotide-based open shell according to the present invention, and contacting said macromolecule-based nanostructure with a medium comprising, or suspected to comprise, said virus, said viral particle or said subviral particle.

In particular embodiments, said method is for removing said virus, said viral particle or said subviral particle from said medium. In particular embodiment, said method is for encapsulating said virus, said viral particle or said subviral particle in order to transport said virus, said viral particle or said subviral particle.

In particular embodiments, said method for removing said virus, said viral particle or said subviral particle relates to a method for the treatment of a patient infected by, suspected to be infected by, or bearing the risk of becoming infected by, said virus, said viral particle or said subviral particle, comprising the step of: administering the three-dimensional polynucleotide-based open shell according to the present invention, or the composition according to the present invention to said patient.

In particular embodiments, said method for the treatment of a patient infected by, or suspected to be infected by, a virus, a viral particle or a subviral particle, comprises the step of: contacting said patient, or a bodily fluid of said patient, with the three-dimensional polynucleotide-based open shell according to the present invention, or the composition according to the present invention.

In another aspect, the disclosure provides a composition comprising a virus, a viral particle or a subviral particle encapsulated by a three-dimensional polynucleotide-based open shell according to the present invention or by a three-dimensional polynucleotide-based open shell from the composition according to the present invention.

In particular embodiments, said composition is formed in a process of removing said virus, said viral particle or said subviral particle from a medium containing said virus, said viral particle or said subviral particle. In particular other embodiments, said composition is formed in a process of incorporating said virus, said viral particle or said subviral particle as cargo in said three-dimensional polynucleotide-based open shell.

In another aspect, the disclosure provides a composition comprising a cargo different from a virus, a viral particle or a subviral particle, where said cargo, such as a complex macromolecule, is encapsulated by a three-dimensional polynucleotide-based open shell according to the present invention. In particular embodiments, said cargo is a cytokine. In particular embodiments, said cytokine is interleukin-6.

In yet another aspect, the disclosure provides a method for encapsulating a cargo different from a virus, a viral particle or a subviral particle, such as a complex macromolecule, comprising the steps of: providing a three-dimensional polynucleotide-based open shell according to the present invention, and contacting said three-dimensional polynucleotide-based open shell with a medium comprising, or suspected to comprise, said cargo. In particular embodiments, said cargo is a cytokine. In particular embodiments, said cytokine is interleukin-6.

### TABLES 1 TO 3: SEQUENCE LISTING

TABLE 1
Template Sequence

| SEQ ID NO: | Description | Sequence / Details |
|---|---|---|
| 1 | M13 8064 | GGCAATGACCTGATAGCCTTTGTAGATCTCTCAAAAATAGCTACCCTCTCCGGCATTAATTT |
| | | ATCAGCTAGAACGGTTGAATATCATATTGATGGTGATTTGACTGTCTCCGGCCTTTCTCACC |
| | | CTTTTGAATCTTTACCTACACATTACTCAGGCATTGCATTTAAAATATATGAGGGTTCTAAAA |
| | | ATTTTTATCCTTGCGTTGAAATAAAGGCTTCTCCCGCAAAAGTATTACAGGGTCATAATGTTT |
| | | TTGGTACAACCGATTTAGCTTTATGCTCTGAGGCTTTATTGCTTAATTTTGCTAATTCTTTGC |
| | | CTTGCCTGTATGATTTATTGGATGTTAATGCTACTACTATTAGTAGAATTGATGCCACCTTTT |
| | | CAGCTCGCGCCCCAAATGAAAATATAGCTAAACAGGTTATTGACCATTTGCGAAATGTATCT |
| | | AATGGTCAAACTAAATCTACTCGTTCGCAGAATTGGGAATCAACTGTTATATGGAATGAAAC |
| | | TTCCAGACACCGTACTTTAGTTGCATATTTAAAACATGTTGAGCTACAGCATTATATTCAGCA |
| | | ATTAAGCTCTAAGCCATCCGCAAAAATGACCTCTTATCAAAAGGAGCAATTAAAGGTACTCT |
| | | CTAATCCTGACCTGTTGGAGTTTGCTTCCGGTCTGGTTCGCTTTGAAGCTCGAATTAAAACG |
| | | CGATATTTGAAGTCTTTCGGGCTTCCTCTTAATCTTTTTGATGCAATCCGCTTTGCTTCTGAC |
| | | TATAATAGTCAGGGTAAAGACCTGATTTTTGATTTATGGTCATTCTCGTTTTCTGAACTGTTT |
| | | AAAGCATTTGAGGGGGATTCAATGAATATTTATGACGATTCCGCAGTATTGGACGCTATCCA |
| | | GTCTAAACATTTTACTATTACCCCCTCTGGCAAAACTTCTTTTGCAAAAGCCTCTCGCTATTT |
| | | TGGTTTTTATCGTCGTCTGGTAAACGAGGGTTATGATAGTGTTGCTCTTACTATGCCTCGTA |
| | | ATTCCTTTTGGCGTTATGTATCTGCATTAGTTGAATGTGGTATTCCTAAATCTCAACTGATGA |
| | | ATCTTTCTACCTGTAATAATGTTGTTCCGTTAGTTCGTTTTATTAACGTAGATTTTTCTTCCCA |
| | | ACGTCCTGACTGGTATAATGAGCCAGTTCTTAAAATCGCATAAGGTAATTCACAATGATTAA |
| | | AGTTGAAATTAAACCATCTCAAGCCCAATTTACTACTCGTTCTGGTGTTTCTCGTCAGGGCA |
| | | AGCCTTATTCACTGAATGAGCAGCTTTGTTACGTTGATTTGGGTAATGAATATCCGGTTCTT |
| | | GTCAAGATTACTCTTGATGAAGGTCAGCCAGCCTATGCGCCTGGTCTGTACACCGTTCATC |
| | | TGTCCTCTTTCAAAGTTGGTCAGTTCGGTTCCCTTATGATTGACCGTCTGCGCCTCGTTCCG |
| | | GCTAAGTAACATGGAGCAGGTCGCGGATTTCGACACAATTTATCAGGCGATGATACAAATC |
| | | TCCGTTGTACTTTGTTTCGCGCTTGGTATAATCGCTGGGGGTCAAAGATGAGTGTTTTAGTG |
| | | TATTCTTTTGCCTCTTTCGTTTTAGGTTGGTGCCTTCGTAGTGGCATTACGTATTTTACCCGT |
| | | TTAATGGAAACTTCCTCATGAAAAAGTCTTTAGTCCTCAAAGCCTCTGTAGCCGTTGCTACC |
| | | CTCGTTCCGATGCTGTCTTTCGCTGCTGAGGGTGACGATCCCGCAAAAGCGGCCTTTAACT |
| | | CCCTGCAAGCCTCAGCGACCGAATATATCGGTTATGCGTGGGCGATGGTTGTTGTCATTGT |
| | | CGGCGCAACTATCGGTATCAAGCTGTTTAAGAAATTCACCTCGAAAGCAAGCTGATAAACC |
| | | GATACAATTAAAGGCTCCTTTTGGAGCCTTTTTTTTGGAGATTTTCAACGTGAAAAAATTATT |
| | | ATTCGCAATTCCTTTAGTTGTTCCTTTCTATTCTCACTCCGCTGAAACTGTTGAAAGTTGTTT |
| | | AGCAAAATCCCATACAGAAAATTCATTTACTAACGTCTGGAAAGACGACAAAACTTTAGATC |
| | | GTTACGCTAACTATGAGGGCTGTCTGTGGAATGCTACAGGCGTTGTAGTTTGTACTGGTGA |
| | | CGAAACTCAGTGTTACGGTACATGGGTTCCTATTGGGCTTGCTATCCCTGAAAATGAGGGT |
| | | GGTGGCTCTGAGGGTGGCGGTTCTGAGGGTGGCGGTTCTGAGGGTGGCGGTACTAAACC |
| | | TCCTGAGTACGGTGATACACCTATTCCGGGCTATACTTATATCAACCCTCTCGACGGCACTT |
| | | ATCCGCCTGGTACTGAGCAAAACCCCGCTAATCCTAATCCTTCTCTTGAGGAGTCTCAGCC |
| | | TCTTAATACTTTCATGTTTCAGAATAATAGGTTCCGAAATAGGCAGGGGGCATTAACTGTTTA |
| | | TACGGGCACTGTTACTCAAGGCACTGACCCCGTTAAAACTTATTACCAGTACACTCCTGTAT |
| | | CATCAAAAGCCATGTATGACGCTTACTGGAACGGTAAATTCAGAGACTGCGCTTTCCATTCT |
| | | GGCTTTAATGAGGATTTATTTGTTTGTGAATATCAAGGCCAATCGTCTGACCTGCCTCAACC |
| | | TCCTGTCAATGCTGGCGGCGGCTCTGGTGGTGGTTCTGGTGGCGGCTCTGAGGGTGGTGG |
| | | CTCTGAGGGTGGCGGTTCTGAGGGTGGCGGCTCTGAGGGAGGCGGTTCCGGTGGTGGCT |
| | | CTGGTTCCGGTGATTTTGATTATGAAAAGATGGCAAACGCTAATAAGGGGGCTATGACCGA |
| | | AAATGCCGATGAAAACGCGCTACAGTCTGACGCTAAAGGCAAACTTGATTCTGTCGCTACT |
| | | GATTACGGTGCTGCTATCGATGGTTTCATTGGTGACGTTTCCGGCCTTGCTAATGGTAATGG |
| | | TGCTACTGGTGATTTTGCTGGCTCTAATTCCCAAATGGCTCAAGTCGGTGACGGTGATAATT |
| | | CACCTTTAATGAATAATTTCCGTCAATATTTACCTTCCCTCCCTCAATCGGTTGAATGTCGCC |
| | | CTTTTGTCTTTGGCGCTGGTAAACCATATGAATTTTCTATTGATTGTGACAAAATAAACTTATT |
| | | CCGTGGTGTCTTTGCGTTTCTTTTATATGTTGCCACCTTTATGTATGTATTTTCTACGTTTGCT |
| | | AACATACTGCGTAATAAGGAGTCTTAATCATGCCAGTTCTTTTGGGTATTCCGTTATTATTGC |
| | | GTTTCCTCGGTTTCCTTCTGGTAACTTTGTTCGGCTATCTGCTTACTTTTCTTAAAAAGGGCT |
| | | TCGGTAAGATAGCTATTGCTATTTCATTGTTTCTTGCTCTTATTATTGGGCTTAACTCAATTCT |
| | | TGTGGGTTATCTCTCTGATATTAGCGCTCAATTACCCTCTGACTTTGTTCAGGGTGTTCAGTT |
| | | AATTCTCCCGTCTAATGCGCTTCCCTGTTTTTATGTTATTCTCTCTGTAAAGGCTGCTATTTT |
| | | CATTTTTGACGTTAAACAAAAAATCGTTTCTTATTTGGATTGGGATAAATAATATGGCTGTTTA |
| | | TTTTGTAACTGGCAAATTAGGCTCTGGAAAGACGCTCGTTAGCGTTGGTAAGATTCAGGATA |
| | | AAATTGTAGCTGGGTGCAAAATAGCAACTAATCTTGATTTAAGGCTTCAAAACCTCCCGCAA |
| | | GTCGGGAGGTTCGCTAAAACGCCTCGCGTTCTTAGAATACCGGATAAGCCTTCTATATCTG |
| | | ATTTGCTTGCTATTGGGCGCGGTAATGATTCCTACGATGAAAATAAAAACGGCTTGCTTGTT |
| | | CTCGATGAGTGCGGTACTTGGTTTAATACCCGTTCTTGGAATGATAAGGAAAGACAGCCGA |
| | | TTATTGATTGGTTTCTACATGCTCGTAAATTAGGATGGGATATTATTTTTCTTGTTCAGGACTT |
| | | ATCTATTGTTGATAAACAGGCGCGTTCTGCATTAGCTGAACATGTTGTTTATTGTCGTCGTCT |
| | | GGACAGAATTACTTTACCTTTTGTCGGTACTTTATATTCTCTTATTACTGGCTCGAAAATGCC |
| | | TCTGCCTAAATTACATGTTGGCGTTGTTAAATATGGCGATTCTCAATTAAGCCCTACTGTTGA |
| | | GCGTTGGCTTTATACTGGTAAGAATTTGTATAACGCATATGATACTAAACAGGCTTTTTCTAG |
| | | TAATTATGATTCCGGTGTTTATTCTTATTTAACGCCTTATTTATCACACGGTCGGTATTTCAAA |
| | | CCATTAAATTTAGGTCAGAAGATGAAATTAACTAAAATATATTTGAAAAAGTTTTCTCGCGTT |
| | | CTTTGTCTTGCGATTGGATTTGCATCAGCATTTACATATAGTTATATAACCCAACCTAAGCCG |
| | | GAGGTTAAAAAGGTAGTCTCTCAGACCTATGATTTTGATAAATTCACTATTGACTCTTCTCAG |
| | | CGTCTTAATCTAAGCTATCGCTATGTTTTCAAGGATTCTAAGGGAAAATTAATTAATAGCGAC |
| | | GATTTACAGAAGCAAGGTTATTCACTCACATATATTGATTTATGTACTGTTTCCATTAAAAAA |
| | | GGTAATTCAAATGAAATTGTTAAATGTAATTAATTTTGTTTTCTTGATGTTTGTTTCATCATCTT |
| | | CTTTTGCTCAGGTAATTGAAATGAATAATTCGCCTCTGCGCGATTTTGTAACTTGGTATTCAA |
| | | AGCAATCAGGCGAATCCGTTATTGTTTCTCCCGATGTAAAAGGTACTGTTACTGTATATTCAT |
| | | CTGACGTTAAACCTGAAAATCTACGCAATTTCTTTATTTCTGTTTTACGTGCAAATAATTTTGA |
| | | TATGGTAGGTTCTAACCCTTCCATTATTCAGAAGTATAATCCAAACAATCAGGATTATATTGA |
| | | TGAATTGCCATCATCTGATAATCAGGAATATGATGATAATTCCGCTCCTTCTGGTGGTTTCTT |
| | | TGTTCCGCAAAATGATAATGTTACTCAAACTTTTAAAATTAATAACGTTCGGGCAAAGGATTT |
| | | AATACGAGTTGTCGAATTGTTTGTAAAGTCTAATACTTCTAAATCCTCAAATGTATTATCTATT |
| | | GACGGCTCTAATCTATTAGTTGTTAGTGCTCCTAAAGATATTTTAGATAACCTTCCTCAATTC |
| | | CTTTCAACTGTTGATTTGCCAACTGACCAGATATTGATTGAGGGTTTGATATTTGAGGTTCA |
| | | GCAAGGTGATGCTTTAGATTTTTCATTTGCTGCTGGCTCTCAGCGTGGCACTGTTGCAGGC |
| | | GGTGTTAATACTGACCGCCTCACCTCTGTTTTATCTTCTGCTGGTGGTTCGTTCGGTATTTTT |
| | | AATGGCGATGTTTTAGGGCTATCAGTTCGCGCATTAAAGACTAATAGCCATTCAAAAATATT |
| | | GTCTGTGCCACGTATTCTTACGCTTTCAGGTCAGAAGGGTTCTATCTCTGTTGGCCAGAATG |
| | | TCCCTTTTATTACTGGTCGTGTGACTGGTGAATCTGCCAATGTAAATAATCCATTTCAGACG |
| | | ATTGAGCGTCAAAATGTAGGTATTTCCATGAGCGTTTTTCCTGTTGCAATGGCTGGCGGTAA |
| | | TATTGTTCTGGATATTACCAGCAAGGCCGATAGTTTGAGTTCTTCTACTCAGGCAAGTGATG |
| | | TTATTACTAATCAAAGAAGTATTGCTACAACGGTTAATTTGCGTGATGGACAGACTCTTTTAC |
| | | TCGGTGGCCTCACTGATTATAAAAACACTTCTCAGGATTCTGGCGTACCGTTCCTGTCTAAA |
| | | ATCCCTTTAATCGGCCTCCTGTTTAGCTCCCGCTCTGATTCTAACGAGGAAAGCACGTTATA |
| | | CGTGCTCGTCAAAGCAACCATAGTACGCGCCCTGTAGCGGCGCATTAAGCGCGGCGGGTG |
| | | TGGTGGTTACGCGCAGCGTGACCGCTACACTTGCCAGCGCCCTAGCGCCCGCTCCTTTCG |
| | | CTTTCTTCCCTTCCTTTCTCGCCACGTTCGCCGGCTTTCCCCGTCAAGCTCTAAATCGGGG |
| | | GCTCCCTTTAGGGTTCCGATTTAGTGCTTTACGGCACCTCGACCCCAAAAAACTTGATTTGG |
| | | GTGATGGTTCACGTAGTGGGCCATCGCCCTGATAGACGGTTTTTCGCCCTTTGACGTTGGA |
| | | GTCCACGTTCTTTAATAGTGGACTCTTGTTCCAAACTGGAACAACACTCAACCCTATCTCGG |
| | | GCTATTCTTTTGATTTATAAGGGATTTTGCCGATTTCGGAACCACCATCAAACAGGATTTTCG |
| | | CCTGCTGGGGCAAACCAGCGTGGACCGCTTGCTGCAACTCTCTCAGGGCCAGGCGGTGAA |
| | | GGGCAATCAGCTGTTGCCCGTCTCACTGGTGAAAAGAAAAACCACCCTGGCGCCCAATAC |
| | | GCAAACCGCCTCTCCCCGCGCGTTGGCCGATTCATTAATGCAGCTGGCACGACAGGTTTC |
| | | CCGACTGGAAAGCGGGCAGTGAGCGCAACGCAATTAATGTGAGTTAGCTCACTCATTAGGC |
| | | ACCCCAGGCTTTACACTTTATGCTTCCGGCTCGTATGTTGTGTGGAATTGTGAGCGGATAAC |
| | | AATTTCACACAGGAAACAGCTATGACCATGATTACGAATTCGAGCTCGGTACCCGGGGATC |
| | | CTCAACTGTGAGGAGGCTCACGGACGCGAAGAACAGGCACGCGTGCTGGCAGAAACCCC |
| | | CGGTATGACCGTGAAAACGGCCCGCCGCATTCTGGCCGCAGCACCACAGAGTGCACAGGC |
| | | GCGCAGTGACACTGCGCTGGATCGTCTGATGCAGGGGGCACCGGCACCGCTGGCTGCAG |
| | | GTAACCCGGCATCTGATGCCGTTAACGATTTGCTGAACACACCAGTGTAAGGGATGTTTAT |
| | | GACGAGCAAAGAAACCTTTACCCATTACCAGCCGCAGGGCAACAGTGACCCGGCTCATAC |
| | | CGCAACCGCGCCCGGCGGATTGAGTGCGAAAGCGCCTGCAATGACCCCGCTGATGCTGG |
| | | ACACCTCCAGCCGTAAGCTGGTTGCGTGGGATGGCACCACCGACGGTGCTGCCGTTGGCA |
| | | TTCTTGCGGTTGCTGCTGACCAGACCAGCACCACGCTGACGTTCTACAAGTCCGGCACGTT |
| | | CCGTTATGAGGATGTGCTCTGGCCGGAGGCTGCCAGCGACGAGACGAAAAAACGGACCGC |
| | | GTTTGCCGGAACGGCAATCAGCATCGTTTAACTTTACCCTTCATCACTAAAGGCCGCCTGT |
| | | GCGGCTTTTTTTACGGGATTTTTTTATGTCGATGTACACAACCGCCCAACTGCTGGCGGCAA |
| | | ATGAGCAGAAATTTAAGTTTGATCCGCTGTTTCTGCGTCTCTTTTTCCGTGAGAGCTATCCC |
| | | TTCACCACGGAGAAAGTCTATCTCTCACAAATTCCGGGACTGGTAAACATGGCGCTGTACG |
| | | TTTCGCCGATTGTTTCCGGTGAGGTTATCCGTTCCCGTGGCGGCTCCACCTCTGAAAGCTT |
| | | GGCACTGGCCGTCGTTTTACAACGTCGTGACTGGGAAAACCCTGGCGTTACCCAACTTAAT |
| | | CGCCTTGCAGCACATCCCCCTTTCGCCAGCTGGCGTAATAGCGAAGAGGCCCGCACCGAT |
| | | CGCCCTTCCCAACAGTTGCGCAGCCTGAATGGCGAATGGCGCTTTGCCTGGTTTCCGGCA |
| | | CCAGAAGCGGTGCCGGAAAGCTGGCTGGAGTGCGATCTTCCTGAGGCCGATACTGTCGTC |
| | | GTCCCCTCAAACTGGCAGATGCACGGTTACGATGCGCCCATCTACACCAACGTGACCTATC |
| | | CCATTACGGTCAATCCGCCGTTTGTTCCCACGGAGAATCCGACGGGTTGTTACTCGCTCAC |
| | | ATTTAATGTTGATGAAAGCTGGCTACAGGAAGGCCAGACGCGAATTATTTTTGATGGCGTTC |
| | | CTATTGGTTAAAAAATGAGCTGATTTAACAAAAATTTAATGCGAATTTTAACAAAATATTAACG |
| | | TTTACAATTTAAATATTTGCTTATACAATCTTCCTGTTTTTGGGGCTTTTCTGATTATCAACCG |
| | | GGGTACATATGATTGACATGCTAGTTTTACGATTACCGTTCATCGATTCTCTTGTTTGCTCCA |
| | | GACTCTCA |

TABLE 2
Staple sequences for triangle 1
TABLE 2A: Staple sequences for triangle 1, version 1

| SEQ | | |
|---|---|---|
| ID NO: | Description | Sequence/Details |
| 2 | core_1 | TTATACTTTCAGCTCATTTTTTAAATATTTTGAGCGGATTATCAAAAATCAGGTCT |
| 3 | core_2 | GCCAGCATTGACATCAAGTACCAGTAGATAAGTCCTGAACCCAGCACGCAGCGCCA |
| 4 | core_3 | ATACCCAAGGCCTTTATGTACATCATAGAAGGTGCCAGTTGAAGCCTTAAAGGAAC |
| 5 | core_4 | TGCGATTTTAAGAACTCCAATGGAAATAAAGACTTCAAATATCGCGTTGTCATTTT |
| 6 | core_5 | TGCCCGCTTTCCAGTCTCAGAACCATGGTTCAGCTAATGCAGTAATAACATTTTGAC |
| 7 | core_6 | ACCTGCAGCCAGCATCAGCGGGGTCATTGCAGGCGAGAACAAGCAAGCCTTAACGTC |
| 8 | core_7 | GTAAATTGTTATGACCCTGTAATAACCCCGGTATAGCGTCAATCCCCCTCAAATGC |
| 9 | core_8 | GTTACCAGATCCAATAGGAGTAACAAAGCTGCTCATTCAGCCTCACCGAATGGTCA |
| 10 | core_9 | AGATTTAGCGCCAAAAGGAATTACATTCAACCGAATTTTTCGATGAACGGTAATCGT |
| 11 | core_10 | CAGAATCAAGCCGCCAAGCACTAAAGTAATTCTCGTCGCTATTTAACAATTTCATT |
| 12 | core_11 | GGGTAATAGTAAAATGTTAAAGAGAGGCTTTTGCTTCAAAGCGAACCAGAGAGTACCT |
| 13 | core_12 | CCTGATTGTTTAGGAACGCCATCATGCGGATGAGCTCAACATGTTTTAAATATGCAA |
| 14 | core_13 | TACGCAGTATGTTAGCAAACGTAGAAAATAAACATCCATACCGGGGGTTTCTG |
| 15 | core_14 | AGCAGCAACTACAATTCGTACAGCGCCATTGAGATAGCCGCAATAATAACGGA |
| 16 | core_15 | TGCGTATTGGGTTTTTGCCAGGGTGGTTTTTCTATCACCACCAGCAGAAAAAAGTTTG |
| 17 | core_16 | AGAAAGCGAAAGGAGACACCCAGCAGGCGAAAATCCTGTTTTTTTGATGGAGTTGCA |
| 18 | core_17 | TTAGAGCCCTTTGATTCGGGTACCGAGCTTCCGCGCTCACCAGCTGCATTAAT |
| 19 | core_18 | TGAATTACAAAAAGCCCGAGGGTTGATATAAGGTCAGACGATTAGGTGCCGTAACCAGAAC |
| | | C |
| 20 | core_19 | TAAAACGAAAGAGGCAAAAGAAGCTTGATACCGATAGCATGTACCGTAACACT |
| 21 | core_20 | TCAGTATTAAAGGGATGCCACCGACAGACAATATTTTTGATTCAAATAGTGTA |
| 22 | core_21 | GATAGCTCCCAGGCGCTATTATTCTGTTTTTAACTAATAAGTTTTTGCAAGACTTGAGCCATT |
| 23 | core_22 | TTTTCAGGTTTAACGTCTAAAAATCATATTAATTTCATCTTCTGGTAAGAAT |
| 24 | core_23 | TGGAAGGATTCATCCCCTTCAGTGAGACGGGCAACACTGAATATAAGGAGCG |
| 25 | core_24 | CTTTACAAACAATTCGACAACTCGTCAGTGAGTTTAGACATTTTTTGAACGGTAAAATA |
| 26 | core_25 | AAAAGCCGCACAGGCAAGAACTGGACGGAAATTATTCATTAAAGAGCGGCGGTTG |
| 27 | core_26 | CGCGCTTATTTTTTGCGCCGCTACAGGGCTGGCAATATTTTAGGGTCTGAG |
| 28 | core_27 | CCGAACGAAGATGATGGCAGTTAGAACGAAATCGGCCAGTTTGGAAAAGGAAGGG |
| 29 | core_28 | CTTTCATCATTTCAACTTTAATCTTTTTTTGTGAATTACCTTACAGGACGT |
| 30 | core_29 | AAGGGCGAGATAGATGAATATACAGTTTTTTACAGTACCTTGAAATTGCGTAGA |
| 31 | core_30 | TTCAGAGGTTTGGGGCTGATTCCCAATTCTGCGAACGAGTTTTTTTCGCCACGGGAACGGA |
| | | TAA |
| 32 | core_31 | AAAATAATAACTGTTGTACGCCAGCTGGCGAAAGGGGGATGCGGAGATGGATCAGTTGTG |
| | | GGAA |
| 33 | core_32 | GTTTTTATAATATTAAATCCTTTTTTTGCCCGAACGTTATCCTGAGAAGT |
| 34 | core_33 | AGAACGCGAGCCTCCTTCACAATCACACCACGGAATAAGTGGGAAACCGCATCACC |
| 35 | core_34 | CGGTGCCCCCTGCATCAGACAAATCCCACGCAACCAGCTTACGGCTGTAGGAATC |
| 36 | core_35 | GAGTCCACTATTATATTATAGTCGGGTTGAGTTGTTAAACTGAATAA |
| 37 | core_36 | GACGCTGATCCCGGGCGCTAGGGCGCGCGTACACGTGGCA |
| 38 | core_37 | ATAAGAGTTAATTTTTTCGAGCAAATTTTTGAAGTTTTATTATACCAGT |
| 39 | core_38 | ATGATGAGGCTTTTTTAGTACAGGTAGTTTTTAAGATTCGCGCATCGTA |
| 40 | core_39 | GAGATAACCCACAAGACATCCTAATTTACGAGGGCCGTTTGATTGAGGG |
| 41 | core_40 | AGAAACGATTTTTTGTGTTTTTATTTTCATCGGAGGTGTCCAGCGGTGC |
| 42 | core_41 | GAAAACATTATTAAGAGCAACACTATCATACAGTCAAATCACCATCAAT |
| 43 | core_42 | AGAGAAGGTAGAACCAGAGCCACCTGGTTTACCGTGCCTGAATATCTG |
| 44 | core_43 | CGGAACGAGCGACCTGCTCCATGTATGTGTAGGTAAAGATTCATATGT |
| 45 | core_44 | CTCCGGCCATAACAGTACAGGTCAGGATTAGACCGGAAGC |
| 46 | core_45 | GAAGGCACACCATCGCCCACGCATAGCCACCACCCTCATTAATAACAT |
| 47 | core_46 | GGAAGGGCGATCGGTGAATGCTGTGCTTAGAGCTTAATTGATCAAAAG |
| 48 | core_47 | AATAAGTTTCATAAATTACTTAGCTATTTTAAATGCAATGCTTTTGCG |
| 49 | core_48 | CGCCACCCTTATTTTGCACAGTTGTTGCTGAAACTAACAACTAATAGA |
| 50 | core_49 | ACGGAACGTGCCGGACTTGTAGAACGTCAGCGCGGCAAACGCGGTCCG |
| 51 | core_50 | GCATTAACATTTCGCAGAAACAATTGCCGTTCTGGTGCTGGTCTGGTC |
| 52 | core_51 | GACATAAAATTTCTGCGTCACCGAAACAATGAAATAGCAATCGGAACC |
| 53 | core_52 | AGCACGTAGTTATCCGTTAATGGTTAAAGTAAAATAGTGAAGATGATG |
| 54 | core_53 | AATGGAAACAGTACATGAGGACTAAAGACTTTCGGCTACA |
| 55 | core_54 | AGACTACCGAATTATTCATTTCAATTACCTGAGCAAAAGAATTTATCA |
| 56 | core_55 | ATTAAGAGAGCCAGAATGGAAATTTTTCGCAGTCTCTGAA |
| 57 | core_56 | CCAGCCAGCTTTCAGTGCCAAGCTAACGAGTAAAACTCCATAAATCGG |
| 58 | core_57 | AATATAATGAATTATCACAAAGAAACCACCAGCGGGCCTCTTCGCTAT |
| 59 | core_58 | AAAAATGAAATTTTTTAGAGTACCGTTTTTACTCATCGCTTTCGCACT |
| 60 | core_59 | ACTCAAACCAATACTTGTCAATAGAAATGAAAAATCTAAATGTCGTGC |
| 61 | core_60 | TGAATAAGTCTACTAAAAATTAAGCAATAAAGTTAACGGGCCCTCATA |
| 62 | core_61 | CATAAAGCGCGAGCTGAAAAGGTGTATTTTCATGGAGCCGTCTCGTCG |
| 63 | core_62 | TGTGATAAATAGCTGTAAAGGTAAATCGGAACTCACCCAAATCAAGTT |
| 64 | core_63 | ACCGTGCATGATTCTCCGTTTTTTGGAACAAACGGTGCTGCAAGGCG |
| 65 | core_64 | AACTAAAGATCTCCAATCGGTTTACGATTATATGTACAGACGCTTTT |
| 66 | core_65 | GTCACCAATGAAACAAAATCACCGGCACCATTACCATTA |
| 67 | core_66 | GCCACCCTCAGAGGTAGTAGCGCGTTTTCATCGGCATTTT |
| 68 | core_67 | TGAGGCAGTATAGCCATATGTGA |
| 69 | core_68 | TTGAGGGGCGGAACAAATCTACGTCAGGAAGATTGTTTTTTTAAGCAAATATTAAACAAGAG |
| | | TTCTAGC |
| 70 | core_69 | CGCCTCCAGCCGCCTAGCAGCACCGTAATCAGTAGCGACGGTCATA |
| 71 | core_70 | CGCAAAGAATAGAAAATTCATAACCGGAACATCAACAAGCGGATAA |
| 72 | core_71 | ATAGGCTGATGAACGGCCAAGCGCGAAACAAAGTACAACTTGCTAA |
| 73 | core_72 | TTGACAAGAACCGAACGTATCATC |
| 74 | core_73 | ATGGGATTGGAGATTTTGACCAAC |
| 75 | core_74 | AGGCTTTTTTTTTTACCTCCGGCTTAGGTTTTACAAAATCGCGCAG |
| 76 | core_75 | GCTCAATTTAGGAGCCCTCAAATATCAAAC |
| 77 | core_76 | AGTAACATTATCATTTTGCGGAATCATATTCCTGATTTTTCACCA |
| 78 | core_77 | ACTAAGCCTGTTGCGTTTCGTTAGCAGCAGC |
| 79 | core_78 | TCATCAAATAGACTTTACCGAAGCCCCATACAGGCAACCGTATAA |
| 80 | core_79 | CACCCAGCCGCAAGGGGTAAAGTTAAACGCGAGGAAACGAACAAA |
| 81 | core_80 | GAGGAAGGTTATCTAAAATATCTCGTCTGAGTCCGTGCCTGTTT |
| 82 | core_81 | TTGTACCATAGACTGGTGATAATCCATGTCAATCAAAAGGGTGA |
| 83 | core_82 | TGGGAATTAGAGATTCAACCTCACGGTCCTTACACTTTTCTTTG |
| 84 | core_83 | AGAAGCAATTAAAATTGGCTATCAAGCTATTT |
| 85 | core_84 | ACAGGAGTCTTGAGTAACAGTGCGGCAAAGA |
| 86 | core_85 | GCCCAATAGGAACCTTGCGCCGATTTTTAATGACAACACAACC |
| 87 | core_86 | GCAAGCGGTCCACGCGGCCGATTAACACCGC |
| 88 | core_87 | CCGGCGAACGGCGAGACAAGAAATAAATTACATCGTTTGAATA |
| 89 | core_88 | ACATAGCGATAGCTTAAAAACCGTGGGGAAAG |
| 90 | core_89 | TGTTTAGTTTAAATAAGAATAAACACACGACC |
| 91 | core_90 | TTCCTGTGAGTCCACCACCCTCAGAGTTTGCCTTTAGCGTCAG |
| 92 | core_91 | GGTGAATTTCTTAAACATACACTAAAACACTC |
| 93 | core_92 | ACATACATAAGACAAAAGGGCGACCCAGCAAA |
| 94 | core_93 | TACCCAAATCAACATTATCTAAAGTTTTCTGT |
| 95 | core_94 | AAAAACAGTCCTTATCATTCCAAGGCCGGGTCTGCCGGGTT |
| 96 | core_95 | GTAAAAGAGTCTGTCCGTATTAGACTGCAACA |
| 97 | core_96 | ACGTTGTAAAACGACGAGAGCACATCCTCATACTGGCAGC |
| 98 | core_97 | CCTCAGAGGGAGAAGCTGACGAGAAACACCAG |
| 99 | Core_98 | AGATTTAGATAACCTGTTTAGCTAGCATCAATGCTTGCCC |
| 100 | core_99 | TATCCCAATCCAAATAGAGTTTCGGTCAGAGGGTA |
| 101 | core_100 | GCAGCGAAAGACAAACGGGTAAAATACGTAATGCCACTAC |
| 102 | core_101 | CTTTGACGAATGAGTGCAACATACGAGCCGGA |
| 103 | core_102 | CGGTGTCTGGAAGTTTCATTCCATGCCCGGCACCGCTTCT |
| 104 | core_103 | GCATCAGAACTGTTGCCCTGCGGCTGGTAATGGGTAAAGG |
| 105 | core_104 | TAGCTATCTTCTCCGTCGCCAGCAGCCTAATTCTTTTTTG |
| 106 | core_105 | GTGCCACGACGCGCGGAGCTAACTCACATTAAGGGTGCCT |
| 107 | core_106 | ATAATACATTTGAGGATTTAGAAATCACGCACGGGAGCT |
| 108 | core_107 | CTTTATTTCAACGCAAGGATACATATTAGCATAGTAGTA |
| 109 | core_108 | GTGATGAAAATGCCAACGGCAGCACCGTCGGTAATCAGAT |
| 110 | core_109 | TAATATCCATTGAGTTTAGCGGGGCCGTACTC |
| 111 | core_110 | AAGAAAAAATCACCAGATTAGGATAGGCCGGAAAC |
| 112 | core_111 | CTCGTCATACAGTTGAAAGGAATTGTCAGTTGGCAAATCA |
| 113 | core_112 | TAAAGGCTCACGGAAAAAGAGACGCAGAAATCACCAGT |
| 114 | core_113 | CTCAGAGCGCCCCCTTATTAGCGTTTCATAATCCATCGA |
| 115 | core_114 | CGATTTAGTAAGAGAACCTTGAAAAAACAAAC |
| 116 | core_115 | AGGGAAGGTCGTGATCCAGCGCAGTATTACCGC |
| 117 | core_116 | AAAATCCAAATATTGCATGATTAAGACTCCTTAT |
| 118 | core_117 | TTGAAATACCGACCGAACAGAGACTTCGAATTCGTAATC |
| 119 | core_118 | TATGGTTGAAACAGGATGGTTTGCGGAGAGGCGGTT |
| 120 | core_119 | AAACTTTATGGCTATTTTTTAGTCTTTAATACGCGAGA |
| 121 | core_120 | CAATCCGCCGGGCGCGGTTGCGGTATGAAACGGGTA |
| 122 | core_121 | AAAAGTAGTGAATTATCACCTCATTTGCGGTGAAGG |
| 123 | core_122 | TTAAACCACAGCCTTTACAGAGAGTTCAGGGATAGCAA |
| 124 | core_123 | TAACTGAGCGCCTGTGCATTTTTTCTGTGGTGCTG |
| 125 | core_124 | GCGATGGCCCACTACTTAGAATTATAAAGACCAGTA |
| 126 | core_125 | ATTAAGTTGGGTAACGCCAGGGTTTTCCGAGTAACA |
| 127 | core_126 | GCAAAGCCCAAATGAAAGTAGGAGGTTGGTAGCAATTCATGAG |
| 128 | NoHandle_1 | TGATAAATTAATGCCGGAGAGGGTGGTCATTG |
| 129 | NoHandle_2 | CCTGAGTAATTCATTGCAATACTGCGGAATCG |
| 130 | NoHandle_3 | GCCTGATAAATTGTGTCGAAATCCGGCGCAGACGGTCAAT |
| 131 | NoHandle_4 | AATAAATCAAATCAATCGGAATAGGTGTATCATTTTGCTC |
| 132 | NoHandle_5 | TTACCCTGTCTACAAACGCATTAAATTGCACGTAAAACA |
| 133 | NoHandle_6 | TTTGAAATCCAGTAATGCCCCCTGCCTATT |
| 134 | NoHandle_7 | TTTAAACAAAAACTAGAGAAAAGCTTTACCAGACT |
| 135 | NoHandle_8 | TTTACCGTGAGGACAGGCTGACCT |
| 136 | NoHandle_9 | CCAAGGGGTTAATCGCAAGACAAAGAGCGCGAAC |
| 137 | NoHandle_10 | CCTGAGAGGACCATAAGCATCAAAAAGGCCAGAGG |
| 138 | NoHandle_11 | ATCAAGAAAACAAAAAAATTCTTTACCGACA |
| 139 | NoHandle_12 | TTGAGAGAGATTCGCCTGATTGCGGAGAAAC |
| 140 | NoHandle_13 | AATAACGACAAGAACGTGGACTCCAACGTCA |
| 141 | NoHandle_14 | ATCTTTGACCCCCAGTCAGCTT |
| 142 | NoHandle_15 | TTTTGGGGTCTGTAAATGTCCAGA |
| 143 | NoHandle_16 | GAGGCTTTCTCATTAAGCTGAGACTCCGGAGGT |
| 144 | NoHandle_17 | ATTTTCCCGTGAACCACCTAAAGGGAGCCCCAGCATAA |
| 145 | NoHandle_18 | GAAGTTTCCATTAGCATCGGAACGAGTAGTACCGCCACCCTC |
| 146 | Handle_1 | GCAGTAGAGTAGGTAGAGATTAGGCATTGCAGTAGAGTAGGTAGAGATTAGGCATGATAAA |
| | | TTAATGCCGGAGAGGGTGGTCATTG |
| 147 | Handle_2 | GCAGTAGAGTAGGTAGAGATTAGGCATTGCAGTAGAGTAGGTAGAGATTAGGCACCTGAGT |
| | | AATTCATTGCAATACTGCGGAATCG |
| 148 | Handle_3 | GCAGTAGAGTAGGTAGAGATTAGGCATTGCAGTAGAGTAGGTAGAGATTAGGCAGCCTGAT |
| | | AAATTGTGTCGAAATCCGGCGCAGACGGTCAAT |
| 149 | Handle_4 | GCAGTAGAGTAGGTAGAGATTAGGCATTGCAGTAGAGTAGGTAGAGATTAGGCAAATAAAT |
| | | CAAATCAATCGGAATAGGTGTATCATTTTGCTC |
| 150 | Handle_5 | GCAGTAGAGTAGGTAGAGATTAGGCATTGCAGTAGAGTAGGTAGAGATTAGGCATTACCCT |
| | | GTCTACAAACGCATTAAATTGCACGTAAAACA |
| 151 | Handle_6 | GCAGTAGAGTAGGTAGAGATTAGGCATTGCAGTAGAGTAGGTAGAGATTAGGCATTTGAAA |
| | | TCCAGTAATGCCCCCTGCCTATT |
| 152 | Handle_7 | GCAGTAGAGTAGGTAGAGATTAGGCATTGCAGTAGAGTAGGTAGAGATTAGGCATTTAAAC |
| | | AAAAACTAGAGAAAAGCTTTACCAGACT |
| 153 | Handle_8 | GCAGTAGAGTAGGTAGAGATTAGGCATTGCAGTAGAGTAGGTAGAGATTAGGCATTTACCG |
| | | TGAGGACAGGCTGACCT |
| 154 | Handle_9 | GCAGTAGAGTAGGTAGAGATTAGGCATTGCAGTAGAGTAGGTAGAGATTAGGCACCAAGG |
| | | GGTTAATCGCAAGACAAAGAGCGCGAAC |
| 155 | Handle_10 | GCAGTAGAGTAGGTAGAGATTAGGCATTGCAGTAGAGTAGGTAGAGATTAGGCACCTGAGA |
| | | GGACCATAAGCATCAAAAAGGCCAGAGG |
| 156 | Handle_11 | GCAGTAGAGTAGGTAGAGATTAGGCATTGCAGTAGAGTAGGTAGAGATTAGGCAATCAAGA |
| | | AAACAAAAAAATTCTTTACCGACA |
| 157 | Handle_12 | GCAGTAGAGTAGGTAGAGATTAGGCATTGCAGTAGAGTAGGTAGAGATTAGGCATTGAGAG |
| | | AGATTCGCCTGATTGCGGAGAAAC |
| 158 | Handle_13 | GCAGTAGAGTAGGTAGAGATTAGGCATTGCAGTAGAGTAGGTAGAGATTAGGCAAATAACG |
| | | ACAAGAACGTGGACTCCAACGTCA |
| 159 | Handle_14 | GCAGTAGAGTAGGTAGAGATTAGGCATTGCAGTAGAGTAGGTAGAGATTAGGCAATCTTTG |
| | | ACCCCCAGTCAGCTT |
| 160 | Handle_15 | GCAGTAGAGTAGGTAGAGATTAGGCATTGCAGTAGAGTAGGTAGAGATTAGGCATTTTGGG |
| | | GTCTGTAAATGTCCAGA |
| 161 | Handle_16 | GCAGTAGAGTAGGTAGAGATTAGGCATTGCAGTAGAGTAGGTAGAGATTAGGCAGAGGCTT |
| | | TCTCATTAAGCTGAGACTCCGGAGGT |
| 162 | Handle_17 | GCAGTAGAGTAGGTAGAGATTAGGCATTGCAGTAGAGTAGGTAGAGATTAGGCAATTTTCC |
| | | CGTGAACCACCTAAAGGGAGCCCCAGCATAA |
| 163 | Handle_18 | GCAGTAGAGTAGGTAGAGATTAGGCATTGCAGTAGAGTAGGTAGAGATTAGGCAGAAGTTT |
| | | CCATTAGCATCGGAACGAGTAGTACCGCCACCCTC |
| 164 | side1_pro_S3_ | TTCTACCTACATCACTTGCCTGAGTAGAAGATGCAACAGTTCTGGCC |
| | 1xT_1 | |
| 165 | side1_pro_S3_ | TGACTTCGAGCCAGCCAACGCTCAACAGTAGCCAACATGTAATTTAGTATTACCGCCAGCC |
| | 1xT_2 | AT |
| 166 | side1_pro_S3_ | TGAAATTTAACGTGTAGAACCGTTGTAGTATCGGCCTTGCTGGTAATGGAT |
| | 1xT_3 | |
| 167 | side1_pro_S3_ | AGGATCCCAGTAATAAAAGGGACAGAAAAACGCTCATGGAAATTACT |
| | 1xT_4 | |
| 168 | side1_pro_S3_ | TCCAGCCATATTTAACAACGGGCTTAATCGTTATACTTAATTACATTAATTA |
| | 1xT_5 | |
| 169 | side1_pro_S3_ | GAATCGGCCACTGAGAGCAATCAGAGAATTAACCCTTCTGACCAGAACAAGCAGAGGCATT |
| | 1xT_6 | GATT |
| 170 | side1_pro_S3_ | TTCAATCCTGAAAGCACCTAAATCTCACAATGAAGAGTCAGCTTGACCTATCAGG |
| | 1xT_7 | |
| 171 | side1_pro_S3_ | CAATAAACAACATGTCATAAGGCGATCATATGTGAGAATCGACT |
| | 1xT_8 | |
| 172 | side1_rec_S3_ | TTTACTAGCTTTTTTGTGAATAACCTTGCTTCGGGCCTTGATATTCACAAACA |
| | 1xT_1 | |
| 173 | side1_rec_S3_ | ATTGAGCGCTAACCCTCAGAACCGATACACCCT |
| | 1xT_2 | |
| 174 | side1_rec_S3_ | TTGGCAGATTCACCAGTCACCGGAAGTGCCGTCGAGACGAACCACCAG |
| | 1xT_3 | |
| 175 | side1_rec_S3_ | AAAGGTGGCAACATATAAAAGAAACCTCAATCTTCTTCGCAATGGATTATT |
| | 1xT_4 | |
| 176 | side1_rec_S3_ | TCCGCCATATCAGAAGAACCGCTATTCGTTTTTTCGCTGAGGCTTGGTCACCCTCA |
| | 1xT_5 | |
| 177 | side1_rec_S3_ | TAATCGATGCGGCGCATGTAGAATTAGACGGGAGAAT |
| | 1xT_6 | |
| 178 | side1_rec_S3_ | GGTGTGTTCAGCAAATCGTTAACGCGGCCAGAGCTGTCTTGGAAGCGCAACCAT |
| | 1xT_7 | |
| 179 | side2_pro_S3_ | GGAGCCTTCAGCCCTCGAGAATAGAAATCAAGATTCATT |
| | 1xT_1 | |
| 180 | side2_pro_S3_ | TTCATTTTTCACGTTGAAAGAATTGCGCAACGCCAACAGCCAACTTAA |
| | 1xT_2 | |
| 181 | side2_pro_S3_ | GTCACTGCACACCCTGAACAAACAGCGGATCAATATTATTGCCCAATATTTAGCGAAATT |
| | 1xT_3 | |
| 182 | side2_pro_S3_ | TCCACAGATAATTGTAAAAAAAGGCTCCAAAAACAACTTTCAACATAAT |
| | 1xT_4 | |
| 183 | side2_pro_S3_ | TTTAAGTTGCTAATCCGGTATTCTAAGAACGGAGGTTTTACAAAATATGTAGCAT |
| | 1xT_5 | |
| 184 | side2_pro_S3_ | TATCACCTCCCGACTTGCGGCGAGGCGTGCAAGCAGGTGCCA |
| | 1xT_6 | |
| 185 | side2_pro_S3_ | TTCAGTTTCAGCGGAGTATAGTTATTCCAGAGTGTTTACCAGTCCCAATTTTTTAAG |
| | 1xT_7 | |
| 186 | side2_pro_S3_ | GCGGGATCCAGGGAGTGCTTTCGAACAAACTAAATAATAAGAAT |
| | 1xT_8 | |
| 187 | side2_rec_S3_ | TAGATCGCACTATGTGAGCCAGTCACGGGTGCCGGAAACCAGGCTAAAGTATCCTTTT |
| | x1T_1 | |
| 188 | side2_rec_S3_ | TCAACGCTAACGAGCGTCTGCGTAACGTGTGAGAGGAGTAATCACAGTTAAGCGTCATA |
| | 1xT_2 | |
| 189 | side2_rec_S3_ | CATGGCTTTTGATGATCATAAGGGAACCGGATTAAATGAATTTTGTCGTCTTTT |
| | 1xT_3 | |
| 190 | side2_rec_S3_ | TGTTAGATTAAAATTTTTAGAAGTCAGTGCGTACTGGT |
| | 1xT_4 | |
| 191 | side2_rec_S3_ | TTTGACCATTAGATACAAGGAAACATGCTGATCGGCGAAATTATCCTGAAT |
| | 1xT_5 | |
| 192 | side2_rec_S3_ | GAAAGGCCGGAGAACCCTCGCCCAAAAATAATAAAAATAGCACAGGCTTGAGAGTATCGGC |
| | 1xT_6 | CT |
| 193 | side2_rec_S3_ | TAAAAACCAAACGAACTAAACGACGACATGGTTTAAACATTAATTAATTGC |
| | 1xT_7 | |
| 194 | side3_rec_S3_ | TTCGCCATTAACGCCAGAATTAATTTTGATAAAACAGATTTTTGTGAGGCGG |
| | 1xT_1 | |
| 195 | side3_rec_S3_ | TCTGATTATCAAAATTATTTTTGTTGTTCAAAATCCCTTATAAGCTGATTG |
| | 1xT_2 | |
| 196 | side3_rec_S3_ | CCGCCTGGCCCTGAGAGTGGTTCCCTACCACCACACCCGCTGATAGCCCTT |
| | 1xT_3 | |
| 197 | side3_rec_S3_ | TTCACGTTGGTGTATTGGGCCTTCCTTTTTTTAGCCAGACCCGTCGCTGCCAGT |
| | 1xT_4 | |
| 198 | side3_rec_S3_ | GCGGTCACGCTGCGCGTAACCAGCAAATCCATATAACTATATGT |
| | 1xT_5 | |
| 199 | side3_rec_S3_ | TTGCAGAAACGTTAGGCTCATTAAGAGGAAGCCCGAGCCCGAGATA |
| | 1xT_6 | |
| 200 | side3_rec_S3_ | CAAAGCGCCATTTTTTCGCCATTCAGGCTGCGCTCGCGTCTACCGTAATGGT |
| | 1xT_7 | |
| 201 | side3_rec_S3_ | GTTCAGAATTTTTTACGAGAATTCTGGAGCTAAATTGTATACATAAGAATACCACATTCT |
| | 1xT_8 | |

TABLE 2B: Staple sequences for triangle 1, version 2

| SEQ ID | | |
|---|---|---|
| NO: | Description | Sequence/Details |
| 202 | core_1 | AGGGGACGAGATTCTCCGTTTTTTGGAACAAACGAAGTTGGGTAACG |
| 203 | core_2 | TAGTCTTCCGCCGCTTTTTCTTAATGC |
| 204 | core_3 | AGTAGGGAATATTTTTTTTTGAATGGCTATCGCTCAAC |
| 205 | core_4 | CAGAGGCATTTTCGACATTCTGGAACATTCGTAATCATG |
| 206 | core_5 | GCCAGTAAGCTGTTTCAAACCAAGAATCGGAATCACCCAAATCAAGT |
| 207 | core | GGCCACCGAGTAAAAGAATAATGTTGAGGAT |
| 208 | core_7 | ATCGTAACCGTTTGCCTTCCTGTTTTTTGCCAGCTACCCGTCGCGACAGTA |
| 209 | core_8 | CATTTTCAGGGATAAAACAGCTTTTTTTATACCGATAGTACG |
| 210 | core_9 | GATTTTCAGGTTTAAGTTGAAAGCTGATTGC |
| 211 | core_10 | AGCCCTAAGTTATACAGATGATGAAACAATGTGTTGTT |
| 212 | core_11 | CAGTAACAACGCCAGCCCAGCAGAGCGAACGCCATCAAA |
| 213 | core_12 | AACAACATGTAAGAGCAACACTATCGTTCTAGGATTTTTTAACGGTAATCGTAAAAC |
| 214 | core_13 | CTGAGAAGTGAAAATTATTTGCTTTTTCGTAAAACAGAAACGCCAGAATC |
| 215 | core_14 | ACAATAAATATAAAGTACCGACAATTCACCAG |
| 216 | core_15 | GACGACGAACAAGTGCCGTCGATTGAGGCAGGTCGAGGTGCCGTAAGCCGCCA |
| 217 | core_16 | AGGCGATTGCGGAGCATCACTAACGGTTTAAG |
| 218 | core_17 | CCAGGGTTTTCCCAGTCACGACGTTGTAGTAACA |
| 219 | core_18 | ACAATGACAGAACCGCCACCCTCAAGAAAAGT |
| 220 | core_19 | TTTATCAGCTTGCTTTCAACCTAAAACGAAAG |
| 221 | core_20 | GGCCAGTGCCAAGCTTATAACGGAACGTGCCCCAGAGC |
| 222 | core_21 | AGCGTGGTGCTGGTCCGTTTTTTCGTCTCGT |
| 223 | core_22 | TAGTAAATATACCAAGCGCAGACG |
| 224 | core_23 | CAAGAATGGCATTAGATTTACCAGTCCCGCAATTTTGTGCGCCAAAGACAAAA |
| 225 | core_24 | GGGCGCGGTTGCGGTATGAGCCGGGTCTCAAGATT |
| 226 | core_25 | TAAACATCAACAAAGAAACCACCATTATCATTTTGCGG |
| 227 | core_26 | TATTACAGGTAGAAAGGTGTAGCTTTAATATAGGCAGAATGTTTTAATT |
| 228 | core_27 | CAATAGATGCCGATTAATCAGTGAAATACGTGGCACAGACCTTAATTGGCAAG |
| 229 | core_28 | TTAGAAGTCGCGGGGATAACTCACATTAATTGTGCCTAAT |
| 230 | core_29 | AGAAGGAGCGGAATTATCATCATGACGCTCCGTGAGCCCGCGCC |
| 231 | core_30 | GATGCCGGCCTGCGGCTGGTAATGGGTAAAGGTTTCTTT |
| 232 | core_31 | AGCCAGCGGGGGGTCATTGCAGGCGCTTTCGCATACAATTTTATCCTGATGAAATAG |
| 233 | core_32 | AGTTGCTCCGAAGCCCTTTTTAGAGCCACCACCCT |
| 234 | core_33 | ACATTTTATTCCTGAGAACGTTATTAATTT |
| 235 | core_34 | AGTTAAACCAGCACCGTCGGTGGTGCCATCCCTTACAAAA |
| 236 | core_35 | ACATCCTCCCAATTCTAGTACCTTTAATTGCTCAGGTCAG |
| 237 | core_36 | TCAGGCTGCGTTTTTAACTGTTGGG |
| 238 | core_37 | GAAGGTTAGAACGGTATAAAGAAACTAATAGATTTTTTAGAGCCGT |
| 239 | core_38 | GTTTAGTATCATATGCGTAAATCGGTGAGTGA |
| 240 | core_39 | CGGGGGTTTCTGCCAAAGGCTTCATAATC |
| 241 | core_40 | TGCGCGCCAGGAAACGCAAACTTAAATTTCTG |
| 242 | core_41 | AAAGCCGTTTGCCTATTGAGGGAGGGAAGGTAAA |
| 243 | core_42 | CCGTAAAAGCCGCCAGTTTCGGTCTACATAAA |
| 244 | core_43 | GAACGGATGCCTCCGGGGACTTGTAGAACGTC |
| 245 | core_44 | GCCACGGCTGAAAAGCGAACGAGTAGATT |
| 246 | core_45 | CGCTTCTGGTGCCGAGGTGGAGCCGAATAAGGGATTAGAGGTACCAAA |
| 247 | core_46 | TTCATCAAAACGAGTAGTAAATTTTTTTGGCTTGAGATGGTTTTATGCGAT |
| 248 | core_47 | ACAAAATCTCTGGTCAAATACCGATATGAAAAATC |
| 249 | core_48 | TAGACAGTCTAAAATATCTTCGCGAATTGAGGAACTGAT |
| 250 | core_49 | GCCGCTGGGCGCTGAGAATCGCAATAAGAATAAACACCGAAAACATAGCGATAG |
| 251 | core_50 | GCGTACTAGGAGCTAAGGTTTGCCGAGGCGGTTTGCGTATTGGGCG |
| 252 | core_51 | TGGTTGCTGAGTGAGCCATACGAGCCGGAAGCCCGATTTA |
| 253 | core_52 | CGGCCTTTAGTGATGTTCAACCGTTAGCGTCAGACTGTAGCGCGTTTTCGACATA |
| 254 | core_53 | AAGAGCAAGAAACAAATCTTACCAACGCTACATCAGCTGCCGGTG |
| 255 | core_54 | AGCAAATCAGATATAGGCACGCGTCAATGAAA |
| 256 | core_55 | ATCCGGTACGCAGTATGAAGGATGAATAGGAACATGA |
| 257 | core_56 | TTCTAAGAACGCGAGGGTTTTCACAGTAGCGAC |
| 258 | core_57 | GAAACCGTGTGCACTCTGTTTTTGGTGCTGCGGCC |
| 259 | core_58 | CAAATCAATAGTAGCATAAAGCCTCAG |
| 260 | core_59 | CTTGCCCTGTAATACTTTTGCGGGGGTTGATATCGTCATAAAACAGTTCAGAAAAC |
| 261 | core_60 | AATTTCAAATGGGCGCGGGATAGGTCACGTTTGGCGAA |
| 262 | core_61 | AGGCGAATGCTCATTTAGTTGAGATACATAACCTGCAA |
| 263 | core_62 | CCTGAGCAGAAATCGGCCAGTTTGGAAGAAAGGAAG |
| 264 | core_63 | CTGTGTGAGTACCTCAGAGCCACCACCCTCAG |
| 265 | core_64 | GGCCTCTTCGCTATTCATGTTTTGCTGAATATAATGCTGATCAAAAG |
| 266 | core_65 | TAGGATTATGAGACTCAAAATCACCGGAACCAAACGTCACGCCTGTTCTGAGTAAC |
| 267 | core_66 | CCCCCTGCATCAGACGATCCAGGCAGCTTACGGCTGGAGGTGTCCAGACGAGCGT |
| 268 | core_67 | GGGAAGCCCAACGGGATGCTGATTGCCGTGTTTACCACACAATCA |
| 269 | core_68 | CAATAGCTATTTTTTTTAATTTTGCTTTTTCCCAGCCTCAATCCGCC |
| 270 | core_69 | AGAAGCCATCGGTTGGTGGCATCAATTCTGGCGCGAG |
| 271 | core_70 | AACTGAAAAATCTTTTACCAGACGACGAAATCACCATCAATATGATAT |
| 272 | core_71 | TGTAGCGGTCACGCTGCGCGAAAATTCTTA |
| 273 | core_72 | TTTCATTTGAATTACCTTAGGCGTTACATATTTAACAACGCACCTGAAA |
| 274 | core_73 | ATGGTTTGACAGAGCGGGCGCTAACAGGGCGCGTAAG |
| 275 | core_74 | AAATACCGGAGCTTGACTATCAGGGCGATGGC |
| 276 | core_75 | TTATCATTTAGTTAATGTCAATAG |
| 277 | core_76 | CGTACTCAGGAGGTTTTTAAGACTCCTTATTA |
| 278 | core_77 | TCGCTGAACGCAGAAACAGCGGATCAATAAACCGTAAC |
| 279 | core_78 | TACAGACTGGTGAAAGAAACGCAAAGGGCAA |
| 280 | core_79 | CAGGCGCATGCCCGTAGTTCCAGTAAGCGTCA |
| 281 | core_80 | CTGATTGTTTGGATTATACTTCTGAGTCTGTTCAGAGCG |
| 282 | core_81 | GCAAATATTTAAAAGAGAATCCTGATAAA |
| 283 | core_82 | GTTAATACATTGTGCTTCAAATATCGCAGCCCGAGATA |
| 284 | core_83 | TTTTGTTAGATTAAGAACCCTGACTATTATAG |
| 285 | core_84 | TCGGCCAACGATTAGACTCGTTAGAA |
| 286 | core_85 | GGAATTAATTAGCAAGGCCGGAGAGCCACCCAATAGCATTGCTCAG |
| 287 | core_86 | AACGCAAGGATAAAAATTTTTCAATAAGCAATTAACATC |
| 288 | core_87 | AATTATCCCATCGATAGCAGCACATCTTTT |
| 289 | core_88 | TATTGACGGAAATTATTCATTAAAGGTGCCTTACAC |
| 290 | core_89 | GGGCGACAAAGGGTAAAAAAAATCTAAACAGCCTAATATCGCAGCCTTTTCAGCGG |
| 291 | core_90 | CATATAAAGGGATAGCCGGTTGTGATTGAGCGCATAAACA |
| 292 | core_91 | CCATTAGACTATATTTTCATTTGGACTAATAGCGTAACAA |
| 293 | core_92 | TAGTTTGACGCTGGCAAACCTCACCGGAAACA |
| 294 | core_93 | TTCATTCCATATAACAGTTGATTCTCAGGAAACCAGGCAA |
| 295 | core_94 | CAGGGTGGTTTTTCTTTATTTAGGAGCACTAACAATTGCGTA |
| 296 | core_95 | AAGCGGTCCACGCTACAGGAGAATACAT |
| 297 | core_96 | CCGCTTTCCAGTCGGGCGCCACCCGTCTTTTCATCGTAGGTCACACGATTAATACCT |
| 298 | core_97 | AGAATCAAGCACAGCGCAGTGTCACCTTTCCAG |
| 299 | core_98 | ATAGAAAACATACAGGATAAAGAACCGGATATTCATTACCATCGGCGACTGTTTAG |
| 300 | core_99 | GCGGATGGCGAATTTTTCAGACGTATTTTTTAGTAAAAAGAAATTACCT |
| 301 | core_100 | CTTAATTAAATATGCAACTAAAGTACGGTGAAGGGCGAGCGTCTGGACCGTAAT |
| 302 | core_101 | CTTCACCGAGACGGGCAACAGCTAGCTCAAGTACCTTT |
| 303 | core_102 | GCCTGGCCCTGAGAGAGTGGTTCCAAAGTAACCACCACACTAATGCGC |
| 304 | core_103 | TCAGAACCGCCACAAGCCTGGGGCGTTGCGCTTTCCTTTACAAA |
| 305 | core_104 | CCGCCTCCCTCAGAGCCAGTAGCAAGTTGAGGCTTTGCCCTTATCAGATGATGGCA |
| 306 | core_105 | ACCGGAAACCACCAGAGCCGCCGCCAGGGCGCGGGGTT |
| 307 | core_106 | ATTAGCGTTTGCCCGTAATCGGTCATACTGGTGTGTGCTCGTCA |
| 308 | core_107 | GAATAAGTTTTCATCGGCATCAGTTGGGTCTCACGG |
| 309 | core_108 | CAATAAATTCAATAACAACGTACAACGCGGTCTGGTCAGCAGCAACCG |
| 310 | core_109 | CGAGCTTCAAAGCTTAGAGAATAATTCTCGGTGCGAGGGGGATGTGCTGCA |
| 311 | core_110 | CAAAATCCCTTATAATGATTGCCACCAAGTTTACATCGGTGAATATA |
| 312 | core_111 | GAAGAAAGCGAAAGCAACCAGCAGGCGAAAATCCTGTTTTTTTGATGGTTGCAGC |
| 313 | core_112 | ATAAAGTAATTGTTCACGTATCCAACAG |
| 314 | core_113 | CTCAAGAGTTAGCAATATTCTGATGTATCACGAAAGACAAGGCTTTG |
| 315 | core_114 | GGTGGCAACCCTGCCTGGACAGATACCGAACTCTCATCTT |
| 316 | core_115 | TCAACCATAATTTTTCCTCGACGTTAATTTTTAAAACGATGCCAGTTTG |
| 317 | core_116 | AGAATTAGTAACAGTAGGCTGGGAACGAGGCGCGAAAC |
| 318 | core_117 | AGCAAAATGAGTAATCTTGACGACCCTCATAGCCAGACGT |
| 319 | core_118 | AGCATGGTAATAAATTTTAAAAATCCGCGATCGCCTG |
| 320 | core_119 | AAAGCTAATTTATTTCAGCTGCTCATTCAGT |
| 321 | core_120 | AACATTATCTGCGGAAATCAGAAACAATCATAGTGAGAAA |
| 322 | core_121 | GCCGGCGAGTGGCGACAAAAAATTAACAATATATTCGCTATT |
| 323 | core_122 | AAGCACTATACCGCACGAACGCGTGAGAGA |
| 324 | core_123 | CAGGAGGGAGGGTTGAATGCTGAGTTGGGT |
| 325 | core_124 | CTGGATAGCGTCCAATAGACTTGCCAGAGGGGCGGAAGCAAACTCCAACCTTTTGA |
| 326 | core_125 | ATCAAAAAAAATTCGCCAGGTCATTTTTGAGA |
| 327 | core_126 | GAGTCCACTATTAAGCGGATTGCGGGTTGAGTTAAATCATATTCATT |
| 328 | core_127 | GAGTAGAAACCGTTGTATATAATCCAATTCGACAACTCGTCGTGCCAG |
| 329 | core_128 | CCTTGGAATTTTTTATAATTACTAGAAAAATTTTCCCTTAGAAT |
| 330 | core_129 | TTTGCGGGATCGTTTTTCATGAGGAAGTTTC |
| 331 | core_130 | TAAAGCATCAATCGTCAATTATTTGAAT |
| 332 | core_131 | AGTGAGAATAATTTTTAAAGGAGCCTAAAACAGACCAACT |
| 333 | core_132 | AAGGGCGAAATTTTTTTAATGGAAACTTTTTGTACATAAATTTACATTTAACAA |
| 334 | core_133 | CTCATTAAAGCCATTTTTAATGGAAAGCGCA |
| 335 | core_134 | TAATGCCACTACGAAGGCACCGAGGTGAATTTCTTGCAAGCCCAATAGGA |
| 336 | core_135 | CCATAAATTTTTTTAAAAATCAAGCAAACATTGTAAACGAGGCATA |
| 337 | core_136 | TCGGCCTTGGGAAGGCTCATTAAAGATTGTATAA |
| 338 | core_137 | AGTTAAGCCCAATAATACCCATGTTAACGGAATAC |
| 339 | core_138 | AAGCAGATGTTTTGAAGCCTTAAAACTGTTGCGTTACCTGC |
| 340 | core_139 | ATTCATCAAGCAATACGTACCGAGCTCGAGTGCTCACTGCCTGCATTAATGAA |
| 341 | core_140 | AAAAAGAGTTGAAAGAATTTCGGAACTTTTTTATACGTAGATTTTTAATACAATAGCCCCCTT |
| 342 | core_141 | TTGACGAGATCCGCTCAATTTAGGTTTTTTCCACCGTGTGTGAGAAGATTCATCTT |
| 343 | core_142 | AATCATTACTCCTCACCCATTACCGAGCCAGCAAAATCACAAACCTGTATTAAATC |
| 344 | core_143 | GAAGGGTTAGAACCTACCATATCTTTTTATAAAGGGATT |
| 345 | side_1_pro_S3_ | TGCCTGCCCCTTCTGCAACATGTACAATTCC |
| | 1xT_1 | |
| 346 | side_1_pro_S3_ | TCTATATCCCATCCTAATTTTGAACAAGTCAGCTAACATAGGTCAGAAAACT |
| | 1xT_2 | |
| 347 | side_1_pro_S3_ | CAAGCCGTTTTTAATATAAGAGAACAACATGTAAAAATAAAAGT |
| | 1xT_3 | |
| 348 | side1_pro_S3_ | ATCCCCGGTTCTTTGACCAGTAATATTGCAACAGGAAAAACGCTGAT |
| | 1xT_4 | |
| 349 | side1_pro_S3_ | TAACTCATGGAAGTAATAACATCACTTGCCTCGCCAGCCAAAAGGGA |
| | ×T_5 | |
| 350 | side1_pro_S3_ | TGAGCGGCTGTCATCAACAATAGATAAGTCCACGAGCATGTAGAAACTCCAGAACAATATTA |
| | 1xT_6 | C |
| 351 | side1_pro_S3_ | CCATCACGAGATAGAATGGTAATACAATCAATAATGCTT |
| | 1xT_7 | |
| 352 | side_1_pro_S3_ | CAAATTAGAACTCAAACTATCGGCCTAGCT |
| | 1xT_8 | |
| 353 | side_1_rec_S3_ | TACCGCGGCATGAAGTACCGATCGCCTTTTTACGCATAACCGATAAAGGCCGCT |
| | 1xT_1 | |
| 354 | side1_rec_S3_ | CCAAAAGAACTCACCCTCAACAACCCCACCT |
| | 1xT_2 | |
| 355 | side_1_rec_S3_ | TCAGCAAATCGTTAACGGCATCAAGAATGCGGCGGGAGAGCCGAAGCGAAT |
| | 1xT_3 | |
| 356 | side1_rec_S3_ | TGACTTGCGGGCCCGTTTTACAAAGTTACCAGAAG |
| | 1xT_4 | |
| 357 | side_1_rec_S3_ | TTTATTTACATTGGCAGAAAGGTAATACCAGGCGGATAAGCCAGAACCT |
| | 1xT_5 | |
| 358 | side1_rec_S3_ | ACCGTCACCGACTTGAGCCATTTGTAAAAGTTTTCGCGTCAATCGTCTGAT |
| | 1xT_6 | |
| 359 | side1_rec_S3_ | TTCTGTCCACGGCTTAGTGCAAAT |
| | 1xT_7 | |
| 360 | side2_pro_S3_ | TGTATCGGACTGAGTTAACAACTAAGAT |
| | 1xT_1 | |
| 361 | side2_pro_S3_ | TCCTTGTTTAACGTCAAAAAATAAGAAATTGCCAGACGCAAC |
| | 1xT_2 | |
| 362 | side2_pro_S3_ | AAGAATTGAGCCTAATCGATTTTTCCCT |
| | 1xT_3 | |
| 363 | side2_pro_S3_ | TAGTTGCTAAACAACTTCATTCCAGAGGGTATAGAATTTGTGAGAGCAAACACCACG |
| | 1xT_4 | |
| 364 | side2_pro_S3_ | TAATTAACATAAATTATTTATCCCAATCCAATGAAAATAAGAGAGATCAGTACAA |
| | 1xT_5 | |
| 365 | side2_pro_S3_ | ACTACAACAGGCTCCATCACGTTGAAAATCTCCTGTATGGGATTTAATT |
| | 1xT_6 | |
| 366 | side2_pro_S3_ | TCTCAGGAATTGCGAATAATAGAAAGGTCGTCACAACCCACCTCATTT |
| | 1xT_7 | |
| 367 | side2_pro_S3_ | CAAAAAAAGCCTGTAGTCAACAGTTACAGAGAGAAGGAT |
| | 1xT_8 | |
| 368 | side2_rec_S3_ | TAAAAACCAAACAGGTACCAGTAAGTTCCTGACGAGAATCGCACTCCAT |
| | 1xT_1 | |
| 369 | side2_rec_S3_ | TTTTTGCATAGAACCCTCATATGTTTTAACGATACAGGAGTGTACT |
| | 1xT_2 | |
| 370 | side2_rec_S3_ | TTTTGCAAAAGCAGGACGTCAGGAAGAACACCAGCATTAAATAAGAGG |
| | 1xT_3 | |
| 371 | side2_rec_S3_ | TACATTTCGCAAATGGTTCATATGTCCGGCAAGCGCCATGCGGGAGAATTT |
| | 1xT_4 | |
| 372 | side2_rec_S3_ | TACACCCTGAACAAAGTCACAGACAGCTTTCTCCG |
| | 1xT_5 | |
| 373 | side2_rec_S3_ | TTTTCCGGCACTGTGAGCGAAAACGACAGCGCCATTCGCCATCTGGAAGTTCATTTT |
| | 1xT_6 | |
| 374 | side2_rec_S3_ | CTGACCTTTCGTCTTTTTAGCGTAACGATT |
| | 1xT_7 | |
| 375 | side3_pro_S3_ | CAGTGAGATTTAGGAAGCCAGCAGCAAATTAACACTAAT |
| | 1xT_1 | |
| 376 | side3_pro_S3_ | TCCAAACCTCAAATATCATCAACACGTCAGAGAGAAACAATAACGGTCACCAGT |
| | 1xT_2 | |
| 377 | side3_pro_S3_ | TACGCGCGCCAAAAGGTAGCTATTGCCTGAG |
| | 1xT_3 | |
| 378 | side3_pro_S3_ | TGAGGTGAGGCGGTCAGACGAACCAGATTCATCTTTAACCA |
| | 1xT_4 | |
| 379 | side3_pro_S3_ | CCATTAAAGTTGGCAAAAACCCTCAATCAATAAGATAAAACAGAGTTAT |
| | 1xT_5 | |
| 380 | side3_pro_S3_ | TTAGAGATACCACAAACCCTTGCTGAATT |
| | 1xT_6 | |
| 381 | side1_pro_S3_ | TTTTTTGCCCCTTCTGCAACATGTACAATTCC |
| | 1xT_1 | |
| 382 | side_1_pro_S3_ | TTTTTTATCCCATCCTAATTTTGAACAAGTCAGCTAACATAGGTCAGAAAACT |
| | 1xT_2 | |
| 383 | side_1_pro_S3_ | CAAGCCGTTTTTAATATAAGAGAACAACATGTAAAAATAATTTTT |
| | 1xT_3 | |
| 384 | side1_pro_S3_ | ATCCCCGGTTCTTTGACCAGTAATATTGCAACAGGAAAAACGCTTTTT |
| | 1xT_4 | |
| 385 | side_1_pro_S3_ | TTTTTTCATGGAAGTAATAACATCACTTGCCTCGCCAGCCAAAAGGGA |
| | 1xT_5 | |
| 386 | side_1_pro_S3_ | TTTTTCGGCTGTCATCAACAATAGATAAGTCCACGAGCATGTAGAAACTCCAGAACAATATT |
| | 1xT_6 | AC |
| 387 | side_1_pro_S3_ | CCATCACGAGATAGAATGGTAATACAATCAATAATTTTTT |
| | 1xT_7 | |
| 388 | side1_pro_S3_ | CAAATTAGAACTCAAACTATCGGCCTTTTTT |
| | 1xT_8 | |
| 389 | side2_pro_S3_ | TGTATCGGACTGAGTTAACAACTATTTTT |
| | 1xT_1 | |
| 390 | side2_pro_S3_ | TTTTTTGTTTAACGTCAAAAAATAAGAAATTGCCAGACGCAAC |
| | 1xT_2 | |
| 391 | side2_pro_S3_ | AAGAATTGAGCCTAATCGATTTTTTTTTT |
| | 1xT_3 | |
| 392 | side2_pro_S3_ | TTTTTTGCTAAACAACTTCATTCCAGAGGGTATAGAATTTGTGAGAGCAAACACCACG |
| | 1xT_4 | |
| 393 | side2_pro_S3_ | TTTTTTAACATAAATTATTTATCCCAATCCAATGAAAATAAGAGAGATCAGTACAA |
| | 1xT_5 | |
| 394 | side2_pro_S3_ | ACTACAACAGGCTCCATCACGTTGAAAATCTCCTGTATGGGATTTTTTTT |
| | 1xT_6 | |
| 395 | side2_pro_S3_ | TTTTTAGGAATTGCGAATAATAGAAAGGTCGTCACAACCCACCTCATTT |
| | 1xT_7 | |
| 396 | side2_pro_S3_ | CAAAAAAAGCCTGTAGTCAACAGTTACAGAGAGAATTTTT |
| | 1xT_8 | |
| 397 | side2_pro_S3_ | TGTATCGGACTGAGTTAACAACTATTTTT |
| | 1xT_1 | |
| 398 | side2_pro_S3_ | TTTTTTGTTTAACGTCAAAAAATAAGAAATTGCCAGACGCAAC |
| | 1xT_2 | |
| 399 | side2_pro_S3_ | AAGAATTGAGCCTAATCGATTTTTTTTTT |
| | 1xT_3 | |
| 400 | side2_pro_S3_ | TTTTTTGCTAAACAACTTCATTCCAGAGGGTATAGAATTTGTGAGAGCAAACACCACG |
| | 1xT_4 | |
| 401 | side2_pro_S3_ | TTTTTTAACATAAATTATTTATCCCAATCCAATGAAAATAAGAGAGATCAGTACAA |
| | 1xT_5 | |
| 402 | side2_pro_S3_ | ACTACAACAGGCTCCATCACGTTGAAAATCTCCTGTATGGGATTTTTTTT |
| | 1xT_6 | |
| 403 | side2_pro_S3_ | TTTTTAGGAATTGCGAATAATAGAAAGGTCGTCACAACCCACCTCATTT |
| | 1xT_7 | |
| 404 | side2_pro_S3_ | CAAAAAAAGCCTGTAGTCAACAGTTACAGAGAGAATTTTT |
| | 1xT_8 | |
| 405 | nohandle_1 | CCAATCGCGTCAGACGATTGGCCTTGATATT |
| 406 | nohandle_2 | TCAGAAGCAAGGCTATATTAAATTAATGCCCACGCTGAAGT |
| 407 | nohandle_3 | GAGAATGATAGCATGTAGCCCCAAAAAATAGCGAT |
| 408 | nohandle_4 | CCCCTCAGTGTCGATGCAATGCCTGAGTA |
| 409 | nohandle_5 | TACATGGCCTTAGCCG |
| 410 | nohandle_6 | GTCTCTGAATAAGGGAGAACGGTG |
| 411 | nohandle_7 | CACAAACATATATGTAATATAAGTATAGCCCG |
| 412 | nohandle_8 | TTTTTGGGAAGACAAATCATCGAG |
| 413 | nohandle_9 | CCACTACTATATTTCCAAGAAGCGCCTG |
| 414 | nohandle_10 | ATAACCTAAAAGAACGTGGACTCCAACGTCA |
| 415 | nohandle_11 | AGTCTGGGGTCTTTGGAAGCCCGAATGTTTAGA |
| 416 | nohandle_12 | ATGTGTAGAATGCTTTAATATTCATTGAATC |
| 417 | nohandle_13 | CATGTTATTTTGATGGGGTCAGTGCCTTG |
| 418 | nohandle_14 | GTCAATCATTTACCTAAACAGTTAATGCC |
| 419 | nohandle_15 | GGCTTGCAGGGAGTTATATTCGG |
| 420 | nohandle_16 | AACGAGGGAATAAATCAAGTATTAAGACATTGA |
| 421 | nohandle_17 | CTTAGATTAAGACGCATAAATAA |
| 422 | nohandle_18 | TTTTCAAAGTGAACCACCCTAAAGGGAGCCC |
| 423 | nohandle_19 | CTGACCTAAAAACCGTCGGGGAAA |
| 424 | nohandle_20 | AATTAAGCCTCCAGTATAAAGCCAA |
| 425 | nohandle_21 | GATCTACATGCTTCTTTCAACTTTACATCAAGAAAAC |
| 426 | nohandle_22 | TTAATGCCGGAGAGGGAATTAC |
| 427 | nohandle_23 | GGCCGGAGACAGTCA |
| 428 | nohandle_24 | ATAAATTGTAAAGATTCAAAAGGTGTACCCC |
| 429 | nohandle_25 | AAAGTACAACGGAGATTTGTATCACCTGCTC |
| 430 | nohandle_26 | TGACCCCCAGCGATTGAATTTT |
| 431 | nohandle_27 | AGGCAAAAGAATACACTTTAAT |
| 432 | nohandle_28 | CATTAAACGGGTAAAATTGCGCCG |
| 433 | nohandle_29 | AGGACTAAAGACTCACCCTCAGCAGC |
| 434 | nohandle_30 | TATATAACTAGCAACGGCTACAGGCATCGG |
| 435 | nohandle_31 | TGAATTTATCAAAATTGCAGAACCGGGTATT |
| 436 | nohandle_32 | CTACCTTTTTAACCTC |
| 437 | handle_32H_1 | GCAGTAGAGTAGGTAGAGATTAGGCACCAATCGCGTCAGACGATTGGCCTTGATATT |
| 438 | handle_32H_2 | GCAGTAGAGTAGGTAGAGATTAGGCATCAGAAGCAAGGCTATATTAAATTAATGCCCACGC |
| | | TGAAGT |
| 439 | handle_32H_3 | GCAGTAGAGTAGGTAGAGATTAGGCAGAGAATGATAGCATGTAGCCCCAAAAAATAGCGAT |
| 440 | handle_32H_4 | GCAGTAGAGTAGGTAGAGATTAGGCACCCCTCAGTGTCGATGCAATGCCTGAGTA |
| 441 | handle_32H_5 | GCAGTAGAGTAGGTAGAGATTAGGCATACATGGCCTTAGCCG |
| 442 | handle_32H_6 | GCAGTAGAGTAGGTAGAGATTAGGCAGTCTCTGAATAAGGGAGAACGGTG |
| 443 | handle_32H_7 | GCAGTAGAGTAGGTAGAGATTAGGCACACAAACATATATGTAATATAAGTATAGCCCG |
| 444 | handle_32H_8 | GCAGTAGAGTAGGTAGAGATTAGGCATTTTTGGGAAGACAAATCATCGAG |
| 445 | handle_32H_9 | GCAGTAGAGTAGGTAGAGATTAGGCACCACTACTATATTTCCAAGAAGCGCCTG |
| 446 | handle_32H_10 | GCAGTAGAGTAGGTAGAGATTAGGCAATAACCTAAAAGAACGTGGACTCCAACGTCA |
| 447 | handle_32H_11 | GCAGTAGAGTAGGTAGAGATTAGGCAAGTCTGGGGTCTTTGGAAGCCCGAATGTTTAGA |
| 448 | handle_32H_12 | GCAGTAGAGTAGGTAGAGATTAGGCAATGTGTAGAATGCTTTAATATTCATTGAATC |
| 449 | handle_32H_13 | GCAGTAGAGTAGGTAGAGATTAGGCACATGTTATTTTGATGGGGTCAGTGCCTTG |
| 450 | handle_32H_14 | GCAGTAGAGTAGGTAGAGATTAGGCAGTCAATCATTTACCTAAACAGTTAATGCC |
| 451 | handle_32H_15 | GCAGTAGAGTAGGTAGAGATTAGGCAGGCTTGCAGGGAGTTATATTCGG |
| 452 | handle_32H_16 | GCAGTAGAGTAGGTAGAGATTAGGCAAACGAGGGAATAAATCAAGTATTAAGACATTGA |
| 453 | handle_32H_17 | GCAGTAGAGTAGGTAGAGATTAGGCACTTAGATTAAGACGCATAAATAA |
| 454 | handle_32H_18 | GCAGTAGAGTAGGTAGAGATTAGGCATTTTCAAAGTGAACCACCCTAAAGGGAGCCC |
| 455 | handle_32H_19 | GCAGTAGAGTAGGTAGAGATTAGGCACTGACCTAAAAACCGTCGGGGAAA |
| 456 | handle_32H_20 | GCAGTAGAGTAGGTAGAGATTAGGCAAATTAAGCCTCCAGTATAAAGCCAA |
| 457 | handle_32H_21 | GCAGTAGAGTAGGTAGAGATTAGGCAGATCTACATGCTTCTTTCAACTTTACATCAAGAAAA |
| | | C |
| 458 | handle_32H_22 | GCAGTAGAGTAGGTAGAGATTAGGCATTAATGCCGGAGAGGGAATTAC |
| 459 | handle_32H_23 | GCAGTAGAGTAGGTAGAGATTAGGCAGGCCGGAGACAGTCA |
| 460 | handle_32H_24 | GCAGTAGAGTAGGTAGAGATTAGGCAATAAATTGTAAAGATTCAAAAGGTGTACCCC |
| 461 | handle_32H_25 | GCAGTAGAGTAGGTAGAGATTAGGCAAAAGTACAACGGAGATTTGTATCACCTGCTC |
| 462 | handle_32H_26 | GCAGTAGAGTAGGTAGAGATTAGGCATGACCCCCAGCGATTGAATTTT |
| 463 | handle_32H_27 | GCAGTAGAGTAGGTAGAGATTAGGCAAGGCAAAAGAATACACTTTAAT |
| 464 | handle_32H_28 | GCAGTAGAGTAGGTAGAGATTAGGCACATTAAACGGGTAAAATTGCGCCG |
| 465 | handle_32H_29 | GCAGTAGAGTAGGTAGAGATTAGGCAAGGACTAAAGACTCACCCTCAGCAGC |
| 466 | handle_32H_30 | GCAGTAGAGTAGGTAGAGATTAGGCATATATAACTAGCAACGGCTACAGGCATCGG |
| 467 | handle_32H_31 | GCAGTAGAGTAGGTAGAGATTAGGCATGAATTTATCAAAATTGCAGAACCGGGTATT |
| 468 | handle_32H_32 | GCAGTAGAGTAGGTAGAGATTAGGCACTACCTTTTTAACCTC |

TABLE 2C: Staple sequences for triangle 1, version 3

| SEQ ID | | |
|---|---|---|
| NO: | Description | Sequence/Details |
| 469 | core_1 | TAACCTCC |
| 470 | core_2 | GAGACGCAGAAACAGCGGATACCGGCGAAAGAGGTGG |
| 471 | core_3 | TACCGGGTAAAGGGAGCCCTGAGACAAGAG |
| 472 | core_4 | GATAGACGCAGGCGACGCCTGTCGATCCAGAAAAAAAT |
| 473 | core_5 | ACTAACAATTTTAGTTTCCTAATTTAAAAGGGACATTCTGATTACCGCTTGAC |
| 474 | core_6 | TCCCCGGGTACCGAGCTCGATGTGAAAGCAACAGGAAAAACGTCACACGA |
| 475 | core_7 | GTATCATAATTAATTTTCCCTTATTTTTAATCCTTGAAAACATCAGGAACG |
| 476 | core_8 | AAGAAAAATAATAATGGAAGGGTTTTTTAGAACCTACCAAAGTCCTGAAC |
| 477 | core_9 | AATTGGGCTTGAGATGGTTTACGACCTGCTCCATGTCGGTCGCTGAGGCTT |
| 478 | core_10 | TCATATTCATTCCAAGAACCCTCATATAATAACGGCGGGTAGATGGGCGC |
| 479 | core_11 | GGCTTATGGTTGCTCAGCCATTTTGTTATCCGCTCACACGTGCCGGACTTGTAG |
| 480 | core_12 | AGGAATTGTGAGAATACTCAAGAGAAGGATTTTTGATGATTTTGCGGCCCGAA |
| 481 | core_13 | CACCCTCACGGGAACGTTTGAATTGAGAGGCTTAGCAACGCATGAGGA |
| 482 | core_14 | GCGTCTTGAGTCTCCAAAAAAAGAACCGCCACCCTGAGGTGCCGTGAATAGG |
| 483 | core_15 | TTATCAACAATAGATTATCAAAAATCTAAAATATTTTTTTTTAGGAGC |
| 484 | core_16 | AGGGCGAATGGTAATGGGTAAAGGTTTTTTTCTTTGCTCGTTCACAGTTGAGGA |
| 485 | core_17 | GCTTCTGTCGTGGTGAAGGGATATCACTGCGAAATCCTGCTTATAAATCAAAAG |
| 486 | core_18 | TAGCCCTAAATGATAGAAAAAGTTTTTCTGTTTAGTATAAAGAACCACC |
| 487 | core_19 | ATTAACCTAATGAATCGGCCAAGGGTAAAGCAGTTTTTGGTGCCGGTGCCCCCT |
| 488 | core_20 | ATTGACCGTAATGGGGGATAAGATGTATCAGAGAGATAAAATCAAGATTTATTC |
| 489 | core_21 | ACCCACACAACGCATCCAGTAAAAGCGCAGTCTCTGAAATAGGTCGACTTTAC |
| 490 | core_22 | ATGAACGCGCTTTTTGGGAGAAAAGAGTTTTTCTGTCCAGCCATTAAAA |
| 491 | core_23 | AAATGCTAAAAGGGAATACCTATTCAGGGCATAGCTCTGGTCTGCTGTTGCC |
| 492 | core_24 | TTGTAAAGGAAACCAGGCAAAGCGCCATTCTGAACCTCATAATTACGAATCGC |
| 493 | core_25 | CCATTACCATTTTTTAGCACGCAATTTTTTATAACGGGTGTCTGGAAG |
| 494 | core_26 | TGCTCCTTGGGCGACAAATTAATTACATTGCCACCCTCAGCCGCCACCAGAAC |
| 495 | core_27 | TTATATAAACTTTTTCCCATCAAACCAGTTTGAGGGGACGACGACAG |
| 496 | core_28 | AAAATCAGGTCTTTAGAGCCGCCCGTATAAACAGTTAATGCCCGCTTTAAACA |
| 497 | core_29 | TTGCTCAGTACCAGGCTTTACCGTAGGATAAAAAACAATTGGAGCGGAATTATCA |
| 498 | core_30 | AAGCAAAAACGGTTTTTAATCGGCCTTTTTTTCACCGCCTGTTTTAGA |
| 499 | core_31 | CGCCTGCAACAGTGGCGAATTATTAACCTTGTACCCCGTTGTGAGA |
| 500 | core_32 | TGCCGTCGAGAGGATTCAACCGTGCCTTCCCGCAAGACCATTTGAG |
| 501 | core_33 | GAATGGAGCGTCATACATGGCTAGGATTAGGTTAAGCCTAATTTTT |
| 502 | core_34 | TCGAGAACTCCTTATCCTGATTAGATTTAGAAGTATTAACGTTGGT |
| 503 | core_35 | TTTTGTTAAATCAGCGAGATCTTCATAGGTTGCTTTCCCTCAAACT |
| 504 | core_36 | AATTCTACTTTTTAAATATGCAACTAAAGTACGAATACCCAAAAGAACCAGTAGCA |
| 505 | core_37 | GAGATAGTTTTTTGTTGAGTGCCGGGTTACACGGTCAAGCTGCAT |
| 506 | core_38 | AATCGATGTATTTAAACGGAATCAAATATCAATATACAGTAACAG |
| 507 | core_39 | AGCCGCCACAAGGCGACATCAAAAAAAGATGAGAGTACCTTTAAT |
| 508 | core_40 | GGAAATTCCTTTTTCACAACATACGAGCCGCACATCCTCATAAC |
| 509 | core_41 | AGCATTAACATCCAATAAATCAATTGCTGAATATAATGCTGTAGCTCCTTAT |
| 510 | core_42 | CACCACCACCCTGACTGTTCAGAAATACATAAAAAGGTGAATAGAAAACCTAAAAC |
| 511 | core_43 | ATAACCGATATATTTACTTAGCCTTTTTGAACGAGGCGAGTA |
| 512 | core_44 | CATGAAAGTATTTTTAACTTGTACCACTGTTTAGACATTTCG |
| 513 | core_45 | ATACCGAACGCCAACGCTCTTTTTACAGTAGGGCACATCGGGAGAAA |
| 514 | core_46 | TAATGCGCCGCTACAGGAAATGCCGGAGAGGGTAGCTATTTTTTTTTGATCATTTTT |
| 515 | core_47 | CACGCTGGTTTGCCCCATTTGCAACAGCTGATTTAAAACTAGCATGTCAGCCCCAAA |
| 516 | core_48 | AAGCCGGCGCGTGGCGAGAAGCCTCCCATAAACGCCTCCGG |
| 517 | core_49 | ACCATCGACCGAACAAAGTTACCAAATTCTGCGGTGGCATC |
| 518 | core_50 | GTTGTAGCAATACTTCTTAATGCGGCTTAGATATAAACAC |
| 519 | core_51 | GACAAAATTGATAAGTCAGAAGCAAAGCGGAACCCTCAGAGAACCGC |
| 520 | core_52 | ACCAGAGCTTGCCAGATCCCCCTCTATTCATTAGGCCAAA |
| 521 | core_53 | ATCATTTTGCGGAACAAAGAAACGCGAGGCGCTTTATTTAGAATTGA |
| 522 | core_54 | ACGTGGCATAGAAGAATCGTTAGCGTCCGTGAAGGAAGGTGGACTCCAACGTCAA |
| 523 | core_55 | GCTGCGCAACTGTTGGGAAGGGCGAGAGCCACGCTGAGA |
| 524 | core_56 | AAACAATCAAAATAGCACCTTTTTTAATGGATGAAACAAATTAAGTT |
| 525 | core_57 | GAAAGAGGCCCCAGCGAGATTTGTATGCGATTAGGAATT |
| 526 | core_58 | ACATTTTGACGCTCAATCGTTTTGAGTAATACTGAACAAAATCGGATCACCCAAATCAAGT |
| 527 | core_59 | AGCCAGCAAAATCACTGGCATGATTAAGACTCAACATGAATAGTAGT |
| 528 | core_60 | CAGAACACCGAGTAGGCGGTTTGCGTATGCACAGGCGGCCTTTAGTG |
| 529 | core_61 | GATTGTTTGGATTATACTTCTGAATATCCCAAATTTCAT |
| 530 | core_62 | AGCGATACGAACTGACATATTTAACAACGCCGTCAGATG |
| 531 | core_63 | ATAGTAAAACGAGGCATGTATGGGATTTTTTTTGGCCATCTTTTTTTTCATAATT |
| | | ATTCTGAAA |
| 532 | core_64 | CAAATGGTAGTTTGAGTAACATTCGTTATTAATTTTAAA |
| 533 | core_65 | ATCGGCCTCAGGAAGAGGAATTGAATGGTTTGGCGCCTGTGTAAGAAT |
| 534 | core_66 | TCAGATGATGGCAATTCATCAATACCAATCAGCGAGAAA |
| 535 | core_67 | GCTCTCACCCCGGAATTTGTAAAACGACGGCTTTCCCAG |
| 536 | core_68 | GAAAGGATAGCGTTTCTAAACAATTTATCAGACAAGAACAAGCTGCT |
| 537 | core_69 | TTTTGTTATGAGAGTCTGAGAAGAAACAGGAGTAATAA |
| 538 | core_70 | CATTAAATCAGCCAGCTTTCCGGCACCGCTTTCTGGTC |
| 539 | core_71 | GTTCTTCGAATCAGAGTGCGCGTTATCAGGTCATTGCC |
| 540 | core_72 | CGTCAATAGCATCTGAATAATTCGCGTCTGTCTAGCTG |
| 541 | core_73 | ATCAATATAAAGATTCGATGCAAAATTTACAT |
| 542 | core_74 | GAACAATGCCAACAGTTTTTGATAGAACCCAATATCCA |
| 543 | core_75 | AACAGGTCAGGATTATAAGAGGAAGCCCGA |
| 544 | core_76 | AACAGTACAGCTTTCACGTACAGCGCC |
| 545 | core_77 | GCTGGCGAGGGTAACGCCAGGGTCAGTGCCAATAAATC |
| 546 | core_78 | GCTGAAAAGAACGAGTAGATTTAGTTTGACCATTAGAT |
| 547 | core_79 | ATGAACGGATGTTTAGACTGGATAGCGTTTATTAAAGG |
| 548 | core_80 | ATTATAGAGGTCATTTTTGCGGATGGCTTAGAAAATAC |
| 549 | core_81 | ATTCGAGCATCGGTGGTTGATAATCAGAAAAATCATAT |
| 550 | core_82 | CCGAGGAAAAGGCCGGAAACGTCAACCATCGCCCACGC |
| 551 | core_83 | CTATATGTATAAATTGACAGTCAAATCACCCCCGATTT |
| 552 | core_84 | CACCGGAGAATTTTCTAGTAAGACGCCAAATTAAGAAC |
| 553 | core_85 | AAATTCGGATAAATAAGTTGGCATTGCGTAG |
| 554 | core_86 | GTGGTCGGGAAACCTCGGCAAACTAACGGCA |
| 555 | core_87 | ATTCGTAAGCCGGGCGCGTACTAGGTTGGGCCAGTAA |
| 556 | core_88 | CAATCATAGCCGACAATGACAACACCAATGAA |
| 557 | core_89 | GAAATAGCCCTTATACAACTAATGTATCGGCTTTCAA |
| 558 | core_90 | ATGACCATAACAGTGCCGCCAGCATTGACAGGAGGTT |
| 559 | core_91 | CGGGGTTCGTACTCAGGAGGTTTAGTAGAGCGAATAA |
| 560 | core_92 | TGACCTTCATCAAAGGCTTGCCCTGACGAG |
| 561 | core_93 | GTTACAAAATCGCGCTTCAAAGCGAACCAGCGTTTTA |
| 562 | core_94 | ATGTTACAGCCCTTCTGCTCAAGGTAGAGAACTAAC |
| 563 | core_95 | TAACCAATAGGAACGAAATATACTAATAG |
| 564 | core_96 | AGTTTCCGTCAGGACCATTCAAC |
| 565 | core_97 | CACCGGGTTTTGTTTACCAGAGAATACCAGTTGGGA |
| 566 | core_98 | ATCAAAAACAAAGGCAACCACCACACCCGCCGCGC |
| 567 | core_99 | CTCAGAGCCCCTGCCTATTTTCATTGAAGGGGGTA |
| 568 | core_100 | GAGGCAGGTCAGACGATTGGCCTTGATACAATAAC |
| 569 | core_101 | CAATAACGGATTCGCCTGATTGCTTTGTCTTACCA |
| 570 | core_102 | ACAGAATATTAGCAAAATTTTTTAAGCAATAAAGC |
| 571 | core_103 | TTTCATTCCATATAACAGTTGATTCCCGAAGGAAA |
| 572 | core_104 | CCATTTGGGAATTAGGCAGGGAGGCGTCAGACTG |
| 573 | core_105 | AGCAGAAGTGAGGCTCCTGAGACGGCCAGAATGC |
| 574 | core_106 | GAGAATATAAAGTGACAAGTGAATTTGACCGTGT |
| 575 | core_107 | AAGCGCATCCGGGTCAGTCAGCAG |
| 576 | core_108 | CAATAGCTATCTTACCTAAATCGGGGGGTCAGT |
| 577 | core_109 | ATCGTAACCGTGATAATAAAAGAAC |
| 578 | core_110 | TATCATAAAAGAAGTTCACCACCGGAACAACCT |
| 579 | core_111 | AAATCTAAAGTTTTTATCACCTTGC |
| 580 | core_112 | CATAAAGTGTAAAGCCGCTGGCAATCCCTTA |
| 581 | core_113 | GGCGGGCCGTTTTCCTGCAGCTTAAACGA |
| 582 | core_114 | TTCTGACCTAAATTTAGGAAGGTTTTATTTGC |
| 583 | core_115 | TCATTTCATCACGACGCGGGCCTCTTCGCTAT |
| 584 | core_116 | ATTACCTGAAATATCGACCGGAAGCAAACTCC |
| 585 | core_117 | GCGAAAGGGGTCACGCCGGGAGCTAGTCAATA |
| 586 | core_118 | GAGCACGTATAACGCTGAGAGATGAAAGC |
| 587 | core_119 | AAACCCTCAATCAATACTGGTGCCCGTTAATA |
| 588 | core_120 | TTCTGACCCTACCTTTTTTTT |
| 589 | core_121 | AAAGCACTCCCTGAATCAATCCGCACCGT |
| 590 | core_122 | TTCACAAAGTGTACTGGTAATAAGTAAGAGGC |
| 591 | core_123 | GCCTTGAGTAAATCATACAGGCAATACGCAGT |
| 592 | core_124 | AGCGGGCGCCAGCACGCAAATCGTGCGGTCC |
| 593 | core_125 | TCAGGGATAGCAAGTTTTTCCAATAGGAACCC |
| 594 | core_126 | TACCTTTTTTAATACATCTCACGCAAGTACGC |
| 595 | core_127 | ACCCTCAAGGCTCCAATCAGCGGAGCTTACG |
| 596 | core_128 | ATTGTGTCGAAATCCGATTTCAACTTTAATC |
| 597 | core_129 | TCCAGAGCGCTACAATTTTATCCAAGCCTT |
| 598 | core_130 | CTTGCTTTCGAGGTGATTTTCGGTCATAGCC |
| 599 | core_131 | CAATAATAAGAGCAATGTAATACTACAGGA |
| 600 | core_132 | TACGAGCATGTAGAAATAATCCTATTAGAGC |
| 601 | core_133 | TAAGACGCTGGAGCAAGCTGGCAAGTGTAGC |
| 602 | core_134 | GGTTTCTGCTAGGGCGTTTGGAACAAGAGT |
| 603 | core_135 | ATTTTCAGGTTTAACAACATGTAAATAAGA |
| 604 | core_136 | CCCTCGTCGTCTTTCGAGTTTCGTCACCAGT |
| 605 | core_137 | TAAGAACCACCAGAACGACAACTCGTATTA |
| 606 | core_138 | TACGCCAAAGACTTCAGCAAAAGAAGATGA |
| 607 | core_139 | TCATGGTAGAGCTTCTATCAGGGCGATGGC |
| 608 | core_140 | GGCAAAGACAAGTTTGCCTCCAATACTGCGG |
| 609 | core_141 | AAAACATTATGACCCGAAACAATTGAGACTC |
| 610 | core_142 | GTGTAGGTGATGTTGATATAAGTATAGCCCG |
| 611 | core_143 | TACCAGTGGAAAAAAATATATGTGAGTGAA |
| 612 | core_144 | AACGAGATAAATATCGGAACCTATCAAAAT |
| 613 | core_145 | ACGTAAAACAGAAATTGCAGAACAAATACC |
| 614 | Side1_pro1 | AATTTTTAGAACGGGTATTAGTTGCAAATCAGATATAGAAGGCGGGT |
| 615 | Side1_pro2 | TTGATTATCCGGAAACCAAGTACCGCACTCAATAGCAAGCTACTGAAATGGATTTCCATTTT |
| | | AAATGCA |
| 616 | Side1_pro3 | TTGATTAACGTCAAAAATGAAGAAACGACCAGTTACAACGGCAGCCGGGCG |
| 617 | Side1_pro4 | ATAATCGTGGCAGATCGTAGGAAGAGAGAATAATTTT |
| 618 | Side1_pro5 | TCGGTCATTCACCAGCTCATGGATGAGAAAG |
| 619 | Side1_pro6 | TTTCCATAAAAAATTTATCCCAATCCAAATAAAATAGCAGCCTTTACATCATTACCGCGCCCA |
| 620 | Side1_pro7 | GCTGTCTTAAGCAAGCCGTTTTTATTTTCAT |
| 621 | Side1_pro8 | AATTGAGCGCTAACCTTGCACCCACTAATTTGTTTTTTGTGGAT |
| 622 | Side1_rec1 | TTACCGCATCGGCAATTTCTTGAACTGTTTTTCCAACTTTGAAAGATAGGCTGGC |
| 623 | Side1_rec2 | CAAATAAATCCTCATTAAAGCCAAATCCTTTGGAGAAGCTTTTAGCGAAT |
| 624 | Side1_rec3 | TGCTAACGACGCAACCGGTCATT |
| 625 | Side1_rec4 | TAGTAAATAAAGCGAAGCCCCCGTAATCAGTAGCG |
| 626 | Side1_rec5 | CTATATTTTCATTTGGGGCGCGACTCAGAGCGCAGATAGTAGCAGCATTTTTT |
| 627 | Side1_rec6 | TGACTTGCGGGAGGTTTTGTGAATCTTTCACGTTGAAAAGGTTGTATCAC |
| 628 | Side1_rec7 | TAGCGCGTTTTATAGTTGCAGGGAACCAAACAT |
| 629 | Side2_pro1 | CTGATAACCGCTTTTTACACTAATGAT |
| 630 | Side2_pro2 | TGCTACACTCATCTTTGACCAAAAGAAGCGGGATCCCGTCACCAATCGTCA |
| 631 | Side2_pro3 | TCCTTACCAGCGTGGCAACATATAAAAGAAACACAATCAATTATCAGTCACCC |
| 632 | Side2_pro4 | TCAGCAGCACAACGGATTATACCAAGCGCGAGGTAAAATACGTAAACT |
| 633 | Side2_pro5 | AACAAAGTGAAAGACAGGCACCAATTCATATGGTTCCCT |
| 634 | Side2_pro6 | TACCATGCCACTACGAAGCATCGGCGGAAATAAATTAACAATTTCAGATCGCCTCC |
| 635 | Side2_pro7 | TAAGAATAAGTTTATTTTGTCGCAAAGAAAACGTAGAGCTTA |
| 636 | Side2_pro8 | GACTTGAGATGTTAGCCACCACGGAAAT |
| 637 | Side2_rec1 | TGTATTAACACTACAAATAATACCAAGCCAGCAGCAAATGAACCATTCAGGATTGTA |
| 638 | Side2_rec2 | TCTAAAAAACAAACTTAAATTCATAGTTTGTAGCATTCCACAG |
| 639 | Side2_rec3 | AAGGGGGATGTGCTGGAGCCACCTTGCATCGAAAACAATTCAACCGATT |
| 640 | Side2_rec4 | CGACGATAGACTTTTTGCTACAGAGGCTTT |
| 641 | Side2_rec5 | TAGGGAAGGTAAATATTGAAACGAGGGTTTGCAA |
| 642 | Side2_rec6 | TGGGCGCCTGGTGCTGAGTGTTTTGACGGCTCAAATCGTCAGAGGTGAGGT |
| 643 | Side2_rec7 | TTCACCAGTGATATAATCAGATAAAACGCTATTATGCGTTAAACAGGAA |
| 644 | Side3_pro1 | GCCTGAGCAACCGACAACATT |
| 645 | Side3_pro2 | AGGCGTTCAATAAACAATGGCTAAGTAATAAGCTCACTGCCCGTAT |
| 646 | Side3_pro3 | TATTTTTGAACATGTTTCTGTCCAGACGACGAAATTTAGGCAGAGTTTT |
| 647 | Side3_pro4 | TAACAAGGTAAAGTAATCAGCTAAAAAGAAAAATCAACAGTTGAAATCGCACTC |
| 648 | Side3_pro5 | TCTCGCTTTCCAAGCTAATGACATCACTTTGCGTTGC |
| 649 | Side3_pro6 | TGGGGCATTTTCGAGCCTTAGTCTTTTGATTAGGCCGAT |
| 650 | Side1_pass_ | AATTTTTAGAACGGGTATTAGTTGCAAATCAGATATAGAAGGCTTTTT |
| | pro1 | |
| 651 | Side1_pass_ | TTTTTTTATCCGGAAACCAAGTACCGCACTCAATAGCAAGCTACTGAAATGGATTTCCATTTT |
| | pro2 | AAATGCA |
| 652 | Side1_pass_ | TTTTTTTAACGTCAAAAATGAAGAAACGACCAGTTACAACGGCAGCCGGGCG |
| | pro3 | |
| 653 | Side1_pass_ | ATAATCGTGGCAGATCGTAGGAAGAGAGAATAATTTTT |
| | pro4 | |
| 654 | Side1_pass_ | TTTTTTCATTCACCAGCTCATGGATGAGAAAG |
| | pro5 | |
| 655 | Side1_pass_ | TTTTTCATAAAAAATTTATCCCAATCCAAATAAAATAGCAGCCTTTACATCATTACCGCGCCC |
| | pro6 | A |
| 656 | Side1_pass_ | GCTGTCTTAAGCAAGCCGTTTTTATTTTTTTT |
| | pro7 | |
| 657 | Side1_pass_ | AATTGAGCGCTAACCTTGCACCCACTAATTTGTTTTTTGTTTTTT |
| | pro8 | |
| 658 | Side2_pass_ | CTGATAACCGCTTTTTACACTAATTTTT |
| | pro1 | |
| 659 | Side2_pass_ | TTTTTACACTCATCTTTGACCAAAAGAAGCGGGATCCCGTCACCAATCGTCA |
| | pro2 | |
| 660 | Side2_pass_ | TTTTTTACCAGCGTGGCAACATATAAAAGAAACACAATCAATTATCAGTCACCC |
| | pro3 | |
| 661 | Side2_pass_ | TCAGCAGCACAACGGATTATACCAAGCGCGAGGTAAAATACGTATTTTT |
| | pro4 | |
| 662 | Side2_pass_ | AACAAAGTGAAAGACAGGCACCAATTCATATGGTTTTTTT |
| | pro5 | |
| 663 | Side2_pass_ | TTTTTATGCCACTACGAAGCATCGGCGGAAATAAATTAACAATTTCAGATCGCCTCC |
| | pro6 | |
| 664 | Side2_pass_ | TTTTTAATAAGTTTATTTTGTCGCAAAGAAAACGTAGAGCTTA |
| | pro7 | |
| 665 | Side2_pass_ | GACTTGAGATGTTAGCCACCACGGTTTTT |
| | pro8 | |
| 666 | nohandle_1 | GGAACAACTGTGTACATCGACATCGCAGTG |
| 667 | nohandle_2 | TGTACAGACCAGGCGCAGGACAG |
| 668 | nohandle_3 | CAACCGCAAGAATGCCAAAATAAGAGAATTA |
| 669 | nohandle_4 | CATTACCCTCATTTCAGTTTCAGCGCCGCC |
| 670 | nohandle_5 | CGGTGGTGCCATCCCA |
| 671 | nohandle_6 | ATTGTGAATTACCTTATCATCGC |
| 672 | nohandle_7 | GCAGGCGCGTCCAGAACCGCCACCCTCAGAG |
| 673 | nohandle_8 | TGGGCGGTCAAAATCCTTTGATGGTGGTTCC |
| 674 | nohandle_9 | CTGCGGCAAACCGTGACGGGGA |
| 675 | nohandle_10 | TAATGCATAACACTCAGACGTTAGTAAAT |
| 676 | nohandle_11 | AGAAAAATCTACGTTAATAAAACAAGATTCA |
| 677 | nohandle_12 | CATTCAGTGAATAGAGTAATCTTG |
| 678 | nohandle_13 | CCCGTAAAAAAAGCC |
| 679 | nohandle_14 | TGGCTCATTATACCAATTAAACG |
| 680 | nohandle_15 | AAACACCAGAACGAGTCAGACGGT |
| 681 | nohandle_16 | CGGTTGCGGTGAACCAACCCTAAAGGGAGCC |
| 682 | nohandle_17 | CCACCACCTGTCCAGCAAAGGAGCCTTTAAT |
| 683 | nohandle_18 | TCAGTTGAACAACGCCAGCGTAACGATCTAAA |
| 684 | nohandle_19 | TGCTGATTGCCGTTCGTCGTGCC |
| 685 | nohandle_20 | CCACTATTTGTTCAGCGTGCCTCCTAACTCACATTAACT |
| 686 | nohandle_21 | AATAGCCCGCATCAGAGCACTCTGAGGGTGGTTT |
| 687 | nohandle_22 | GAAATCGGATTATTACTTTGCCGCCAGCAGT |
| 688 | nohandle_23 | ACAAACTGATTTAG |
| 689 | nohandle_24 | ATGTACCGGATACATAAGCAACAC |
| 690 | nohandle_25 | TCAGATGTTGTTCCAAGAGTTGCAGCAAGCGGTC |
| 691 | nohandle_26 | TTTTTGGGTTTCGCACCAAAGTCA |
| 692 | nohandle_27 | CCACTACGTATGAGTAGACGGACAGCCAT |
| 693 | nohandle_28 | CACTGGTGAAAGAACGGAAGAAA |
| 694 | nohandle_29 | CCAGAGGAAGATCGGCCTTGCTGGT |
| 695 | nohandle_30 | GCTGGAGGCAAATCAACGTAACACGGATATT |
| 696 | nohandle_31 | GTTTTTTCGTCTCGTCTGGGGTG |
| 697 | nohandle_32 | AACGTCAGCGTGGTGGTTTCCTG |
| 698 | handle_32LH_1 | GCAGTAGAGTAGGTAGAGATTAGGCATTTTTTTTTTTTTTTTTTTTGGAACAACTGTGTACAT |
| | | CGACATCGCAGTG |
| 699 | handle_32LH_2 | GCAGTAGAGTAGGTAGAGATTAGGCATTTTTTTTTTTTTTTTTTTTGTACAGACCAGGCGCA |
| | | GGACAG |
| 700 | handle_32LH_3 | GCAGTAGAGTAGGTAGAGATTAGGCATTTTTTTTTTTTTTTTTTTTCAACCGCAAGAATGCCA |
| | | AAATAAGAGAATTA |
| 701 | handle_32LH_4 | GCAGTAGAGTAGGTAGAGATTAGGCATTTTTTTTTTTTTTTTTTTTCATTACCCTCATTTCAGT |
| | | TTCAGCGCCGCC |
| 702 | handle_32LH_5 | GCAGTAGAGTAGGTAGAGATTAGGCATTTTTTTTTTTTTTTTTTTTCGGTGGTGCCATCCCA |
| 703 | handle_32LH_6 | GCAGTAGAGTAGGTAGAGATTAGGCATTTTTTTTTTTTTTTTTTTTATTGTGAATTACCTTATC |
| | | ATCGC |
| 704 | handle_32LH_7 | GCAGTAGAGTAGGTAGAGATTAGGCATTTTTTTTTTTTTTTTTTTTGCAGGCGCGTCCAGAAC |
| | | CGCCACCCTCAGAG |
| 705 | handle_32LH_8 | GCAGTAGAGTAGGTAGAGATTAGGCATTTTTTTTTTTTTTTTTTTTGGGCGGTCAAAATCCT |
| | | TTGATGGTGGTTCC |
| 706 | handle_32LH_9 | GCAGTAGAGTAGGTAGAGATTAGGCATTTTTTTTTTTTTTTTTTTTCTGCGGCAAACCGTGAC |
| | | GGGGA |
| 707 | handle_32LH_10 | GCAGTAGAGTAGGTAGAGATTAGGCATTTTTTTTTTTTTTTTTTTTAATGCATAACACTCAGA |
| | | CGTTAGTAAAT |
| 708 | handle_32LH_11 | GCAGTAGAGTAGGTAGAGATTAGGCATTTTTTTTTTTTTTTTTTTTAGAAAAATCTACGTTAAT |
| | | AAAACAAGATTCA |
| 709 | handle_32LH_12 | GCAGTAGAGTAGGTAGAGATTAGGCATTTTTTTTTTTTTTTTTTTTCATTCAGTGAATAGAGTA |
| | | ATCTTG |
| 710 | handle_32LH_13 | GCAGTAGAGTAGGTAGAGATTAGGCATTTTTTTTTTTTTTTTTTTTCCCGTAAAAAAAGCC |
| 711 | handle_32LH_14 | GCAGTAGAGTAGGTAGAGATTAGGCATTTTTTTTTTTTTTTTTTTTGGCTCATTATACCAATT |
| | | AAACG |
| 712 | handle_32LH_15 | GCAGTAGAGTAGGTAGAGATTAGGCATTTTTTTTTTTTTTTTTTTTAAACACCAGAACGAGTC |
| | | AGACGGT |
| 713 | handle_32LH_16 | GCAGTAGAGTAGGTAGAGATTAGGCATTTTTTTTTTTTTTTTTTTTCGGTTGCGGTGAACCAA |
| | | CCCTAAAGGGAGCC |
| 714 | handle_32LH_17 | GCAGTAGAGTAGGTAGAGATTAGGCATTTTTTTTTTTTTTTTTTTTCCACCACCTGTCCAGCA |
| | | AAGGAGCCTTTAAT |
| 715 | handle_32LH_18 | GCAGTAGAGTAGGTAGAGATTAGGCATTTTTTTTTTTTTTTTTTTTCAGTTGAACAACGCCA |
| | | GCGTAACGATCTAAA |
| 716 | handle_32LH_19 | GCAGTAGAGTAGGTAGAGATTAGGCATTTTTTTTTTTTTTTTTTTTGCTGATTGCCGTTCGT |
| | | CGTGCC |
| 717 | handle_32LH_20 | GCAGTAGAGTAGGTAGAGATTAGGCATTTTTTTTTTTTTTTTTTTTCCACTATTTGTTCAGCGT |
| | | GCCTCCTAACTCACATTAACT |
| 718 | handle_32LH_21 | GCAGTAGAGTAGGTAGAGATTAGGCATTTTTTTTTTTTTTTTTTTTAATAGCCCGCATCAGAG |
| | | CACTCTGAGGGTGGTTT |
| 719 | handle_32LH_22 | GCAGTAGAGTAGGTAGAGATTAGGCATTTTTTTTTTTTTTTTTTTTGAAATCGGATTATTACTT |
| | | TGCCGCCAGCAGT |
| 720 | handle_32LH_23 | GCAGTAGAGTAGGTAGAGATTAGGCATTTTTTTTTTTTTTTTTTTTACAAACTGATTTAG |
| 721 | handle_32LH_24 | GCAGTAGAGTAGGTAGAGATTAGGCATTTTTTTTTTTTTTTTTTTTATGTACCGGATACATAA |
| | | GCAACAC |
| 722 | handle_32LH_25 | GCAGTAGAGTAGGTAGAGATTAGGCATTTTTTTTTTTTTTTTTTTTCAGATGTTGTTCCAAGA |
| | | GTTGCAGCAAGCGGTC |
| 723 | handle_32LH_26 | GCAGTAGAGTAGGTAGAGATTAGGCATTTTTTTTTTTTTTTTTTTTGGGTTTCGCACCA |
| | | AAGTCA |
| 724 | handle_32LH_27 | GCAGTAGAGTAGGTAGAGATTAGGCATTTTTTTTTTTTTTTTTTTTCCACTACGTATGAGTAG |
| | | ACGGACAGCCAT |
| 725 | handle_32LH_28 | GCAGTAGAGTAGGTAGAGATTAGGCATTTTTTTTTTTTTTTTTTTTCACTGGTGAAAGAACGG |
| | | AAGAAA |
| 726 | handle_32LH_29 | GCAGTAGAGTAGGTAGAGATTAGGCATTTTTTTTTTTTTTTTTTTTCCAGAGGAAGATCGGCC |
| | | TTGCTGGT |
| 727 | handle_32LH_30 | GCAGTAGAGTAGGTAGAGATTAGGCATTTTTTTTTTTTTTTTTTTTGCTGGAGGCAAATCAAC |
| | | GTAACACGGATATT |
| 728 | handle_32LH_31 | GCAGTAGAGTAGGTAGAGATTAGGCATTTTTTTTTTTTTTTTTTTTGTTTTTTCGTCTCGTCTG |
| | | GGGTG |
| 729 | handle_32LH_32 | GCAGTAGAGTAGGTAGAGATTAGGCATTTTTTTTTTTTTTTTTTTTAACGTCAGCGTGGTGGT |
| | | TTCCTG |

TABLE 3
Staple sequences for triangle 2
TABLE 3A: Staple sequences for triangle 2, version 1

| SEQ | | |
|---|---|---|
| ID NO: | Description | Sequence/Details |
| 730 | core_1 | TGACCGTATTTGAGGGGACGACGGCAGAAGTTTTGTTTAACGTG |
| 731 | Core_2 | TCAGTTGAGGAGCAAAATAACCGACCGACCGTAATAATTTTTTCACGTT |
| 732 | core_3 | GATTTAGGATCTACGTTAATAAAAAGAGCCAGGCCCACGCTTTACATTGGCAGATTT |
| 733 | core_4 | AAGCAATAAAGCCTCAGAGCATAAATGCAGAACAAAGGC |
| 734 | core_5 | GTAAGCAAGCTGGCGGTTATCCGGTATTCGCTGAGACTCCTTTGCCCCAGCA |
| 735 | core_6 | AGAGCACATCCAACAATCGCGGAAAAAGCGCTAGGTTTTTCGCTGGGTGGTGAAGGGAT |
| 736 | core_7 | CATGTTACATTCCATAGCGAACCAGACCGGAAGCCTGATTTCGCAAGA |
| 737 | core_8 | TTAGCCGGTCGCCTGAAACGGCTAATATATTTTAGTTAATTTTTTTCATCTTCTGACCTAAAT |
| 738 | core_9 | AACGAGGCCATTCAGTGAATAAGGGAATTGAGTCTTAAACAGCTTGATAT |
| 739 | core_10 | GTAAGAGCTGAATTACCTTATGCGAATCTTTTTAATAGTAAATTGGGCTTGAGGAGGGGGT |
| 740 | core_11 | CAGCCTTTACAGAGAGAATAACGAGGAAACAGCCCAATAATAAGAGTTTGCTCA |
| 741 | core_12 | TAACTTTAATCATTGAACACTAT |
| 742 | core_13 | TCATTACCCAAATCAAGGAGCACTATCGGTTTAGTAATAAACAATTCGAGGAAAT |
| 743 | core_1 | TAATCTTGCAATAGATTTAAAAGTTCCTTTGCTTTCTGTATGGGATT |
| 744 | core_15 | GAATCTTACCAACGCTTGGCCTTCCGCACTCGCCATCTTAATATCCCAACATGTTCAGC |
| 745 | core_16 | GCTGCGCAAGGAGCGGAGAACGTCTCCTGTTTGAGTTGCAGCAAGCGGTCCACGCT |
| 746 | core_17 | TCATACAACCAGGCAAAGCTTTTTCCATTCGCCATTCAGTTTTCACGG |
| 747 | core_18 | GAGGTGAGGCGGTCAGACCGTGCAGGTTGATATAGCATGT |
| 748 | core_19 | TATTAACAACGTTAATATTTTGACGGAGTGAGTAGTATCATATGCGTTCTAGAAAA |
| 747 | core_20 | CCGCCTGCCTGATAGCCCTAAAACCTGAGAAGATACCTACGAGCTAAATTGA |
| 750 | core_21 | CCAGACGACGATAAAATTTTTCCAAAATAGCGAGAG |
| 751 | core_22 | AGAGCCGTACAAGAACAACGGGTATGACCCCCAAGGGTT |
| 752 | core_23 | AATAGTAAGAACGAGTTCAAATATCGCGTTTT |
| 753 | core_24 | AAGCCAGAAAATCAAGATTAGTTGTTTTTTATTTTGCACCCATCATTA |
| 754 | core_25 | AGGCGCATAACGCAAAGACACCTTAGAGCTTAATTGCTGAGGACAGATCTCATCTT |
| 755 | core_26 | CAATATTGAAAAATCTAAAGCATGACGTTGGGGCTTGCATATCAGGT |
| 756 | core_27 | CTTTACAAAAGGGACAAACCGTTGTAGCAATA |
| 757 | core_28 | CCTCAGAGCCACCACACTCGTCACCAATGA |
| 758 | core_29 | CCACCGGAACCGCCTACAAACAAATAAATCCGCTACAATATTTTCATACTGAACA |
| 759 | core_30 | TCCAGCGCAGCAGATTTTTGCCGGGTTACCTGCAGCCAGCCATTCAGGAAA |
| 760 | core_31 | TGCGGTATTCATAAACATCCCTTAAAACCGGGTCGAGGTGCCGTAAA |
| 761 | core_32 | GTCCATCACGCAAATTTTCTGGCCACACGACC |
| 762 | core_33 | ATCAACAGTTGAAAGCTTGCCCTAGGGTAGCTAAATTGTAAATCCAA |
| 763 | core_34 | AACTTAAATTTCCGTTTTTTCGTCTCGTTTTTTGCTGGCAGCCTCCGGCC |
| 764 | core_35 | AAAAATCCCGCGTCTGGTCGGATTGAAGATCGCACTCCAGTTCGCGTC |
| 765 | core_36 | CTCCGTGGGAACAAACCTCGCACAGGCGGCCTTTAGTGATGAAGGGTAAACAACCC |
| 766 | core_37 | GGCGGATTAGGAACAAATCAGAGTGAGC |
| 767 | core_38 | GGTGAGATACTTTTGCGGGAGAAGCCTTTATGGTGTAGCGTAAAAC |
| 768 | core_39 | AAGGCCGGAGACAGTCCAGTACATTCAATAGTGAATTTATTTGAAATA |
| 769 | core_40 | TGACTATTATAGTCATTTTTAAGCAAAGCGGATTGCAAATGGTCAATAACCT |
| 770 | core_41 | GAAAGACTAGATTTAGTTTGACCATTAGATACAAAAGAAGATTGAT |
| 771 | core_42 | GCTTCAAATAACAGTTGATTCCCAATTCTGCAATGTTTAGACTGGCGAACACCAG |
| 772 | core_43 | GCTCCTTCTGTAGCTCAACATGTTTTAAATTAAGGGAA |
| 773 | core_44 | TTCATATGGTTTACCATAGGTGTAAACCGCCA |
| 774 | core_45 | TAGCAATAGCTATCTTGGTCAAGAGAAGGATTAGGATTA |
| 775 | core_46 | ACCGAAGCGCTGGTCTGAAAGGGGGAAGGGCG |
| 776 | core_47 | TTTCTCCCAAGTGTACCGAGATTTTTTGGGTTGAGTGTTGTTCCGGCGAAAA |
| 777 | core_48 | GCCTTCCTCACACCCGTCACTGCCTTCCACAC |
| 778 | core_49 | TATCCCCCTCAAATGAAATCAAATCATACAGGCAAGGCA |
| 779 | core_50 | CGCGAAACTTTTCATGATGGCAATAAAAGGCTCCAAAAGGCAACTTT |
| 780 | core_51 | AGAATACATGGCACGGAATAAGTTTATTTTGTACCGAAATAAAGAAAT |
| 781 | core_52 | CGATTGAAATAAACAGAGCCTAATTTGCCCATTGACAGTCACCGA |
| 782 | core_53 | AGCGTGGTCCTTGCCGGACTTGTGAGACGCACCCTGAGAGATGGTGGT |
| 783 | core_54 | AGCTCTCAGCGAAACGTACAGTTTTTGCCATGTTTACCAGTCCCG |
| 784 | core_55 | GATATTCCCCTCAGAGCCGCCAACCGTAATTTTTCGGTCATAGCCC |
| 785 | core_56 | CTCATTTTCATTAAATATAAAACACCGAACGAACCACCAGCTTTTTAGACAGGA |
| 786 | core_57 | ATCAGAAAGAATAAACAATAGAAAGGAACAACGTTTCAGCGCTCAATC |
| 787 | core_58 | AAGATTGTCAAGAGAAGAGAAGAGAAATCAATATATGTGAGTGAATA |
| 788 | core_59 | CGGTGCCCACGCGTGCCTGTTCCCAGCTTTAACCACGTAGCCA |
| 789 | core_60 | GGGAGTTAAAGGCCGCTTTTGCGAACTGGCTCGCCAAAAGGAATTAC |
| 790 | core_61 | GACAGCATCGGAACGGACGAGAACCTGCTC |
| 791 | core_62 | CAGAGGCTGGTGAATTGAAGGTTAATAGAACC |
| 792 | core_63 | AAATACGTGAATTATCAAAGAAACCACCAGAAATGAAT |
| 793 | core_64 | ATAACATCAATATTACCGCCAGCCATTGCAACACAACTCTATTAGAATAGATT |
| 794 | core_65 | AAGCTAAAGATATTCAACCGTTCTATTTCAT |
| 795 | core_66 | AATATTGAGTCTTTCCAGCCATATTATTTATCTACATACA |
| 796 | core_67 | CGGAAATTAAGGGCGAACCCTCAGTCACCGTACTCAGGAGGTTTAGTCACAATCA |
| 797 | core_68 | CCGTTTTTTTTATCCTAACGATTTTTTGTTTATATTACGCAGCGCTAATATCAGAG |
| 798 | core_69 | AATCATTACACAGGGAAGCGCATTTTTTTGACGGAATGAAAATAG |
| 799 | core_70 | TTCAAAAGACGGTAATATGATTACAGGTAGAAAGATTCACCCTGTAA |
| 800 | core_71 | GCCGGCGAGCTGCGCGTCCGGCACTAAATGTGAGCGAGT |
| 801 | core_72 | TATCCGCAGCTGTTTTTTTTGGTCTATCAGGGCGATGGCCCACTAC |
| 802 | core_73 | AGTATGTTAGCAAACGTAGAAAACCAATCCAAAAGACAAATCGAGAA |
| 803 | core_74 | CGTAAAAAAAGCCATATATTTTAATGGGGAGAGGCGGTTTGCGTATTGGGCGCC |
| 804 | core_75 | GCAAAATTGCCGGAGAAAACGAGACATCAAGATGGGTTTTTTTATAGGCGAATTATTCATT |
| 805 | core_76 | TTGAGTAACATTACTTCCATTACGGATATACCAACTTTGAAAGA |
| 806 | core_77 | GTCTTTCCGCAGTCTCTGCCCGTAACCAGAGCCA |
| 807 | core_78 | GCACCGTCGGTGGCCTATTTGCTCAAATCAGTTCCGGGAGGTTTTGAACGTTGTA |
| 808 | core_79 | AAGGGAAGAAGAGCTTTTTCCCGATTTAGAGCTTGACGGCAACGCTTT |
| 809 | core_80 | GCTACAGGGCCATCAATCGGCCAACGCGCTTTCCCTTAGGCCTAATG |
| 810 | core_81 | TCACCAGTCAACAGAGTCTAAAATATCTTTACGTAACAATAAAGACTAAAGTACA |
| 811 | core_82 | CCGAACGTTCCAGAACACTTGCCTGAGTAGAA |
| 812 | core_83 | TCACCAGTGAGCCAGTGTAATTTAGGCAGAGGACGACGACCTATTAGCAAG |
| 813 | core_84 | AGCACCATTACCTATACCGCCACGAGGCAGGTCAGACGATAACGAGC |
| 814 | core_85 | TAGCGTTTATTCATTACAAAAGGTAACGCCTG |
| 815 | core_86 | TCCCACCCATTATCAACAATAGATAAGTCTCGCGGAACCTTATTAAAG |
| 816 | core_87 | AAGGTTTCCTTTGACGTACCGAGCTCCTCACAGTTGAGGAACAGTATC |
| 817 | core_88 | CGATTAAAGGGAGGTAATGGGTAGTGAAAGCCAACGCTCAA |
| 818 | core_89 | TCATGGAATGTTTTTATAATCAG |
| 819 | core_90 | TAACGCGTCTGAAGCGCGAAAACAGTGCCACGCTGCGAACTAA |
| 820 | core_91 | GCCGGAAAATCGGCCTTGCGCCAACATTTTCCAG |
| 821 | core_92 | GCGTTTTTTTTCATCGGCACAGTAGCGACAGAATCTTTTTAGTTTGCCTTT |
| 822 | core_93 | TTAATTACATTTAACAAGCTGATGAGATCTTACATAACATTATA |
| 823 | core_94 | TCAAAAGAAGATGATGAAACAAAATGACCATCTTTAAAC |
| 824 | core_95 | TCAATTACGGAAGCCCAAATATTCTTTTGCCAATGGTTTAATTTC |
| 825 | core_96 | TCGCGCAGATAACTATATGTAAATGCTGATGCGTCGAAA |
| 826 | core_97 | GCTTTGAATACCAAGTTACAAAAAATTCGATCCGATAGCGTCCAATCAGCGAAA |
| 827 | Core_98 | CAGATGAATATACAGTAACAGTACTTTAATTATACCAAGCCGAACTG |
| 828 | core_99 | TGCGTAGATTTTCAGGTTTGCGGACTAAAACAGAACGGTGATCAAGAG |
| 829 | core_100 | TCGAGAGGTTCAGGTTTTTATAGCAAGGTACCAGGCGGATAA |
| 830 | core_101 | TGCCGAGATAACCCATTTTTAAGAATTGAGTTAGCAATAATAACGGAATACCCA |
| 831 | core_102 | GCGGGGTCAAGAAACGAGGCGTTTTAAAACGCGCCCAATGTACCGTA |
| 832 | core_103 | TCCGAATGATTGCCCTTCTTTTTCCGCCTGGGAAACAGCGGATCA |
| 833 | core_104 | CCTTATAAATCGGCTTTCATGGCGGTTGACGATGCTGATTGCCG |
| 834 | core_105 | GGAAACCTTTTCTTTTCACCAGTGAGACGGAGCAGTTGCAAATAGAC |
| 835 | core_106 | AAAACATATCTGTAAATCGTCGCTATTAATTAAATGCAATTTAACCAA |
| 836 | core_107 | GCGATAGCTTAGATTATTAAATAAAGCCCCAAAATTGTAA |
| 837 | core_108 | TAGGTCTGTTGAATTACCTTTTTTAATGGAAAAAATCACCGAGAGTCT |
| 838 | core_109 | AGAGACTACCTTTTTAACCTCCGATTTTTGAAAATTAAT |
| 839 | core_110 | CAAAGAACGCGAGATCACGGAGATCAACAGGTAAAGTACGGTGTCTGGAAGTTTC |
| 840 | core_111 | CTGATTGTATCGGGAGAAACAATAACGGATTCGCAAACTCTTGTATCA |
| 841 | core_112 | CCCTCAGACCAGTACAAGCAAGACCAAGTA |
| 842 | core_113 | CCCTCATTGTTGATATAAGTATAGCCCGGAAGCCAGAGGGTAATTG |
| 843 | core_114 | CCCAATAGGAAAAAGTATTAAGAGTAAGAACGCAATGAAAAACAAAGTTACCAGA |
| 844 | core_115 | CCAGTCAAAAGAATAGCGCGGTCACACGTGGCGTCTGCCAGCCCTGCATCAGACG |
| 845 | core_116 | CGTTGCGCCCGCGCTTGAAATTGTCGTGAGCCTCGAATTCGTAATCAT |
| 846 | core_117 | CATAATTAAATCCTTGCAATCATAGCCTGAGTCAAGGATAAAAATTTTTAGAACC |
| 847 | core_118 | TTAATGGTCAAAATCACATTGCCTATCAATATTCGGTTGTACCAAAAACATTATGA |
| 848 | core_119 | ATCAGATGAGGAAGTTTTAATTGTAACAACTA |
| 849 | core_120 | CACAGACGAAGGTATTATCACCGGAGGTT |
| 850 | core_121 | TAGCATTCGAACCGCCCATTCAACATAGAAAATAAAGGTGGCAACAT |
| 851 | core_122 | CAAACTACATCCTAATTTACGAGCATGTAGATTTTTACCAATCAATAACTGAA |
| 852 | core_123 | ACACTGAGTTTCGTCAGCCACCACCCTGAACAAAGTGCCAATAAGA |
| 853 | core_124 | TATATTCGCAACCATCCAGCAAATTTTGAATG |
| 854 | core_125 | AACATACGAAATCAAGTCCTGTGTTTTGCTCGGAGCCGGGTCACTGTT |
| 855 | core_126 | GCATAAAGTAACTCACATTAATTGTTAATGAAAAATAATT |
| 856 | core_127 | TGTAAAGCCTGGGGTATACAAATCTTTCCTC |
| 857 | core_128 | TTGCGAATGTGATAAATAAGGCGAGACGCTTCGATGA |
| 858 | core_129 | TTGAAAATGCTTTCGATTGAGGACAGCTGCTGCAGACGGTCAATCA |
| 859 | core_130 | TTGTCGTCTAGTTAGCGTAACGATAAATTATT |
| 860 | core_131 | TTTCAACGAATGTGTAGGTAAAGAACCTTGCT |
| 861 | core_132 | TCTTACCAGTATAACCATCACCCAGCCGGAATATGGTTGAATGCGCCGGCCTCAG |
| 862 | core_133 | TTCAACATAAAGGAAATATTTAAAACAGGCGGAACAACATTGGCGCATCGTA |
| 863 | core_134 | TTGCTAAAAGCTCATTTTGCGGAACATCATATTCCTGATTAATAATGGAGCGATT |
| 864 | core_135 | ACGTTAGTCTAAAGTTATTTGGGATACGAAGGTGACCTTCTACAGACC |
| 865 | core_136 | CAAGAAAAATTTCATAATCAAAATCATTTTTCGGATAAACAGT |
| 866 | core_137 | GCACTAAATCACTGCATCGTTAACGGCATTGTCACTGAGAATGCGGCGGGCCG |
| 867 | core_138 | GCGAACACTGGTGTTTTTGTTCAGCAAAACCGGGGTCAT |
| 868 | core_139 | CAGTAGGGCTTAACAGGAGGCGTTAGAATTAAAAATA |
| 869 | core_140 | GAATCGCCATATTTAACAACTGGTAATAATTAGAGCCCGCCACCAGAACACATTTGAGGA |
| 870 | core_141 | CAATAAACAAAGTAATTCTGTCCAAGTACCGAAAGGTGAA |
| 871 | core_142 | AAGTTAATGTACATCGACATAAAGGGTGGTTGTCGTGCCAGCTGCA |
| 872 | core_143 | TTCCGGCAGAATTTGTGAGAGCATCGCTTCTGGTGCCGGACGGGGGTTAGAAAGG |
| 873 | core_144 | CACCAACCTAAAAAGGAGCCCTCAAATAAGAGAATATAAGCATTTTC |
| 874 | core_145 | AATGCCACTATTAATTAATCACCACCAGAGCCGCCGCCAGAGGCTGGC |
| 875 | core_146 | ATCAGCTTCTCCAAAATCATCAATATAAAAACTTTTTCAA |
| 876 | core_147 | CAGAGGTGGAGCTGGGATTAACCTCACCGGATCATAACGGAACGTTTTAAGAAAA |
| 877 | core_148 | GCTTTTGATGCACTCCATCAGCAGCTTAC |
| 878 | core_149 | AAACGACGGCCAGTGCTTGGGTAAATTACGCCGATAGCCG |
| 879 | core_150 | AAGAATTAGAGGCATAAGTTCAGAACCCTTTTTTCAGACTGCGGAATCGTCAT |
| 880 | core_151 | ATATAATGTTGATAAGAGGTCATTTTTAACGTAGAACCTACCATATCAAAGGAGCG |
| 881 | core_152 | AAAGAACTGGCATGATTAAGACTCCTACGTCAAAGAGAATTACGTAGG |
| 882 | core_153 | ATAAAAGAAGTTACAAGGGCGAAAGAGGCAAATGCACGTAAAACAGCCACCCTCA |
| 883 | core_154 | AAAATTCGTGTACCCCTCTGCCAGATGGGATAGGTCACGT |
| 884 | core_155 | ATGCAACTCAGGATTAGAGAGTACCCTTTTACTTGGATTATACTTCTG |
| 885 | core_156 | TCCGATAGTTGCGCCGACAATGACAAGTCGCTGAGAAGAAAAAATACCACATTCAACT |
| 886 | core_157 | ATATAGAAGGCTCAGACAAGAGTCCACATTATTCTGAAACATGCCCATAGCAAG |
| 887 | core_158 | AACGCGGTCTGCTCATTTGCCGCCGCAACAGCATCGGCAAAATC |
| 888 | core_159 | CAGAGCGGATTTTGTTAGCCTGTTACCGGAAT |
| 889 | side3_S3_1 | TAATGCAACTGTAGCTAATGCCCAGTAACAGTGAATTTACGGGGTCAATGT |
| 890 | side3_S3_2 | AGTTTGGACAGCAACCGCAAGGAAACTGTCGCCACGAGGT |
| 891 | side3_S3_3 | AACGTGGGGAACCCTAAAGGAGCAATGCCAACGGCACTGCGGCCCGCGCCTGTGATACAG |
| | | TAAT |
| 892 | side3_S3_4 | TAAAACGAGCCTTATGGAAAGCTTATCATTCCTTTTTAGAACGGGTATTAA |
| 893 | side3_S3_5 | AGGAAACCATAGCGAACCTCCCGACTTGGCTCGCCAGGGTTTTCCCAGTCACAT |
| 894 | side3_S3_6 | TAACGAGTGTACTGGTTTATCGGTGTGTGGTG |
| 895 | side3_S3_7 | TTAAGTGCCTTGCATACATGAGTTTTAACCGTTCCACGGGCCTC |
| 896 | side3_S3_8 | TGATGGAACGGAGTGCTGCAAGGCGATTAAGCAAGCTAATA |
| 897 | NoHandle_1 | AGCGTCAGGTGTCCAGGTAAGCGTCCTGTGCCA |
| 898 | NoHandle_2 | GGCTGGAGGAACGCGCCTGTACGTCAAAGG |
| 899 | NoHandle_3 | CATAACCCCCAATAAAAATCAGGTCTTTACCC |
| 900 | NoHandle_4 | AAACCCTCATTTTAAGGGATCGTCGGGTAGCTGCTTAGGTAAACAAAA |
| 901 | NoHandle_5 | GAACTCAACCTCAGAGCAGCAAAACTTGAGCC |
| 902 | NoHandle_6 | TGAGGCCATCTTTAATATGGATTAATAAGCAA |
| 903 | NoHandle_7 | GCCCTGCGTCCCCGGGAGCACGTAGCGCGTAC |
| 904 | NoHandle_8 | GTTTAGCTATATTTTCATTTGGGGCTTTTTCGAGCTGAAA |
| 905 | NoHandle_9 | AGGTGGCATCAATTCTACTAATAGTAGTAGCATTAACATTCGTTTA |
| 906 | NoHandle_10 | ACGGTACGCCAGAATCATCGCCAT |
| 907 | NoHandle_11 | AACCATCGATAGCAGCCCCTCAGA |
| 908 | NoHandle_12 | CTTCTTTGATTAGTATTTAGAAGGTATTAAA |
| 909 | NoHandle_13 | CTTCTGACCTGAAAGCGTTTTTTAGAATACGTGGCACAGA |
| 910 | NoHandle_14 | CCAGTCAGCACCTTGCTGAACCTCAAATATC |
| 911 | NoHandle_15 | GCTTTTGCATTTCGCATCAAAAAGATTAAGACTGAGT |
| 912 | NoHandle_16 | TGCAGGCGCTTTTTTTTGCACTCAATCCGCCGGGCGCGGTGGTGGTGC |
| 913 | NoHandle_17 | GCTATTAGCCGAGTAAAAGAGTCT |
| 914 | NoHandle_18 | AACGAGTATCTGGTCAGTTGGCAA |
| 915 | Handle_1 | GCAGTAGAGTAGGTAGAGATTAGGCATTGCAGTAGAGTAGGTAGAGATTAGGCAAGCGTCA |
| | | GGTGTCCAGGTAAGCGTCCTGTGCCA |
| 916 | Handle_2 | GCAGTAGAGTAGGTAGAGATTAGGCATTGCAGTAGAGTAGGTAGAGATTAGGCAGGCTGG |
| | | AGGAACGCGCCTGTACGTCAAAGG |
| 917 | Handle_3 | GCAGTAGAGTAGGTAGAGATTAGGCATTGCAGTAGAGTAGGTAGAGATTAGGCACATAACC |
| | | CCCAATAAAAATCAGGTCTTTACCC |
| 918 | Handle_4 | GCAGTAGAGTAGGTAGAGATTAGGCATTGCAGTAGAGTAGGTAGAGATTAGGCAAAACCCT |
| | | CATTTTAAGGGATCGTCGGGTAGCTGCTTAGGTAAACAAAA |
| 919 | Handle_5 | GCAGTAGAGTAGGTAGAGATTAGGCATTGCAGTAGAGTAGGTAGAGATTAGGCAGAACTCA |
| | | ACCTCAGAGCAGCAAAACTTGAGCC |
| 920 | Handle_6 | GCAGTAGAGTAGGTAGAGATTAGGCATTGCAGTAGAGTAGGTAGAGATTAGGCATGAGGC |
| | | CATCTTTAATATGGATTAATAAGCAA |
| 921 | Handle_7 | GCAGTAGAGTAGGTAGAGATTAGGCATTGCAGTAGAGTAGGTAGAGATTAGGCAGCCCTG |
| | | CGTCCCCGGGAGCACGTAGCGCGTAC |
| 922 | Handle_8 | GCAGTAGAGTAGGTAGAGATTAGGCATTGCAGTAGAGTAGGTAGAGATTAGGCAGTTTAGC |
| | | TATATTTTCATTTGGGGCTTTTTCGAGCTGAAA |
| 923 | Handle_9 | GCAGTAGAGTAGGTAGAGATTAGGCATTGCAGTAGAGTAGGTAGAGATTAGGCAAGGTGG |
| | | CATCAATTCTACTAATAGTAGTAGCATTAACATTCGTTTA |
| 924 | Handle_10 | GCAGTAGAGTAGGTAGAGATTAGGCATTGCAGTAGAGTAGGTAGAGATTAGGCAACGGTAC |
| | | GCCAGAATCATCGCCAT |
| 925 | Handle_11 | GCAGTAGAGTAGGTAGAGATTAGGCATTGCAGTAGAGTAGGTAGAGATTAGGCAAACCATC |
| | | GATAGCAGCCCCTCAGA |
| 926 | Handle_12 | GCAGTAGAGTAGGTAGAGATTAGGCATTGCAGTAGAGTAGGTAGAGATTAGGCACTTCTTT |
| | | GATTAGTATTTAGAAGGTATTAAA |
| 927 | Handle_13 | GCAGTAGAGTAGGTAGAGATTAGGCATTGCAGTAGAGTAGGTAGAGATTAGGCACTTCTGA |
| | | CCTGAAAGCGTTTTTTAGAATACGTGGCACAGA |
| 928 | Handle_14 | GCAGTAGAGTAGGTAGAGATTAGGCATTGCAGTAGAGTAGGTAGAGATTAGGCACCAGTCA |
| | | GCACCTTGCTGAACCTCAAATATC |
| 929 | Handle_15 | GCAGTAGAGTAGGTAGAGATTAGGCATTGCAGTAGAGTAGGTAGAGATTAGGCAGCTTTTG |
| | | CATTTCGCATCAAAAAGATTAAGACTGAGT |
| 930 | Handle_16 | GCAGTAGAGTAGGTAGAGATTAGGCATTGCAGTAGAGTAGGTAGAGATTAGGCATGCAGG |
| | | CGCTTTTTTTTGCACTCAATCCGCCGGGCGCGGTGGTGGTGC |
| 931 | Handle_17 | GCAGTAGAGTAGGTAGAGATTAGGCATTGCAGTAGAGTAGGTAGAGATTAGGCAGCTATTA |
| | | GCCGAGTAAAAGAGTCT |
| 932 | Handle_18 | GCAGTAGAGTAGGTAGAGATTAGGCATTGCAGTAGAGTAGGTAGAGATTAGGCAAACGAGT |
| | | ATCTGGTCAGTTGGCAA |

TABLE 3B: Staple sequences for triangle 2, version 2

| SEQ | | |
|---|---|---|
| ID NO: | Description | Sequence/Details |
| 933 | core_1 | TAGTTGCAAGGAATTGCGAATACCCTCGTTCCAATACTGCGG |
| 934 | core_2 | AGTAACAGTTAATTTTCCAGCTTACGGCTGGGTGATGAAATTTAC |
| 935 | core_3 | ATCCTGAGGCGGGCCAGCAAAACAGATAAATC |
| 936 | core_4 | TGATGGCAATCATCATATTCCTGA |
| 937 | core_5 | TTGTTTGGATCATTTTGCGGAACAAAGTTTGA |
| 938 | core_6 | CAGAAGGAGCACGCGTGCCTGTTTTTTTTTCGCGTCCGTGGTTTT |
| 939 | core_7 | ATTTGCACTAAACAGTTAATAGTACAGCGAAA |
| 940 | core_8 | TTATCAGAGCATCAGAAGCCAGCGGCTAAACA |
| 941 | core_9 | TATTAATTTTAAAAGAATTTTTCCACGCCCGAACAAATCGCG |
| 942 | core_10 | CAAACAATTTACCTGATCTTTAGGTTGCTTTGCACACCCGAAGAAAGCGAAAGGA |
| 943 | core_11 | TCAAAATTATGGAAGGCAGAACCAGACATAAAGAAATT |
| 944 | core_12 | TTTCAGGTTTAACGTCAGATGAATATTTTAACACCGCCTGGAGTGAGC |
| 945 | core_13 | GTACCTTTTATAGATAATACATTTGAGATTAGCAGAGGC |
| 946 | core_14 | ATTTCGGAGATACAGGGAGCCACCAGTACCGCCACCCTCA |
| 947 | core_15 | GTTTCCTAATTCGTGCTATTATTATTTA |
| 948 | core_16 | TAGAATCATACTATGGAGCACTAAACAGTTGAAAGGAATTGAGGAAGGTAACCAC |
| 949 | core_17 | GTGTGAAACATTAATGTTCACCGCCTGGCCCTTGACGGGGCTTATAAATCAAAAG |
| 950 | core_18 | TCTTTTCACGCCACCCTCAGAACCTTGCCGCCGCACAGGCGGCCTTTAAGGTGTCC |
| 951 | core_19 | GATACCGAAGACGTTAGTAAAGTGCACCAGTACAAACTACGACAAGAATTTGATA |
| 952 | core_20 | GAGGTGAGAAAATCTCCAAAAAACGAGGCAATTCATCAGTTGAGATACGAACTA |
| 953 | core_21 | TCAGTAGCAGGCAGGTTAAGCCCATTTGTGAGGCCTTTTTTCCGACCGGAAACAATCGGC |
| 954 | core_22 | CGTCAGACCAGAGCCGCCGCCAGCGGATAGC |
| 955 | core_23 | GACAGAATCAAGTTTGCCTTTAGCAGAGCCTCAAACAAA |
| 956 | core_24 | ACTTGAGCCATTTGGGAATTAGAGCTTTTTAGCAAAATCACCAGTAG |
| 957 | core_25 | CACCGTAAAGCCATAAATTGAGTCTTCTTTTTTGACAACAAAGTCAGAGGGT |
| 958 | core_26 | CACCATTACCATTAGCAAGGCCGGAAACGTCAACGATT |
| 959 | core_27 | GCGGGCGCGGTGGTTTCGTAAGCCAACGCTCA |
| 960 | core_28 | GAAAATTCATTACGCAGTATGTTATCACGACGTTCGCCATTCAGGCTGGCACCGC |
| 961 | core_29 | ACGGAATAACATACATAAAGGTGGATTAAGTTAGGGCGATCGGTGCGGCTCAGGAA |
| 962 | core_30 | ATCAGGGCGATGAAAGAACGTGGACTCCTTTGCTGAGAGCCAGCAGCAAAT |
| 963 | core_31 | AAAGCCGGCGAACGTGGCGAGAAAACAGCTG |
| 964 | core_32 | TAAAGGGAGCCCCCGATTTAGAGCTGAGAGAGGAAAATCCGCTCA |
| 965 | core_33 | GTACTCAGACTCCTACATGAAGAAGAAA |
| 966 | core_34 | CGATATATTCGGTCGCGCCGATCGTCTTTCAGCGCAAAATAGAGACTGGAT |
| 967 | core_35 | GAATAGGTGTATCACCGCCTAATTGTATCGGTTTATCAGCTTGCTTTCATATAAGT |
| 968 | core_36 | TACCAACGTTTTGAAGCCTTAAATCAAAAACAGCTGATGCGCAATGCCTGAGTAATT |
| 969 | core_37 | GGAGGTTTACCCTCATTTTCAGGAACGGTATTCTGAACCATTAG |
| 970 | core_38 | CTGAATCTGAAAGCGCAAATCCAACCACGCAACCCTTAGAATCCTTGAT |
| 971 | core_39 | CTTTACAGAAGGTGAAGTAAGCAGATAGCCGCAGCGT |
| 972 | core_40 | TTATTTATAAGGCTTATCCGGTACGCATTTTTTTAATAGGAATCATTACCGCACAGGGAA |
| 973 | core_41 | TTTTGTTTAACGTCAATTTTTAATGAAAATAGCAGC |
| 974 | core_42 | TCCAGTCTTTTTGGAAACCTGTCGTCTGATA |
| 975 | core_43 | ATCTTAACGCCTGTAGCTTTTTTTCCACAGACAGCCCTCAAGAGTA |
| 976 | core_44 | ACTAGCATGTCAATCACACCCTCAAGTGTACTATACATGG |
| 977 | core_45 | TCATTACCCAAATCAAACACTGTACGGTGTCAGTAC |
| 978 | core_46 | CATTCAGTGAATAAGGGATAGCAAAGTTGATTCAAGAGAA |
| 979 | core_47 | TTAAAATTTTCTAAGAAATTTCATCAGACGATCGCTGGCAAGATAGAC |
| 980 | core_48 | CTCATTTTTTAACCAGAGAACAACGACCGTGATATAAAGACGTGCCG |
| 981 | core_49 | TATGTACCTTAATGCCAAAATTTTTAACCTTCGCCGGGCGCGGTTGCGCTTTCGC |
| 982 | core_50 | TGCGCGAAGCCAGCTGTTGTTATCCTGTTTGA |
| 983 | core_51 | GCCCTAAAACCCGCTTAATTCCACGCGTTGCGTGCCCCATTTTTCAGGCTTGCAGCAAGC |
| 984 | core_52 | AAATATTTCCCGACTTGCGAGAAATAAATCCT |
| 985 | core_53 | AATCGGCCAACGCGCTTTGAATGAATCATGGGGAAAAACCCGGGGGTTCACTGCGCGCC |
| 986 | core_54 | TCAACATTATAATCGGGCCTGTTTGCCATATTGTTGGGA |
| 987 | core_55 | AGGAACAACTAGCCGACAATGACAACAATTTTTCATCGCCCACGCATAAC |
| 988 | core_56 | AAAGGCTCATTAGCGGATAGCCCGGGAACCCATGTACCGTACGTAACA |
| 989 | core_57 | CAAAAGGAGCCTTACCACCGGAAGAAATGCAGATACATAACGCCAAAAGGAATT |
| 990 | core_58 | GCCACCCTCGTTCCAGACCCACCCAGCTACAATTTTATCTCGGTCAT |
| 991 | core_59 | CAGAGCCAGCCCCCTTATTAGCGTTTGCCAGAACCGCCTAAGCGTC |
| 992 | core_60 | ACCACCCTCAGAGCCGAACAGCGGGTTAAACGATGCTGAGTGCCATC |
| 993 | core_61 | TCTAGCTGATAAATTAATGCCGGAGGTATAAGC |
| 994 | core_62 | TACCAGAAAATATTGACGGAAATTATTCATTAAGAGAATAGCTAAT |
| 995 | core_63 | GGTCCACGCTGGTTCTCACTGGGTGCCGT |
| 996 | core_64 | TCCTTATCATTCCAAGGGCCTTCCGTGAATTTAAAAGGGTCGGCGGATAGAAGC |
| 997 | core_65 | ACCAATCAAAATGTGACATCGTAAAGGTCACGTTTTTTAATGGAAAC |
| 998 | core_66 | CCGCGCTTAATGCGCCGCAAATCACAACTAATAGGATTTATCGTATTAAATCCTTT |
| 999 | core_67 | TTGTTCCAGTTTGGAACTAAAGCATTCGCCTGATACAGTAACA |
| 1000 | core_68 | ATAGCTATCTTACCGTTTTTAGCCCTTTTTAAGAAAATTATCACCGTCACCG |
| 1001 | core_69 | AGGTGCGGGCTTTGCGAAAAACCGTCT |
| 1002 | core_70 | TTCTGTACATGTTTTATCCCCCTTTTTCAAATGCTTGTA |
| 1003 | core_71 | GGTTTTGCTCTGGAAGACGGAACATGAATTAC |
| 1004 | core_72 | TTATCAGAGAGATAAGAGCAAGAAACCATCGATAGCAG |
| 1005 | core_73 | GTAATTTACGGAATCATTCTGGTGTTAGATTAAGACGCTGAATCAAT |
| 1006 | core_74 | ACGAGCATTCTTTTCGTATTGGGCGCCAGAAGCGTAA |
| 1007 | core_75 | AGGGCGCGTCATAGCTGAATTATTTTCTGCCAGCGGAATTATTCATCAATATA |
| 1008 | core_76 | ACCAGGTCATAGTTTTCAACAGAATGCTGTTTTTTGCTCAATGGGATTTTGCT |
| 1009 | core_77 | AAACAACTAGCGTAACGATCTTTTTTAAGTTTTGTCGTCTTTCC |
| 1010 | core_78 | AAATATGCTTGCTCCTCCGGATATACGGTGTA |
| 1011 | core_79 | AACTAAAGAGTTTCGTCCGTCGAGAGGGTTG |
| 1012 | core_80 | TTTCATTCTAATTTCAAAGCTGCTACACCAGA |
| 1013 | core_81 | AGTATTAAACCTATTAATCGTAAAACAAGAGA |
| 1014 | core_82 | GGTAATAAAGCATCAGGCTTCTGTAAATCGTC |
| 1015 | core_83 | GTTTTAACATAAACAGCCGGTTGACTATCAGG |
| 1016 | core_84 | GTGCCTTGAGTCTCTGAGGGTAAAATCAAACTTAAATTTCTGCTCAT |
| 1017 | core_85 | TCGCAAGATGTAAATGGAAGATTAGGGTAGC |
| 1018 | core_86 | TCAAATCAGATATAGCCCAATCCAAATAAGAACCAATGAAACAATGAAATAGCA |
| 1019 | core_87 | AATGGTTTGAAATACGCAAGCCTAAAGTA |
| 1020 | core_88 | GCGAACCTAAATTGTAAACGTTAATATTTTG |
| 1021 | core_89 | TAATTACTAGTCAATATGTAGCCAGGAACAAA |
| 1022 | core_90 | AGTATCATGATCGCAGACGACAGTATCGGC |
| 1023 | core_91 | ATGCGTTAGATGGGCGGCGATAGAACCCTTCTGACCTGATCCTAATT |
| 1024 | core_92 | TCTTACCAGTATAATAAAGATGATTGCCGGGTTACCTGCCGTTAACG |
| 1025 | core_93 | CGGTCACGCTGCGCGTTCTGGCGAAAGGGG |
| 1026 | core_94 | TTCCTCGTGCGGTTTGCACCAGTGAGACGGGCAGGAAGGG |
| 1027 | core_95 | AACGGAATCGACATTCAACCGATTGAGGGAG |
| 1028 | core_96 | AGAAAATAGTTTATTTTGTCACAATCAATAAATGCAGA |
| 1029 | core_97 | ACTTTTTCAAATATATTTTAGTTACGCGAGGCAGTTACAAAATAAAC |
| 1030 | core_98 | ACAACATATTGAATACCTTTTTAGTTACA |
| 1031 | core_99 | AGTGTAAAGCCTAACCGAGAATGACCAGGTGTAACATT |
| 1032 | core_100 | ACTTTAAGGGCTTGTTCAAATAAGCAAAGCGGATTGCA |
| 1033 | core_101 | CCCAATTCAACTGGCTAATCTACGTTAATAAATTAGGAATGGATTAGG |
| 1034 | core_102 | AGAACGTCAGCGTGCACATTTTCGGACTCCTTATATGGTTTACCAGCGCCAAAGA |
| 1035 | core_103 | AAGCGCCATTGTAAAACGACGGCCAGTGCCAAATGATTAAAGCCAGTA |
| 1036 | core_104 | TGCTTAGGTTGGGTTATATAACTATACAAAGAACGCGGGAGGCTAACGAGCGTCTTTC |
| 1037 | core_105 | ATCAAAATGCGATAGCCCGGAAACCAGGGTGCTGGTCTGG |
| 1038 | core_106 | CCGTGCATCTGCCAGAGAAAAACTGTCTTGTTTATCAACAATAG |
| 1039 | core_107 | GATTTTAGAATTACATGAAACAAACAAAGAAAGCCTCTTCACAGTAGAAAGTGTAG |
| 1040 | core_108 | ATTAAAGGCTGAGAAGGCATCAGAGTGTGTTCAGCAAAT |
| 1041 | core_109 | AGAGGTCATTGCAATTTTTCTCCAACAGGTCAGGA |
| 1042 | core_110 | CCAACTTTGAAAATTTCAAGAGTACCTTTAA |
| 1043 | core_111 | GTCAATAACCCTGCCTACTCAATCCGGGGTCA |
| 1044 | core_112 | TGTGTAGGTAGTCAAAAAATAATTCGCGTCTAACGGGTAATAAACACGGCAGAGG |
| 1045 | core_113 | TGTTTTTATAATCCAAGTCACACTGGCACAGACAATATTGGGGAGAG |
| 1046 | core_114 | TTGCAACAGAGCGGGAGTGCCGGTCATTTCAATCGACAACGAAGTATTAGACTTTA |
| 1047 | core_115 | TTTCTCCGTGGTGAAGATTGACAATATTCAAATTTGCCGTTTTA |
| 1048 | core_116 | TCCATGTTTACCAGTCCCGGAAATAATAACCCACAAG |
| 1049 | core_117 | GAAACGTAAACAAAGTAATTGAGCACATAAAAGCCCAATAGCAAG |
| 1050 | core_118 | ATAACCTCGCCAGAGCACATCCTCATAACGGATACCGAC |
| 1051 | core_119 | AGGTGGAGCCGCCACGGGAACGGCAATAATAAAACTGAACACCCTGCTAAATTT |
| 1052 | core_120 | GGGTAACGCCAGGGTTTTCCCAGGCAAACGTGCCAACATACGCGCCT |
| 1053 | core_121 | GATGTGCTGCAAGGCGCAACATATGGCTTAATTGAACAAGATGTAGAA |
| 1054 | core_122 | ATCAAACCAGAAACTTTTTATAACGGATCACCTTGCTGAACCT |
| 1055 | core_123 | CAAATAATAGCCCGATTTTTATAGGGTTGAGTGAAAGCACTAAATCGGAACCC |
| 1056 | core_124 | GAAAAATCAAGAGTTAACTCACATTAATT |
| 1057 | core_125 | AAAGAAGTAACGTCAAGACAGCATCGGGGGGTGCCTAAT |
| 1058 | core_126 | AGCGTTACCAGACGACTTTTTATAAAAACGAGTGAGAATAGAA |
| 1059 | core_127 | AATCGTCATAGGCAGGCGGATTCACGTTATTTCTTAAACAGCTT |
| 1060 | core_128 | TAGAAAGTAGTAAGAGCAACACTATCATAAATAATTTTTAATGAATT |
| 1061 | core_129 | ACCACATTCAACTCATAAAAAAAGACGTTGG |
| 1062 | core_130 | AAAAAGCCAGCAGTTGGGCGGTTGTGTACATCCCGCCTCCGAGGCTGA |
| 1063 | core_131 | CCGGCAAATCTCACGGAAAAAGAGACGCAGACCACCAGACCAGAATG |
| 1064 | core_132 | CGCGGTCCGTTTTTTCGTCTCGTTGGCCTTGGGAGGTTG |
| 1065 | core_133 | TGAGTGAATAGAACCCTTTCAACGGGGCGCGA |
| 1066 | core_134 | CCGGGTCACTGTAAGAGGAAGCCATTTTAAGTGCGAACGCATATAACGCCCAATA |
| 1067 | core_135 | GCTATTACGCCAGATCTAAAATAGCAAAAG |
| 1068 | core_136 | AGCCGTCATGGTGGTTCCGAATACATTGCCC |
| 1069 | core_137 | ACATCGGGCTCAATCAATATCTGGTCAGTTGGCATCGGCAAAATCC |
| 1070 | core_138 | CAACAGTGCCACGCCGCGTAGATCTACCATA |
| 1071 | core_139 | AGAGGGGGTCAGAAAAGAGTAGAGCTTAATTGCTGAATAT |
| 1072 | core_140 | TTGCAGGCTCCCGTAACTTTTGATCTCAGAGCTAATCAAAATCACCGGAACCAGA |
| 1073 | core_141 | GTCGGTGTTGCCGTTCATTAAAGACCACCACTGTAGCGCGTTTTCATCGGCATTT |
| 1074 | core_142 | ACGTGCTGGAGGCCGGAATACGGACCAGT |
| 1075 | core_143 | TTAGTTGACATTATTACAAATATTCATTGA |
| 1076 | core_144 | CTTATGCGCGAAAGACAGATGGTTTCCGCGACCTCATCTT |
| 1077 | core_145 | CATTATACCAGTCAGGGTATGAGATACATTTTTGTATCACGCGAAAC |
| 1078 | core_146 | TAAACATACATAGGTCAAATAAGATTAAACCACAACATGTTCAGCT |
| 1079 | core_147 | AAAAATGTTAGAACATTATACTTCTGAATA |
| 1080 | core_148 | ATTCTGTCCAAAAGGGACCCAAAAGAACTGGCGCTTTCAGGACTTGT |
| 1081 | core_149 | GACGGGAGAATTAAGGGTTTTTATTTTCATCGATTTTTGTTAAATCAG |
| 1082 | core_150 | TTATATGGCTATTAATGCCCGTGGGGTCAGTTGCTATTTTGCTCAGAACCGC |
| 1083 | core_151 | AGTACATAAGAAGTTTGAGGGGACCTCCAGCCAGCTTTCCGCGCAACTTAACAAC |
| 1084 | core_152 | GAATTACCTTGGTGTAAAACATTAAAGCAATA |
| 1085 | core_153 | TTTCATTTACAAAATTACAGGAATACAAATTACGAGCAAAAATA |
| 1086 | core_154 | CACGGTCATAGCTCATGGAAATACCTTTTTTCATTATACCGAATGCTGTTTAATAACAT |
| 1087 | core_155 | CAGACGACATAAGAGATGATAAATTCAGCAGCAACCGCAATTTTTAATGCCAACGGCAGCA |
| | | CC |
| 1088 | core_156 | TAGTAGCATTAACATCCCGGAGACAAAGATTC |
| 1089 | core_157 | TCTTTATCATATAACAAAGGTAATCAGAAAAGCCCCAAGATTA |
| 1090 | core_158 | TTTTTGAGGCGCAGATAACGGGTAAA |
| 1091 | core_159 | TGTGCACATCGGCCTCGAACCACCAGAGAGGCTTTGAGTTTCGGCCAGAATGC |
| 1092 | core_160 | ATATCCCATAGGGCGCTGGCAAAGAAACGCAAAGACACCATAAGTCCTGAGAATC |
| 1093 | core_161 | GACAATAAAAGTACCGCACTCATCATAGGAACTACCTTTTTAACCTCCGT |
| 1094 | core_162 | GCGCATTAGGAAGGTAGGAAACCGAGGAAACG |
| 1095 | core_163 | AAGGCGTTTGAGAGACGCCATCAATCACCATC |
| 1096 | side3_rec_S3_ | TAACACTTAAATTGACGCTCATTTTTTCGTCTGAAATGGAGTCTTTAA |
| | 1xT_1 | |
| 1097 | side3_rec_S3_ | GAGGCGGTCAGTAGCTCGAGCCGGCTCACAGTTTAGCCTGAGT |
| | 1xT_2 | |
| 1098 | side3_rec_S3_ | TTGCCAGGATGGCTGGTAGCAACGGCTACCAGAAGATCTCAAGCATAA |
| | 1xT_3 | |
| 1099 | side3_rec_S3_ | CCACTATTGCCCACTACGTGAACCATGTCGATTGGGCACT |
| | 1xT_4 | |
| 1100 | side3_rec_S3_ | TCAAGTTTTCATCGCCATGAGGATCCCTTTTTGGGTACCGAGCTCG |
| | 1xT_5 | |
| 1101 | side3_rec_S3_ | AAACAGAACGGAATTTGCCTGCTGACCTTCACTTGCAGGT |
| | 1xT_6 | |
| 1102 | side3_rec_S3_ | CTTCAAAGCGGTCGACCGGTCAATCATAACTGACGAAATT |
| | 1xT_7 | |
| 1103 | side3_rec_S3_ | TAAGTGCTCAGAAATGTTTCGAGAGGCTTTTGCA |
| | 1xT_8 | |
| 1104 | nohandle_1 | CCAATCGCGTCAGACGATTGGCCTTGATATT |
| 1105 | nohandle_2 | TCAGAAGCAAGGCTATATTAAATTAATGCCCACGCTGAAGT |
| 1106 | nohandle_3 | GAGAATGATAGCATGTAGCCCCAAAAAATAGCGAT |
| 1107 | nohandle_4 | CCCCTCAGTGTCGATGCAATGCCTGAGTA |
| 1108 | nohandle_5 | TACATGGCCTTAGCCG |
| 1109 | nohandle_6 | GTCTCTGAATAAGGGAGAACGGTG |
| 1110 | nohandle_7 | CACAAACATATATGTAATATAAGTATAGCCCG |
| 1111 | nohandle_8 | TTTTTGGGAAGACAAATCATCGAG |
| 1112 | nohandle_9 | CCACTACTATATTTCCAAGAAGCGCCTG |
| 1113 | nohandle_10 | ATAACCTAAAAGAACGTGGACTCCAACGTCA |
| 1114 | nohandle_11 | AGTCTGGGGTCTTTGGAAGCCCGAATGTTTAGA |
| 1115 | nohandle_12 | ATGTGTAGAATGCTTTAATATTCATTGAATC |
| 1116 | nohandle_13 | CATGTTATTTTGATGGGGTCAGTGCCTTG |
| 1117 | nohandle_14 | GTCAATCATTTACCTAAACAGTTAATGCC |
| 1118 | nohandle_15 | GGCTTGCAGGGAGTTATATTCGG |
| 1119 | nohandle_16 | AACGAGGGAATAAATCAAGTATTAAGACATTGA |
| 1120 | nohandle_17 | CTTAGATTAAGACGCATAAATAA |
| 1121 | nohandle_18 | TTTTCAAAGTGAACCACCCTAAAGGGAGCCC |
| 1122 | nohandle_19 | CTGACCTAAAAACCGTCGGGGAAA |
| 1123 | nohandle_20 | AATTAAGCCTCCAGTATAAAGCCAA |
| 1124 | nohandle_21 | GATCTACATGCTTCTTTCAACTTTACATCAAGAAAAC |
| 1125 | nohandle_22 | TTAATGCCGGAGAGGGAATTAC |
| 1126 | nohandle_23 | GGCCGGAGACAGTCA |
| 1127 | nohandle_24 | ATAAATTGTAAAGATTCAAAAGGTGTACCCC |
| 1128 | nohandle_25 | AAAGTACAACGGAGATTTGTATCACCTGCTC |
| 1129 | nohandle_26 | TGACCCCCAGCGATTGAATTTT |
| 1130 | nohandle_27 | AGGCAAAAGAATACACTTTAAT |
| 1131 | nohandle_28 | CATTAAACGGGTAAAATTGCGCCG |
| 1132 | nohandle_29 | AGGACTAAAGACTCACCCTCAGCAGC |
| 1133 | nohandle_30 | TATATAACTAGCAACGGCTACAGGCATCGG |
| 1134 | nohandle_31 | TGAATTTATCAAAATTGCAGAACCGGGTATT |
| 1135 | nohandle_32 | CTACCTTTTTAACCTC |
| 1136 | handle_32H_1 | GCAGTAGAGTAGGTAGAGATTAGGCACCAATCGCGTCAGACGATTGGCCTTGATATT |
| 1137 | handle_32H_2 | GCAGTAGAGTAGGTAGAGATTAGGCATCAGAAGCAAGGCTATATTAAATTAATGCCCACGC |
| | | TGAAGT |
| 1138 | handle_32H_3 | GCAGTAGAGTAGGTAGAGATTAGGCAGAGAATGATAGCATGTAGCCCCAAAAAATAGCGAT |
| 1139 | handle_32H_4 | GCAGTAGAGTAGGTAGAGATTAGGCACCCCTCAGTGTCGATGCAATGCCTGAGTA |
| 1140 | handle_32H_5 | GCAGTAGAGTAGGTAGAGATTAGGCATACATGGCCTTAGCCG |
| 1141 | handle_32H_6 | GCAGTAGAGTAGGTAGAGATTAGGCAGTCTCTGAATAAGGGAGAACGGTG |
| 1142 | handle_32H_7 | GCAGTAGAGTAGGTAGAGATTAGGCACACAAACATATATGTAATATAAGTATAGCCCG |
| 1143 | handle_32H_8 | GCAGTAGAGTAGGTAGAGATTAGGCATTTTTGGGAAGACAAATCATCGAG |
| 1144 | handle_32H_9 | GCAGTAGAGTAGGTAGAGATTAGGCACCACTACTATATTTCCAAGAAGCGCCTG |
| 1145 | handle_32H_10 | GCAGTAGAGTAGGTAGAGATTAGGCAATAACCTAAAAGAACGTGGACTCCAACGTCA |
| 1146 | handle_32H_11 | GCAGTAGAGTAGGTAGAGATTAGGCAAGTCTGGGGTCTTTGGAAGCCCGAATGTTTAGA |
| 1147 | handle_32H_12 | GCAGTAGAGTAGGTAGAGATTAGGCAATGTGTAGAATGCTTTAATATTCATTGAATC |
| 1148 | handle_32H_13 | GCAGTAGAGTAGGTAGAGATTAGGCACATGTTATTTTGATGGGGTCAGTGCCTTG |
| 1149 | handle_32H_14 | GCAGTAGAGTAGGTAGAGATTAGGCAGTCAATCATTTACCTAAACAGTTAATGCC |
| 1150 | handle_32H_15 | GCAGTAGAGTAGGTAGAGATTAGGCAGGCTTGCAGGGAGTTATATTCGG |
| 1151 | handle_32H_16 | GCAGTAGAGTAGGTAGAGATTAGGCAAACGAGGGAATAAATCAAGTATTAAGACATTGA |
| 1152 | handle_32H_17 | GCAGTAGAGTAGGTAGAGATTAGGCACTTAGATTAAGACGCATAAATAA |
| 1153 | handle_32H_18 | GCAGTAGAGTAGGTAGAGATTAGGCATTTTCAAAGTGAACCACCCTAAAGGGAGCCC |
| 1154 | handle_32H_19 | GCAGTAGAGTAGGTAGAGATTAGGCACTGACCTAAAAACCGTCGGGGAAA |
| 1155 | handle_32H_20 | GCAGTAGAGTAGGTAGAGATTAGGCAAATTAAGCCTCCAGTATAAAGCCAA |
| 1156 | handle_32H_21 | GCAGTAGAGTAGGTAGAGATTAGGCAGATCTACATGCTTCTTTCAACTTTACATCAAGAAAA |
| | | C |
| 1157 | handle_32H_22 | GCAGTAGAGTAGGTAGAGATTAGGCATTAATGCCGGAGAGGGAATTAC |
| 1158 | handle_32H_23 | GCAGTAGAGTAGGTAGAGATTAGGCAGGCCGGAGACAGTCA |
| 1159 | handle_32H_24 | GCAGTAGAGTAGGTAGAGATTAGGCAATAAATTGTAAAGATTCAAAAGGTGTACCCC |
| 1160 | handle_32H_25 | GCAGTAGAGTAGGTAGAGATTAGGCAAAAGTACAACGGAGATTTGTATCACCTGCTC |
| 1161 | handle_32H_26 | GCAGTAGAGTAGGTAGAGATTAGGCATGACCCCCAGCGATTGAATTTT |
| 1162 | handle_32H_27 | GCAGTAGAGTAGGTAGAGATTAGGCAAGGCAAAAGAATACACTTTAAT |
| 1163 | handle_32H_28 | GCAGTAGAGTAGGTAGAGATTAGGCACATTAAACGGGTAAAATTGCGCCG |
| 1164 | handle_32H_29 | GCAGTAGAGTAGGTAGAGATTAGGCAAGGACTAAAGACTCACCCTCAGCAGC |
| 1165 | handle_32H_30 | GCAGTAGAGTAGGTAGAGATTAGGCATATATAACTAGCAACGGCTACAGGCATCGG |
| 1166 | handle_32H_31 | GCAGTAGAGTAGGTAGAGATTAGGCATGAATTTATCAAAATTGCAGAACCGGGTATT |
| 1167 | handle_32H_32 | GCAGTAGAGTAGGTAGAGATTAGGCACTACCTTTTTAACCTC |

TABLE 3C: Staple sequences for triangle 2, version 3

| SEQ | | |
|---|---|---|
| ID NO: | Description | Sequence/Details |
| 1168 | core_1 | CCGTTTTTTTAGCGTTAGAGCCAGCAAAATAGGGAGCCGCTTTCCAGTCGGGAA |
| 1169 | core_2 | GAATAACCTGTCGTGTTTTTCAGCTGCATTAATGTTTTTTGGGGTCGAGGTGCC |
| 1170 | core_3 | AGTCAGAATCTTGACATTACGAGACCAACGCCACGTTGGTGTAGATCCGTAATG |
| 1171 | core_4 | AAAATCCTGTTTAACGGGTAAAAACATGCTTTGAATACCAAGTTACAAAATCGC |
| 1172 | core_5 | TTCTTTGCTCGTCATTTAGATGGTGGTTCCGAAATCGGCAAAATCCGGTAATGG |
| 1173 | core_6 | GTGAGAACTCATAGTTAGCGTATTGCGGGAAGTAGTAGCATTAACGGGCGCGA |
| 1174 | core_7 | CGGATTGAGAACGGTCTGACCAATGCCACTAACTTTTTCATGAGGAAGTTTCCA |
| 1175 | core_8 | CTTATAAAACGCTGGTTTGCCCCGCAGAGGCTTACATCGGGAGAAAGTTTAAC |
| 1176 | core_9 | TTTATCCCAGAGCCTCTTCATCGACCAGGCTTACCGTTCCAGTCATCAGATGC |
| 1177 | core_10 | TTTCATCACCCAAAAAGACCTGCTTTGACCGCAGCGAAAGACAGCATCGGAAC |
| 1178 | core_11 | TCATTGACAGGAGGTTGCCCCCTGCCTATTTAACCGATCGCCACCCTCAGAAC |
| 1179 | core_12 | ACCCAGCTAACTTGACCATTAGACTATATTTTCATTTGATCCAATAAGCAAAC |
| 1180 | core_13 | GAGATAGGTGAGGATCCCCGCACTCTGTGGTGCTGCGGAAAGTTTGCAGTTGG |
| 1181 | core_14 | CGCCACCCTCAGAGCTTTTTACCACCCTCATTTTCACGGTTTATCAGCTTGC |
| 1182 | core_15 | TTTTTTAACCAAATTGGCAACATAGACAGCCTAGAAAGGAACAACTAAAGGAAT |
| 1183 | core_16 | CGTAACCGTGCACACGACGTTGTCCTACCACTGCTCATAACCAGCTCAGACGA |
| 1184 | core_17 | CCCTTCACCGCGTTGTTCCAGTTTGGAATTTTTAAGAGTCCACTATTAAAG |
| 1185 | core_18 | ATAACGGAATACCACTACGCGAGGCAGGCAAATATATTTTGTTTGAAATAC |
| 1186 | core_19 | CCGCCGCGTCCGGCAGATATAGTGTTTACCAACGCTCGCGTTAAATAAGA |
| 1187 | core_20 | TTCATATGCACCCTCAGAACCGCACCTTTAACAGAGAGAATAACATAAAT |
| 1188 | core_21 | GGCTTGCACGTATAACAGGAGGTAAGTTTTTCCCACGTCGAGAGGGTTGA |
| 1189 | core_22 | TGACGACGAAAAGAAGTTTTTGATAAGAGGTCACTCCCTCTTAAGAAATAAAGGT |
| 1190 | core_23 | GATGATACACTTAGCCTGAACAAATGTGAGCCAGTTACAAAATAAACT |
| 1191 | core_24 | AGCGAACCGATTGGCCAATTGAGTGATTTGTATGTAAACGATCACCAT |
| 1192 | core_25 | TAGCCATATAAATAGCAAGCCCTTTAGAGCCGCGTTTACCAGCGCCAA |
| 1193 | core_26 | AACGTGGACTACGCGTGGCTGTTTCTCTGGTCATTTTTCAGCAAGAATTCGTAATCA |
| 1194 | core_27 | TTGCTAAAGTCGTCTTTCCAGACGGTACCAAACAGGCAAGGCAAAGAATCAATAAC |
| 1195 | core_28 | TTTTTAGAACCCTCATATATTTTCGTCACCGTGGCGGATAAGTGCATAATAAG |
| 1196 | core_29 | CGCTGCGCGTAACCAAATTTTCCGTCAATACTACCTTTAACAGTAGGGCTTAAT |
| 1197 | core_30 | GAGCTAAATGCGGGAGCGTTTTAGCGAACCTTTAGCAAATGATTAAATGGAGCGGG |
| 1198 | core_31 | AGGAGTGTAATGGAAAGCGCAGTCATTAAGAGAACACCCTAACCCTCGTTTACCAT |
| 1199 | core_32 | ATTTTACCAGAATGCGGCGTTTTTGCCGTTTTCACGGTACGTTATTA |
| 1200 | core_33 | CAGACTGTAGCGCGTTTAAAGCATTGCCGTAATATTTAGCGCCATCTTCGCTATTA |
| 1201 | core_34 | AGCACGTACTTAATGTGAGAAGACTTAGAATCCTTGAAAACATAGCGCAAGTGT |
| 1202 | core_35 | ACTAAAGCGAAGGCACCAACCTATCAGGTAACTAGCATGTCAATGGATTCTC |
| 1203 | core_36 | CCACCACCGGAACCGCTTTTTGCTAACGTCAAAATAGTACGGTGTTAATGC |
| 1204 | core_37 | AATTATTCAAGAAAGCGAAAGAATTTTCTGTATGGGATTGAGGGAAGTATTACGC |
| 1205 | core_38 | GTAAAGCACTAAATCGGAACCCTAACACCAGTAAGTGAGCTTCGCTG |
| 1206 | core_39 | CTACTAATGAAGCCTTTATTTCAACGCAAGGCATTCCACATAAAAGA |
| 1207 | core_40 | TCAGGATTAGAGAGTCACCCTCAAATAGCAAAGACACCATTGTTAAA |
| 1208 | core_41 | CGAGGAAACCCAATTCTGCAACACCGCCTGCAACAGTGCATTAGCGT |
| 1209 | core_42 | TTTTTTGTAAATGCTGATGCAAATCCTTTCAAAGCGCCATTCTGGTG |
| 1210 | core_43 | TATAAAGCCAGTCCCGGAATTTGTTTTTTAGAGATAGACTTATCGG |
| 1211 | core_44 | AGAGGCAACAACGGCTACAGAGGCTTTGAGGTCGTTAACCATAAGG |
| 1212 | core_45 | TTTCGAGGTGAATTTCTTAAACAGTTTTTTTGATACCGATAGTTGC |
| 1213 | core_46 | TGGCTGACAATTTGCGAGTAACAACCCGTCCATATGTAGGCGCAGA |
| 1214 | core_47 | ACAATCAATGCGAATACTACAACGCCTGTAGATAAAAATCAGCTCA |
| 1215 | core_48 | TTGCTCAGTACCATTACCCTCAGAGCCGCCAGCAAACTCCAACAGG |
| 1216 | core_49 | AAAACGAACGGTCAATGGAAGCGTCATACATGGCTTTTGAGGGTAG |
| 1217 | core_50 | TCAGCTAATCTGTAAATCGTCGCTATTAATTCCACGCTCACTGCCC |
| 1218 | core_51 | GAACGGTACGCCGTGAACCACCACAGCAAATGAAAAATCTTCATCG |
| 1219 | core_52 | GCATAGGCGGAACGACCCCGGTTTTTTTGAGAGATCTACAAAGGCT |
| 1220 | core_53 | TAGGTAAAATAGGAACTATAAGTAGAAGGATTAGAGCCGCCGCCAG |
| 1221 | core_54 | CAGGAAGGCTGATAAATTAATGCCGGAGAGCACTCATCTCCATGTT |
| 1222 | core_55 | CCATGTAAAAGGCTCCAAAAGGAGCCTTTACCTCAAGATAGCCCT |
| 1223 | core_56 | TTATTTGCACGTAAAGGGCGCATCGAGAAACCTACATTAGGAAAA |
| 1224 | core_57 | TAGATACAGAGCAACTTGAATCGGATTGCATCAAAAAGTCTGAAT |
| 1225 | core_58 | AAGAATACACTAAAAGGTAGCTAGATAATCAGAAAAGCACATTAA |
| 1226 | core_59 | AGTAGATTTAGTGATGAACAAAGACCAGAGTTCAACCGATTGAGG |
| 1227 | core_60 | CTTACACAGCGGTGCCGGTGCCTCAAAAATATCAACGTGCCCTGA |
| 1228 | core_61 | TGGTCATACCTGTTCTTCGCGTTTTTCCGTGAGCCTCCTCACAGT |
| 1229 | core_62 | AATCGTAACATTGCCTGAGAGTCTGGAGCAATACGTAAAGCGGGG |
| 1230 | core_63 | GCACAGACTAACGTGCCAGGCTGCAAGGCTTAGACAAAGTTAACCTC |
| 1231 | core_64 | AACATTATGACCCTGTAATACTTACGATCTAGTAGAAAAGGGCGACA |
| 1232 | core_65 | CAATCAGAAGATGATGAATTTTTCAAACATCCAACAGCTGATTG |
| 1233 | core_66 | CGAGCTCCCGCAAGACGGGTATTTTTTAAACCAAGTGTATTTTT |
| 1234 | core_67 | TACATACAAGTAAGCGCTGAAACCAATCCAAATAAGAACAATTT |
| 1235 | core_68 | TAACAATTGGGTGGTTATCCGCTCACAATTCCCGGACTTAACAAGCA |
| 1236 | core_69 | AAAGCTAAATCGGTTTTAGTAAGACTCCTGTAAATATTTTCAT |
| 1237 | core_70 | CAATATGATATTCAAAGCGCGATGATAAATCAGTGCCACAAAC |
| 1238 | core_71 | ACCTTGCTTGCAGATTTTTCGCGCCTGATCAATATATGTGAGT |
| 1239 | core_72 | CATATTTAACAATTCTTTTTTTCCAGTGAGAATCAATAGCAA |
| 1240 | core_73 | CTTTCCTTAACATGAGCCGAGCGGTCCTCAAAAGAATAGCCC |
| 1241 | core_74 | ATATCAGAGAGATAACCCACAAGTTGATATTCTTGAGTAACAGTGCC |
| 1242 | core_75 | AATCAAAAGAATATAATGCGAACGCATATAACTAGTTGCTATTTTGC |
| 1243 | core_76 | TTTTTATAAGAGAGAGACACATCGACTCAGCGTGGTGCTGG |
| 1244 | core_77 | GTTGAGTCTGGCCCTGAGAGAGAGCAAAAATAATCGGCTGT |
| 1245 | core_78 | ACAACATGTCATAGGTCTGAGAGAGTGAATTTGCAAATCA |
| 1246 | core_79 | TAAGACGCCGCCGCTAAGCGGTCACGAACGTGGCGAGAAA |
| 1247 | core_80 | GGTTAGAAAAAACGACCCTGATTGGATTATTTCCAGAACA |
| 1248 | core_81 | AGCAGGCGAATCCGCCGTAAAGGTTCCAGCGCAGTGTCACCAAAGAA |
| 1249 | core_82 | TACCCTGACTATTATCGGGTTACAGAGGACAGAACCGAA |
| 1250 | core_83 | TAACAAAGTATCAAAATTGCGTAGATTTTCAGCAATAACGCGCACTC |
| 1251 | core_84 | AGTACCTTGAATTATTCATTTCAATTACCTGTTGCAGCAGGTGGTAC |
| 1252 | core_85 | GAATCGGCCAACGCGTACATAATTTATCAAGACGACAATAA |
| 1253 | core_86 | CAGAGCGGTTAGACAGCCGGAAACCTTTCCGGCACCGCT |
| 1254 | core_87 | ATTGTATAAGCAAATATTTAAATTCATCGCCAACAAAGT |
| 1255 | core_88 | GGGAGTTACAACGGATTTAGTACAGTCAATTAATTTTTTATTTGCCTGAGTAATGTG |
| 1256 | core_89 | TAAAGGGTGAGAAAGGCCGGAGACCGCCACCACCGTACT |
| 1257 | core_90 | AAAAATGATATTTATCAGGTGGCATCATAGGAACGCCAT |
| 1258 | core_91 | GCCGACAATGACAACAACCATCGCCCACGCATCGGAACC |
| 1259 | core_92 | AAAGGCCGCTTTTGCGGGATCGTTAACGGGGTTGTGTCG |
| 1260 | core_93 | TAACAGGGAAGCGCATTAGACGGGAGAGGTAATTGAAGCCAGACTGGTAATAAGTTT |
| 1261 | core_94 | CAGGGCGCGTGAATTAGGTCATAGCCCCCTTCACGCTGA |
| 1262 | core_95 | GATGAACGCGTGGGAATAACGAGCGTCTTTC |
| 1263 | core_96 | GATATTCACTTTGAACTGCAGCCTGGTGTGTTCAGCAAA |
| 1264 | core_97 | AAATGCAATTGTTAAAATTCGCATTAAATTTCGGAATAA |
| 1265 | core_98 | AATCAGTTTTTTGCGACAGAATCAAAAACCC |
| 1266 | core_99 | CTTTGACGGCATTTTCTCACCGTCACCGACTAAGCCGG |
| 1267 | core_100 | AAATAAGAACGCGAGAAAACTTAATCGCAATCCGGTAT |
| 1268 | core_101 | TATACTTCAATATAATGGCCAGTTTGGGTAACGCCAGG |
| 1269 | core_102 | ATAAACACGGAAACATCTCCGTGCCGCACATCATAACG |
| 1270 | core_103 | AAGGAGCGGAATTATCCCTGCATTACGGCTGGCGCTTT |
| 1271 | core_104 | TTTTTTGACCTAAGCCTTAACCCGACTCAGGAGGCGCAATATTGCCATCTTGACGGA |
| 1272 | core_105 | TGGAATAGGTGTATCCTCAGAACATATTCGGTCGCTGA |
| 1273 | core_106 | GATAGCTCTAGTATCACATAATTACTAGAAAATTTTTT |
| 1274 | core_107 | AAGTTTTCAACTTTCAACAGTTTCAGCGGAAGACAAAA |
| 1275 | core_108 | TCGAGCCAACCGCACTCCTAATTTAATTGTT |
| 1276 | core_109 | TTATCATTTTGCGGAATGCGCGCCCGTCGGTTTGCGGT |
| 1277 | core_110 | AATTGCTTCACCGGATTACCAGAGTATGTTAATCATA |
| 1278 | core_111 | GAGCCGCCACGGGGAGCACTAGTAATAAACATCACTT |
| 1279 | core_112 | CGGCAGCACTGTGCACTGTTGCCCTGCGGCT |
| 1280 | core_113 | GGGCGCGGGGTGCCAGTCAGATGAATGGAAG |
| 1281 | core_114 | CGACCGTGTGATTGGGAAGGTCGCCATTTTTCCTCG |
| 1282 | core_115 | GGCATTTAGCCTGTTTCACGGAAAAAATATAAAGTA |
| 1283 | core_116 | AAATAAATTCGCGTTTTAATTCGAGCTTCAA |
| 1284 | core_117 | TATTATTCTGAAACATTTTTTAAAGTATTAAGAGGC |
| 1285 | core_118 | TGAGACTATTGTATGGGATAGCAAGCCCAGATTCAT |
| 1286 | core_119 | GCGGTTGTGTGCAGTTTTTAACAGCGGATCAAACT |
| 1287 | core_120 | TAATGGAAACAGCGGGGAGACGGAAGCATAAAGTG |
| 1288 | core_121 | TGTTTAGACTGGATAGCGTCCAATACGAAAGACT |
| 1289 | core_122 | AATTAAGCAATAATAGTTATATAACTATATTTTT |
| 1290 | core_123 | GAACGTGCACACAACGAACAAGAATTACCTTTTT |
| 1291 | core_124 | CGCTAGGGCGCTGGAAGCCTCAGAGCAT |
| 1292 | core_125 | GCGGGGTTTCCAAAAACCGTAACACTGAGTTT |
| 1293 | core_126 | CCAGCATCTTACCCAACAGGTCTTGAGAATGA |
| 1294 | core_127 | ATTAGCAAGGCCGGAAACGTCAAATCAA |
| 1295 | core_128 | ACAGTTAATGAGGCAGGTCAGACAGACTTTTTCGGAACCAGAACCACCACCAGGATTA |
| 1296 | core_129 | TTTCTTTAAAGGGCGAAAAACCGTCT |
| 1297 | core_130 | AGATAGCCTTTTTGTTGGATGGCTAACTAAAG |
| 1298 | core_131 | ATGGTTGTTAGAATGAGCCAGGCAGAAG |
| 1299 | core_132 | TTACCGAGCCTTTATTGCTCCTTTGCCA |
| 1300 | core_133 | TGAATCTTGCATAGTAATAACGCCTTTCAACT |
| 1301 | core_134 | ATCAAGATAGTTGATTTCAGTTGAAAAACGAA |
| 1302 | core_135 | GGCCAGAGGAATCATTATTTTTCGCGCCC |
| 1303 | core_136 | AAGAACTGGCTCATTACTTTTGCATAAAAACC |
| 1304 | core_137 | CACCCTCACCCAGCGATTATACCACCGTTCTA |
| 1305 | core_138 | AACGCGGTGATGCTGATCACCTTGATCGCCAT |
| 1306 | core_139 | AAGGAAACCTGTTTAGTACATTTCGCAAATGG |
| 1307 | core_140 | CAAATATCGTTTGCCTTCGTCTCGAACTCACA |
| 1308 | core_141 | ATCAAAATTTAATTGCGTTGCACTTGGGAAT |
| 1309 | core_142 | AATCCCGTATCTTTAGAACGGATAGGGG |
| 1310 | core_143 | AGTACAAAATAATTTTTTCACGTTGAAAATC |
| 1311 | core_144 | CGGCTTAGGTTGGGCTTAGATTCTAAGA |
| 1312 | core_145 | ATCAGGGCGATGGCGTATTGGGCGCCATCATTTGAAAAATAACCGACAAATAGGCAGA |
| 1313 | core_146 | AAAAAAAGGTGAAGGAGCCGTTTATGTAATT |
| 1314 | core_147 | AGCAAGAAACAATGAGAGCCACCATTTTGTC |
| 1315 | core_148 | ATTTAATGAGTTAATTTCATCTTCTTTTT |
| 1316 | core_149 | GATTCGCCTGATAGAGAATCGATACAGAAAT |
| 1317 | core_150 | TCAATAGATAAATCTGATTCTGCTCATTT |
| 1318 | core_151 | CCGGAATTATGCGTTATACAAACGCCAAC |
| 1319 | core_152 | ATGCCAAGCCGCCAGAGTAACATTTAGAAG |
| 1320 | core_153 | CGAAACGTACTTGAATGGCTATTAGTTTTTTTTTAAATTGAGGTAGAATTTGTTGTAGC |
| 1321 | core_154 | TCAAATACCTCATTAAGCGCTAAAATCCGC |
| 1322 | core_155 | AAACATCCTCATTGCAGGAGGTGTAAAGAAA |
| 1323 | core_156 | TTATTTTTAAGTCCTATACGAGC |
| 1324 | core_157 | CCCGATTTAGAGCTTGACGGGGATGAGCCAT |
| 1325 | core_158 | TCCCACGCTTTGGATACCACCAGATCAGAT |
| 1326 | core_159 | CGAGTAGTAGAACCGGGATAGGTCAAACGG |
| 1327 | core_160 | CGCCAGCAACAGAGAAAGGTTATCTAAAAT |
| 1328 | core_161 | CTGTGTGAACGAGCAAAAATTAATTACATT |
| 1329 | core_162 | TAAATATAATATACAGTATCATTCCAAGAA |
| 1330 | core_163 | GGAAGGGATTAAAGGTCAAAAGAACTGGC |
| 1331 | core_164 | AGTCAGAGATTAACTGGAAGCCCTGCGGAA |
| 1332 | core_165 | GTGTACAAAGAGTAAGCAAAGCCCCCTCAA |
| 1333 | core_166 | TATCCCATCATCGAGGTAGAACGATAAAAA |
| 1334 | core_167 | AGGTAAAGTAATTCTGTCCAGACCAATAGA |
| 1335 | core_168 | TCAATCAATGCACCGTCAGCCTCCTAAAGCCTGGGGTGCTTTTTTAATGGCACCATTACC |
| 1336 | core_169 | TAGAAAAAACGCAATAGCTATCCAAAAATAATTCGCGTCTTTTTGGCCTTCCTGTAGCCAGC |
| 1337 | Side3_rec1 | TCTGCCAGCCCAACGTCTCACCAGTGAGACGGGAAGAAAACTGTAGAAAC |
| 1338 | Side3_rec2 | TTTAAATCCTTTGCCCGACATACCGGT |
| 1339 | Side3_rec3 | TCGATAGCAATCTGGTTTAGTGATGATTTTTGGGTAAAGTTAAAC |
| 1340 | Side3_rec4 | TTTTTGACCAACAACTAAATTCGACAAT |
| 1341 | Side3_rec5 | CATCGTAGCACATCCGGCGGCCTCAGTTGGCAAATCAATT |
| 1342 | Side3_rec6 | GGCGGTTTGCCCACTACGTGAACCATCACCCACCAATGAT |
| 1343 | Side3_rec7 | TAGTTGAAAGGATGCGCGAACTTTTTGATAGCCCTAAAACCTGAACCT |
| 1344 | nohandle_1 | AAATTGGGATCGTTTTTCCTCAGGAAGA |
| 1345 | nohandle_2 | AATACCACATTCAACCTGGAAGTAAATATGCTAGAGCTT |
| 1346 | nohandle_3 | CAGATTCACGATTAAGGCCAAGCTTTCAGAGGTG |
| 1347 | nohandle_4 | AGGTAGAACCAGCCAGGTTTTGAA |
| 1348 | nohandle_5 | CAATCGTAGTTCTGCCAGTTTGAGGGGACG |
| 1349 | nohandle_6 | CGTAAGAATGCGGGCCGCAACTGT |
| 1350 | nohandle_7 | GTTTTCCCCTGAAATGTCAGTGATCCTGATT |
| 1351 | nohandle_8 | TGGGAAGAAAAATCTATGTTTTTTCATTC |
| 1352 | nohandle_9 | TCGCACTAGATTCACCGATTAGCGGTCAGTATTTGT |
| 1353 | nohandle_10 | ACGCAAATTTTTTTACCTTCCTTCTGACTGTCCATC |
| 1354 | nohandle_11 | ACTATCGGCCTTGCTTTCTACATTTGAGGA |
| 1355 | nohandle_12 | ACGACAGTGGAAATACACCAGAACAGAAAAC |
| 1356 | nohandle_13 | TTAATCATTGTGAATAATATTCAACTATCA |
| 1357 | nohandle_14 | CCATAAAATTGCAACTTGACGCT |
| 1358 | nohandle_15 | AGCTCAACACGTTAATGATTTAGG |
| 1359 | nohandle_16 | GCGATCGGTACAGAATCCTGAGAAGTGTTAC |
| 1360 | nohandle_17 | GATGGCAAGGTAATATACATTGG |
| 1361 | nohandle_18 | CGAGAGGTACCAGTCAGGACGT |
| 1362 | nohandle_19 | ATAAAACATATTTTTATAATCAG |
| 1363 | nohandle_20 | ATGCTTTAGGTTTAAAAAAGGAA |
| 1364 | nohandle_21 | AATACTTCTTTGATTAGTAATAAAGGGACAT |
| 1365 | nohandle_22 | TCGTCATATACCTTATGCGATTTT |
| 1366 | nohandle_23 | GAGGGGGTAATAGTAAAA |
| 1367 | nohandle_24 | TATTAGATTTTTTTTACAAACATAGTTTTTTTAGAGCCG |
| 1368 | nohandle_25 | GCCTGAGTAGATTTTTGAACTCAA |
| 1369 | nohandle_26 | TGAGGCCACCGAGTAAACCGAACG |
| 1370 | nohandle_27 | TAAAAATAAGAGTCCTGAAAG |
| 1371 | nohandle_28 | ACGCTCATCTTGAGATAACAGTT |
| 1372 | nohandle_29 | CTAACGGAACAACATGAGGTGAGAAGGGATT |
| 1373 | nohandle_30 | ATATTACCGCCAGCCCATCATATATAAGGCT |
| 1374 | nohandle_31 | GATGTGCTGCTTTTTAAGGCCAGTCACA |
| 1375 | nohandle_32 | TCTGGCCTGGCGAAAACCTCAC |
| 1376 | handle_32LH_1 | GCAGTAGAGTAGGTAGAGATTAGGCATTTTTTTTTTTTTTTTTTTTAAATTGGGATCGTTTTTC |
| | | CTCAGGAAGA |
| 1377 | handle_32LH_2 | GCAGTAGAGTAGGTAGAGATTAGGCATTTTTTTTTTTTTTTTTTTTAATACCACATTCAACCTG |
| | | GAAGTAAATATGCTAGAGCTT |
| 1378 | handle_32LH_3 | GCAGTAGAGTAGGTAGAGATTAGGCATTTTTTTTTTTTTTTTTTTTCAGATTCACGATTAAGG |
| | | CCAAGCTTTCAGAGGTG |
| 1379 | handle_32LH_4 | GCAGTAGAGTAGGTAGAGATTAGGCATTTTTTTTTTTTTTTTTTTTAGGTAGAACCAGCCAGG |
| | | TTTTGAA |
| 1380 | handle_32LH_5 | GCAGTAGAGTAGGTAGAGATTAGGCATTTTTTTTTTTTTTTTTTTTCAATCGTAGTTCTGCCA |
| | | GTTTGAGGGGACG |
| 1381 | handle_32LH_6 | GCAGTAGAGTAGGTAGAGATTAGGCATTTTTTTTTTTTTTTTTTTTCGTAAGAATGCGGGCCG |
| | | CAACTGT |
| 1382 | handle_32LH_7 | GCAGTAGAGTAGGTAGAGATTAGGCATTTTTTTTTTTTTTTTTTTTGTTTTCCCCTGAAATGTC |
| | | AGTGATCCTGATT |
| 1383 | handle_32LH_8 | GCAGTAGAGTAGGTAGAGATTAGGCATTTTTTTTTTTTTTTTTTTTGGGAAGAAAAATCTAT |
| | | GTTTTTTCATTC |
| 1384 | handle_32LH_9 | GCAGTAGAGTAGGTAGAGATTAGGCATTTTTTTTTTTTTTTTTTTTCGCACTAGATTCACCG |
| | | ATTAGCGGTCAGTATTTGT |
| 1385 | handle_32LH_ | GCAGTAGAGTAGGTAGAGATTAGGCATTTTTTTTTTTTTTTTTTTTACGCAAATTTTTTTACCT |
| | 10 | TCCTTCTGACTGTCCATC |
| 1386 | handle_32LH_ | GCAGTAGAGTAGGTAGAGATTAGGCATTTTTTTTTTTTTTTTTTTTACTATCGGCCTTGCTTTC |
| | 11 | TACATTTGAGGA |
| 1387 | handle_32LH_ | GCAGTAGAGTAGGTAGAGATTAGGCATTTTTTTTTTTTTTTTTTTTACGACAGTGGAAATACA |
| | 12 | CCAGAACAGAAAAC |
| 1388 | handle_32LH_ | GCAGTAGAGTAGGTAGAGATTAGGCATTTTTTTTTTTTTTTTTTTTAATCATTGTGAATAAT |
| | 13 | ATTCAACTATCA |
| 1389 | handle_32LH_ | GCAGTAGAGTAGGTAGAGATTAGGCTTTTTTTTTTTTTTTTTTTTCCATAAAATTGCAACTTG |
| | 14 | ACGCT |
| 1390 | handle_32LH_ | GCAGTAGAGTAGGTAGAGATTAGGCATTTTTTTTTTTTTTTTTTTTAGCTCAACACGTTAATGA |
| | 15 | TTTAGG |
| 1391 | handle_32LH_ | GCAGTAGAGTAGGTAGAGATTAGGCATTTTTTTTTTTTTTTTTTTTGCGATCGGTACAGAATC |
| | 16 | CTGAGAAGTGTTAC |
| 1392 | handle_32LH_ | GCAGTAGAGTAGGTAGAGATTAGGCATTTTTTTTTTTTTTTTTTTTGATGGCAAGGTAATATA |
| | 17 | CATTGG |
| 1393 | handle_32LH_ | GCAGTAGAGTAGGTAGAGATTAGGCATTTTTTTTTTTTTTTTTTTTCGAGAGGTACCAGTCAG |
| | 18 | GACGT |
| 1394 | handle_32LH_ | GCAGTAGAGTAGGTAGAGATTAGGCATTTTTTTTTTTTTTTTTTTTATAAAACATATTTTTATAA |
| | 19 | TCAG |
| 1395 | handle_32LH_ | GCAGTAGAGTAGGTAGAGATTAGGCATTTTTTTTTTTTTTTTTTTTATGCTTTAGGTTTAAAAA |
| | 20 | AGGAA |
| 1396 | handle_32LH_ | GCAGTAGAGTAGGTAGAGATTAGGCATTTTTTTTTTTTTTTTTTTTAATACTTCTTTGATTAGT |
| | 21 | AATAAAGGGACAT |
| 1397 | handle_32LH_ | GCAGTAGAGTAGGTAGAGATTAGGCATTTTTTTTTTTTTTTTTTTTCGTCATATACCTTATGC |
| | 22 | GATTTT |
| 1398 | handle_32LH_ | GCAGTAGAGTAGGTAGAGATTAGGCATTTTTTTTTTTTTTTTTTTTGAGGGGGTAATAGTAAA |
| | 23 | A |
| 1399 | handle_32LH_ | GCAGTAGAGTAGGTAGAGATTAGGCATTTTTTTTTTTTTTTTTTTTATTAGATTTTTTTTACAA |
| | 24 | ACATAGTTTTTTTAGAGCCG |
| 1400 | handle_32LH_ | GCAGTAGAGTAGGTAGAGATTAGGCATTTTTTTTTTTTTTTTTTTTGCCTGAGTAGATTTTTGA |
| | 25 | ACTCAA |
| 1401 | handle_32LH_ | GCAGTAGAGTAGGTAGAGATTAGGCATTTTTTTTTTTTTTTTTTTTGAGGCCACCGAGTAAA |
| | 26 | CCGAACG |
| 1402 | handle_32LH_ | GCAGTAGAGTAGGTAGAGATTAGGCATTTTTTTTTTTTTTTTTTTTAAAAATAAGAGTCCTGA |
| | 27 | AAG |
| 1403 | handle_32LH_ | GCAGTAGAGTAGGTAGAGATTAGGCATTTTTTTTTTTTTTTTTTTTACGCTCATCTTGAGATAA |
| | 28 | CAGTT |
| 1404 | handle_32LH_ | GCAGTAGAGTAGGTAGAGATTAGGCATTTTTTTTTTTTTTTTTTTTCTAACGGAACAACATGA |
| | 29 | GGTGAGAAGGGATT |
| 1405 | handle_32LH_ | GCAGTAGAGTAGGTAGAGATTAGGCATTTTTTTTTTTTTTTTTTTTATATTACCGCCAGCCCA |
| | 30 | TCATATATAAGGCT |
| 1406 | handle_32LH_ | GCAGTAGAGTAGGTAGAGATTAGGCATTTTTTTTTTTTTTTTTTTTGATGTGCTGCTTTTTAAG |
| | 31 | GCCAGTCACA |
| 1407 | handle_32LH_ | GCAGTAGAGTAGGTAGAGATTAGGCATTTTTTTTTTTTTTTTTTTTCTGGCCTGGCGAAAAC |
| | 32 | CTCAC |

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable sub-combination. All combinations of the embodiments pertaining to the invention are specifically embraced by the present invention and are disclosed herein just as if each and every combination was individually and explicitly disclosed. In addition, all sub-combinations of the various embodiments and elements thereof are also specifically embraced by the present invention and are disclosed herein just as if each and every such sub-combination was individually and explicitly disclosed herein.

The present invention is not to be limited in scope by the specific embodiments described herein. Indeed, various modifications of the invention in addition to those described herein will become apparent to those skilled in the art from the foregoing description. Such modifications are intended to fall within the scope of the appended claims.

To the extent possible under the respective patent law, all patents, applications, publications, test methods, literature, and other materials cited herein are hereby incorporated by reference.

The following Examples illustrates the invention described above, but is not, however, intended to limit the scope of the invention in any way. Other test models known as such to the person skilled in the pertinent art can also determine the beneficial effects of the claimed invention.

### EXAMPLES

### Introduction

Virus-enveloping macromolecular shells or tilings can in principle prevent viruses from entering cells. Here we describe the design and assembly of a cone-shaped DNA origami higher-order assembly that can engulf and tile the surface of pleomorphic virus samples larger than 100 nm. We determine the structures of subunits and of complete cone assemblies using cryo-EM; and establish stabilization treatments to enable usage in in vivo conditions. We use the cones exemplarily to engulf Influenza A virus particles, and SARS-COV-2, Chikungunya and Zika virus-like particles. Depending on the relative dimensions of cone to virus particles, multiple virus particles may be trapped per single cone, and multiple cones can also tile and adapt to the surface of aspherical virus particles. The cone assemblies form with high yields, require little purification, and are amenable for mass production, which is a key requirement for future real-world uses including as an antiviral agent.

To overcome the limitations referred to in the section describing the background of the invention, we here describe an efficiently assembling DNA origami based macromolecular shell system that can engulf pleomorphic viral pathogens larger than 100 nm in diameter as exemplified by Influenza A viruses. Our design concept considers the self-limiting oligomerization of wedge-shaped building blocks into cones. This expansion of a previous implementation of planar finite size assemblies¹⁴ uses a minimized number of subunit types which reduces the complexity of the assembly process. The resulting high yields of assembly make the cone system amenable to mass production as needed for future real-world uses as an antiviral.

### Results and Discussion

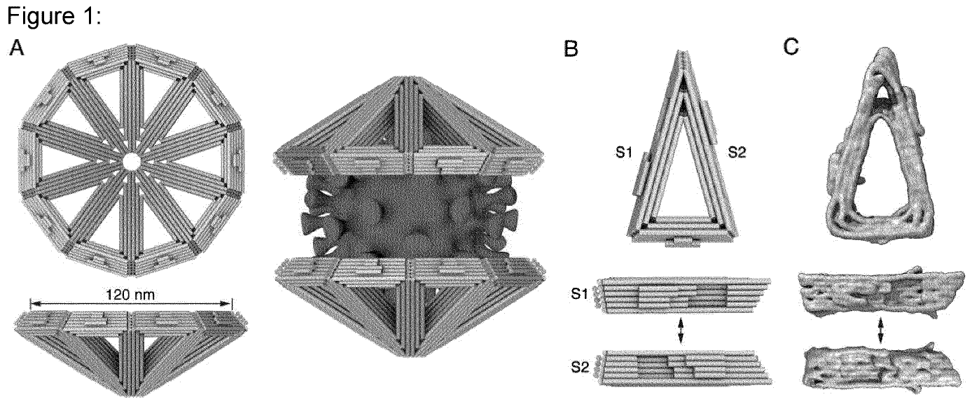

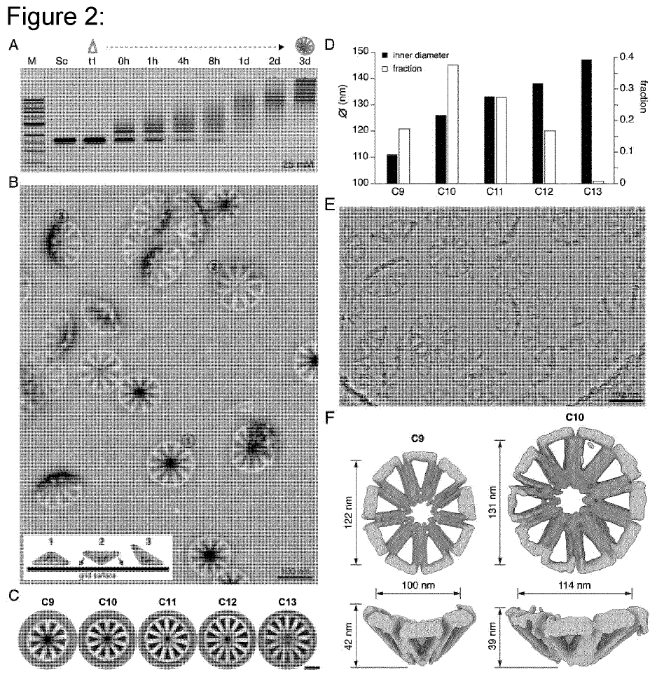

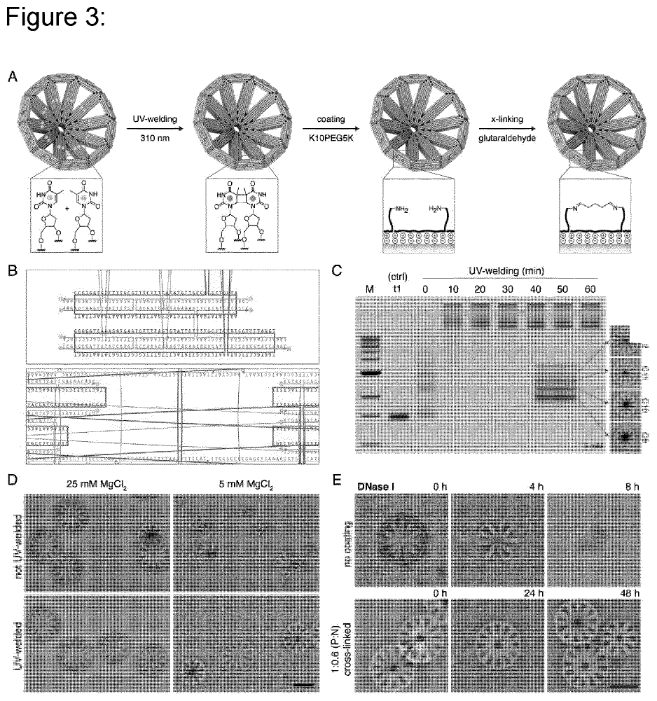

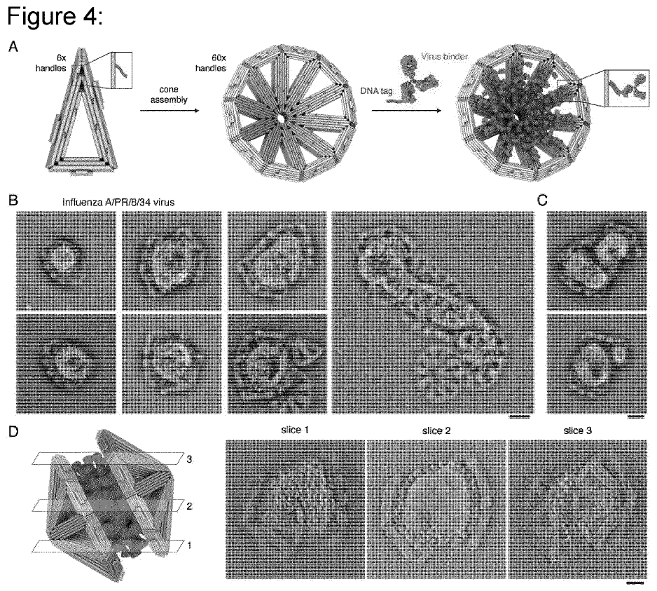

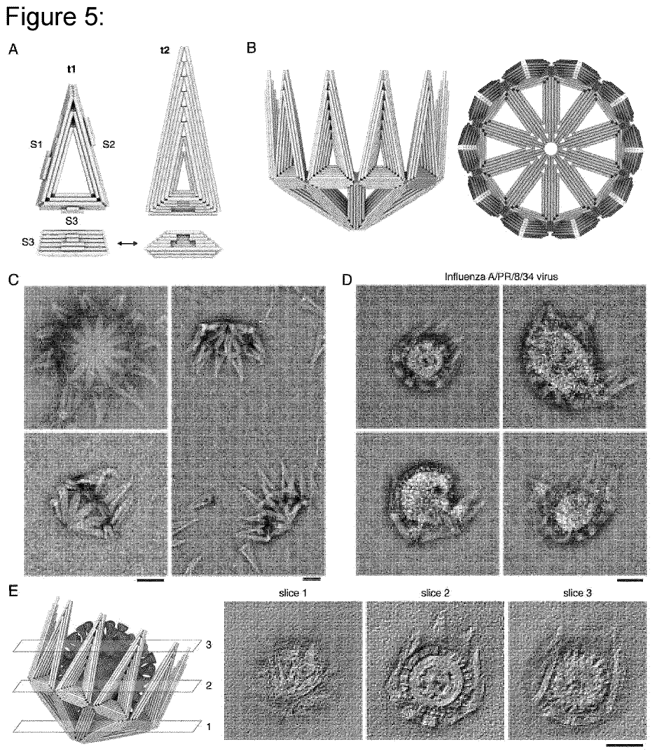

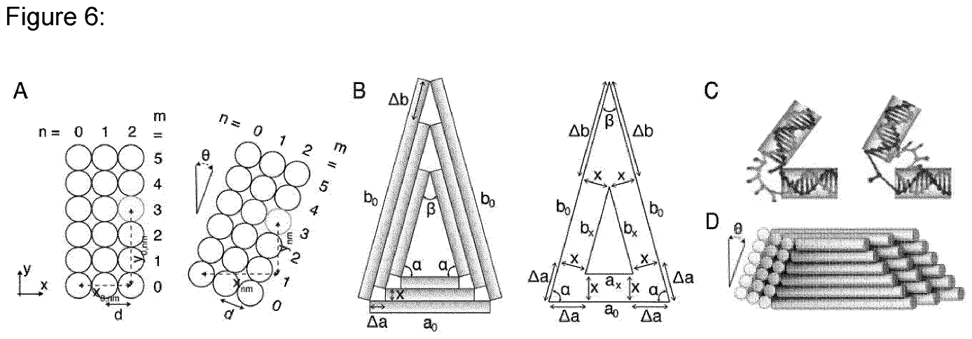

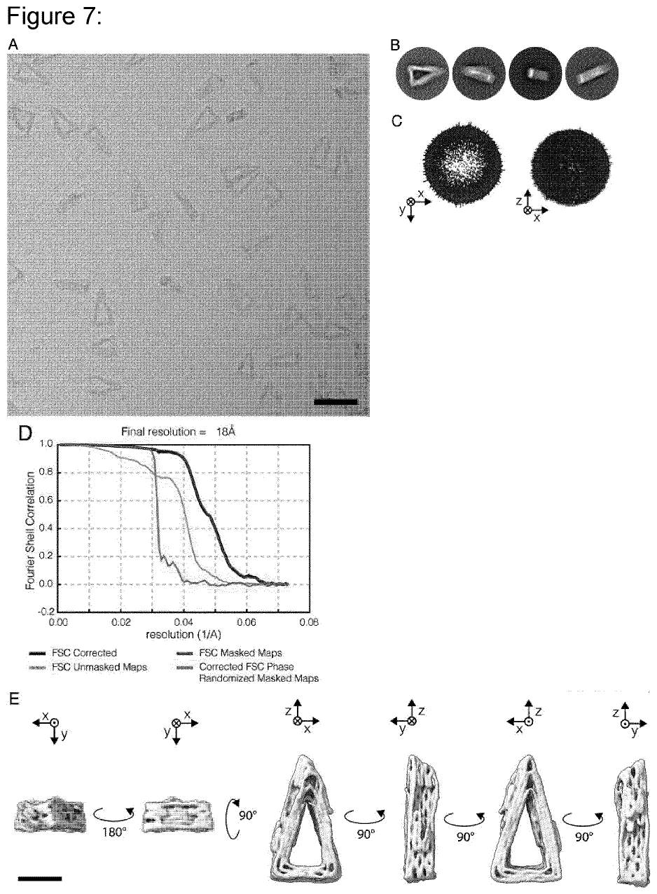

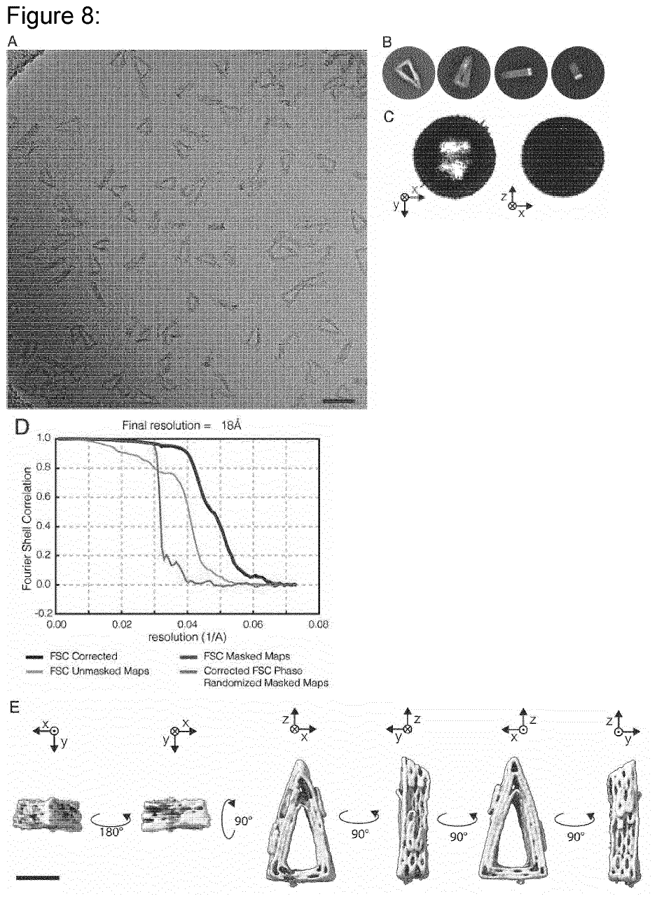

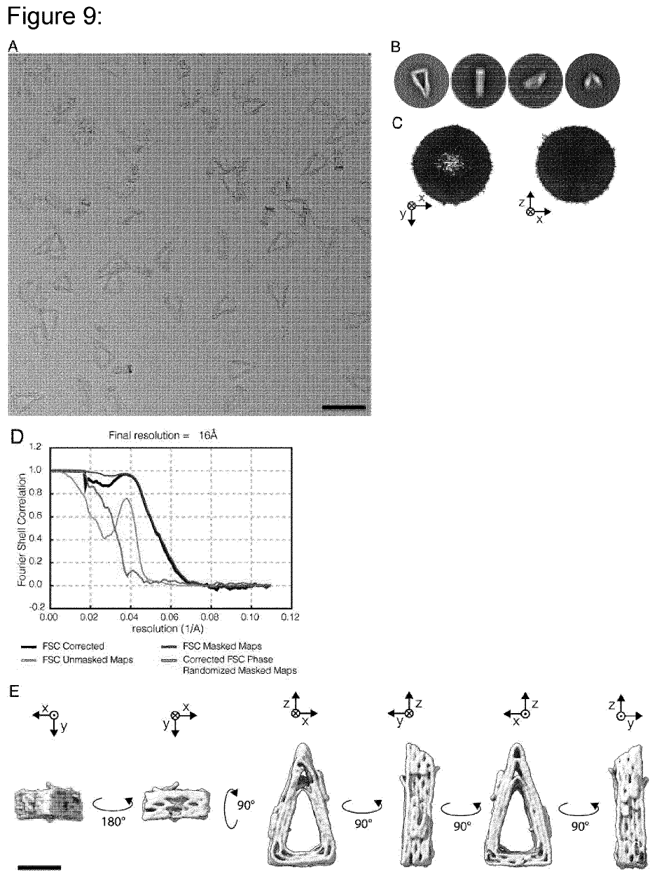

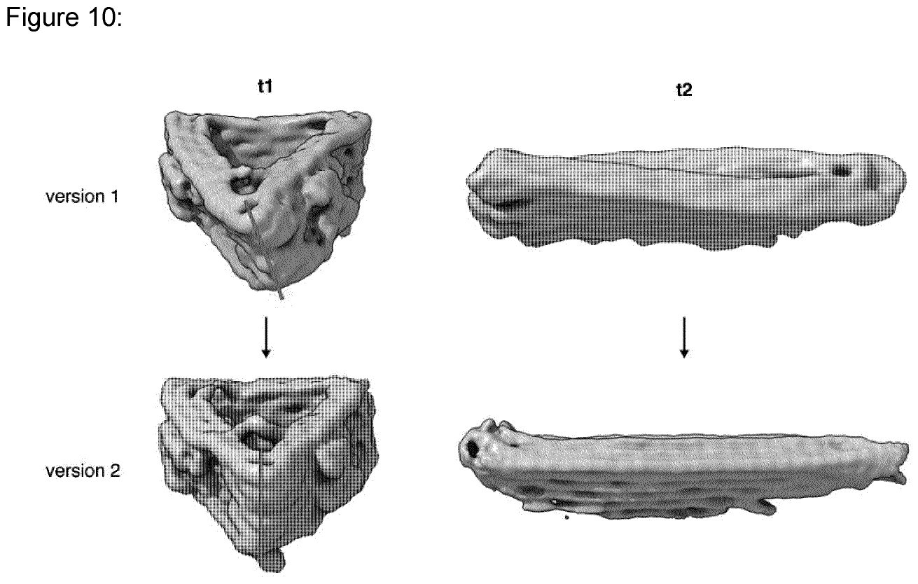

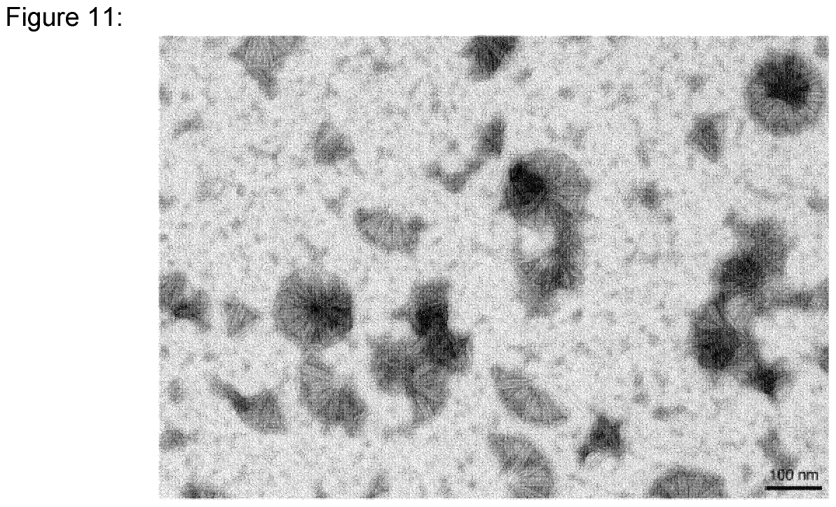

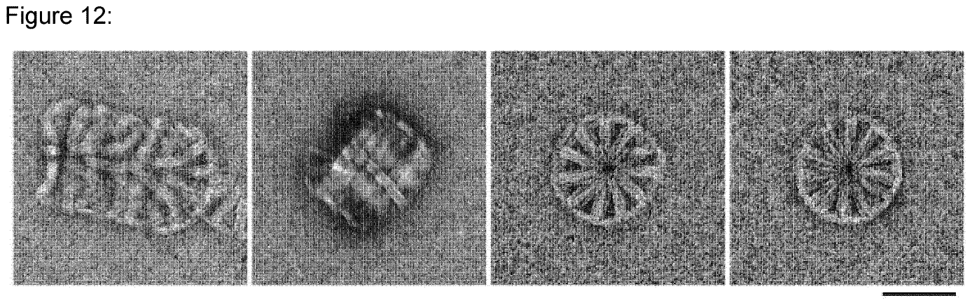

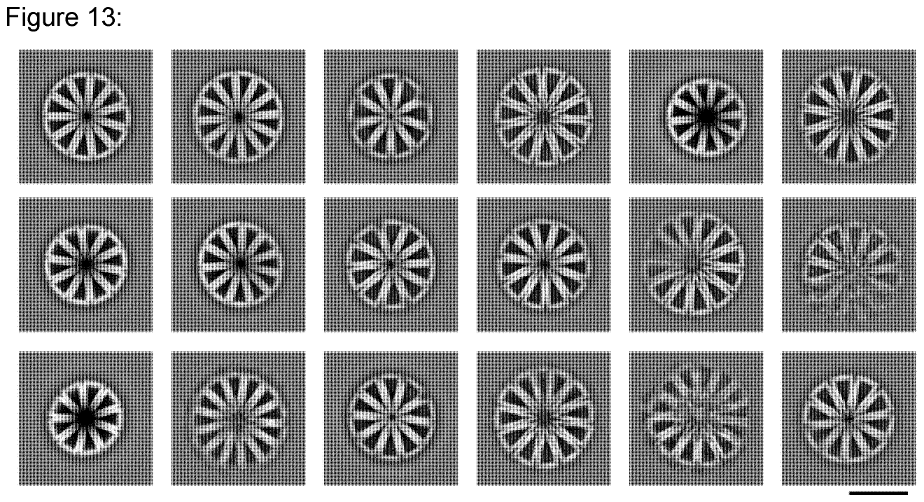

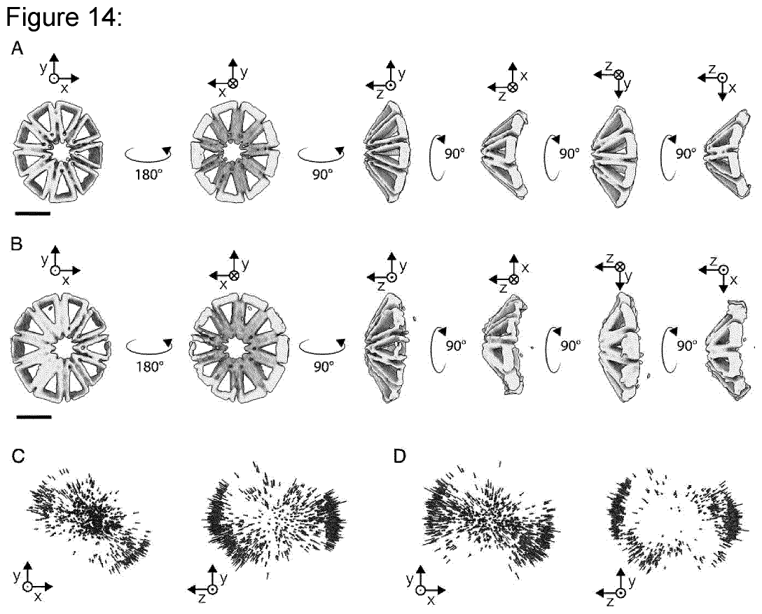

Our cone assemblies are designed to form from multiple copies of a wedge-shaped building block (t1) (see supporting information for design details). The wedge building block can oligomerize via two distinct self-complementary edges at opposite faces. Oligomerization of the wedges leads to circular assemblies that close upon themselves. Given the designed geometry of the wedge, we expect the cone to have ten facets (FIG. 1A, B). The diameter of the base of the cone made of the ten wedges was designed to measure ˜120 nm, so that two copies of a cone would, for example, be sufficiently large to enclose an Influenza virus particle (˜80-200 nm) in a sandwich-like assembly (FIG. 1A, right).

We implemented the wedge building block with multi-layer DNA origami in square-lattice helical packing.^{15,16} We assembled the objects using the methods of DNA origami and used single-particle cryogenic electron microscopy (cryo-EM) to improve and validate the design of our wedge subunit in an iterative process (FIG. 7-9). The 3D electron density maps we determined for the single wedge particles revealed the designed overall shape of the triangular building block and the shape-complementary docking features (FIG. 1C). Our initial wedge design displayed a pronounced global twist deformation, which we then corrected to give a nearly twist-free shape (FIG. 10).

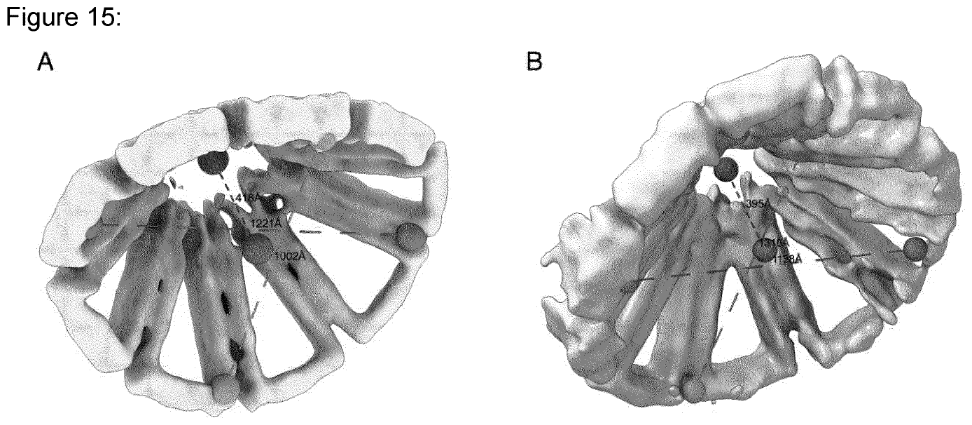

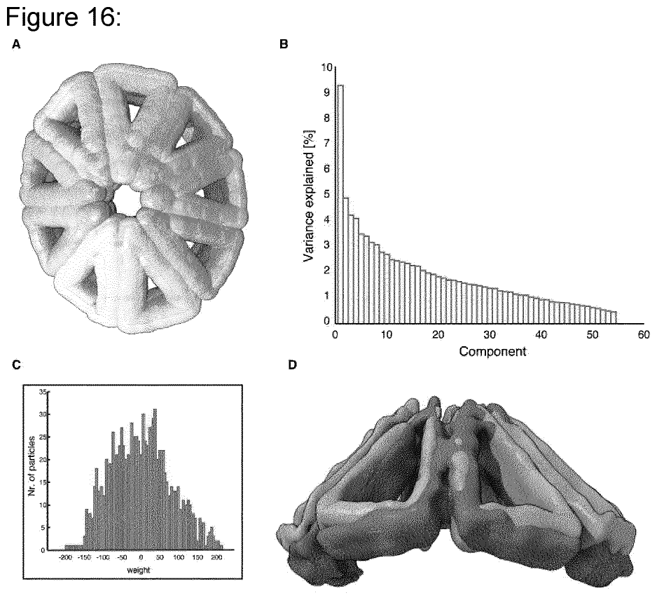

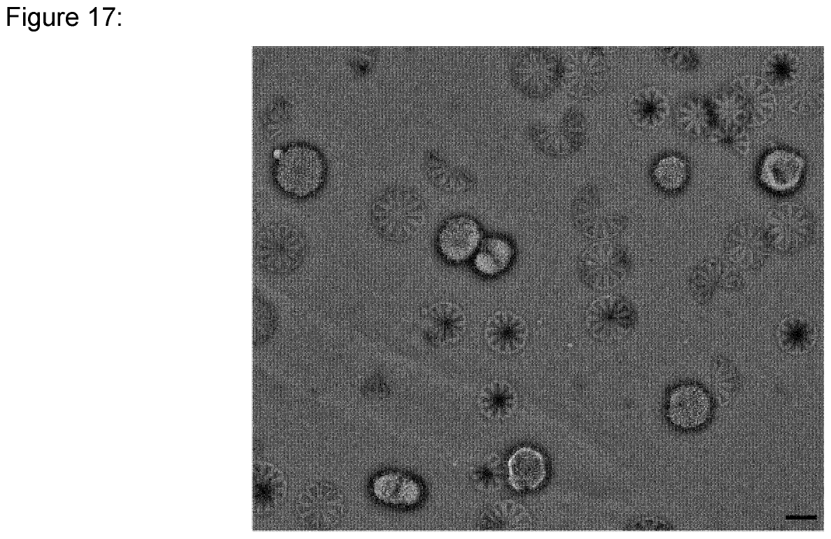

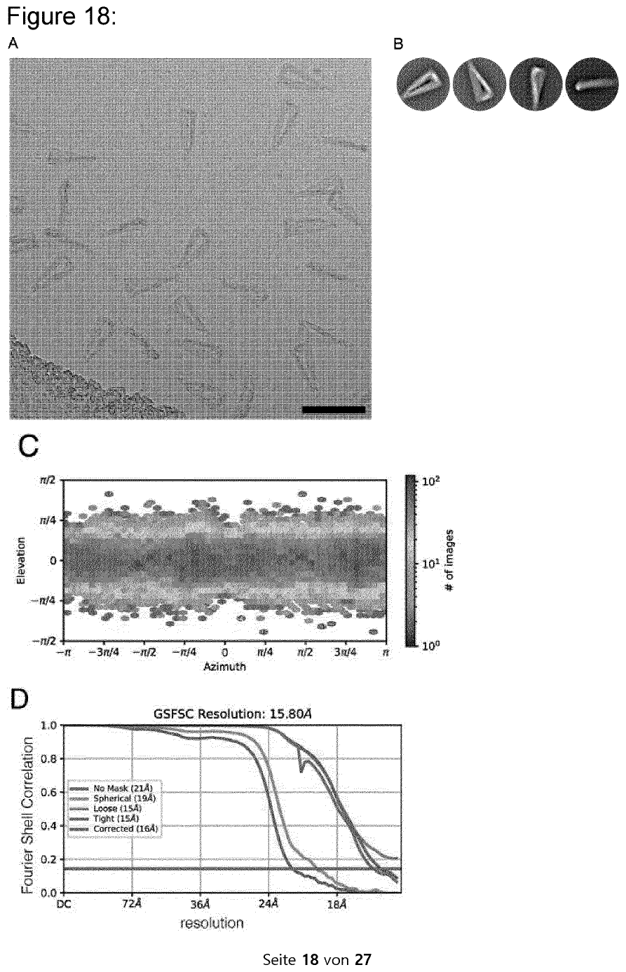

We triggered the oligomerization of wedge subunits into cones by increasing the ionic strength of the solution after folding of the wedge building blocks from its constituent DNA staple and scaffold strands. Oligomerization can also occur concomitantly during the wedge assembly reaction, depending on the ionic condition used (FIG. 11). We monitored the formation of cones in a time-dependent fashion by gel-electrophoretic mobility analysis (FIG. 2A), where the appearance and disappearance of bands towards increasingly lower electrophoretic mobilities reflected the progressive oligomerization of the wedge subunits as a function of incubation time. Eventually, the oligomerizing material accumulated in a comparably broad low electrophoretic mobility band.

We imaged the final oligomerization products using negative stain transmission electron microscopy (TEM). The micrographs revealed predominantly circular structures with cone-shaped appearance (FIG. 2B) consisting of 9, 10, 11, 12, and rarely, 13 copies of wedge subunits, respectively. The extent of heterogeneity seen in the cone oligomers with respect to how many wedges are included per cone is presumably linked to the finite elasticity of the wedge building blocks and their interaction interfaces. These properties may be tuned, if so desired, analogously as previously described with planar ring assemblies. 14 However, in the present case for the target application, the distribution of cone products covering species ranging from 9 up to 13 wedges appears advantageous for dealing with pleomorphic virus samples.

The distribution of cone products seen by TEM explains in part the comparably broad product band in the gel electrophoretic analysis. We also found that in the presence of elevated magnesium concentration such as those used in the gel electrophoresis, the cones have the tendency to stack onto each other (FIG. 12), which explains the smearing and formation of aggregates in the high-magnesium gel electrophoresis such as those shown in FIG. 2A. The cone-to-cone stacking was absent at low magnesium conditions as we show further below.

We observed three key preferred orientations of the cones in the TEM micrographs (FIG. 2B, inset) including cones adsorbed with their bases on the surface (1), cones that landed on their vertex (2), and cones that adhered on their lateral facets (3). Vertex-adhered cones had larger diameters and frayed circumferences compared to base-adhered cones containing the same number of wedge building blocks. Presumably, in the vertex-adhered orientation, adhesion forces flatten the cones which then causes the wedges to splay apart. In the based-adhered orientation, the cones remained intact buttressed by their base.

We computed two-dimensional (2D) class averages from TEM micrographs, which revealed several classes corresponding to different views (FIG. 13) and to different cone species. FIG. 2C shows exemplarily the class averages obtained for base-adhered cones featuring nine to thirteen wedge subunits, respectively. We measured the diameters from the non-deformed base-adhered particles (1) in the respective averaged classes and they closely matched our expectation (FIG. 2D). Accordingly, the C9 cone species had an average inner diameter of 110 nm. The C10 had 126 nm, and largest C13 species had 147 nm. From the 2D class averages we also quantified the relative frequency of occurrence of the different cone species. The most abundant cone was the C10 with a 37% of the population, followed by C11 (27%), C9 (20%), C12 (15%) and C13 (1%).

We performed cryo-EM studies of the cones in free-standing ice in order to gain 3D information of the assembled products. The exemplary cryo-EM field of view (FIG. 2E) shows different orientations of partial and fully assembled cones. We determined 3D reconstructions for the C9 and C10 cone species, which confirmed the overall 3D conical shape (FIG. 2F and FIG. 14). The electron density maps of both cone species have elliptical, undulated bases. The ellipticity is more pronounced for the C9 cone map. We measured the lengths of interior short and long axes to be 100 nm and 122 nm for the C9, and 114 nm and 131 nm for the C10 species (FIG. 15). The C9 cone's cavity was 42 nm deep, whereas the C10's was shallower (39 nm). The circumferences of the base-adhered cones from negative stain data and in solution cryo-EM reconstructions are in good agreement (Table 8). We assume that the electrostatic interactions between the cones and the carbon surface of the grids used for negative stain TEM leads to a flattening effect and therefore a rounder shape of the rings. It is also possible that surface interaction at the sample-air interface prior to plunge-freezing resulted in deformation of the particles seen in the 3D maps. Reconstructions of subsets of the particle ensemble of the cryo-EM data and multibody refinement and principal component analysis indicate a certain level of flexibility of the cones (FIG. 16), which is desirable for the intended application.

At the salt concentrations present in physiological fluids, DNA origami higher-order assemblies such as those presented in this work would normally dissociate.¹⁷ The wedge monomers would also be prone to denature due to insufficiently screened internal repulsive electrostatic forces. Physiological environments may also contain nucleases capable of degrading exogeneous DNA molecules by catalyzing the hydrolytic cleavage of phosphodiester bonds in the DNA backbone.¹⁸ To make our cone assemblies last in in vivo-like conditions, we established a three-step post-assembly stabilization treatment as illustrated schematically in FIG. 3A. The first step utilizes UV-light-induced cross-linking of thymidine bases placed in close proximity within DNA nanostructures.¹⁹ Through irradiation at a wavelength of 310-nm, the double bonds of adjacent pyrimidines undergo a [2+2] cycloaddition reaction yielding a cyclobutane pyrimidine dimer. To UV cross-link (“UV-point-weld”) the cone assemblies, we placed additional unpaired thymidine bases at the helical interfaces of the wedge-wedge subunit interaction sites (yellow dots in FIG. 3A, B). We tested the efficacy of UV cross-linking of cones as a function of time of exposure to irradiation with a 310 nm light source (FIG. 3C). Once properly UV welded, the cones remained intact when exposed to low Mg²⁺ concentrations, whereas the non-irradiated or insufficiently irradiated control samples rapidly dissociated into the constituent wedge subunits (FIG. 3C, D). The UV-linked cones now appear as five distinct bands in a low ionic strength gel (3 mM MgCl₂).

To protect the cone assemblies against nuclease-mediated degradation, we utilized the previously described oligolysine-PEG copolymer-based coating²⁰ followed by glutaraldehyde-crosslinking of this coating²¹ (FIG. 3A). We treated the UV-point-welded cones with K10PEG5K (N: P ratio of nitrogen in lysine to phosphorus in DNA of 1:0.6, and 2% (v/v) glutaraldehyde). To test for protection against nuclease activity, we subjected the samples to DNase I (0.001 U/μl, which corresponds to 2.6× of typical blood concentration of DNase I). We analyzed the digestion products using direct imaging with negative stain TEM. When uncoated, the cone assemblies were completely digested after 8 hours of incubation with DNase I, whereas the cones remained stable without obvious structural damage for up to 48 hours when oligolysine-PEG coated and cross-linked with glutaraldehyde (FIG. 3E).

For the intended application to tile and occlude the surface of virus particles, the inward-facing surface of the cones must be functionalized with additional virus-binding moieties. To this end, we introduced single-stranded DNA overhangs (termed ‘handles’) on the wedge subunit's inner surface that can hybridize with sequence-complementary oligonucleotides modified with the virus-binding moiety of choice. The positioning and the number of handles displayed on the wedge surface may be controlled by design. When using strong virus binders such as antibodies, a rather low density of handles may be sufficient for virus trapping (FIG. 4A); whereas weak and more broadly binding virus binders such as heparan sulfate (HS) polymers²² may benefit from a higher density of handles to exploit multivalency and avidity effects. To covalently conjugate DNA strands to the virus binders we used sulfo-SMCC linker (sulfosuccinimidyl 4-(N-maleimidomethyl)cyclohexane-1-carboxylate) for antibody conjugation,¹¹ and a copper-free click chemistry approach for HS derivatives.¹²

With the cone assemblies stabilized and functionalized, we tested the cones' ability to assemble around viruses with Influenza A/Puerto Rico/8/1934 viruses. Hemagglutinin (HA) and neuraminidase (NA) are the two most abundant proteins on the surface of Influenza A virus particles. We selected an antibody (see materials & methods) that targets a conserved epitope of the stem region withing the HA trimer as the virus-binding moiety and used it with a calculated density of six copies per wedge subunit. The antibody-functionalized cones successfully assembled around Influenza virus particles as we observed by direct TEM imaging (FIG. 4B). The cones adapted and molded around the diversely shaped Influenza particles (FIG. 4C). To gain more detailed information on the extent of 3D surface coverage by selected cone-virus assemblies, we performed negative stain electron microscopy tomography (FIG. 4D). The slices of the exemplary 3D tomogram reveal an Influenza virus particle enclosed by two cones in a sandwich-like assembly. Cones without antibodies did not associate with Influenza viruses (FIG. 17).

To further increase the surface area that will be occluded on virus particles, we designed a spiked cone assembly, in which a second wedge subunit (t2) is assembled on the base of the cone (FIG. 5A, B). The t2 wedge has a bevel angle of 45° and binds to the rim of the t1 wedge via a second set of shape-complementary pattern of protrusion and recesses (FIG. 5A, FIG. 18-20 for cryo-EM validation and S21-22 for assembly characterization). With the addition of the t2 building block, a single spiked cone assembly has an overall cavity depth and diameter of approx. 125 nm (FIG. 5B). A single copy of a spiked cone thus would in principle be sufficiently large to fully engulf Influenza viruses. FIG. 5C shows exemplarily negative stain TEM micrographs that we acquired of spiked-cone Influenza assemblies. The images reveal the flexibility and the different conformations the spiked cone can adopt. The t2 subunit also incorporated handle positions in its inner surface to place virus binding moieties. Similar to the cone assemblies, also the spiked cone variant successfully formed complexes with the Influenza A/Puerto Rico/8/1934 when functionalized with antibodies, as we saw by TEM imaging (FIG. 5D). Single copies of spiked cones were now sufficient to fully enclose entire virus particles of varying sizes. Negative stain TEM tomography was again used to obtain detailed 3D information. FIG. 5E shows tomogram slices through a 3D tomogram acquired of an exemplary spiked-cone Influenza assembly, revealing clearly that the Influenza virus “guest” sits deep within the cavity of the spiked cone “host”.

To illustrate the modular functionalization with virus-binding moieties, we trapped different virus particles with the spiked cone assemblies using the more broadly binding heparan sulfate (HS) derivative as internal coating. When using 12 copies of HS per wedge subunit, Chikungunya, SARS-COV-2 and Zika virus-like particles (VLP) were also trapped successfully within the spiked cone assemblies, as we established by direct imaging with negative staining TEM (FIG. 23). Depending on the rigidity of the virus particle, either the cone host or the guest virus particle adapted to one another. For instance, the Zika particles completely flattened out when adhered to the cones, whereas the cones deformed to match the curvature of the rather spherical and apparently more rigid Chikungunya particles.

### Conclusions

We presented cone-shaped DNA origami higher-order assemblies that form efficiently and with high yields from a single building block. In comparison to our previous prototypes which took weeks to assemble, required multiple building blocks, and had inferior yields (<50%), we achieved substantially improved assembly yields of above 80% in one-pot reaction mixtures over the time course of 72 hours. We developed the cone assemblies primarily for trapping and engulfing large and pleomorphic virus particles. To this end, we demonstrated modular functionalization with user-defined virus-binding moieties. In one instance, we used antibodies to engulf Influenza viruses with the cones, with up to 60 antibodies displayed per cone. In another instance, we used heparan sulfate to trap Zika, Chikungunya and SARS-COV-2 VLPs using cone assemblies displaying up to 120 HS polymer copies per cone. The cone assemblies can deform and adapt to the shape of the trapped virus particles, as we saw here with pleomorphic Influenza virus samples, which is advantageous for our envisioned target application. We have also established a post-assembly stabilization treatment of the cones so that they can persist in low-salt environments and survive the attack of nucleases for at least 48 hours. All DNA components needed for our cones can in principle be biotechnologically mass-produced.²³ The present work thus contributes to setting the stage for testing the therapeutic potential of a large-virus-engulfing DNA nanoarchitecture in vivo. Beyond trapping large viruses, the cone assemblies, or variants of it, could be of use in artificial light-harvesting antenna complexes,^{24,25} and as a candidate structure for placement on nanostructured surfaces.^{26,27}

### Materials and Methods

Staple strands for origami folding reactions were purchased from Integrated DNA Technologies (IDT) and used with standard desalting purification. SH-modified handle strands were purchased from Biomers at HPLC grade. PEG-polyLysine coatings were purchased from Alamanda Polymers. Chikungunya VLPs were purchased from The Native Antigen Company, SARS-COV-2 VLPs from Creative Biolabs, Zika VLPs from Creative Biostructure, and inactivated Influenza A/PR/8/34 virus from Charles River Laboratories.

### DNA Origami Design

The cross-sections of both triangular building blocks t1 and t2 are 3×6 arranged in square lattices of DNA helices.

The DNA origami designs of the t1 and t2 isosceles triangles involve corners of different angles as well as a beveled angle. A schematic representation of the important parameters can be found in FIG. 6, A and B. To create a corner in a DNA origami object, specific deletions are necessary depending on the angle of interest. The length difference in between two DNA double helices (Δa) is dependent on the angle (α) and the distance between the two helices (x) following equation (1).

$$Δ a ( x ) = \frac{x}{tan ( α / 2 )} \qquad ( 1 )$$

$$x = n * d \qquad ( 2 )$$

The distance between the two helices (x) is the diameter of a DNA double helix (d). The effective diameter of a DNA double helix is 2.1 nm,³⁹ but considering that in a DNA origami structure the helices are not tightly packed due to electrostatic repulsion forces, d is averaged to be 2.6 nm.⁴⁰ Depending on the position of each helix (n), x varies and Aa has to be re-calculated using equations (1) and (2). With these design parameters, the DNA helices get shorter the closer they are to the center.

Isosceles triangles have two different angles (α and β) and therefore require two different corner designs. The length differences of the helices at such corners will be different (Δa and Δb), and need to be calculated separately using equations (1) and (2). The length of any helix (aₓ or bₓ) can be calculated by subtracting Δa/b from the length of the reference helix (a₀ or b₀). Also, a helical rise of 0.34 nm/bp can be used to convert lengths of DNA helices from base pairs to nanometers.

$${a}_{x} = {a}_{0} - 2 Δ a ( x ) \qquad ( 3.1 )$$

$${b}_{x} = {b}_{0} - Δ a ( x ) - Δ b ( x ) \qquad ( 3.2 )$$

For corner designs, it is important to know the double helix orientation of the DNA strands at the nick position. In order to reach the other side of the nick, the DNA strand facing the outer side of the corner needs to have a single stranded segment (FIG. 7C). When the staple strand (yellow) faces the outer side of the nick, we give it 5 thymidine single stranded bases, whereas when it is the scaffold strand (blue), we only give it one single stranded base.

In order to get assemblies with curvature, the sides of the triangles need to be tilted by a certain bevel angle. FIG. 7D shows a schematic representation of how a corner design looks with a certain beveled angle. By rotating each DNA helix by an angle θ, the original coordinates of a helix (n,m) change from x_{0,nm} and y_{0,nm} to xₙₘ and yₙₘ (FIG. 7A). The new coordinates can be calculated using a two-dimensional rotation matrix (4). All triangles in this work were designed such that the three edges always have the same bevel angle and only different lengths.

$$[ \frac{{x}_{n m}}{{y}_{n m}} ] = [ \begin{matrix}cos θ & sin θ \\ - sin θ & cos θ\end{matrix} ] * [ \frac{{x}_{0 , n m}}{{y}_{0 , n m}} ] with [ \frac{{x}_{0 , n m}}{{y}_{0 , n m}} ] = [ \frac{n * d}{m ⋆ d} ] \qquad ( 4 )$$

The length differences needed to apply to achieve the desired bevel angle can be calculated using (5.1) and (5.2):

$$Δ a ( {x}_{n m} ) = \frac{n cos θ + m sin θ}{tan ( α / 2 )} * d \qquad ( 5.1 )$$

$$Δ b ( {x}_{n m} ) = \frac{n cos θ + m sin θ}{tan ( β / 2 )} * d \qquad ( 5.2 )$$

If the bevel angle is designed to be pronounced, the resulting assembly will feature a deep cavity at the cost of a smaller cone diameter; whereas if it is less prominent, the product will have shallower depth but display a larger diameter.

The actual values of corner angles (α and β), beveled angles (θ) and lengths of the reference helices (aₓ and bₓ) are summarized in Table 2.

TABLE 4
Design parameters of t1 and t2 referencing FIG. 7.

| | t1 | t2 |
|---|---|---|
| α | 74.6° | 78.3° |
| β | 30.7° | 23.5° |
| θ | 9.5° | 45° |
| a₀ | 122 bp | 122 bp |
| b₀ | 232 bp | 300 bp |

### Folding of DNA Origami Triangular Subunits:

DNA origami structures were self-assembled (“folded”) in one-pot reaction mixtures containing 50 nM of single-stranded scaffold DNA (M13, 8064 bases) and 250 nM of each staple strand in a standardized “folding buffer” (FoB15) containing 15 mM MgCl₂, 5 mM Tris Base, 1 mM EDTA and 5 mM NaCl at pH 8.00. Scaffold M13 was produced as previously described (Supplementary Note 1 for sequence).28 The folding reactions were subjected to thermal annealing ramps (60 to 44° C. with a decrease of 1° C./h) in a Tetrad (Bio-Rad) thermal cycling device.

### Purification of Triangle Subunits and Self-Assembly of Cones:

All objects were purified using agarose gel extraction (1.5% agarose containing 0.5×TBE and 5.5 mM MgCl₂) and centrifuged for 60 min at maximum speed for residual agarose pelleting. Typical subunit concentrations ranged from 5 to 50 nM, while assembly times ranged from 3 to 5 days. Cone assembly proceeded well at a MgCl₂ concentration of 25 mM and incubation at 40° C. for at least 72 hours. The assembly of the spiked cone with t2 required 40 mM MgCl₂ and a longer incubation time (approx. 4 days).

### Cones Stabilization for In Vivo Applications:

The assembled cones were UV cross-linked19 for at least 20 min at 310 nm using Asahi Spectra Xenon Light source 300W MAX-303. The cones were incubated in a 0.6:1 ratio of N/P with a mixture of K10-oligolysine and K10-PEG5K-oligolysine (1:1) for 1 h at room temperature as similarly described previously.²⁰ For chemical cross-linking, appropriate amounts of a 50% glutaraldehyde stock were added for a final concentration of 2% (v/v), incubated for 1 h at room temperature, and filtered with 0.5 ml Zeba spin desalting columns (7K MWCO). Dnase I activity assays were performed at 0.001 U/μL (2.6-fold increase of blood concentration) and incubated at 37° C. for different time points in 1×PBS buffer containing 10 mM MgCl₂.

### Generation of Recombinant Antibody:

Sequences of the heavy variable chain and the lambda light variable chain of the broadly reactive monoclonal antibody CR9114 specifically targeting the stem region of the Influenza A and B hemagglutinin (HA)²⁹ were derived from RCSB protein data bank 4FQI, modified with suitable restriction sites for cloning and ordered as strings from Geneart™. DNA fragments encoding the variable domain of the heavy and light chain were cloned into a pAbHC or pAbLC_lambda vector respectively, both pBR322 based human IgG1 expression vectors. Correct cloning was confirmed by Sanger sequencing performed by MicrosynthSeqlab. Antibodies were expressed in 40 ml HEK293F Expi cells. Cells were grown to 2.5×10⁶ cells/ml at the point of transfection. The transfection uses ThermoFisher ExpiFectamine transfection kit and follows the included protocols. 40 μg DNA (20 μg heavy chain plasmid, 20 μg light chain plasmid) were transfected using 107 μl ExpiFectamine™. After 16-18 h 200 μl Enhancer1 and 2 ml Enhancer2 were added to the transfected cells. Cells were left to express the antibodies for 5 days at 37° C., 8% CO₂ on an incubator shaking at 125 rpm. Supernatant was cleared by centrifugation at 1,000 g for 10 min, followed by 4,000 g for 15 min. Cleared supernatant was sterile filtered (0.2 μm milipore steritop filter) and when stored added with 0.05% NaN₃. HiTrap rProtein A FF 1 ml columns were loaded with the supernatant overnight at 4° C. at a flowrate of 1 ml/min. Columns were then washed with 50 ml PBS to wash away any unbound leftovers. Antibodies were eluted using 0.1 M Glycine, pH 3.2 and fractionated 4 times in 2.5 ml. Each fraction was immediately neutralized with 1 M Tris/HCl, pH 9 to a final pH of 7.3. Using pD10 columns the buffer was exchanged to PBS. For storage preparation the antibody was concentrated or diluted to the wanted concentration and centrifuged at 14,000 g for 30 min before being sterile filtered (22 μm).

### Antibody Conjugation to DNA:

An oligonucleotide with a sequence complementary to the origami handles (5′-TGCCTAATCTCTACCTACTCTACTGC-3′; SEQ ID NO: 1408) and modified with a thiol group at the 3′ end was coupled to the antibody anti-HA CR9114 (100 μg) using a sulfosuccinimidyl-4-(N-maleimidomethyl)cyclohexane-1-carboxylate crosslinker. The product was purified using proFIRE (Dynamic Biosensors). The DNA-modified antibody was added to the assembled and UV-welded cones with 1:1 stoichiometry to the number of handles and incubated for 1 h at room temperature.

### Heparan Sulfate Conjugation to DNA:

Experimental protocol was as previously described by Monferrer et. al.¹²

### Viruses and VLPs Encapsulation:

Pre-assembled and UV-welded cones in 1×PBS containing 10 mM MgCl₂ were mixed with a virus or VLP sample in the appropriate ratio. The samples were incubated at r.t. for 2 h. Usual amounts of sample for TEM analysis range from 5-10 μL total solution at ˜10 nM triangle origami concentration. Negative stain TEM grids were prepared immediately after the 2 h incubation.

### Negative Staining TEM:

Samples were incubated on glow discharged (45 s, 35 mA) forrmvar carbon-coated Cu400 TEM grids (Electron Microscopy Sciences) for 90 to 120 s depending on origami and MgCl₂ concentrations. Next, the grids were stained for 30 s with 2% aqueous uranyl formate containing 25 mM NaOH. Imaging was performed with magnifications in between 10000× and 42000× in a SerialEM at a FEI Tecnai T12 microscope operated at 120 KV with a Tietz TEMCAM-F416 camera. TEM micrographs were high-pass filtered to remove long-range staining gradients and the contrast was auto-leveled using Adobe Photoshop. To obtain TEM statistics in an unbiased fashion, automatic grid montages were acquired. For detailed information on selected particles, negative stain EM tomography was used as a visualization technique. The tilt series were performed from −30° to +30° and micrographs were acquired in 2° increments. Tilt series were processed with Etomo (IMOD) to acquire tomograms.³⁰ The micrographs were aligned to each other by calculating a cross correlation of the consecutive tilt series images. The tomogram is then generated using a filtered back-projection. The Gaussian-Filter used a cutoff between 0.25 and 0.5, and a fall-off of 0.035.

### Negative Stain Data Processing:

We processed the micrographs in CryoSparc³¹ and estimated the contrast transfer function (CTF) with CTFFIND4.³² We used a combination of manual picking and TOPAZ auto-picking³³ and extracted the particles consisting of different numbers of monomers. We subjected the particles to multiple rounds of 2D classification to sort them and to create class-averaged images at increased signal-to-noise ratio. We evaluated the distribution of assemblies via the assignment of particles in certain 2D classes and manual inspection. We measured the dimensions of different types of assemblies based on the 2D class-averaged images data using FIJI.³⁴

### Cryo-Grid Preparation and Cryo-EM Image Acquisition:

For the triangle DNA constructs, we vitrified each cryo-EM sample with a Vitrobot Mark IV (Thermo Scientific). We applied 4 μl of sample to a glow discharged C-Flat grid (Protochips) (Table 6), blotted, and plunge-froze it using the following Vitrobot settings: temperature of 22° C., relative humidity of 100%, 2-2.5 s blot time, −1 blot force. For the cone assemblies we used double blotting consisting of 4 μl sample application, 60 s incubation on the grid, manual blotting, followed by a second round of sample application, semi-automatic blotting and plunge-freezing as described above. We acquired movies consisting of 10-13 frames with a Falcon 3 direct detector (Thermo Scientific) on a Cs-corrected (CEOS) Titan Krios G2 electron microscope (Thermo Scientific) operated at 300 kV using the EPU software (Thermo Scientific) at an accumulated dose of ˜50 e/sqÅ and a magnified pixel size of 2.28 Å and 1.79 Å (Table 5). Acquisition with a tilted stage was used to reduce orientation bias of the particles.

### Cryo-EM Data Processing:

We processed the cryo-EM data mostly in the Relion 4 software suite.^{35,36} For motion-correction of the movies and CTF-estimation we used the Relion implementation and CTFFIND4,³² respectively. We semi-automatically picked particles using TOPAZ,³³ extracted the particles, and removed falsely picked grid contaminations damaged particles via multiple rounds of 2D. Using a low-resolution ab-initio initial model created in Relion we addressed structural heterogeneity via 3D classification and reconstructed a 3D-refined map. We applied per-particle motion correction and dose weighting to receive a set of polished particles and reconstructed a 3D-refined map at higher resolution. We post-processed the map by applying a low-resolution mask as well as Fourier shell correlation (FSC) estimation-based low-pass filtering and sharpening using the 0.143 FSC criterium. For the triangle 2 version 1, we reconstructed the final map including post-processing using CryoSparc.³¹ We 3D-measured the dimensions of the electron density maps in 3D and rendered images using ChimeraX.³⁷

TABLE 5
Estimation of C9 and C10's inner diameters.

| | shape and inner | | approximated |
|---|---|---|---|
| Object | dimensions of base | formula | circumference |
| C9_negative-stain-TEM | circular | C = 2rTT | 691 nm |
| | r = 110 nm | | |
| C9_cryo-EM | elliptic, r1 = 100 nm, r2 = 122 nm | $C = π ( a + b ) ( 1 + \frac{3 h}{10 + \sqrt{4 - 3 h}} ) , with h = {( a - b )}^{2} / {( a + b )}^{2}$ | 699 nm |
| C10_negative-stain-TEM | circular, | C = 2mTT | 791 nm |
| | r = 126 nm | | |
| C10_cryo-EM | elliptic, r1 = 114 nm, r2 = 131 nm | $C = ( a + b ) ( 1 + \frac{3 h}{10 + \sqrt{4 - 3 h}} ) , with h = {( a - b )}^{2} / {( a + b )}^{2}$ | 770 nm |

TABLE 6
Cryo-EM grid preparation, data acquisition
and data processing details.

| | Triangle | | | # used | # particles |
|---|---|---|---|---|---|
| | concen- | | Mag. | micro- | in final |
| Structure | tration | Grid Type | Pix. | graphs | refinement |
| Triangle | 927 nM | C-Flat 1.2/1.3 | 2.28 | 1092 | 14959 |
| 1 v1 | | 4C | | | |
| Triangle | 953 nM | C-Flat 2/1 4C | 1.79 | 6234 | 58237 |
| 2 v1 | | | | | |
| Triangle | 1074 nM | C-Flat 1.2/1.3 | 2.28 | 4951 | 51748 |
| 1 v2 | | 4C thick | | | |
| Triangle | 648 nM | C-Flat 1.2/1.3 | 2.28 | 1861 | 21999 |
| 2 v2 | | 4C | | | |
| Triangle | 984 nM | C-Flat 1.2/1.3 | 2.28 | 1853 | 29197 |
| 1 v3 | | 4C thick | | | |
| Triangle | 1020 nM | C-Flat 1.2/1.3 | 2.28 | 3670 | 99057 |
| 2 v3 | | 4C thick | | | |
| Cones | 200 nM | C-Flat 1.2/1.3 | 2.28 | 3376 | 1166 for C9 |
| | | 4C thick | | | 1358 for C10 |

### REFERENCES

(1) Esté, J.; Telenti, A. HIV Entry Inhibitors. The Lancet 2007, 370 (9581), 81-88. https://doi.org/10.1016/S0140-6736 (07) 61052-6.
(2) Pelegrin, M.; Naranjo-Gomez, M.; Piechaczyk, M. Antiviral Monoclonal Antibodies: Can They Be More Than Simple Neutralizing Agents? Trends Microbiol. 2015, 23 (10), 653-665. https://doi.org/10.1016/j.tim.2015.07.005.
(3) Tyssen, D.; Henderson, S. A.; Johnson, A.; Sterjovski, J.; Moore, K.; La, J.; Zanin, M.; Sonza, S.; Karellas, P.; Giannis, M. P.; Krippner, G.; Wesselingh, S.; Mccarthy, T.; Gorry, P. R.; Ramsland, P. A.; Cone, R.; Paull, J. R. A.; Lewis, G. R.; Tachedjian, G. Structure Activity Relationship of Dendrimer Microbicides with Dual Action Antiviral Activity. PLOS ONE 2010, 5 (8), e12309. https://doi.org/10.1371/journal.pone.0012309.
(4) Price, C. F.; Tyssen, D.; Sonza, S.; Davie, A.; Evans, S.; Lewis, G. R.; Xia, S.; Spelman, T.; Hodsman, P.; Moench, T. R.; Humberstone, A.; Paull, J. R. A.; Tachedjian, G. SPL7013 Gel (VivaGel®) Retains Potent HIV-1 and HSV-2 Inhibitory Activity Following Vaginal Administration in Humans. PLOS ONE 2011, 6 (9), e24095. https://doi.org/10.1371/journal.pone.0024095.
(5) Zelikin, A. N.; Stellacci, F. Broad-Spectrum Antiviral Agents Based on Multivalent Inhibitors of Viral Infectivity. Adv. Healthc. Mater. 2021, 10 (6), 2001433. https://doi.org/10.1002/adhm.202001433.
(6) Cagno, V.; Andreozzi, P.; D'Alicarnasso, M.; Jacob Silva, P.; Mueller, M.; Galloux, M.; Le Goffic, R.; Jones, S. T.; Vallino, M.; Hodek, J.; Weber, J.; Sen, S.; Janeček, E.-R.; Bekdemir, A.; Sanavio, B.; Martinelli, C.; Donalisio, M.; Rameix Welti, M.-A.; Eleouet, J.-F.; Han, Y.; Kaiser, L.; Vukovic, L.; Tapparel, C.; Král, P.; Krol, S.; Lembo, D.; Stellacci, F. Broad-Spectrum Non-Toxic Antiviral Nanoparticles with a Virucidal Inhibition Mechanism. Nat. 2018, Mater. 17 (2), 195-203. https://doi.org/10.1038/nmat5053.
(7) Al-Mahtab, M.; Bazinet, M.; Vaillant, A. Safety and Efficacy of Nucleic Acid Polymers in Monotherapy and Combined with Immunotherapy in Treatment-Naive Bangladeshi Patients with HBeAg+Chronic Hepatitis B Infection. PLOS ONE 2016, 11 (6), e0156667. https://doi.org/10.1371/journal.pone.0156667.
(8) Kwon, P. S.; Ren, S.; Kwon, S.-J.; Kizer, M. E.; Kuo, L.; Xie, M.; Zhu, D.; Zhou, F.; Zhang, F.; Kim, D.; Fraser, K.; Kramer, L. D.; Seeman, N. C.; Dordick, J. S.; Linhardt, R. J.; Chao, J.; Wang, X. Designer DNA Architecture Offers Precise and Multivalent Spatial Pattern-Recognition for Viral Sensing and Inhibition. Nat. Chem. 2020, 12 (1), 26-35. https://doi.org/10.1038/s41557-019-0369-8.
(9) Ren, S.; Fraser, K.; Kuo, L.; Chauhan, N.; Adrian, A. T.; Zhang, F.; Linhardt, R. J.; Kwon, P. S.; Wang, X. Designer DNA Nanostructures for Viral Inhibition. Nat. Protoc. 2022, 17 (2), 282-326. https://doi.org/10.1038/s41596-021-00641-y.
(10) Chauhan, N.; Xiong, Y.; Ren, S.; Dwivedy, A.; Magazine, N.; Zhou, L.; Jin, X.; Zhang, T.; Cunningham, B. T.; Yao, S.; Huang, W.; Wang, X. Net-Shaped DNA Nanostructures Designed for Rapid/Sensitive Detection and Potential Inhibition of the SARS-COV-2 Virus. J. Am. Chem. Soc. 2022, jacs.2c04835. https://doi.org/10.1021/jacs.2c04835.
(11) Sigl, C.; Willner, E. M.; Engelen, W.; Kretzmann, J. A.; Sachenbacher, K.; Liedl, A.; Kolbe, F.; Wilsch, F.; Aghvami, S. A.; Protzer, U.; Hagan, M. F.; Fraden, S.; Dietz, H. Programmable Icosahedral Shell System for Virus Trapping. Nat. Mater. 2021, 20 (9), 1281-1289. https://doi.org/10.1038/s41563-021-01020-4.
(12) Monferrer, A.; Kretzmann, J. A.; Sigl, C.; Sapelza, P.; Liedl, A.; Wittmann, B.; Dietz, H. Broad-Spectrum Virus Trapping with Heparan Sulfate-Modified DNA Origami Shells. https://doi.org/10.1021/acsnano.1c11328.
(13) Dadonaite, B.; Vijayakrishnan, S.; Fodor, E.; Bhella, D.; Hutchinson, E. C. Filamentous Influenza Viruses. J. Gen. Virol. 2016, 97 (8), 1755-1764. https://doi.org/10.1099/jgv.0.000535.
(14) Wagenbauer, K. F.; Sigl, C.; Dietz, H. Gigadalton-Scale Shape-Programmable DNA Assemblies. Nature 2017, 552 (7683), 78-83. https://doi.org/10.1038/nature24651.
(15) Douglas, S. M. Self-Assembly of DNA into Nanoscale Three-Dimensional Shapes. Nature 459 (2009) 6.
(16) Simmons, C. R.; Zhang, F.; Birktoft, J. J.; Qi, X.; Han, D.; Liu, Y.; Abdallah, H.; Hernandez, C.; Ohayon, Y.; Seeman, N. C.; Yan, H. Construction and Structure Determination of a Three-Dimensional DNA Crystal. J. Am. Chem. Soc. 23.
(17) Hahn, J.; Wickham, S. F. J.; Shih, W. M.; Perrault, S. D. Addressing the Instability of DNA Nanostructures in Tissue Culture. 2014, 8 (9), 11.
(18) Lauková, L.; Konečná, B.; Janovičová, Ľ.; Vlková, B.; Celec, P. Deoxyribonucleases and Their Applications in Biomedicine. Biomolecules 2020, 10 (7), 1036. https://doi.org/10.3390/biom10071036.
(19) Gerling, T.; Kube, M.; Kick, B.; Dietz, H. Sequence-Programmable Covalent Bonding of Designed DNA Assemblies. Sci. Adv. 2018, 4 (8), eaau1157. https://doi.org/10.1126/sciadv.aau1157.
(20) Ponnuswamy, N.; Bastings, M. M. C.; Nathwani, B.; Ryu, J. H.; Chou, L. Y. T.; Vinther, M.; Li, W. A.; Anastassacos, F. M.; Mooney, D. J.; Shih, W. M. Oligolysine-Based Coating Protects DNA Nanostructures from Low-Salt Denaturation and Nuclease Degradation. Nat. Commun. 2017, 8 (1) 15654. https://doi.org/10.1038/ncomms15654.
(21) Anastassacos, F. M.; Zhao, Z.; Zeng, Y.; Shih, W. M. Glutaraldehyde Cross-Linking of Oligolysines Coating DNA Origami Greatly Reduces Susceptibility to Nuclease Degradation. J. Am. Chem. Soc. 2020, 142 (7), 3311-3315. https://doi.org/10.1021/jacs.9b11698.
(22) Dreyfuss, J. L.; Regatieri, C. V.; Jarrouge, T. R.; Cavalheiro, R. P.; Sampaio, L. O.; Nader, H. B. Heparan Sulfate Proteoglycans: Structure, Protein Interactions and Cell Signaling. An. Acad. Bras. Ciênc. 2009, 81 (3), 409-429. https://doi.org/10.1590/S0001-37652009000300007.
(23) Praetorius, F.; Kick, B.; Behler, K. L.; Honemann, M. N.; Weuster-Botz, D.; Dietz, H. Biotechnological Mass Production of DNA Origami. Nature 2017, 552 (7683), 84-87. https://doi.org/10.1038/nature24650.
(24) Fu, J.; Liu, M.; Liu, Y.; Woodbury, N. W.; Yan, H. Interenzyme Substrate Diffusion for an Enzyme Cascade Organized on Spatially Addressable DNA Nanostructures. J. Am. Chem. Soc. 2012, 134 (12), 5516-5519. https://doi.org/10.1021/ja300897h.
(25) Pan, K.; Boulais, E.; Yang, L.; Bathe, M. Structure-Based Model for Light-Harvesting Properties of Nucleic Acid Nanostructures. Nucleic Acids Res. 2014, 42 (4), 2159-2170. https://doi.org/10.1093/nar/gkt1269.
(26) Gopinath, A.; Miyazono, E.; Faraon, A.; Rothemund, P. W. K. Engineering and Mapping Nanocavity Emission via Precision Placement of DNA Origami. Nature 2016, 535 (7612), 401-405. https://doi.org/10.1038/nature18287.
(27) Gopinath, A.; Thachuk, C.; Mitskovets, A.; Atwater, H. A.; Kirkpatrick, D.; Rothemund, P. W. K. Absolute and Arbitrary Orientation of Single-Molecule Shapes. Science 2021, 371 (6531), eabd6179. https://doi.org/10.1126/science.abd6179.
(28) Engelhardt, F. A. S.; Praetorius, F.; Wachauf, C. H.; Bruggenthies, G.; Kohler, F.; Kick, B.; Kadletz, K. L.; Pham, P. N.; Behler, K. L.; Gerling, T.; Dietz, H. Custom-Size, Functional, and Durable DNA Origami with Design-Specific Scaffolds. ACS Nano 2019, 13 (5), 5015-5027. https://doi.org/10.1021/acsnano.9b01025.
(29) Dreyfus, C.; Laursen, N. S.; Kwaks, T.; Zuijdgeest, D.; Khayat, R.; Ekiert, D. C.; Lee, J. H.; Metlagel, Z.; Bujny, M. V.; Jongeneelen, M.; van der Vlugt, R.; Lamrani, M.; Korse, H. J. W. M.; Geelen, E.; Sahin, O.; Sieuwerts, M.; Brakenhoff, J. P. J.; Vogels, R.; Li, O. T. W.; Poon, L. L. M.; Peiris, M.; Koudstaal, W.; Ward, A. B.; Wilson, I. A.; Goudsmit, J.; Friesen, R. H. E. Highly Conserved Protective Epitopes on Influenza B Viruses. Science 2012, 337 (6100), 1343-1348. https://doi.org/10.1126/science. 1222908.
(30) Kremer, J. R.; Mastronarde, D. N.; Mcintosh, J. R. Computer Visualization of Three-Dimensional Image Data Using IMOD. J. Struct. Biol. 1996, 116 (1), 71-76. https://doi.org/10.1006/jsbi. 1996.0013.
(31) Punjani, A.; Rubinstein, J. L.; Fleet, D. J.; Brubaker, M. A. CryoSPARC: Algorithms for Rapid Unsupervised Cryo-EM Structure Determination. Nat. Methods 2017, 8.
(32) Rohou, A.; Grigorieff, N. CTFFIND4: Fast and Accurate Defocus Estimation from Electron Micrographs. J. Struct. Biol. 2015, 192 (2), 216-221. https://doi.org/10.1016/j.jsb.2015.08.008.
(33) Bepler, T.; Morin, A.; Rapp, M.; Brasch, J.; Shapiro, L.; Noble, A. J.; Berger, B. Positive-Unlabeled Convolutional Neural Networks for Particle Picking in Cryo-Electron Micrographs. Nat. 2019, 16 Methods (11), 1153-1160. https://doi.org/10.1038/s41592-019-0575-8.
(34) Schindelin, J.; Arganda-Carreras, I.; Frise, E.; Kaynig, V.; Longair, M.; Pietzsch, T.; Preibisch, S.; Rueden, C.; Saalfeld, S.; Schmid, B.; Tinevez, J.-Y.; White, D. J.; Hartenstein, V.; Eliceiri, K.; Tomancak, P.; Cardona, A. Fiji: An Open-Source Platform for Biological-Image Analysis. Nat. Methods 2012, 9 (7), 676-682. https://doi.org/10.1038/nmeth.2019.
(35) Scheres, S. H. W. A Bayesian View on Cryo-EM Structure Determination. J. Mol. Biol. 2012, 415 (2), 406-418. https://doi.org/10.1016/j.jmb.2011.11.010.
(36) Kimanius, D.; Dong, L.; Sharov, G.; Nakane, T.; Scheres, S. H. W. New Tools for Automated Cryo-EM Single-Particle Analysis in RELION-4.0. Biochem. J. 2021, 478 (24), 4169-4185. https://doi.org/10.1042/BCJ20210708.
(37) Pettersen, E. F.; Goddard, T. D.; Huang, C. C.; Meng, E. C.; Couch, G. S.; Croll, T. I.; Morris, J. H.; Ferrin, T. E. UCSF ChimeraX: Structure Visualization for Researchers, Educators, and Developers. 13.
(38) Douglas, S. M.; Marblestone, A. H.; Teerapittayanon, S.; Vazquez, A.; Church, G. M.; Shih, W. M. Rapid Prototyping of 3D DNA-Origami Shapes with CaDNAno. Nucleic Acids Res. 2009, 37 (15), 5001-5006. https://doi.org/10.1093/nar/gkp436.
(39) Zimmerman, S. B. The Three-Dimensional Structure of DNA. Annu. Rev. Biochem. 1982, 51 (1), 395-427.
(40) X. C. Bai, T. G. Martin, S. H. Scheres, H. Dietz, Cryo-EM structure of a 3D DNA-origami object. Proceedings of the National Academy of Sciences of the United States of America 109, 20012-20017 (2012).
(41. C. Gortazar et al., Crossing the interspecies barrier: opening the door to zoonotic pathogens. PLOS pathogens 10, e1004129 (2014).
(42) S. S. Morse et al., Prediction and prevention of the next pandemic zoonosis. Lancet 380, 1956-1965 (2012).
(43) J. J. O'Brien, D. M. Campoli-Richards, Acyclovir. An updated review of its antiviral activity, pharmacokinetic properties and therapeutic efficacy. Drugs 37, 233-309 (1989).
(44) J. LaBonte, J. Lebbos, P. Kirkpatrick, Enfuvirtide. Nat Rev Drug Discov 2, 345-346 (2003).
(45) W. L. Davies et al., Antiviral Activity of 1-Adamantanamine (Amantadine). Science 144, 862-863 (1964).
(46) Y. K. Gupta, M. Meenu, P. Mohan, The Tamiflu fiasco and lessons learnt. Indian J Pharmacol 47, 11-16 (2015).
(47) J. M. Steichen et al., A generalized HIV vaccine design strategy for priming of broadly neutralizing antibody responses. Science 366, (2019).
(48) K. O. Saunders et al., Targeted selection of HIV-specific antibody mutations by engineering B cell maturation. Science 366, (2019).
(49) R. Iinuma et al., Polyhedra self-assembled from DNA tripods and characterized with 3D DNA-PAINT. Science 344, 65-69 (2014).
(50) P. W. K. Rothemund, Folding DNA to create nanoscale shapes and patterns. Nature 440, 297-302 (2006).
(51) S. M. Douglas et al., Self-assembly of DNA into nanoscale three-dimensional shapes. Nature 459, 414-418 (2009).
(52) C. E. Castro et al., A primer to scaffolded DNA origami. Nature methods 8, 221-229 (2011).
(53) R. Veneziano et al., Designer nanoscale DNA assemblies programmed from the top down. Science 352, 1534 (2016).
(54) E. Benson et al., DNA rendering of polyhedral meshes at the nanoscale. Nature 523, 441-444 (2015).
(55) K. E. Dunn et al., Guiding the folding pathway of DNA origami. Nature 525, 82-86 (2015).
(56) J. J. Funke, H. Dietz, Placing molecules with Bohr radius resolution using DNA origami. Nature nanotechnology 11, 47-52 (2016).
(57) R. Jungmann et al., DNA origami-based nanoribbons: assembly, length distribution, and twist. Nanotechnology 22, 275301 (2011).
(58) W. Liu, H. Zhong, R. Wang, N. C. Seeman, Crystalline two-dimensional DNA-origami arrays. Angewandte Chemie 50, 264-267 (2011).
(59) Y. Suzuki, M. Endo, H. Sugiyama, Lipid-bilayer-assisted two-dimensional self-assembly of DNA origami nanostructures. Nature communications 6, 8052 (2015).
(60) Y. Ke et al., DNA brick crystals with prescribed depths. Nature chemistry 6, 994-1002 (2014).
(61) C. Maffeo, J. Yoo, A. Aksimentiev, De novo reconstruction of DNA origami structures through atomistic molecular dynamics simulation. Nucleic acids research 44, 3013-3019 (2016).
(62) T. Gerling, K. F. Wagenbauer, A. M. Neuner, H. Dietz, Dynamic DNA devices and assemblies formed by shape-complementary, non-base pairing 3D components. Science 347, 1446-1452 (2015).
(63) V. Cagno, E. D. Tseligka, S. T. Jones, C. Tapparel, Heparan Sulfate Proteoglycans and Viral Attachment: True Receptors or Adaptation Bias? Viruses 11, (2019) 596.
(64) Zhang, Q. et al., Cell Discov. 6 (2020) 1-14.
(65) Vaillant, A., Antiviral Res. 133 (2016) 32-40.
(66) Cagno, V. et al., Antimicrob. Agents Chemother. 64 (2020) e02001-20.
(67) T. Gerling, H. Dietz, Reversible Covalent Stabilization of Stacking Contacts in DNA Assemblies. Angewandte Chemie 58, 2680-2684 (2019).
(68) M. Tan and X. Jiang, Subviral particle as vaccine and vaccine platform. Curr Opin Virol. 2014 June; 6:24-33.

1. A three-dimensional polynucleotide-based open shell [1] (FIG. 26) encasing a cavity [2] and comprising an opening [3] for accessing said cavity, comprising
an n-gonal pyramid [4] formed by n identical copies of a first type of an acute isosceles triangular prismoid t1 [5], wherein n is an integer selected from 7, 8, 9, 10, 11, 12, 13, 14 and 15, wherein the base plane [6] of each prismoid points to the outside of said open shell, and the upper plane [7] points to said cavity, wherein the two large side planes [8,9] of each prismoid contain a first pattern [10] and a second pattern [11] of one or more protrusions and/or one or more receptacles, wherein said first and said second patterns are complementary to each other, and wherein the small side plane [12] comprises a third pattern [13] of one or more protrusions and/or one or more receptacles; wherein said first type of an acute isosceles triangular prismoid is a self-assembling DNA-based building block.

2. The three-dimensional polynucleotide-based open shell of claim 1, further comprising n copies of a second type of an acute isosceles triangular prismoid t2 [14], wherein a first side [15] of each prismoid points to the outside of said open shell, and the opposite side [16] points to said cavity and/or to said opening for accessing said cavity, wherein one plane [17] of said second type of prismoid structure [14] comprises a fourth pattern [18] of one or more protrusions and/or one or more receptacles which is complementary to said third pattern [13].

3. The three-dimensional polynucleotide-based open shell of claim 1 or 2, wherein said self-assembling DNA-based building block comprise between 7,500 and 10,500 base pairs and/or wherein the molecular weight of each self-assembling DNA-based building block is between 4.5 and 7 MDa.

4. The three-dimensional polynucleotide-based open shell of any one of claims 1 to 3, wherein said upper plane [7] and/or, when present, said opposite side [16] comprise one or more attachment sites for the attachment of one or more binding molecules.

5. The three-dimensional polynucleotide-based open shell of claim 4, wherein said binding molecules are selected from antibodies and antigen-binding fragments thereof and constructs comprising at least one sulfonated or sulfated polysaccharide group.

6. The three-dimensional polynucleotide-based open shell of claim 5, wherein said binding molecules are scFv fragments.

7. The three-dimensional polynucleotide-based open shell of claim 5, wherein said binding molecules are constructs comprising one or two sulfonated or sulfated polysaccharide groups.

8. The three-dimensional polynucleotide-based open shell of claim 7, wherein said binding molecules are independently selected from the list of heparin, heparan sulfate, hybrid heparan sulfates, carrageenans, cellulose sulfate, dextrin 2-sulfate, aptamers, peptides, host-receptor domains, and sialic acid.

9. The three-dimensional polynucleotide-based open shell of any one of claims 3 to 8, wherein each prismoid comprises between 1 and 45 of said attachment sites.

10. The three-dimensional polynucleotide-based open shell of claim 9, wherein each prismoid comprises between 3 and 10 attachment sites.

11. The three-dimensional polynucleotide-based open shell of any one of claims 3 to 10, wherein said attachment sites are first single-stranded oligonucleotides.

12. The three-dimensional polynucleotide-based open shell of claim 11, wherein said binding molecules are attached to said attachment sites by second single-stranded oligonucleotides, which are linked to said binding molecules and are complementary to said first single-stranded oligonucleotides.

13. The three-dimensional polynucleotide-based open shell of any one of claims 1 to 12, wherein each of said first and, if present, of said second types of said acute isosceles triangular prismoids is a DNA-based nanostructure formed by self-assembling DNA-based building blocks.

14. The three-dimensional polynucleotide-based open shell of claim 13, wherein said DNA-based nanostructure is formed by a single-stranded DNA template strand and a set of oligonucleotides complementary to said single-stranded DNA template, wherein each of said oligonucleotides is either complementary to one contiguous DNA sequence stretch or to at least two non-contiguous DNA sequence stretches on said single-stranded DNA template.

15. The three-dimensional polynucleotide-based open shell of any one of claims 1 to 14, wherein n is an integer selected from 9, 10, 11, 12 and 13.

16. The three-dimensional polynucleotide-based open shell of any one of claims 1 to 15, further comprising chemical crosslinks between different prismoids.

17. The three-dimensional polynucleotide-based open shell of claim 16, wherein said chemical crosslinks are obtained by UV irradiation.

18. The three-dimensional polynucleotide-based open shell of any one of claims 1 to 17, further comprising a coating of the outer surface of said open shell with a polycationic molecule.

19. The three-dimensional polynucleotide-based open shell of claim 18, wherein said polycationic molecule is a polylysine.

20. The three-dimensional polynucleotide-based open shell of claim 19, wherein said polycationic molecule is polylysine-PEG.

21. The three-dimensional polynucleotide-based open shell of claim 19 or 20, further comprising cross-links of free amino groups of said polylysine.

22. The three-dimensional polynucleotide-based open shell of claim 21, wherein said cross-links are with an alkane dialdehyde.

23. The three-dimensional polynucleotide-based open shell of claim 22, wherein said cross-links are with glutaraldehyde.

24. The three-dimensional polynucleotide-based open shell of any one of claims 1 to 23 for use in the treatment of a patient infected by, suspected to be infected by, or bearing the risk of becoming infected by, a virus, a viral particle or a subviral particle.

25. A composition comprising a mixture of a three-dimensional polynucleotide-based open shells according to any one of claims 1 to 23, wherein said mixture comprises three-dimensional polynucleotide-based open shells having values of n ranging from 7 to 15.

26. The composition of claim 25, wherein said mixture comprises three-dimensional polynucleotide-based open shells having values of n ranging from 9 to 13, with a maximum in the range of 9 to 11.

27. The composition of claim 25 or 26 for use in the treatment of a patient infected by, suspected to be infected by, or bearing the risk of becoming infected by, a virus, a viral particle or a subviral particle.

28. A method for encapsulating a virus, a viral particle or a subviral particle, comprising the steps of: providing a three-dimensional polynucleotide-based open shell according to any one of claims 1 to 23, or a composition according to claim 25 of 26, and contacting said three-dimensional polynucleotide-based open shell or said composition with a medium comprising, or suspected to comprise, said virus, said viral particle or said subviral particle.

29. A method for the treatment of a patient infected by, suspected to be infected by, or bearing the risk of becoming infected by, a virus, a viral particle or a subviral particle, comprising the step of: administering the three-dimensional polynucleotide-based open shell according to any one of claims 1 to 23, or the composition according to claim 25 of 26 to said patient.

30. A method for the treatment of a patient infected by, or suspected to be infected by, a virus, a viral particle or a subviral particle, comprising the step of:
contacting said patient, or a bodily fluid of said patient, with the three-dimensional polynucleotide-based open shell according to any one of claims 1 to 23, or the composition according to claim 25 of 26.

31. A composition comprising a virus, a viral particle or a subviral particle encapsulated by a three-dimensional polynucleotide-based open shell according to any one of claims 1 to 23 or by a three-dimensional polynucleotide-based open shell from the composition according to claim 25 or 26.


SEQUENCE LISTING (1408 sequences)

SEQ ID NO: 1 - DNA, 8064 bp, synthetic construct
  source 1..8064: mol_type = other DNA
ggcaatgacctgatagcctttgtagatctctcaaaaatagctaccctctccggcattaatttatcagctagaacggttgaatatcatattgatggtgatttgactgtctccggcctttctcacccttttgaatctttacctacacattactcaggcattgcatttaaaatatatgagggttctaaaaatttttatccttgcgttgaaataaaggcttctcccgcaaaagtattacagggtcataatgtttttggtacaaccgatttagctttatgctctgaggctttattgcttaattttgctaattctttgccttgcctgtatgatttattggatgttaatgctactactattagtagaattgatgccaccttttcagctcgcgccccaaatgaaaatatagctaaacaggttattgaccatttgcgaaatgtatctaatggtcaaactaaatctactcgttcgcagaattgggaatcaactgttatatggaatgaaacttccagacaccgtactttagttgcatatttaaaacatgttgagctacagcattatattcagcaattaagctctaagccatccgcaaaaatgacctcttatcaaaaggagcaattaaaggtactctctaatcctgacctgttggagtttgcttccggtctggttcgctttgaagctcgaattaaaacgcgatatttgaagtctttcgggcttcctcttaatctttttgatgcaatccgctttgcttctgactataatagtcagggtaaagacctgatttttgatttatggtcattctcgttttctgaactgtttaaagcatttgagggggattcaatgaatatttatgacgattccgcagtattggacgctatccagtctaaacattttactattaccccctctggcaaaacttcttttgcaaaagcctctcgctattttggtttttatcgtcgtctggtaaacgagggttatgatagtgttgctcttactatgcctcgtaattccttttggcgttatgtatctgcattagttgaatgtggtattcctaaatctcaactgatgaatctttctacctgtaataatgttgttccgttagttcgttttattaacgtagatttttcttcccaacgtcctgactggtataatgagccagttcttaaaatcgcataaggtaattcacaatgattaaagttgaaattaaaccatctcaagcccaatttactactcgttctggtgtttctcgtcagggcaagccttattcactgaatgagcagctttgttacgttgatttgggtaatgaatatccggttcttgtcaagattactcttgatgaaggtcagccagcctatgcgcctggtctgtacaccgttcatctgtcctctttcaaagttggtcagttcggttcccttatgattgaccgtctgcgcctcgttccggctaagtaacatggagcaggtcgcggatttcgacacaatttatcaggcgatgatacaaatctccgttgtactttgtttcgcgcttggtataatcgctgggggtcaaagatgagtgttttagtgtattcttttgcctctttcgttttaggttggtgccttcgtagtggcattacgtattttacccgtttaatggaaacttcctcatgaaaaagtctttagtcctcaaagcctctgtagccgttgctaccctcgttccgatgctgtctttcgctgctgagggtgacgatcccgcaaaagcggcctttaactccctgcaagcctcagcgaccgaatatatcggttatgcgtgggcgatggttgttgtcattgtcggcgcaactatcggtatcaagctgtttaagaaattcacctcgaaagcaagctgataaaccgatacaattaaaggctccttttggagccttttttttggagattttcaacgtgaaaaaattattattcgcaattcctttagttgttcctttctattctcactccgctgaaactgttgaaagttgtttagcaaaatcccatacagaaaattcatttactaacgtctggaaagacgacaaaactttagatcgttacgctaactatgagggctgtctgtggaatgctacaggcgttgtagtttgtactggtgacgaaactcagtgttacggtacatgggttcctattgggcttgctatccctgaaaatgagggtggtggctctgagggtggcggttctgagggtggcggttctgagggtggcggtactaaacctcctgagtacggtgatacacctattccgggctatacttatatcaaccctctcgacggcacttatccgcctggtactgagcaaaaccccgctaatcctaatccttctcttgaggagtctcagcctcttaatactttcatgtttcagaataataggttccgaaataggcagggggcattaactgtttatacgggcactgttactcaaggcactgaccccgttaaaacttattaccagtacactcctgtatcatcaaaagccatgtatgacgcttactggaacggtaaattcagagactgcgctttccattctggctttaatgaggatttatttgtttgtgaatatcaaggccaatcgtctgacctgcctcaacctcctgtcaatgctggcggcggctctggtggtggttctggtggcggctctgagggtggtggctctgagggtggcggttctgagggtggcggctctgagggaggcggttccggtggtggctctggttccggtgattttgattatgaaaagatggcaaacgctaataagggggctatgaccgaaaatgccgatgaaaacgcgctacagtctgacgctaaaggcaaacttgattctgtcgctactgattacggtgctgctatcgatggtttcattggtgacgtttccggccttgctaatggtaatggtgctactggtgattttgctggctctaattcccaaatggctcaagtcggtgacggtgataattcacctttaatgaataatttccgtcaatatttaccttccctccctcaatcggttgaatgtcgcccttttgtctttggcgctggtaaaccatatgaattttctattgattgtgacaaaataaacttattccgtggtgtctttgcgtttcttttatatgttgccacctttatgtatgtattttctacgtttgctaacatactgcgtaataaggagtcttaatcatgccagttcttttgggtattccgttattattgcgtttcctcggtttccttctggtaactttgttcggctatctgcttacttttcttaaaaagggcttcggtaagatagctattgctatttcattgtttcttgctcttattattgggcttaactcaattcttgtgggttatctctctgatattagcgctcaattaccctctgactttgttcagggtgttcagttaattctcccgtctaatgcgcttccctgtttttatgttattctctctgtaaaggctgctattttcatttttgacgttaaacaaaaaatcgtttcttatttggattgggataaataatatggctgtttattttgtaactggcaaattaggctctggaaagacgctcgttagcgttggtaagattcaggataaaattgtagctgggtgcaaaatagcaactaatcttgatttaaggcttcaaaacctcccgcaagtcgggaggttcgctaaaacgcctcgcgttcttagaataccggataagccttctatatctgatttgcttgctattgggcgcggtaatgattcctacgatgaaaataaaaacggcttgcttgttctcgatgagtgcggtacttggtttaatacccgttcttggaatgataaggaaagacagccgattattgattggtttctacatgctcgtaaattaggatgggatattatttttcttgttcaggacttatctattgttgataaacaggcgcgttctgcattagctgaacatgttgtttattgtcgtcgtctggacagaattactttaccttttgtcggtactttatattctcttattactggctcgaaaatgcctctgcctaaattacatgttggcgttgttaaatatggcgattctcaattaagccctactgttgagcgttggctttatactggtaagaatttgtataacgcatatgatactaaacaggctttttctagtaattatgattccggtgtttattcttatttaacgccttatttatcacacggtcggtatttcaaaccattaaatttaggtcagaagatgaaattaactaaaatatatttgaaaaagttttctcgcgttctttgtcttgcgattggatttgcatcagcatttacatatagttatataacccaacctaagccggaggttaaaaaggtagtctctcagacctatgattttgataaattcactattgactcttctcagcgtcttaatctaagctatcgctatgttttcaaggattctaagggaaaattaattaatagcgacgatttacagaagcaaggttattcactcacatatattgatttatgtactgtttccattaaaaaaggtaattcaaatgaaattgttaaatgtaattaattttgttttcttgatgtttgtttcatcatcttcttttgctcaggtaattgaaatgaataattcgcctctgcgcgattttgtaacttggtattcaaagcaatcaggcgaatccgttattgtttctcccgatgtaaaaggtactgttactgtatattcatctgacgttaaacctgaaaatctacgcaatttctttatttctgttttacgtgcaaataattttgatatggtaggttctaacccttccattattcagaagtataatccaaacaatcaggattatattgatgaattgccatcatctgataatcaggaatatgatgataattccgctccttctggtggtttctttgttccgcaaaatgataatgttactcaaacttttaaaattaataacgttcgggcaaaggatttaatacgagttgtcgaattgtttgtaaagtctaatacttctaaatcctcaaatgtattatctattgacggctctaatctattagttgttagtgctcctaaagatattttagataaccttcctcaattcctttcaactgttgatttgccaactgaccagatattgattgagggtttgatatttgaggttcagcaaggtgatgctttagatttttcatttgctgctggctctcagcgtggcactgttgcaggcggtgttaatactgaccgcctcacctctgttttatcttctgctggtggttcgttcggtatttttaatggcgatgttttagggctatcagttcgcgcattaaagactaatagccattcaaaaatattgtctgtgccacgtattcttacgctttcaggtcagaagggttctatctctgttggccagaatgtcccttttattactggtcgtgtgactggtgaatctgccaatgtaaataatccatttcagacgattgagcgtcaaaatgtaggtatttccatgagcgtttttcctgttgcaatggctggcggtaatattgttctggatattaccagcaaggccgatagtttgagttcttctactcaggcaagtgatgttattactaatcaaagaagtattgctacaacggttaatttgcgtgatggacagactcttttactcggtggcctcactgattataaaaacacttctcaggattctggcgtaccgttcctgtctaaaatccctttaatcggcctcctgtttagctcccgctctgattctaacgaggaaagcacgttatacgtgctcgtcaaagcaaccatagtacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaactggaacaacactcaaccctatctcgggctattcttttgatttataagggattttgccgatttcggaaccaccatcaaacaggattttcgcctgctggggcaaaccagcgtggaccgcttgctgcaactctctcagggccaggcggtgaagggcaatcagctgttgcccgtctcactggtgaaaagaaaaaccaccctggcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgaattcgagctcggtacccggggatcctcaactgtgaggaggctcacggacgcgaagaacaggcacgcgtgctggcagaaacccccggtatgaccgtgaaaacggcccgccgcattctggccgcagcaccacagagtgcacaggcgcgcagtgacactgcgctggatcgtctgatgcagggggcaccggcaccgctggctgcaggtaacccggcatctgatgccgttaacgatttgctgaacacaccagtgtaagggatgtttatgacgagcaaagaaacctttacccattaccagccgcagggcaacagtgacccggctcataccgcaaccgcgcccggcggattgagtgcgaaagcgcctgcaatgaccccgctgatgctggacacctccagccgtaagctggttgcgtgggatggcaccaccgacggtgctgccgttggcattcttgcggttgctgctgaccagaccagcaccacgctgacgttctacaagtccggcacgttccgttatgaggatgtgctctggccggaggctgccagcgacgagacgaaaaaacggaccgcgtttgccggaacggcaatcagcatcgtttaactttacccttcatcactaaaggccgcctgtgcggctttttttacgggatttttttatgtcgatgtacacaaccgcccaactgctggcggcaaatgagcagaaatttaagtttgatccgctgtttctgcgtctctttttccgtgagagctatcccttcaccacggagaaagtctatctctcacaaattccgggactggtaaacatggcgctgtacgtttcgccgattgtttccggtgaggttatccgttcccgtggcggctccacctctgaaagcttggcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgctttgcctggtttccggcaccagaagcggtgccggaaagctggctggagtgcgatcttcctgaggccgatactgtcgtcgtcccctcaaactggcagatgcacggttacgatgcgcccatctacaccaacgtgacctatcccattacggtcaatccgccgtttgttcccacggagaatccgacgggttgttactcgctcacatttaatgttgatgaaagctggctacaggaaggccagacgcgaattatttttgatggcgttcctattggttaaaaaatgagctgatttaacaaaaatttaatgcgaattttaacaaaatattaacgtttacaatttaaatatttgcttatacaatcttcctgtttttggggcttttctgattatcaaccggggtacatatgattgacatgctagttttacgattaccgttcatcgattctcttgtttgctccagactctca

SEQ ID NO: 2 - DNA, 56 bp, synthetic construct
  source 1..56: mol_type = other DNA
ttatactttcagctcattttttaaatattttgagcggattatcaaaaatcaggtct

SEQ ID NO: 3 - DNA, 56 bp, synthetic construct
  source 1..56: mol_type = other DNA
gccagcattgacatcaagtaccagtagataagtcctgaacccagcacgcagcgcca

SEQ ID NO: 4 - DNA, 56 bp, synthetic construct
  source 1..56: mol_type = other DNA
atacccaaggcctttatgtacatcatagaaggtgccagttgaagccttaaaggaac

SEQ ID NO: 5 - DNA, 56 bp, synthetic construct
  source 1..56: mol_type = other DNA
tgcgattttaagaactccaatggaaataaagacttcaaatatcgcgttgtcatttt

SEQ ID NO: 6 - DNA, 57 bp, synthetic construct
  source 1..57: mol_type = other DNA
tgcccgctttccagtctcagaaccatggttcagctaatgcagtaataacattttgac

SEQ ID NO: 7 - DNA, 57 bp, synthetic construct
  source 1..57: mol_type = other DNA
acctgcagccagcatcagcggggtcattgcaggcgagaacaagcaagccttaacgtc

SEQ ID NO: 8 - DNA, 56 bp, synthetic construct
  source 1..56: mol_type = other DNA
gtaaattgttatgaccctgtaataaccccggtatagcgtcaatccccctcaaatgc

SEQ ID NO: 9 - DNA, 56 bp, synthetic construct
  source 1..56: mol_type = other DNA
gttaccagatccaataggagtaacaaagctgctcattcagcctcaccgaatggtca

SEQ ID NO: 10 - DNA, 57 bp, synthetic construct
  source 1..57: mol_type = other DNA
agatttagcgccaaaaggaattacattcaaccgaatttttcgatgaacggtaatcgt

SEQ ID NO: 11 - DNA, 56 bp, synthetic construct
  source 1..56: mol_type = other DNA
cagaatcaagccgccaagcactaaagtaattctcgtcgctatttaacaatttcatt

SEQ ID NO: 12 - DNA, 58 bp, synthetic construct
  source 1..58: mol_type = other DNA
gggtaatagtaaaatgttaaagagaggcttttgcttcaaagcgaaccagagagtacct

SEQ ID NO: 13 - DNA, 57 bp, synthetic construct
  source 1..57: mol_type = other DNA
cctgattgtttaggaacgccatcatgcggatgagctcaacatgttttaaatatgcaa

SEQ ID NO: 14 - DNA, 53 bp, synthetic construct
  source 1..53: mol_type = other DNA
tacgcagtatgttagcaaacgtagaaaataaacatccataccgggggtttctg

SEQ ID NO: 15 - DNA, 53 bp, synthetic construct
  source 1..53: mol_type = other DNA
agcagcaactacaattcgtacagcgccattgagatagccgcaataataacgga

SEQ ID NO: 16 - DNA, 58 bp, synthetic construct
  source 1..58: mol_type = other DNA
tgcgtattgggtttttgccagggtggtttttctatcaccaccagcagaaaaaagtttg

SEQ ID NO: 17 - DNA, 57 bp, synthetic construct
  source 1..57: mol_type = other DNA
agaaagcgaaaggagacacccagcaggcgaaaatcctgtttttttgatggagttgca

SEQ ID NO: 18 - DNA, 53 bp, synthetic construct
  source 1..53: mol_type = other DNA
ttagagccctttgattcgggtaccgagcttccgcgctcaccagctgcattaat

SEQ ID NO: 19 - DNA, 62 bp, synthetic construct
  source 1..62: mol_type = other DNA
tgaattacaaaaagcccgagggttgatataaggtcagacgattaggtgccgtaaccagaacc

SEQ ID NO: 20 - DNA, 53 bp, synthetic construct
  source 1..53: mol_type = other DNA
taaaacgaaagaggcaaaagaagcttgataccgatagcatgtaccgtaacact

SEQ ID NO: 21 - DNA, 53 bp, synthetic construct
  source 1..53: mol_type = other DNA
tcagtattaaagggatgccaccgacagacaatatttttgattcaaatagtgta

SEQ ID NO: 22 - DNA, 63 bp, synthetic construct
  source 1..63: mol_type = other DNA
gatagctcccaggcgctattattctgtttttaactaataagtttttgcaagacttgagccatt

SEQ ID NO: 23 - DNA, 52 bp, synthetic construct
  source 1..52: mol_type = other DNA
ttttcaggtttaacgtctaaaaatcatattaatttcatcttctggtaagaat

SEQ ID NO: 24 - DNA, 52 bp, synthetic construct
  source 1..52: mol_type = other DNA
tggaaggattcatccccttcagtgagacgggcaacactgaatataaggagcg

SEQ ID NO: 25 - DNA, 59 bp, synthetic construct
  source 1..59: mol_type = other DNA
ctttacaaacaattcgacaactcgtcagtgagtttagacattttttgaacggtaaaata

SEQ ID NO: 26 - DNA, 55 bp, synthetic construct
  source 1..55: mol_type = other DNA
aaaagccgcacaggcaagaactggacggaaattattcattaaagagcggcggttg

SEQ ID NO: 27 - DNA, 51 bp, synthetic construct
  source 1..51: mol_type = other DNA
cgcgcttattttttgcgccgctacagggctggcaatattttagggtctgag

SEQ ID NO: 28 - DNA, 55 bp, synthetic construct
  source 1..55: mol_type = other DNA
ccgaacgaagatgatggcagttagaacgaaatcggccagtttggaaaaggaaggg

SEQ ID NO: 29 - DNA, 51 bp, synthetic construct
  source 1..51: mol_type = other DNA
ctttcatcatttcaactttaatctttttttgtgaattaccttacaggacgt

SEQ ID NO: 30 - DNA, 54 bp, synthetic construct
  source 1..54: mol_type = other DNA
aagggcgagatagatgaatatacagttttttacagtaccttgaaattgcgtaga

SEQ ID NO: 31 - DNA, 64 bp, synthetic construct
  source 1..64: mol_type = other DNA
ttcagaggtttggggctgattcccaattctgcgaacgagtttttttcgccacgggaacggataa

SEQ ID NO: 32 - DNA, 64 bp, synthetic construct
  source 1..64: mol_type = other DNA
aaaataataactgttgtacgccagctggcgaaagggggatgcggagatggatcagttgtgggaa

SEQ ID NO: 33 - DNA, 50 bp, synthetic construct
  source 1..50: mol_type = other DNA
gtttttataatattaaatcctttttttgcccgaacgttatcctgagaagt

SEQ ID NO: 34 - DNA, 56 bp, synthetic construct
  source 1..56: mol_type = other DNA
agaacgcgagcctccttcacaatcacaccacggaataagtgggaaaccgcatcacc

SEQ ID NO: 35 - DNA, 55 bp, synthetic construct
  source 1..55: mol_type = other DNA
cggtgccccctgcatcagacaaatcccacgcaaccagcttacggctgtaggaatc

SEQ ID NO: 36 - DNA, 47 bp, synthetic construct
  source 1..47: mol_type = other DNA
gagtccactattatattatagtcgggttgagttgttaaactgaataa

SEQ ID NO: 37 - DNA, 40 bp, synthetic construct
  source 1..40: mol_type = other DNA
gacgctgatcccgggcgctagggcgcgcgtacacgtggca

SEQ ID NO: 38 - DNA, 49 bp, synthetic construct
  source 1..49: mol_type = other DNA
ataagagttaattttttcgagcaaatttttgaagttttattataccagt

SEQ ID NO: 39 - DNA, 49 bp, synthetic construct
  source 1..49: mol_type = other DNA
atgatgaggcttttttagtacaggtagtttttaagattcgcgcatcgta

SEQ ID NO: 40 - DNA, 49 bp, synthetic construct
  source 1..49: mol_type = other DNA
gagataacccacaagacatcctaatttacgagggccgtttgattgaggg

SEQ ID NO: 41 - DNA, 49 bp, synthetic construct
  source 1..49: mol_type = other DNA
agaaacgattttttgtgtttttattttcatcggaggtgtccagcggtgc

SEQ ID NO: 42 - DNA, 49 bp, synthetic construct
  source 1..49: mol_type = other DNA
gaaaacattattaagagcaacactatcatacagtcaaatcaccatcaat

SEQ ID NO: 43 - DNA, 48 bp, synthetic construct
  source 1..48: mol_type = other DNA
agagaaggtagaaccagagccacctggtttaccgtgcctgaatatctg

SEQ ID NO: 44 - DNA, 48 bp, synthetic construct
  source 1..48: mol_type = other DNA
cggaacgagcgacctgctccatgtatgtgtaggtaaagattcatatgt

SEQ ID NO: 45 - DNA, 40 bp, synthetic construct
  source 1..40: mol_type = other DNA
ctccggccataacagtacaggtcaggattagaccggaagc

SEQ ID NO: 46 - DNA, 48 bp, synthetic construct
  source 1..48: mol_type = other DNA
gaaggcacaccatcgcccacgcatagccaccaccctcattaataacat

SEQ ID NO: 47 - DNA, 48 bp, synthetic construct
  source 1..48: mol_type = other DNA
ggaagggcgatcggtgaatgctgtgcttagagcttaattgatcaaaag

SEQ ID NO: 48 - DNA, 48 bp, synthetic construct
  source 1..48: mol_type = other DNA
aataagtttcataaattacttagctattttaaatgcaatgcttttgcg

SEQ ID NO: 49 - DNA, 48 bp, synthetic construct
  source 1..48: mol_type = other DNA
cgccacccttattttgcacagttgttgctgaaactaacaactaataga

SEQ ID NO: 50 - DNA, 48 bp, synthetic construct
  source 1..48: mol_type = other DNA
acggaacgtgccggacttgtagaacgtcagcgcggcaaacgcggtccg

SEQ ID NO: 51 - DNA, 48 bp, synthetic construct
  source 1..48: mol_type = other DNA
gcattaacatttcgcagaaacaattgccgttctggtgctggtctggtc

SEQ ID NO: 52 - DNA, 48 bp, synthetic construct
  source 1..48: mol_type = other DNA
gacataaaatttctgcgtcaccgaaacaatgaaatagcaatcggaacc

SEQ ID NO: 53 - DNA, 48 bp, synthetic construct
  source 1..48: mol_type = other DNA
agcacgtagttatccgttaatggttaaagtaaaatagtgaagatgatg

SEQ ID NO: 54 - DNA, 40 bp, synthetic construct
  source 1..40: mol_type = other DNA
aatggaaacagtacatgaggactaaagactttcggctaca

SEQ ID NO: 55 - DNA, 48 bp, synthetic construct
  source 1..48: mol_type = other DNA
agactaccgaattattcatttcaattacctgagcaaaagaatttatca

SEQ ID NO: 56 - DNA, 40 bp, synthetic construct
  source 1..40: mol_type = other DNA
attaagagagccagaatggaaatttttcgcagtctctgaa

SEQ ID NO: 57 - DNA, 48 bp, synthetic construct
  source 1..48: mol_type = other DNA
ccagccagctttcagtgccaagctaacgagtaaaactccataaatcgg

SEQ ID NO: 58 - DNA, 48 bp, synthetic construct
  source 1..48: mol_type = other DNA
aatataatgaattatcacaaagaaaccaccagcgggcctcttcgctat

SEQ ID NO: 59 - DNA, 48 bp, synthetic construct
  source 1..48: mol_type = other DNA
aaaaatgaaattttttagagtaccgtttttactcatcgctttcgcact

SEQ ID NO: 60 - DNA, 48 bp, synthetic construct
  source 1..48: mol_type = other DNA
actcaaaccaatacttgtcaatagaaatgaaaaatctaaatgtcgtgc

SEQ ID NO: 61 - DNA, 48 bp, synthetic construct
  source 1..48: mol_type = other DNA
tgaataagtctactaaaaattaagcaataaagttaacgggccctcata

SEQ ID NO: 62 - DNA, 48 bp, synthetic construct
  source 1..48: mol_type = other DNA
cataaagcgcgagctgaaaaggtgtattttcatggagccgtctcgtcg

SEQ ID NO: 63 - DNA, 48 bp, synthetic construct
  source 1..48: mol_type = other DNA
tgtgataaatagctgtaaaggtaaatcggaactcacccaaatcaagtt

SEQ ID NO: 64 - DNA, 47 bp, synthetic construct
  source 1..47: mol_type = other DNA
accgtgcatgattctccgttttttggaacaaacggtgctgcaaggcg

SEQ ID NO: 65 - DNA, 47 bp, synthetic construct
  source 1..47: mol_type = other DNA
aactaaagatctccaatcggtttacgattatatgtacagacgctttt

SEQ ID NO: 66 - DNA, 39 bp, synthetic construct
  source 1..39: mol_type = other DNA
gtcaccaatgaaacaaaatcaccggcaccattaccatta

SEQ ID NO: 67 - DNA, 40 bp, synthetic construct
  source 1..40: mol_type = other DNA
gccaccctcagaggtagtagcgcgttttcatcggcatttt

SEQ ID NO: 68 - DNA, 23 bp, synthetic construct
  source 1..23: mol_type = other DNA
tgaggcagtatagccatatgtga

SEQ ID NO: 69 - DNA, 69 bp, synthetic construct
  source 1..69: mol_type = other DNA
ttgaggggcggaacaaatctacgtcaggaagattgtttttttaagcaaatattaaacaagagttctagc

SEQ ID NO: 70 - DNA, 46 bp, synthetic construct
  source 1..46: mol_type = other DNA
cgcctccagccgcctagcagcaccgtaatcagtagcgacggtcata

SEQ ID NO: 71 - DNA, 46 bp, synthetic construct
  source 1..46: mol_type = other DNA
cgcaaagaatagaaaattcataaccggaacatcaacaagcggataa

SEQ ID NO: 72 - DNA, 46 bp, synthetic construct
  source 1..46: mol_type = other DNA
ataggctgatgaacggccaagcgcgaaacaaagtacaacttgctaa

SEQ ID NO: 73 - DNA, 24 bp, synthetic construct
  source 1..24: mol_type = other DNA
ttgacaagaaccgaacgtatcatc

SEQ ID NO: 74 - DNA, 24 bp, synthetic construct
  source 1..24: mol_type = other DNA
atgggattggagattttgaccaac

SEQ ID NO: 75 - DNA, 46 bp, synthetic construct
  source 1..46: mol_type = other DNA
aggcttttttttttacctccggcttaggttttacaaaatcgcgcag

SEQ ID NO: 76 - DNA, 30 bp, synthetic construct
  source 1..30: mol_type = other DNA
gctcaatttaggagccctcaaatatcaaac

SEQ ID NO: 77 - DNA, 45 bp, synthetic construct
  source 1..45: mol_type = other DNA
agtaacattatcattttgcggaatcatattcctgatttttcacca

SEQ ID NO: 78 - DNA, 31 bp, synthetic construct
  source 1..31: mol_type = other DNA
actaagcctgttgcgtttcgttagcagcagc

SEQ ID NO: 79 - DNA, 45 bp, synthetic construct
  source 1..45: mol_type = other DNA
tcatcaaatagactttaccgaagccccatacaggcaaccgtataa

SEQ ID NO: 80 - DNA, 45 bp, synthetic construct
  source 1..45: mol_type = other DNA
cacccagccgcaaggggtaaagttaaacgcgaggaaacgaacaaa

SEQ ID NO: 81 - DNA, 44 bp, synthetic construct
  source 1..44: mol_type = other DNA
gaggaaggttatctaaaatatctcgtctgagtccgtgcctgttt

SEQ ID NO: 82 - DNA, 44 bp, synthetic construct
  source 1..44: mol_type = other DNA
ttgtaccatagactggtgataatccatgtcaatcaaaagggtga

SEQ ID NO: 83 - DNA, 44 bp, synthetic construct
  source 1..44: mol_type = other DNA
tgggaattagagattcaacctcacggtccttacacttttctttg

SEQ ID NO: 84 - DNA, 32 bp, synthetic construct
  source 1..32: mol_type = other DNA
agaagcaattaaaattggctatcaagctattt

SEQ ID NO: 85 - DNA, 31 bp, synthetic construct
  source 1..31: mol_type = other DNA
acaggagtcttgagtaacagtgcggcaaaga

SEQ ID NO: 86 - DNA, 43 bp, synthetic construct
  source 1..43: mol_type = other DNA
gcccaataggaaccttgcgccgatttttaatgacaacacaacc

SEQ ID NO: 87 - DNA, 31 bp, synthetic construct
  source 1..31: mol_type = other DNA
gcaagcggtccacgcggccgattaacaccgc

SEQ ID NO: 88 - DNA, 43 bp, synthetic construct
  source 1..43: mol_type = other DNA
ccggcgaacggcgagacaagaaataaattacatcgtttgaata

SEQ ID NO: 89 - DNA, 32 bp, synthetic construct
  source 1..32: mol_type = other DNA
acatagcgatagcttaaaaaccgtggggaaag

SEQ ID NO: 90 - DNA, 32 bp, synthetic construct
  source 1..32: mol_type = other DNA
tgtttagtttaaataagaataaacacacgacc

SEQ ID NO: 91 - DNA, 43 bp, synthetic construct
  source 1..43: mol_type = other DNA
ttcctgtgagtccaccaccctcagagtttgcctttagcgtcag

SEQ ID NO: 92 - DNA, 32 bp, synthetic construct
  source 1..32: mol_type = other DNA
ggtgaatttcttaaacatacactaaaacactc

SEQ ID NO: 93 - DNA, 32 bp, synthetic construct
  source 1..32: mol_type = other DNA
acatacataagacaaaagggcgacccagcaaa

SEQ ID NO: 94 - DNA, 32 bp, synthetic construct
  source 1..32: mol_type = other DNA
tacccaaatcaacattatctaaagttttctgt

SEQ ID NO: 95 - DNA, 41 bp, synthetic construct
  source 1..41: mol_type = other DNA
aaaaacagtccttatcattccaaggccgggtctgccgggtt

SEQ ID NO: 96 - DNA, 32 bp, synthetic construct
  source 1..32: mol_type = other DNA
gtaaaagagtctgtccgtattagactgcaaca

SEQ ID NO: 97 - DNA, 40 bp, synthetic construct
  source 1..40: mol_type = other DNA
acgttgtaaaacgacgagagcacatcctcatactggcagc

SEQ ID NO: 98 - DNA, 32 bp, synthetic construct
  source 1..32: mol_type = other DNA
cctcagagggagaagctgacgagaaacaccag

SEQ ID NO: 99 - DNA, 40 bp, synthetic construct
  source 1..40: mol_type = other DNA
agatttagataacctgtttagctagcatcaatgcttgccc

SEQ ID NO: 100 - DNA, 35 bp, synthetic construct
  source 1..35: mol_type = other DNA
tatcccaatccaaatagagtttcggtcagagggta

SEQ ID NO: 101 - DNA, 40 bp, synthetic construct
  source 1..40: mol_type = other DNA
gcagcgaaagacaaacgggtaaaatacgtaatgccactac

SEQ ID NO: 102 - DNA, 32 bp, synthetic construct
  source 1..32: mol_type = other DNA
ctttgacgaatgagtgcaacatacgagccgga

SEQ ID NO: 103 - DNA, 40 bp, synthetic construct
  source 1..40: mol_type = other DNA
cggtgtctggaagtttcattccatgcccggcaccgcttct

SEQ ID NO: 104 - DNA, 40 bp, synthetic construct
  source 1..40: mol_type = other DNA
gcatcagaactgttgccctgcggctggtaatgggtaaagg

SEQ ID NO: 105 - DNA, 40 bp, synthetic construct
  source 1..40: mol_type = other DNA
tagctatcttctccgtcgccagcagcctaattcttttttg

SEQ ID NO: 106 - DNA, 40 bp, synthetic construct
  source 1..40: mol_type = other DNA
gtgccacgacgcgcggagctaactcacattaagggtgcct

SEQ ID NO: 107 - DNA, 39 bp, synthetic construct
  source 1..39: mol_type = other DNA
ataatacatttgaggatttagaaatcacgcacgggagct

SEQ ID NO: 108 - DNA, 39 bp, synthetic construct
  source 1..39: mol_type = other DNA
ctttatttcaacgcaaggatacatattagcatagtagta

SEQ ID NO: 109 - DNA, 40 bp, synthetic construct
  source 1..40: mol_type = other DNA
gtgatgaaaatgccaacggcagcaccgtcggtaatcagat

SEQ ID NO: 110 - DNA, 32 bp, synthetic construct
  source 1..32: mol_type = other DNA
taatatccattgagtttagcggggccgtactc

SEQ ID NO: 111 - DNA, 35 bp, synthetic construct
  source 1..35: mol_type = other DNA
aagaaaaaatcaccagattaggataggccggaaac

SEQ ID NO: 112 - DNA, 40 bp, synthetic construct
  source 1..40: mol_type = other DNA
ctcgtcatacagttgaaaggaattgtcagttggcaaatca

SEQ ID NO: 113 - DNA, 38 bp, synthetic construct
  source 1..38: mol_type = other DNA
taaaggctcacggaaaaagagacgcagaaatcaccagt

SEQ ID NO: 114 - DNA, 39 bp, synthetic construct
  source 1..39: mol_type = other DNA
ctcagagcgcccccttattagcgtttcataatccatcga

SEQ ID NO: 115 - DNA, 32 bp, synthetic construct
  source 1..32: mol_type = other DNA
cgatttagtaagagaaccttgaaaaaacaaac

SEQ ID NO: 116 - DNA, 33 bp, synthetic construct
  source 1..33: mol_type = other DNA
agggaaggtcgtgatccagcgcagtattaccgc

SEQ ID NO: 117 - DNA, 34 bp, synthetic construct
  source 1..34: mol_type = other DNA
aaaatccaaatattgcatgattaagactccttat

SEQ ID NO: 118 - DNA, 39 bp, synthetic construct
  source 1..39: mol_type = other DNA
ttgaaataccgaccgaacagagacttcgaattcgtaatc

SEQ ID NO: 119 - DNA, 36 bp, synthetic construct
  source 1..36: mol_type = other DNA
tatggttgaaacaggatggtttgcggagaggcggtt

SEQ ID NO: 120 - DNA, 38 bp, synthetic construct
  source 1..38: mol_type = other DNA
aaactttatggctattttttagtctttaatacgcgaga

SEQ ID NO: 121 - DNA, 36 bp, synthetic construct
  source 1..36: mol_type = other DNA
caatccgccgggcgcggttgcggtatgaaacgggta

SEQ ID NO: 122 - DNA, 36 bp, synthetic construct
  source 1..36: mol_type = other DNA
aaaagtagtgaattatcacctcatttgcggtgaagg

SEQ ID NO: 123 - DNA, 38 bp, synthetic construct
  source 1..38: mol_type = other DNA
ttaaaccacagcctttacagagagttcagggatagcaa

SEQ ID NO: 124 - DNA, 35 bp, synthetic construct
  source 1..35: mol_type = other DNA
taactgagcgcctgtgcattttttctgtggtgctg

SEQ ID NO: 125 - DNA, 36 bp, synthetic construct
  source 1..36: mol_type = other DNA
gcgatggcccactacttagaattataaagaccagta

SEQ ID NO: 126 - DNA, 36 bp, synthetic construct
  source 1..36: mol_type = other DNA
attaagttgggtaacgccagggttttccgagtaaca

SEQ ID NO: 127 - DNA, 43 bp, synthetic construct
  source 1..43: mol_type = other DNA
gcaaagcccaaatgaaagtaggaggttggtagcaattcatgag

SEQ ID NO: 128 - DNA, 32 bp, synthetic construct
  source 1..32: mol_type = other DNA
tgataaattaatgccggagagggtggtcattg

SEQ ID NO: 129 - DNA, 32 bp, synthetic construct
  source 1..32: mol_type = other DNA
cctgagtaattcattgcaatactgcggaatcg

SEQ ID NO: 130 - DNA, 40 bp, synthetic construct
  source 1..40: mol_type = other DNA
gcctgataaattgtgtcgaaatccggcgcagacggtcaat

SEQ ID NO: 131 - DNA, 40 bp, synthetic construct
  source 1..40: mol_type = other DNA
aataaatcaaatcaatcggaataggtgtatcattttgctc

SEQ ID NO: 132 - DNA, 39 bp, synthetic construct
  source 1..39: mol_type = other DNA
ttaccctgtctacaaacgcattaaattgcacgtaaaaca

SEQ ID NO: 133 - DNA, 30 bp, synthetic construct
  source 1..30: mol_type = other DNA
tttgaaatccagtaatgccccctgcctatt

SEQ ID NO: 134 - DNA, 35 bp, synthetic construct
  source 1..35: mol_type = other DNA
tttaaacaaaaactagagaaaagctttaccagact

SEQ ID NO: 135 - DNA, 24 bp, synthetic construct
  source 1..24: mol_type = other DNA
tttaccgtgaggacaggctgacct

SEQ ID NO: 136 - DNA, 34 bp, synthetic construct
  source 1..34: mol_type = other DNA
ccaaggggttaatcgcaagacaaagagcgcgaac

SEQ ID NO: 137 - DNA, 35 bp, synthetic construct
  source 1..35: mol_type = other DNA
cctgagaggaccataagcatcaaaaaggccagagg

SEQ ID NO: 138 - DNA, 31 bp, synthetic construct
  source 1..31: mol_type = other DNA
atcaagaaaacaaaaaaattctttaccgaca

SEQ ID NO: 139 - DNA, 31 bp, synthetic construct
  source 1..31: mol_type = other DNA
ttgagagagattcgcctgattgcggagaaac

SEQ ID NO: 140 - DNA, 31 bp, synthetic construct
  source 1..31: mol_type = other DNA
aataacgacaagaacgtggactccaacgtca

SEQ ID NO: 141 - DNA, 22 bp, synthetic construct
  source 1..22: mol_type = other DNA
atctttgacccccagtcagctt

SEQ ID NO: 142 - DNA, 24 bp, synthetic construct
  source 1..24: mol_type = other DNA
ttttggggtctgtaaatgtccaga

SEQ ID NO: 143 - DNA, 33 bp, synthetic construct
  source 1..33: mol_type = other DNA
gaggctttctcattaagctgagactccggaggt

SEQ ID NO: 144 - DNA, 38 bp, synthetic construct
  source 1..38: mol_type = other DNA
attttcccgtgaaccacctaaagggagccccagcataa

SEQ ID NO: 145 - DNA, 42 bp, synthetic construct
  source 1..42: mol_type = other DNA
gaagtttccattagcatcggaacgagtagtaccgccaccctc

SEQ ID NO: 146 - DNA, 86 bp, synthetic construct
  source 1..86: mol_type = other DNA
gcagtagagtaggtagagattaggcattgcagtagagtaggtagagattaggcatgataaattaatgccggagagggtggtcattg

SEQ ID NO: 147 - DNA, 86 bp, synthetic construct
  source 1..86: mol_type = other DNA
gcagtagagtaggtagagattaggcattgcagtagagtaggtagagattaggcacctgagtaattcattgcaatactgcggaatcg

SEQ ID NO: 148 - DNA, 94 bp, synthetic construct
  source 1..94: mol_type = other DNA
gcagtagagtaggtagagattaggcattgcagtagagtaggtagagattaggcagcctgataaattgtgtcgaaatccggcgcagacggtcaat

SEQ ID NO: 149 - DNA, 94 bp, synthetic construct
  source 1..94: mol_type = other DNA
gcagtagagtaggtagagattaggcattgcagtagagtaggtagagattaggcaaataaatcaaatcaatcggaataggtgtatcattttgctc

SEQ ID NO: 150 - DNA, 93 bp, synthetic construct
  source 1..93: mol_type = other DNA
gcagtagagtaggtagagattaggcattgcagtagagtaggtagagattaggcattaccctgtctacaaacgcattaaattgcacgtaaaaca

SEQ ID NO: 151 - DNA, 84 bp, synthetic construct
  source 1..84: mol_type = other DNA
gcagtagagtaggtagagattaggcattgcagtagagtaggtagagattaggcatttgaaatccagtaatgccccctgcctatt

SEQ ID NO: 152 - DNA, 89 bp, synthetic construct
  source 1..89: mol_type = other DNA
gcagtagagtaggtagagattaggcattgcagtagagtaggtagagattaggcatttaaacaaaaactagagaaaagctttaccagact

SEQ ID NO: 153 - DNA, 78 bp, synthetic construct
  source 1..78: mol_type = other DNA
gcagtagagtaggtagagattaggcattgcagtagagtaggtagagattaggcatttaccgtgaggacaggctgacct

SEQ ID NO: 154 - DNA, 88 bp, synthetic construct
  source 1..88: mol_type = other DNA
gcagtagagtaggtagagattaggcattgcagtagagtaggtagagattaggcaccaaggggttaatcgcaagacaaagagcgcgaac

SEQ ID NO: 155 - DNA, 89 bp, synthetic construct
  source 1..89: mol_type = other DNA
gcagtagagtaggtagagattaggcattgcagtagagtaggtagagattaggcacctgagaggaccataagcatcaaaaaggccagagg

SEQ ID NO: 156 - DNA, 85 bp, synthetic construct
  source 1..85: mol_type = other DNA
gcagtagagtaggtagagattaggcattgcagtagagtaggtagagattaggcaatcaagaaaacaaaaaaattctttaccgaca

SEQ ID NO: 157 - DNA, 85 bp, synthetic construct
  source 1..85: mol_type = other DNA
gcagtagagtaggtagagattaggcattgcagtagagtaggtagagattaggcattgagagagattcgcctgattgcggagaaac

SEQ ID NO: 158 - DNA, 85 bp, synthetic construct
  source 1..85: mol_type = other DNA
gcagtagagtaggtagagattaggcattgcagtagagtaggtagagattaggcaaataacgacaagaacgtggactccaacgtca

SEQ ID NO: 159 - DNA, 76 bp, synthetic construct
  source 1..76: mol_type = other DNA
gcagtagagtaggtagagattaggcattgcagtagagtaggtagagattaggcaatctttgacccccagtcagctt

SEQ ID NO: 160 - DNA, 78 bp, synthetic construct
  source 1..78: mol_type = other DNA
gcagtagagtaggtagagattaggcattgcagtagagtaggtagagattaggcattttggggtctgtaaatgtccaga

SEQ ID NO: 161 - DNA, 87 bp, synthetic construct
  source 1..87: mol_type = other DNA
gcagtagagtaggtagagattaggcattgcagtagagtaggtagagattaggcagaggctttctcattaagctgagactccggaggt

SEQ ID NO: 162 - DNA, 92 bp, synthetic construct
  source 1..92: mol_type = other DNA
gcagtagagtaggtagagattaggcattgcagtagagtaggtagagattaggcaattttcccgtgaaccacctaaagggagccccagcataa

SEQ ID NO: 163 - DNA, 96 bp, synthetic construct
  source 1..96: mol_type = other DNA
gcagtagagtaggtagagattaggcattgcagtagagtaggtagagattaggcagaagtttccattagcatcggaacgagtagtaccgccaccctc

SEQ ID NO: 164 - DNA, 47 bp, synthetic construct
  source 1..47: mol_type = other DNA
ttctacctacatcacttgcctgagtagaagatgcaacagttctggcc

SEQ ID NO: 165 - DNA, 63 bp, synthetic construct
  source 1..63: mol_type = other DNA
tgacttcgagccagccaacgctcaacagtagccaacatgtaatttagtattaccgccagccat

SEQ ID NO: 166 - DNA, 51 bp, synthetic construct
  source 1..51: mol_type = other DNA
tgaaatttaacgtgtagaaccgttgtagtatcggccttgctggtaatggat

SEQ ID NO: 167 - DNA, 47 bp, synthetic construct
  source 1..47: mol_type = other DNA
aggatcccagtaataaaagggacagaaaaacgctcatggaaattact

SEQ ID NO: 168 - DNA, 52 bp, synthetic construct
  source 1..52: mol_type = other DNA
tccagccatatttaacaacgggcttaatcgttatacttaattacattaatta

SEQ ID NO: 169 - DNA, 65 bp, synthetic construct
  source 1..65: mol_type = other DNA
gaatcggccactgagagcaatcagagaattaacccttctgaccagaacaagcagaggcattgatt

SEQ ID NO: 170 - DNA, 55 bp, synthetic construct
  source 1..55: mol_type = other DNA
ttcaatcctgaaagcacctaaatctcacaatgaagagtcagcttgacctatcagg

SEQ ID NO: 171 - DNA, 44 bp, synthetic construct
  source 1..44: mol_type = other DNA
caataaacaacatgtcataaggcgatcatatgtgagaatcgact

SEQ ID NO: 172 - DNA, 53 bp, synthetic construct
  source 1..53: mol_type = other DNA
tttactagcttttttgtgaataaccttgcttcgggccttgatattcacaaaca

SEQ ID NO: 173 - DNA, 33 bp, synthetic construct
  source 1..33: mol_type = other DNA
attgagcgctaaccctcagaaccgatacaccct

SEQ ID NO: 174 - DNA, 48 bp, synthetic construct
  source 1..48: mol_type = other DNA
ttggcagattcaccagtcaccggaagtgccgtcgagacgaaccaccag

SEQ ID NO: 175 - DNA, 51 bp, synthetic construct
  source 1..51: mol_type = other DNA
aaaggtggcaacatataaaagaaacctcaatcttcttcgcaatggattatt

SEQ ID NO: 176 - DNA, 56 bp, synthetic construct
  source 1..56: mol_type = other DNA
tccgccatatcagaagaaccgctattcgttttttcgctgaggcttggtcaccctca

SEQ ID NO: 177 - DNA, 37 bp, synthetic construct
  source 1..37: mol_type = other DNA
taatcgatgcggcgcatgtagaattagacgggagaat

SEQ ID NO: 178 - DNA, 54 bp, synthetic construct
  source 1..54: mol_type = other DNA
ggtgtgttcagcaaatcgttaacgcggccagagctgtcttggaagcgcaaccat

SEQ ID NO: 179 - DNA, 39 bp, synthetic construct
  source 1..39: mol_type = other DNA
ggagccttcagccctcgagaatagaaatcaagattcatt

SEQ ID NO: 180 - DNA, 48 bp, synthetic construct
  source 1..48: mol_type = other DNA
ttcatttttcacgttgaaagaattgcgcaacgccaacagccaacttaa

SEQ ID NO: 181 - DNA, 60 bp, synthetic construct
  source 1..60: mol_type = other DNA
gtcactgcacaccctgaacaaacagcggatcaatattattgcccaatatttagcgaaatt

SEQ ID NO: 182 - DNA, 49 bp, synthetic construct
  source 1..49: mol_type = other DNA
tccacagataattgtaaaaaaaggctccaaaaacaactttcaacataat

SEQ ID NO: 183 - DNA, 55 bp, synthetic construct
  source 1..55: mol_type = other DNA
tttaagttgctaatccggtattctaagaacggaggttttacaaaatatgtagcat

SEQ ID NO: 184 - DNA, 42 bp, synthetic construct
  source 1..42: mol_type = other DNA
tatcacctcccgacttgcggcgaggcgtgcaagcaggtgcca

SEQ ID NO: 185 - DNA, 57 bp, synthetic construct
  source 1..57: mol_type = other DNA
ttcagtttcagcggagtatagttattccagagtgtttaccagtcccaattttttaag

SEQ ID NO: 186 - DNA, 44 bp, synthetic construct
  source 1..44: mol_type = other DNA
gcgggatccagggagtgctttcgaacaaactaaataataagaat

SEQ ID NO: 187 - DNA, 58 bp, synthetic construct
  source 1..58: mol_type = other DNA
tagatcgcactatgtgagccagtcacgggtgccggaaaccaggctaaagtatcctttt

SEQ ID NO: 188 - DNA, 59 bp, synthetic construct
  source 1..59: mol_type = other DNA
tcaacgctaacgagcgtctgcgtaacgtgtgagaggagtaatcacagttaagcgtcata

SEQ ID NO: 189 - DNA, 54 bp, synthetic construct
  source 1..54: mol_type = other DNA
catggcttttgatgatcataagggaaccggattaaatgaattttgtcgtctttt

SEQ ID NO: 190 - DNA, 38 bp, synthetic construct
  source 1..38: mol_type = other DNA
tgttagattaaaatttttagaagtcagtgcgtactggt

SEQ ID NO: 191 - DNA, 51 bp, synthetic construct
  source 1..51: mol_type = other DNA
tttgaccattagatacaaggaaacatgctgatcggcgaaattatcctgaat

SEQ ID NO: 192 - DNA, 63 bp, synthetic construct
  source 1..63: mol_type = other DNA
gaaaggccggagaaccctcgcccaaaaataataaaaatagcacaggcttgagagtatcggcct

SEQ ID NO: 193 - DNA, 51 bp, synthetic construct
  source 1..51: mol_type = other DNA
taaaaaccaaacgaactaaacgacgacatggtttaaacattaattaattgc

SEQ ID NO: 194 - DNA, 52 bp, synthetic construct
  source 1..52: mol_type = other DNA
ttcgccattaacgccagaattaattttgataaaacagatttttgtgaggcgg

SEQ ID NO: 195 - DNA, 51 bp, synthetic construct
  source 1..51: mol_type = other DNA
tctgattatcaaaattatttttgttgttcaaaatcccttataagctgattg

SEQ ID NO: 196 - DNA, 51 bp, synthetic construct
  source 1..51: mol_type = other DNA
ccgcctggccctgagagtggttccctaccaccacacccgctgatagccctt

SEQ ID NO: 197 - DNA, 54 bp, synthetic construct
  source 1..54: mol_type = other DNA
ttcacgttggtgtattgggccttcctttttttagccagacccgtcgctgccagt

SEQ ID NO: 198 - DNA, 44 bp, synthetic construct
  source 1..44: mol_type = other DNA
gcggtcacgctgcgcgtaaccagcaaatccatataactatatgt

SEQ ID NO: 199 - DNA, 46 bp, synthetic construct
  source 1..46: mol_type = other DNA
ttgcagaaacgttaggctcattaagaggaagcccgagcccgagata

SEQ ID NO: 200 - DNA, 52 bp, synthetic construct
  source 1..52: mol_type = other DNA
caaagcgccattttttcgccattcaggctgcgctcgcgtctaccgtaatggt

SEQ ID NO: 201 - DNA, 60 bp, synthetic construct
  source 1..60: mol_type = other DNA
gttcagaattttttacgagaattctggagctaaattgtatacataagaataccacattct

SEQ ID NO: 202 - DNA, 47 bp, synthetic construct
  source 1..47: mol_type = other DNA
aggggacgagattctccgttttttggaacaaacgaagttgggtaacg

SEQ ID NO: 203 - DNA, 27 bp, synthetic construct
  source 1..27: mol_type = other DNA
tagtcttccgccgctttttcttaatgc

SEQ ID NO: 204 - DNA, 38 bp, synthetic construct
  source 1..38: mol_type = other DNA
agtagggaatatttttttttgaatggctatcgctcaac

SEQ ID NO: 205 - DNA, 39 bp, synthetic construct
  source 1..39: mol_type = other DNA
cagaggcattttcgacattctggaacattcgtaatcatg

SEQ ID NO: 206 - DNA, 47 bp, synthetic construct
  source 1..47: mol_type = other DNA
gccagtaagctgtttcaaaccaagaatcggaatcacccaaatcaagt

SEQ ID NO: 207 - DNA, 31 bp, synthetic construct
  source 1..31: mol_type = other DNA
ggccaccgagtaaaagaataatgttgaggat

SEQ ID NO: 208 - DNA, 51 bp, synthetic construct
  source 1..51: mol_type = other DNA
atcgtaaccgtttgccttcctgttttttgccagctacccgtcgcgacagta

SEQ ID NO: 209 - DNA, 42 bp, synthetic construct
  source 1..42: mol_type = other DNA
cattttcagggataaaacagctttttttataccgatagtacg

SEQ ID NO: 210 - DNA, 31 bp, synthetic construct
  source 1..31: mol_type = other DNA
gattttcaggtttaagttgaaagctgattgc

SEQ ID NO: 211 - DNA, 38 bp, synthetic construct
  source 1..38: mol_type = other DNA
agccctaagttatacagatgatgaaacaatgtgttgtt

SEQ ID NO: 212 - DNA, 39 bp, synthetic construct
  source 1..39: mol_type = other DNA
cagtaacaacgccagcccagcagagcgaacgccatcaaa

SEQ ID NO: 213 - DNA, 57 bp, synthetic construct
  source 1..57: mol_type = other DNA
aacaacatgtaagagcaacactatcgttctaggattttttaacggtaatcgtaaaac

SEQ ID NO: 214 - DNA, 50 bp, synthetic construct
  source 1..50: mol_type = other DNA
ctgagaagtgaaaattatttgctttttcgtaaaacagaaacgccagaatc

SEQ ID NO: 215 - DNA, 32 bp, synthetic construct
  source 1..32: mol_type = other DNA
acaataaatataaagtaccgacaattcaccag

SEQ ID NO: 216 - DNA, 53 bp, synthetic construct
  source 1..53: mol_type = other DNA
gacgacgaacaagtgccgtcgattgaggcaggtcgaggtgccgtaagccgcca

SEQ ID NO: 217 - DNA, 32 bp, synthetic construct
  source 1..32: mol_type = other DNA
aggcgattgcggagcatcactaacggtttaag

SEQ ID NO: 218 - DNA, 34 bp, synthetic construct
  source 1..34: mol_type = other DNA
ccagggttttcccagtcacgacgttgtagtaaca

SEQ ID NO: 219 - DNA, 32 bp, synthetic construct
  source 1..32: mol_type = other DNA
acaatgacagaaccgccaccctcaagaaaagt

SEQ ID NO: 220 - DNA, 32 bp, synthetic construct
  source 1..32: mol_type = other DNA
tttatcagcttgctttcaacctaaaacgaaag

SEQ ID NO: 221 - DNA, 38 bp, synthetic construct
  source 1..38: mol_type = other DNA
ggccagtgccaagcttataacggaacgtgccccagagc

SEQ ID NO: 222 - DNA, 31 bp, synthetic construct
  source 1..31: mol_type = other DNA
agcgtggtgctggtccgttttttcgtctcgt

SEQ ID NO: 223 - DNA, 24 bp, synthetic construct
  source 1..24: mol_type = other DNA
tagtaaatataccaagcgcagacg

SEQ ID NO: 224 - DNA, 53 bp, synthetic construct
  source 1..53: mol_type = other DNA
caagaatggcattagatttaccagtcccgcaattttgtgcgccaaagacaaaa

SEQ ID NO: 225 - DNA, 35 bp, synthetic construct
  source 1..35: mol_type = other DNA
gggcgcggttgcggtatgagccgggtctcaagatt

SEQ ID NO: 226 - DNA, 38 bp, synthetic construct
  source 1..38: mol_type = other DNA
taaacatcaacaaagaaaccaccattatcattttgcgg

SEQ ID NO: 227 - DNA, 49 bp, synthetic construct
  source 1..49: mol_type = other DNA
tattacaggtagaaaggtgtagctttaatataggcagaatgttttaatt

SEQ ID NO: 228 - DNA, 53 bp, synthetic construct
  source 1..53: mol_type = other DNA
caatagatgccgattaatcagtgaaatacgtggcacagaccttaattggcaag

SEQ ID NO: 229 - DNA, 40 bp, synthetic construct
  source 1..40: mol_type = other DNA
ttagaagtcgcggggataactcacattaattgtgcctaat

SEQ ID NO: 230 - DNA, 44 bp, synthetic construct
  source 1..44: mol_type = other DNA
agaaggagcggaattatcatcatgacgctccgtgagcccgcgcc

SEQ ID NO: 231 - DNA, 39 bp, synthetic construct
  source 1..39: mol_type = other DNA
gatgccggcctgcggctggtaatgggtaaaggtttcttt

SEQ ID NO: 232 - DNA, 57 bp, synthetic construct
  source 1..57: mol_type = other DNA
agccagcggggggtcattgcaggcgctttcgcatacaattttatcctgatgaaatag

SEQ ID NO: 233 - DNA, 35 bp, synthetic construct
  source 1..35: mol_type = other DNA
agttgctccgaagccctttttagagccaccaccct

SEQ ID NO: 234 - DNA, 30 bp, synthetic construct
  source 1..30: mol_type = other DNA
acattttattcctgagaacgttattaattt

SEQ ID NO: 235 - DNA, 40 bp, synthetic construct
  source 1..40: mol_type = other DNA
agttaaaccagcaccgtcggtggtgccatcccttacaaaa

SEQ ID NO: 236 - DNA, 40 bp, synthetic construct
  source 1..40: mol_type = other DNA
acatcctcccaattctagtacctttaattgctcaggtcag

SEQ ID NO: 237 - DNA, 25 bp, synthetic construct
  source 1..25: mol_type = other DNA
tcaggctgcgtttttaactgttggg

SEQ ID NO: 238 - DNA, 46 bp, synthetic construct
  source 1..46: mol_type = other DNA
gaaggttagaacggtataaagaaactaatagattttttagagccgt

SEQ ID NO: 239 - DNA, 32 bp, synthetic construct
  source 1..32: mol_type = other DNA
gtttagtatcatatgcgtaaatcggtgagtga

SEQ ID NO: 240 - DNA, 29 bp, synthetic construct
  source 1..29: mol_type = other DNA
cgggggtttctgccaaaggcttcataatc

SEQ ID NO: 241 - DNA, 32 bp, synthetic construct
  source 1..32: mol_type = other DNA
tgcgcgccaggaaacgcaaacttaaatttctg

SEQ ID NO: 242 - DNA, 34 bp, synthetic construct
  source 1..34: mol_type = other DNA
aaagccgtttgcctattgagggagggaaggtaaa

SEQ ID NO: 243 - DNA, 32 bp, synthetic construct
  source 1..32: mol_type = other DNA
ccgtaaaagccgccagtttcggtctacataaa

SEQ ID NO: 244 - DNA, 32 bp, synthetic construct
  source 1..32: mol_type = other DNA
gaacggatgcctccggggacttgtagaacgtc

SEQ ID NO: 245 - DNA, 29 bp, synthetic construct
  source 1..29: mol_type = other DNA
gccacggctgaaaagcgaacgagtagatt

SEQ ID NO: 246 - DNA, 48 bp, synthetic construct
  source 1..48: mol_type = other DNA
cgcttctggtgccgaggtggagccgaataagggattagaggtaccaaa

SEQ ID NO: 247 - DNA, 51 bp, synthetic construct
  source 1..51: mol_type = other DNA
ttcatcaaaacgagtagtaaatttttttggcttgagatggttttatgcgat

SEQ ID NO: 248 - DNA, 35 bp, synthetic construct
  source 1..35: mol_type = other DNA
acaaaatctctggtcaaataccgatatgaaaaatc

SEQ ID NO: 249 - DNA, 39 bp, synthetic construct
  source 1..39: mol_type = other DNA
tagacagtctaaaatatcttcgcgaattgaggaactgat

SEQ ID NO: 250 - DNA, 54 bp, synthetic construct
  source 1..54: mol_type = other DNA
gccgctgggcgctgagaatcgcaataagaataaacaccgaaaacatagcgatag

SEQ ID NO: 251 - DNA, 46 bp, synthetic construct
  source 1..46: mol_type = other DNA
gcgtactaggagctaaggtttgccgaggcggtttgcgtattgggcg

SEQ ID NO: 252 - DNA, 40 bp, synthetic construct
  source 1..40: mol_type = other DNA
tggttgctgagtgagccatacgagccggaagcccgattta

SEQ ID NO: 253 - DNA, 55 bp, synthetic construct
  source 1..55: mol_type = other DNA
cggcctttagtgatgttcaaccgttagcgtcagactgtagcgcgttttcgacata

SEQ ID NO: 254 - DNA, 45 bp, synthetic construct
  source 1..45: mol_type = other DNA
aagagcaagaaacaaatcttaccaacgctacatcagctgccggtg

SEQ ID NO: 255 - DNA, 32 bp, synthetic construct
  source 1..32: mol_type = other DNA
agcaaatcagatataggcacgcgtcaatgaaa

SEQ ID NO: 256 - DNA, 37 bp, synthetic construct
  source 1..37: mol_type = other DNA
atccggtacgcagtatgaaggatgaataggaacatga

SEQ ID NO: 257 - DNA, 33 bp, synthetic construct
  source 1..33: mol_type = other DNA
ttctaagaacgcgagggttttcacagtagcgac

SEQ ID NO: 258 - DNA, 35 bp, synthetic construct
  source 1..35: mol_type = other DNA
gaaaccgtgtgcactctgtttttggtgctgcggcc

SEQ ID NO: 259 - DNA, 27 bp, synthetic construct
  source 1..27: mol_type = other DNA
caaatcaatagtagcataaagcctcag

SEQ ID NO: 260 - DNA, 56 bp, synthetic construct
  source 1..56: mol_type = other DNA
cttgccctgtaatacttttgcgggggttgatatcgtcataaaacagttcagaaaac

SEQ ID NO: 261 - DNA, 38 bp, synthetic construct
  source 1..38: mol_type = other DNA
aatttcaaatgggcgcgggataggtcacgtttggcgaa

SEQ ID NO: 262 - DNA, 38 bp, synthetic construct
  source 1..38: mol_type = other DNA
aggcgaatgctcatttagttgagatacataacctgcaa

SEQ ID NO: 263 - DNA, 36 bp, synthetic construct
  source 1..36: mol_type = other DNA
cctgagcagaaatcggccagtttggaagaaaggaag

SEQ ID NO: 264 - DNA, 32 bp, synthetic construct
  source 1..32: mol_type = other DNA
ctgtgtgagtacctcagagccaccaccctcag

SEQ ID NO: 265 - DNA, 47 bp, synthetic construct
  source 1..47: mol_type = other DNA
ggcctcttcgctattcatgttttgctgaatataatgctgatcaaaag

SEQ ID NO: 266 - DNA, 56 bp, synthetic construct
  source 1..56: mol_type = other DNA
taggattatgagactcaaaatcaccggaaccaaacgtcacgcctgttctgagtaac

SEQ ID NO: 267 - DNA, 55 bp, synthetic construct
  source 1..55: mol_type = other DNA
ccccctgcatcagacgatccaggcagcttacggctggaggtgtccagacgagcgt

SEQ ID NO: 268 - DNA, 45 bp, synthetic construct
  source 1..45: mol_type = other DNA
gggaagcccaacgggatgctgattgccgtgtttaccacacaatca

SEQ ID NO: 269 - DNA, 47 bp, synthetic construct
  source 1..47: mol_type = other DNA
caatagctattttttttaattttgctttttcccagcctcaatccgcc

SEQ ID NO: 270 - DNA, 37 bp, synthetic construct
  source 1..37: mol_type = other DNA
agaagccatcggttggtggcatcaattctggcgcgag

SEQ ID NO: 271 - DNA, 48 bp, synthetic construct
  source 1..48: mol_type = other DNA
aactgaaaaatcttttaccagacgacgaaatcaccatcaatatgatat

SEQ ID NO: 272 - DNA, 30 bp, synthetic construct
  source 1..30: mol_type = other DNA
tgtagcggtcacgctgcgcgaaaattctta

SEQ ID NO: 273 - DNA, 49 bp, synthetic construct
  source 1..49: mol_type = other DNA
tttcatttgaattaccttaggcgttacatatttaacaacgcacctgaaa

SEQ ID NO: 274 - DNA, 37 bp, synthetic construct
  source 1..37: mol_type = other DNA
atggtttgacagagcgggcgctaacagggcgcgtaag

SEQ ID NO: 275 - DNA, 32 bp, synthetic construct
  source 1..32: mol_type = other DNA
aaataccggagcttgactatcagggcgatggc

SEQ ID NO: 276 - DNA, 24 bp, synthetic construct
  source 1..24: mol_type = other DNA
ttatcatttagttaatgtcaatag

SEQ ID NO: 277 - DNA, 32 bp, synthetic construct
  source 1..32: mol_type = other DNA
cgtactcaggaggtttttaagactccttatta

SEQ ID NO: 278 - DNA, 38 bp, synthetic construct
  source 1..38: mol_type = other DNA
tcgctgaacgcagaaacagcggatcaataaaccgtaac

SEQ ID NO: 279 - DNA, 31 bp, synthetic construct
  source 1..31: mol_type = other DNA
tacagactggtgaaagaaacgcaaagggcaa

SEQ ID NO: 280 - DNA, 32 bp, synthetic construct
  source 1..32: mol_type = other DNA
caggcgcatgcccgtagttccagtaagcgtca

SEQ ID NO: 281 - DNA, 39 bp, synthetic construct
  source 1..39: mol_type = other DNA
ctgattgtttggattatacttctgagtctgttcagagcg

SEQ ID NO: 282 - DNA, 29 bp, synthetic construct
  source 1..29: mol_type = other DNA
gcaaatatttaaaagagaatcctgataaa

SEQ ID NO: 283 - DNA, 38 bp, synthetic construct
  source 1..38: mol_type = other DNA
gttaatacattgtgcttcaaatatcgcagcccgagata

SEQ ID NO: 284 - DNA, 32 bp, synthetic construct
  source 1..32: mol_type = other DNA
ttttgttagattaagaaccctgactattatag

SEQ ID NO: 285 - DNA, 26 bp, synthetic construct
  source 1..26: mol_type = other DNA
tcggccaacgattagactcgttagaa

SEQ ID NO: 286 - DNA, 46 bp, synthetic construct
  source 1..46: mol_type = other DNA
ggaattaattagcaaggccggagagccacccaatagcattgctcag

SEQ ID NO: 287 - DNA, 39 bp, synthetic construct
  source 1..39: mol_type = other DNA
aacgcaaggataaaaatttttcaataagcaattaacatc

SEQ ID NO: 288 - DNA, 30 bp, synthetic construct
  source 1..30: mol_type = other DNA
aattatcccatcgatagcagcacatctttt

SEQ ID NO: 289 - DNA, 36 bp, synthetic construct
  source 1..36: mol_type = other DNA
tattgacggaaattattcattaaaggtgccttacac

SEQ ID NO: 290 - DNA, 56 bp, synthetic construct
  source 1..56: mol_type = other DNA
gggcgacaaagggtaaaaaaaatctaaacagcctaatatcgcagccttttcagcgg

SEQ ID NO: 291 - DNA, 40 bp, synthetic construct
  source 1..40: mol_type = other DNA
catataaagggatagccggttgtgattgagcgcataaaca

SEQ ID NO: 292 - DNA, 40 bp, synthetic construct
  source 1..40: mol_type = other DNA
ccattagactatattttcatttggactaatagcgtaacaa

SEQ ID NO: 293 - DNA, 32 bp, synthetic construct
  source 1..32: mol_type = other DNA
tagtttgacgctggcaaacctcaccggaaaca

SEQ ID NO: 294 - DNA, 40 bp, synthetic construct
  source 1..40: mol_type = other DNA
ttcattccatataacagttgattctcaggaaaccaggcaa

SEQ ID NO: 295 - DNA, 42 bp, synthetic construct
  source 1..42: mol_type = other DNA
cagggtggtttttctttatttaggagcactaacaattgcgta

SEQ ID NO: 296 - DNA, 28 bp, synthetic construct
  source 1..28: mol_type = other DNA
aagcggtccacgctacaggagaatacat

SEQ ID NO: 297 - DNA, 57 bp, synthetic construct
  source 1..57: mol_type = other DNA
ccgctttccagtcgggcgccacccgtcttttcatcgtaggtcacacgattaatacct

SEQ ID NO: 298 - DNA, 33 bp, synthetic construct
  source 1..33: mol_type = other DNA
agaatcaagcacagcgcagtgtcacctttccag

SEQ ID NO: 299 - DNA, 56 bp, synthetic construct
  source 1..56: mol_type = other DNA
atagaaaacatacaggataaagaaccggatattcattaccatcggcgactgtttag

SEQ ID NO: 300 - DNA, 49 bp, synthetic construct
  source 1..49: mol_type = other DNA
gcggatggcgaatttttcagacgtattttttagtaaaaagaaattacct

SEQ ID NO: 301 - DNA, 54 bp, synthetic construct
  source 1..54: mol_type = other DNA
cttaattaaatatgcaactaaagtacggtgaagggcgagcgtctggaccgtaat

SEQ ID NO: 302 - DNA, 38 bp, synthetic construct
  source 1..38: mol_type = other DNA
cttcaccgagacgggcaacagctagctcaagtaccttt

SEQ ID NO: 303 - DNA, 48 bp, synthetic construct
  source 1..48: mol_type = other DNA
gcctggccctgagagagtggttccaaagtaaccaccacactaatgcgc

SEQ ID NO: 304 - DNA, 44 bp, synthetic construct
  source 1..44: mol_type = other DNA
tcagaaccgccacaagcctggggcgttgcgctttcctttacaaa

SEQ ID NO: 305 - DNA, 56 bp, synthetic construct
  source 1..56: mol_type = other DNA
ccgcctccctcagagccagtagcaagttgaggctttgcccttatcagatgatggca

SEQ ID NO: 306 - DNA, 38 bp, synthetic construct
  source 1..38: mol_type = other DNA
accggaaaccaccagagccgccgccagggcgcggggtt

SEQ ID NO: 307 - DNA, 44 bp, synthetic construct
  source 1..44: mol_type = other DNA
attagcgtttgcccgtaatcggtcatactggtgtgtgctcgtca

SEQ ID NO: 308 - DNA, 36 bp, synthetic construct
  source 1..36: mol_type = other DNA
gaataagttttcatcggcatcagttgggtctcacgg

SEQ ID NO: 309 - DNA, 48 bp, synthetic construct
  source 1..48: mol_type = other DNA
caataaattcaataacaacgtacaacgcggtctggtcagcagcaaccg

SEQ ID NO: 310 - DNA, 51 bp, synthetic construct
  source 1..51: mol_type = other DNA
cgagcttcaaagcttagagaataattctcggtgcgagggggatgtgctgca

SEQ ID NO: 311 - DNA, 47 bp, synthetic construct
  source 1..47: mol_type = other DNA
caaaatcccttataatgattgccaccaagtttacatcggtgaatata

SEQ ID NO: 312 - DNA, 55 bp, synthetic construct
  source 1..55: mol_type = other DNA
gaagaaagcgaaagcaaccagcaggcgaaaatcctgtttttttgatggttgcagc

SEQ ID NO: 313 - DNA, 28 bp, synthetic construct
  source 1..28: mol_type = other DNA
ataaagtaattgttcacgtatccaacag

SEQ ID NO: 314 - DNA, 47 bp, synthetic construct
  source 1..47: mol_type = other DNA
ctcaagagttagcaatattctgatgtatcacgaaagacaaggctttg

SEQ ID NO: 315 - DNA, 40 bp, synthetic construct
  source 1..40: mol_type = other DNA
ggtggcaaccctgcctggacagataccgaactctcatctt

SEQ ID NO: 316 - DNA, 49 bp, synthetic construct
  source 1..49: mol_type = other DNA
tcaaccataatttttcctcgacgttaatttttaaaacgatgccagtttg

SEQ ID NO: 317 - DNA, 38 bp, synthetic construct
  source 1..38: mol_type = other DNA
agaattagtaacagtaggctgggaacgaggcgcgaaac

SEQ ID NO: 318 - DNA, 40 bp, synthetic construct
  source 1..40: mol_type = other DNA
agcaaaatgagtaatcttgacgaccctcatagccagacgt

SEQ ID NO: 319 - DNA, 37 bp, synthetic construct
  source 1..37: mol_type = other DNA
agcatggtaataaattttaaaaatccgcgatcgcctg

SEQ ID NO: 320 - DNA, 31 bp, synthetic construct
  source 1..31: mol_type = other DNA
aaagctaatttatttcagctgctcattcagt

SEQ ID NO: 321 - DNA, 40 bp, synthetic construct
  source 1..40: mol_type = other DNA
aacattatctgcggaaatcagaaacaatcatagtgagaaa

SEQ ID NO: 322 - DNA, 42 bp, synthetic construct
  source 1..42: mol_type = other DNA
gccggcgagtggcgacaaaaaattaacaatatattcgctatt

SEQ ID NO: 323 - DNA, 30 bp, synthetic construct
  source 1..30: mol_type = other DNA
aagcactataccgcacgaacgcgtgagaga

SEQ ID NO: 324 - DNA, 30 bp, synthetic construct
  source 1..30: mol_type = other DNA
caggagggagggttgaatgctgagttgggt

SEQ ID NO: 325 - DNA, 56 bp, synthetic construct
  source 1..56: mol_type = other DNA
ctggatagcgtccaatagacttgccagaggggcggaagcaaactccaaccttttga

SEQ ID NO: 326 - DNA, 32 bp, synthetic construct
  source 1..32: mol_type = other DNA
atcaaaaaaaattcgccaggtcatttttgaga

SEQ ID NO: 327 - DNA, 47 bp, synthetic construct
  source 1..47: mol_type = other DNA
gagtccactattaagcggattgcgggttgagttaaatcatattcatt

SEQ ID NO: 328 - DNA, 48 bp, synthetic construct
  source 1..48: mol_type = other DNA
gagtagaaaccgttgtatataatccaattcgacaactcgtcgtgccag

SEQ ID NO: 329 - DNA, 44 bp, synthetic construct
  source 1..44: mol_type = other DNA
ccttggaattttttataattactagaaaaattttcccttagaat

SEQ ID NO: 330 - DNA, 31 bp, synthetic construct
  source 1..31: mol_type = other DNA
tttgcgggatcgtttttcatgaggaagtttc

SEQ ID NO: 331 - DNA, 28 bp, synthetic construct
  source 1..28: mol_type = other DNA
taaagcatcaatcgtcaattatttgaat

SEQ ID NO: 332 - DNA, 40 bp, synthetic construct
  source 1..40: mol_type = other DNA
agtgagaataatttttaaaggagcctaaaacagaccaact

SEQ ID NO: 333 - DNA, 54 bp, synthetic construct
  source 1..54: mol_type = other DNA
aagggcgaaatttttttaatggaaactttttgtacataaatttacatttaacaa

SEQ ID NO: 334 - DNA, 31 bp, synthetic construct
  source 1..31: mol_type = other DNA
ctcattaaagccatttttaatggaaagcgca

SEQ ID NO: 335 - DNA, 50 bp, synthetic construct
  source 1..50: mol_type = other DNA
taatgccactacgaaggcaccgaggtgaatttcttgcaagcccaatagga

SEQ ID NO: 336 - DNA, 46 bp, synthetic construct
  source 1..46: mol_type = other DNA
ccataaatttttttaaaaatcaagcaaacattgtaaacgaggcata

SEQ ID NO: 337 - DNA, 34 bp, synthetic construct
  source 1..34: mol_type = other DNA
tcggccttgggaaggctcattaaagattgtataa

SEQ ID NO: 338 - DNA, 35 bp, synthetic construct
  source 1..35: mol_type = other DNA
agttaagcccaataatacccatgttaacggaatac

SEQ ID NO: 339 - DNA, 41 bp, synthetic construct
  source 1..41: mol_type = other DNA
aagcagatgttttgaagccttaaaactgttgcgttacctgc

SEQ ID NO: 340 - DNA, 53 bp, synthetic construct
  source 1..53: mol_type = other DNA
attcatcaagcaatacgtaccgagctcgagtgctcactgcctgcattaatgaa

SEQ ID NO: 341 - DNA, 63 bp, synthetic construct
  source 1..63: mol_type = other DNA
aaaaagagttgaaagaatttcggaacttttttatacgtagatttttaatacaatagccccctt

SEQ ID NO: 342 - DNA, 56 bp, synthetic construct
  source 1..56: mol_type = other DNA
ttgacgagatccgctcaatttaggttttttccaccgtgtgtgagaagattcatctt

SEQ ID NO: 343 - DNA, 56 bp, synthetic construct
  source 1..56: mol_type = other DNA
aatcattactcctcacccattaccgagccagcaaaatcacaaacctgtattaaatc

SEQ ID NO: 344 - DNA, 39 bp, synthetic construct
  source 1..39: mol_type = other DNA
gaagggttagaacctaccatatctttttataaagggatt

SEQ ID NO: 345 - DNA, 31 bp, synthetic construct
  source 1..31: mol_type = other DNA
tgcctgccccttctgcaacatgtacaattcc

SEQ ID NO: 346 - DNA, 52 bp, synthetic construct
  source 1..52: mol_type = other DNA
tctatatcccatcctaattttgaacaagtcagctaacataggtcagaaaact

SEQ ID NO: 347 - DNA, 44 bp, synthetic construct
  source 1..44: mol_type = other DNA
caagccgtttttaatataagagaacaacatgtaaaaataaaagt

SEQ ID NO: 348 - DNA, 47 bp, synthetic construct
  source 1..47: mol_type = other DNA
atccccggttctttgaccagtaatattgcaacaggaaaaacgctgat

SEQ ID NO: 349 - DNA, 47 bp, synthetic construct
  source 1..47: mol_type = other DNA
taactcatggaagtaataacatcacttgcctcgccagccaaaaggga

SEQ ID NO: 350 - DNA, 63 bp, synthetic construct
  source 1..63: mol_type = other DNA
tgagcggctgtcatcaacaatagataagtccacgagcatgtagaaactccagaacaatattac

SEQ ID NO: 351 - DNA, 39 bp, synthetic construct
  source 1..39: mol_type = other DNA
ccatcacgagatagaatggtaatacaatcaataatgctt

SEQ ID NO: 352 - DNA, 30 bp, synthetic construct
  source 1..30: mol_type = other DNA
caaattagaactcaaactatcggcctagct

SEQ ID NO: 353 - DNA, 54 bp, synthetic construct
  source 1..54: mol_type = other DNA
taccgcggcatgaagtaccgatcgcctttttacgcataaccgataaaggccgct

SEQ ID NO: 354 - DNA, 31 bp, synthetic construct
  source 1..31: mol_type = other DNA
ccaaaagaactcaccctcaacaaccccacct

SEQ ID NO: 355 - DNA, 51 bp, synthetic construct
  source 1..51: mol_type = other DNA
tcagcaaatcgttaacggcatcaagaatgcggcgggagagccgaagcgaat

SEQ ID NO: 356 - DNA, 35 bp, synthetic construct
  source 1..35: mol_type = other DNA
tgacttgcgggcccgttttacaaagttaccagaag

SEQ ID NO: 357 - DNA, 49 bp, synthetic construct
  source 1..49: mol_type = other DNA
tttatttacattggcagaaaggtaataccaggcggataagccagaacct

SEQ ID NO: 358 - DNA, 51 bp, synthetic construct
  source 1..51: mol_type = other DNA
accgtcaccgacttgagccatttgtaaaagttttcgcgtcaatcgtctgat

SEQ ID NO: 359 - DNA, 24 bp, synthetic construct
  source 1..24: mol_type = other DNA
ttctgtccacggcttagtgcaaat

SEQ ID NO: 360 - DNA, 28 bp, synthetic construct
  source 1..28: mol_type = other DNA
tgtatcggactgagttaacaactaagat

SEQ ID NO: 361 - DNA, 42 bp, synthetic construct
  source 1..42: mol_type = other DNA
tccttgtttaacgtcaaaaaataagaaattgccagacgcaac

SEQ ID NO: 362 - DNA, 28 bp, synthetic construct
  source 1..28: mol_type = other DNA
aagaattgagcctaatcgatttttccct

SEQ ID NO: 363 - DNA, 57 bp, synthetic construct
  source 1..57: mol_type = other DNA
tagttgctaaacaacttcattccagagggtatagaatttgtgagagcaaacaccacg

SEQ ID NO: 364 - DNA, 55 bp, synthetic construct
  source 1..55: mol_type = other DNA
taattaacataaattatttatcccaatccaatgaaaataagagagatcagtacaa

SEQ ID NO: 365 - DNA, 49 bp, synthetic construct
  source 1..49: mol_type = other DNA
actacaacaggctccatcacgttgaaaatctcctgtatgggatttaatt

SEQ ID NO: 366 - DNA, 48 bp, synthetic construct
  source 1..48: mol_type = other DNA
tctcaggaattgcgaataatagaaaggtcgtcacaacccacctcattt

SEQ ID NO: 367 - DNA, 39 bp, synthetic construct
  source 1..39: mol_type = other DNA
caaaaaaagcctgtagtcaacagttacagagagaaggat

SEQ ID NO: 368 - DNA, 49 bp, synthetic construct
  source 1..49: mol_type = other DNA
taaaaaccaaacaggtaccagtaagttcctgacgagaatcgcactccat

SEQ ID NO: 369 - DNA, 46 bp, synthetic construct
  source 1..46: mol_type = other DNA
tttttgcatagaaccctcatatgttttaacgatacaggagtgtact

SEQ ID NO: 370 - DNA, 48 bp, synthetic construct
  source 1..48: mol_type = other DNA
ttttgcaaaagcaggacgtcaggaagaacaccagcattaaataagagg

SEQ ID NO: 371 - DNA, 51 bp, synthetic construct
  source 1..51: mol_type = other DNA
tacatttcgcaaatggttcatatgtccggcaagcgccatgcgggagaattt

SEQ ID NO: 372 - DNA, 35 bp, synthetic construct
  source 1..35: mol_type = other DNA
tacaccctgaacaaagtcacagacagctttctccg

SEQ ID NO: 373 - DNA, 57 bp, synthetic construct
  source 1..57: mol_type = other DNA
ttttccggcactgtgagcgaaaacgacagcgccattcgccatctggaagttcatttt

SEQ ID NO: 374 - DNA, 30 bp, synthetic construct
  source 1..30: mol_type = other DNA
ctgacctttcgtctttttagcgtaacgatt

SEQ ID NO: 375 - DNA, 39 bp, synthetic construct
  source 1..39: mol_type = other DNA
cagtgagatttaggaagccagcagcaaattaacactaat

SEQ ID NO: 376 - DNA, 54 bp, synthetic construct
  source 1..54: mol_type = other DNA
tccaaacctcaaatatcatcaacacgtcagagagaaacaataacggtcaccagt

SEQ ID NO: 377 - DNA, 31 bp, synthetic construct
  source 1..31: mol_type = other DNA
tacgcgcgccaaaaggtagctattgcctgag

SEQ ID NO: 378 - DNA, 41 bp, synthetic construct
  source 1..41: mol_type = other DNA
tgaggtgaggcggtcagacgaaccagattcatctttaacca

SEQ ID NO: 379 - DNA, 49 bp, synthetic construct
  source 1..49: mol_type = other DNA
ccattaaagttggcaaaaaccctcaatcaataagataaaacagagttat

SEQ ID NO: 380 - DNA, 29 bp, synthetic construct
  source 1..29: mol_type = other DNA
ttagagataccacaaacccttgctgaatt

SEQ ID NO: 381 - DNA, 32 bp, synthetic construct
  source 1..32: mol_type = other DNA
ttttttgccccttctgcaacatgtacaattcc

SEQ ID NO: 382 - DNA, 53 bp, synthetic construct
  source 1..53: mol_type = other DNA
ttttttatcccatcctaattttgaacaagtcagctaacataggtcagaaaact

SEQ ID NO: 383 - DNA, 45 bp, synthetic construct
  source 1..45: mol_type = other DNA
caagccgtttttaatataagagaacaacatgtaaaaataattttt

SEQ ID NO: 384 - DNA, 48 bp, synthetic construct
  source 1..48: mol_type = other DNA
atccccggttctttgaccagtaatattgcaacaggaaaaacgcttttt

SEQ ID NO: 385 - DNA, 48 bp, synthetic construct
  source 1..48: mol_type = other DNA
ttttttcatggaagtaataacatcacttgcctcgccagccaaaaggga

SEQ ID NO: 386 - DNA, 64 bp, synthetic construct
  source 1..64: mol_type = other DNA
tttttcggctgtcatcaacaatagataagtccacgagcatgtagaaactccagaacaatattac

SEQ ID NO: 387 - DNA, 40 bp, synthetic construct
  source 1..40: mol_type = other DNA
ccatcacgagatagaatggtaatacaatcaataatttttt

SEQ ID NO: 388 - DNA, 31 bp, synthetic construct
  source 1..31: mol_type = other DNA
caaattagaactcaaactatcggcctttttt

SEQ ID NO: 389 - DNA, 29 bp, synthetic construct
  source 1..29: mol_type = other DNA
tgtatcggactgagttaacaactattttt

SEQ ID NO: 390 - DNA, 43 bp, synthetic construct
  source 1..43: mol_type = other DNA
ttttttgtttaacgtcaaaaaataagaaattgccagacgcaac

SEQ ID NO: 391 - DNA, 29 bp, synthetic construct
  source 1..29: mol_type = other DNA
aagaattgagcctaatcgatttttttttt

SEQ ID NO: 392 - DNA, 58 bp, synthetic construct
  source 1..58: mol_type = other DNA
ttttttgctaaacaacttcattccagagggtatagaatttgtgagagcaaacaccacg

SEQ ID NO: 393 - DNA, 56 bp, synthetic construct
  source 1..56: mol_type = other DNA
ttttttaacataaattatttatcccaatccaatgaaaataagagagatcagtacaa

SEQ ID NO: 394 - DNA, 50 bp, synthetic construct
  source 1..50: mol_type = other DNA
actacaacaggctccatcacgttgaaaatctcctgtatgggatttttttt

SEQ ID NO: 395 - DNA, 49 bp, synthetic construct
  source 1..49: mol_type = other DNA
tttttaggaattgcgaataatagaaaggtcgtcacaacccacctcattt

SEQ ID NO: 396 - DNA, 40 bp, synthetic construct
  source 1..40: mol_type = other DNA
caaaaaaagcctgtagtcaacagttacagagagaattttt

SEQ ID NO: 397 - DNA, 29 bp, synthetic construct
  source 1..29: mol_type = other DNA
tgtatcggactgagttaacaactattttt

SEQ ID NO: 398 - DNA, 43 bp, synthetic construct; identical to SEQ ID NO: 390
  source 1..43: mol_type = other DNA

SEQ ID NO: 399 - DNA, 29 bp, synthetic construct
  source 1..29: mol_type = other DNA
aagaattgagcctaatcgatttttttttt

SEQ ID NO: 400 - DNA, 58 bp, synthetic construct; identical to SEQ ID NO: 392
  source 1..58: mol_type = other DNA

SEQ ID NO: 401 - DNA, 56 bp, synthetic construct; identical to SEQ ID NO: 393
  source 1..56: mol_type = other DNA

SEQ ID NO: 402 - DNA, 50 bp, synthetic construct; identical to SEQ ID NO: 394
  source 1..50: mol_type = other DNA

SEQ ID NO: 403 - DNA, 49 bp, synthetic construct; identical to SEQ ID NO: 395
  source 1..49: mol_type = other DNA

SEQ ID NO: 404 - DNA, 40 bp, synthetic construct; identical to SEQ ID NO: 396
  source 1..40: mol_type = other DNA

SEQ ID NO: 405 - DNA, 31 bp, synthetic construct
  source 1..31: mol_type = other DNA
ccaatcgcgtcagacgattggccttgatatt

SEQ ID NO: 406 - DNA, 41 bp, synthetic construct
  source 1..41: mol_type = other DNA
tcagaagcaaggctatattaaattaatgcccacgctgaagt

SEQ ID NO: 407 - DNA, 35 bp, synthetic construct
  source 1..35: mol_type = other DNA
gagaatgatagcatgtagccccaaaaaatagcgat

SEQ ID NO: 408 - DNA, 29 bp, synthetic construct
  source 1..29: mol_type = other DNA
cccctcagtgtcgatgcaatgcctgagta

SEQ ID NO: 409 - DNA, 16 bp, synthetic construct
  source 1..16: mol_type = other DNA
tacatggccttagccg

SEQ ID NO: 410 - DNA, 24 bp, synthetic construct
  source 1..24: mol_type = other DNA
gtctctgaataagggagaacggtg

SEQ ID NO: 411 - DNA, 32 bp, synthetic construct
  source 1..32: mol_type = other DNA
cacaaacatatatgtaatataagtatagcccg

SEQ ID NO: 412 - DNA, 24 bp, synthetic construct
  source 1..24: mol_type = other DNA
tttttgggaagacaaatcatcgag

SEQ ID NO: 413 - DNA, 28 bp, synthetic construct
  source 1..28: mol_type = other DNA
ccactactatatttccaagaagcgcctg

SEQ ID NO: 414 - DNA, 31 bp, synthetic construct
  source 1..31: mol_type = other DNA
ataacctaaaagaacgtggactccaacgtca

SEQ ID NO: 415 - DNA, 33 bp, synthetic construct
  source 1..33: mol_type = other DNA
agtctggggtctttggaagcccgaatgtttaga

SEQ ID NO: 416 - DNA, 31 bp, synthetic construct
  source 1..31: mol_type = other DNA
atgtgtagaatgctttaatattcattgaatc

SEQ ID NO: 417 - DNA, 29 bp, synthetic construct
  source 1..29: mol_type = other DNA
catgttattttgatggggtcagtgccttg

SEQ ID NO: 418 - DNA, 29 bp, synthetic construct
  source 1..29: mol_type = other DNA
gtcaatcatttacctaaacagttaatgcc

SEQ ID NO: 419 - DNA, 23 bp, synthetic construct
  source 1..23: mol_type = other DNA
ggcttgcagggagttatattcgg

SEQ ID NO: 420 - DNA, 33 bp, synthetic construct
  source 1..33: mol_type = other DNA
aacgagggaataaatcaagtattaagacattga

SEQ ID NO: 421 - DNA, 23 bp, synthetic construct
  source 1..23: mol_type = other DNA
cttagattaagacgcataaataa

SEQ ID NO: 422 - DNA, 31 bp, synthetic construct
  source 1..31: mol_type = other DNA
ttttcaaagtgaaccaccctaaagggagccc

SEQ ID NO: 423 - DNA, 24 bp, synthetic construct
  source 1..24: mol_type = other DNA
ctgacctaaaaaccgtcggggaaa

SEQ ID NO: 424 - DNA, 25 bp, synthetic construct
  source 1..25: mol_type = other DNA
aattaagcctccagtataaagccaa

SEQ ID NO: 425 - DNA, 37 bp, synthetic construct
  source 1..37: mol_type = other DNA
gatctacatgcttctttcaactttacatcaagaaaac

SEQ ID NO: 426 - DNA, 22 bp, synthetic construct
  source 1..22: mol_type = other DNA
ttaatgccggagagggaattac

SEQ ID NO: 427 - DNA, 15 bp, synthetic construct
  source 1..15: mol_type = other DNA
ggccggagacagtca

SEQ ID NO: 428 - DNA, 31 bp, synthetic construct
  source 1..31: mol_type = other DNA
ataaattgtaaagattcaaaaggtgtacccc

SEQ ID NO: 429 - DNA, 31 bp, synthetic construct
  source 1..31: mol_type = other DNA
aaagtacaacggagatttgtatcacctgctc

SEQ ID NO: 430 - DNA, 22 bp, synthetic construct
  source 1..22: mol_type = other DNA
tgacccccagcgattgaatttt

SEQ ID NO: 431 - DNA, 22 bp, synthetic construct
  source 1..22: mol_type = other DNA
aggcaaaagaatacactttaat

SEQ ID NO: 432 - DNA, 24 bp, synthetic construct
  source 1..24: mol_type = other DNA
cattaaacgggtaaaattgcgccg

SEQ ID NO: 433 - DNA, 26 bp, synthetic construct
  source 1..26: mol_type = other DNA
aggactaaagactcaccctcagcagc

SEQ ID NO: 434 - DNA, 30 bp, synthetic construct
  source 1..30: mol_type = other DNA
tatataactagcaacggctacaggcatcgg

SEQ ID NO: 435 - DNA, 31 bp, synthetic construct
  source 1..31: mol_type = other DNA
tgaatttatcaaaattgcagaaccgggtatt

SEQ ID NO: 436 - DNA, 16 bp, synthetic construct
  source 1..16: mol_type = other DNA
ctacctttttaacctc

SEQ ID NO: 437 - DNA, 57 bp, synthetic construct
  source 1..57: mol_type = other DNA
gcagtagagtaggtagagattaggcaccaatcgcgtcagacgattggccttgatatt

SEQ ID NO: 438 - DNA, 67 bp, synthetic construct
  source 1..67: mol_type = other DNA
gcagtagagtaggtagagattaggcatcagaagcaaggctatattaaattaatgcccacgctgaagt

SEQ ID NO: 439 - DNA, 61 bp, synthetic construct
  source 1..61: mol_type = other DNA
gcagtagagtaggtagagattaggcagagaatgatagcatgtagccccaaaaaatagcgat

SEQ ID NO: 440 - DNA, 55 bp, synthetic construct
  source 1..55: mol_type = other DNA
gcagtagagtaggtagagattaggcacccctcagtgtcgatgcaatgcctgagta

SEQ ID NO: 441 - DNA, 42 bp, synthetic construct
  source 1..42: mol_type = other DNA
gcagtagagtaggtagagattaggcatacatggccttagccg

SEQ ID NO: 442 - DNA, 50 bp, synthetic construct
  source 1..50: mol_type = other DNA
gcagtagagtaggtagagattaggcagtctctgaataagggagaacggtg

SEQ ID NO: 443 - DNA, 58 bp, synthetic construct
  source 1..58: mol_type = other DNA
gcagtagagtaggtagagattaggcacacaaacatatatgtaatataagtatagcccg

SEQ ID NO: 444 - DNA, 50 bp, synthetic construct
  source 1..50: mol_type = other DNA
gcagtagagtaggtagagattaggcatttttgggaagacaaatcatcgag

SEQ ID NO: 445 - DNA, 54 bp, synthetic construct
  source 1..54: mol_type = other DNA
gcagtagagtaggtagagattaggcaccactactatatttccaagaagcgcctg

SEQ ID NO: 446 - DNA, 57 bp, synthetic construct
  source 1..57: mol_type = other DNA
gcagtagagtaggtagagattaggcaataacctaaaagaacgtggactccaacgtca

SEQ ID NO: 447 - DNA, 59 bp, synthetic construct
  source 1..59: mol_type = other DNA
gcagtagagtaggtagagattaggcaagtctggggtctttggaagcccgaatgtttaga

SEQ ID NO: 448 - DNA, 57 bp, synthetic construct
  source 1..57: mol_type = other DNA
gcagtagagtaggtagagattaggcaatgtgtagaatgctttaatattcattgaatc

SEQ ID NO: 449 - DNA, 55 bp, synthetic construct
  source 1..55: mol_type = other DNA
gcagtagagtaggtagagattaggcacatgttattttgatggggtcagtgccttg

SEQ ID NO: 450 - DNA, 55 bp, synthetic construct
  source 1..55: mol_type = other DNA
gcagtagagtaggtagagattaggcagtcaatcatttacctaaacagttaatgcc

SEQ ID NO: 451 - DNA, 49 bp, synthetic construct
  source 1..49: mol_type = other DNA
gcagtagagtaggtagagattaggcaggcttgcagggagttatattcgg

SEQ ID NO: 452 - DNA, 59 bp, synthetic construct
  source 1..59: mol_type = other DNA
gcagtagagtaggtagagattaggcaaacgagggaataaatcaagtattaagacattga

SEQ ID NO: 453 - DNA, 49 bp, synthetic construct
  source 1..49: mol_type = other DNA
gcagtagagtaggtagagattaggcacttagattaagacgcataaataa

SEQ ID NO: 454 - DNA, 57 bp, synthetic construct
  source 1..57: mol_type = other DNA
gcagtagagtaggtagagattaggcattttcaaagtgaaccaccctaaagggagccc

SEQ ID NO: 455 - DNA, 50 bp, synthetic construct
  source 1..50: mol_type = other DNA
gcagtagagtaggtagagattaggcactgacctaaaaaccgtcggggaaa

SEQ ID NO: 456 - DNA, 51 bp, synthetic construct
  source 1..51: mol_type = other DNA
gcagtagagtaggtagagattaggcaaattaagcctccagtataaagccaa

SEQ ID NO: 457 - DNA, 63 bp, synthetic construct
  source 1..63: mol_type = other DNA
gcagtagagtaggtagagattaggcagatctacatgcttctttcaactttacatcaagaaaac

SEQ ID NO: 458 - DNA, 48 bp, synthetic construct
  source 1..48: mol_type = other DNA
gcagtagagtaggtagagattaggcattaatgccggagagggaattac

SEQ ID NO: 459 - DNA, 41 bp, synthetic construct
  source 1..41: mol_type = other DNA
gcagtagagtaggtagagattaggcaggccggagacagtca

SEQ ID NO: 460 - DNA, 57 bp, synthetic construct
  source 1..57: mol_type = other DNA
gcagtagagtaggtagagattaggcaataaattgtaaagattcaaaaggtgtacccc

SEQ ID NO: 461 - DNA, 57 bp, synthetic construct
  source 1..57: mol_type = other DNA
gcagtagagtaggtagagattaggcaaaagtacaacggagatttgtatcacctgctc

SEQ ID NO: 462 - DNA, 48 bp, synthetic construct
  source 1..48: mol_type = other DNA
gcagtagagtaggtagagattaggcatgacccccagcgattgaatttt

SEQ ID NO: 463 - DNA, 48 bp, synthetic construct
  source 1..48: mol_type = other DNA
gcagtagagtaggtagagattaggcaaggcaaaagaatacactttaat

SEQ ID NO: 464 - DNA, 50 bp, synthetic construct
  source 1..50: mol_type = other DNA
gcagtagagtaggtagagattaggcacattaaacgggtaaaattgcgccg

SEQ ID NO: 465 - DNA, 52 bp, synthetic construct
  source 1..52: mol_type = other DNA
gcagtagagtaggtagagattaggcaaggactaaagactcaccctcagcagc

SEQ ID NO: 466 - DNA, 56 bp, synthetic construct
  source 1..56: mol_type = other DNA
gcagtagagtaggtagagattaggcatatataactagcaacggctacaggcatcgg

SEQ ID NO: 467 - DNA, 57 bp, synthetic construct
  source 1..57: mol_type = other DNA
gcagtagagtaggtagagattaggcatgaatttatcaaaattgcagaaccgggtatt

SEQ ID NO: 468 - DNA, 42 bp, synthetic construct
  source 1..42: mol_type = other DNA
gcagtagagtaggtagagattaggcactacctttttaacctc

SEQ ID NO: 469 - skipped

SEQ ID NO: 470 - DNA, 37 bp, synthetic construct
  source 1..37: mol_type = other DNA
gagacgcagaaacagcggataccggcgaaagaggtgg

SEQ ID NO: 471 - DNA, 30 bp, synthetic construct
  source 1..30: mol_type = other DNA
taccgggtaaagggagccctgagacaagag

SEQ ID NO: 472 - DNA, 38 bp, synthetic construct
  source 1..38: mol_type = other DNA
gatagacgcaggcgacgcctgtcgatccagaaaaaaat

SEQ ID NO: 473 - DNA, 53 bp, synthetic construct
  source 1..53: mol_type = other DNA
actaacaattttagtttcctaatttaaaagggacattctgattaccgcttgac

SEQ ID NO: 474 - DNA, 50 bp, synthetic construct
  source 1..50: mol_type = other DNA
tccccgggtaccgagctcgatgtgaaagcaacaggaaaaacgtcacacga

SEQ ID NO: 475 - DNA, 51 bp, synthetic construct
  source 1..51: mol_type = other DNA
gtatcataattaattttcccttatttttaatccttgaaaacatcaggaacg

SEQ ID NO: 476 - DNA, 50 bp, synthetic construct
  source 1..50: mol_type = other DNA
aagaaaaataataatggaagggttttttagaacctaccaaagtcctgaac

SEQ ID NO: 477 - DNA, 51 bp, synthetic construct
  source 1..51: mol_type = other DNA
aattgggcttgagatggtttacgacctgctccatgtcggtcgctgaggctt

SEQ ID NO: 478 - DNA, 50 bp, synthetic construct
  source 1..50: mol_type = other DNA
tcatattcattccaagaaccctcatataataacggcgggtagatgggcgc

SEQ ID NO: 479 - DNA, 54 bp, synthetic construct
  source 1..54: mol_type = other DNA
ggcttatggttgctcagccattttgttatccgctcacacgtgccggacttgtag

SEQ ID NO: 480 - DNA, 53 bp, synthetic construct
  source 1..53: mol_type = other DNA
aggaattgtgagaatactcaagagaaggatttttgatgattttgcggcccgaa

SEQ ID NO: 481 - DNA, 48 bp, synthetic construct
  source 1..48: mol_type = other DNA
caccctcacgggaacgtttgaattgagaggcttagcaacgcatgagga

SEQ ID NO: 482 - DNA, 52 bp, synthetic construct
  source 1..52: mol_type = other DNA
gcgtcttgagtctccaaaaaaagaaccgccaccctgaggtgccgtgaatagg

SEQ ID NO: 483 - DNA, 48 bp, synthetic construct
  source 1..48: mol_type = other DNA
ttatcaacaatagattatcaaaaatctaaaatatttttttttaggagc

SEQ ID NO: 484 - DNA, 54 bp, synthetic construct
  source 1..54: mol_type = other DNA
agggcgaatggtaatgggtaaaggtttttttctttgctcgttcacagttgagga

SEQ ID NO: 485 - DNA, 54 bp, synthetic construct
  source 1..54: mol_type = other DNA
gcttctgtcgtggtgaagggatatcactgcgaaatcctgcttataaatcaaaag

SEQ ID NO: 486 - DNA, 49 bp, synthetic construct
  source 1..49: mol_type = other DNA
tagccctaaatgatagaaaaagtttttctgtttagtataaagaaccacc

SEQ ID NO: 487 - DNA, 54 bp, synthetic construct
  source 1..54: mol_type = other DNA
attaacctaatgaatcggccaagggtaaagcagtttttggtgccggtgccccct

SEQ ID NO: 488 - DNA, 54 bp, synthetic construct
  source 1..54: mol_type = other DNA
attgaccgtaatgggggataagatgtatcagagagataaaatcaagatttattc

SEQ ID NO: 489 - DNA, 53 bp, synthetic construct
  source 1..53: mol_type = other DNA
acccacacaacgcatccagtaaaagcgcagtctctgaaataggtcgactttac

SEQ ID NO: 490 - DNA, 49 bp, synthetic construct
  source 1..49: mol_type = other DNA
atgaacgcgctttttgggagaaaagagtttttctgtccagccattaaaa

SEQ ID NO: 491 - DNA, 52 bp, synthetic construct
  source 1..52: mol_type = other DNA
aaatgctaaaagggaatacctattcagggcatagctctggtctgctgttgcc

SEQ ID NO: 492 - DNA, 53 bp, synthetic construct
  source 1..53: mol_type = other DNA
ttgtaaaggaaaccaggcaaagcgccattctgaacctcataattacgaatcgc

SEQ ID NO: 493 - DNA, 48 bp, synthetic construct
  source 1..48: mol_type = other DNA
ccattaccattttttagcacgcaattttttataacgggtgtctggaag

SEQ ID NO: 494 - DNA, 53 bp, synthetic construct
  source 1..53: mol_type = other DNA
tgctccttgggcgacaaattaattacattgccaccctcagccgccaccagaac

SEQ ID NO: 495 - DNA, 47 bp, synthetic construct
  source 1..47: mol_type = other DNA
ttatataaactttttcccatcaaaccagtttgaggggacgacgacag

SEQ ID NO: 496 - DNA, 53 bp, synthetic construct
  source 1..53: mol_type = other DNA
aaaatcaggtctttagagccgcccgtataaacagttaatgcccgctttaaaca

SEQ ID NO: 497 - DNA, 55 bp, synthetic construct
  source 1..55: mol_type = other DNA
ttgctcagtaccaggctttaccgtaggataaaaaacaattggagcggaattatca

SEQ ID NO: 498 - DNA, 48 bp, synthetic construct
  source 1..48: mol_type = other DNA
aagcaaaaacggtttttaatcggcctttttttcaccgcctgttttaga

SEQ ID NO: 499 - DNA, 46 bp, synthetic construct
  source 1..46: mol_type = other DNA
cgcctgcaacagtggcgaattattaaccttgtaccccgttgtgaga

SEQ ID NO: 500 - DNA, 46 bp, synthetic construct
  source 1..46: mol_type = other DNA
tgccgtcgagaggattcaaccgtgccttcccgcaagaccatttgag

SEQ ID NO: 501 - DNA, 46 bp, synthetic construct
  source 1..46: mol_type = other DNA
gaatggagcgtcatacatggctaggattaggttaagcctaattttt

SEQ ID NO: 502 - DNA, 46 bp, synthetic construct
  source 1..46: mol_type = other DNA
tcgagaactccttatcctgattagatttagaagtattaacgttggt

SEQ ID NO: 503 - DNA, 46 bp, synthetic construct
  source 1..46: mol_type = other DNA
ttttgttaaatcagcgagatcttcataggttgctttccctcaaact

SEQ ID NO: 504 - DNA, 56 bp, synthetic construct
  source 1..56: mol_type = other DNA
aattctactttttaaatatgcaactaaagtacgaatacccaaaagaaccagtagca

SEQ ID NO: 505 - DNA, 45 bp, synthetic construct
  source 1..45: mol_type = other DNA
gagatagttttttgttgagtgccgggttacacggtcaagctgcat

SEQ ID NO: 506 - DNA, 45 bp, synthetic construct
  source 1..45: mol_type = other DNA
aatcgatgtatttaaacggaatcaaatatcaatatacagtaacag

SEQ ID NO: 507 - DNA, 45 bp, synthetic construct
  source 1..45: mol_type = other DNA
agccgccacaaggcgacatcaaaaaaagatgagagtacctttaat

SEQ ID NO: 508 - DNA, 44 bp, synthetic construct
  source 1..44: mol_type = other DNA
ggaaattcctttttcacaacatacgagccgcacatcctcataac

SEQ ID NO: 509 - DNA, 52 bp, synthetic construct
  source 1..52: mol_type = other DNA
agcattaacatccaataaatcaattgctgaatataatgctgtagctccttat

SEQ ID NO: 510 - DNA, 56 bp, synthetic construct
  source 1..56: mol_type = other DNA
caccaccaccctgactgttcagaaatacataaaaaggtgaatagaaaacctaaaac

SEQ ID NO: 511 - DNA, 42 bp, synthetic construct
  source 1..42: mol_type = other DNA
ataaccgatatatttacttagcctttttgaacgaggcgagta

SEQ ID NO: 512 - DNA, 42 bp, synthetic construct
  source 1..42: mol_type = other DNA
catgaaagtatttttaacttgtaccactgtttagacatttcg

SEQ ID NO: 513 - DNA, 47 bp, synthetic construct
  source 1..47: mol_type = other DNA
ataccgaacgccaacgctctttttacagtagggcacatcgggagaaa

SEQ ID NO: 514 - DNA, 57 bp, synthetic construct
  source 1..57: mol_type = other DNA
taatgcgccgctacaggaaatgccggagagggtagctatttttttttgatcattttt

SEQ ID NO: 515 - DNA, 57 bp, synthetic construct
  source 1..57: mol_type = other DNA
cacgctggtttgccccatttgcaacagctgatttaaaactagcatgtcagccccaaa

SEQ ID NO: 516 - DNA, 41 bp, synthetic construct
  source 1..41: mol_type = other DNA
aagccggcgcgtggcgagaagcctcccataaacgcctccgg

SEQ ID NO: 517 - DNA, 41 bp, synthetic construct
  source 1..41: mol_type = other DNA
accatcgaccgaacaaagttaccaaattctgcggtggcatc

SEQ ID NO: 518 - DNA, 40 bp, synthetic construct
  source 1..40: mol_type = other DNA
gttgtagcaatacttcttaatgcggcttagatataaacac

SEQ ID NO: 519 - DNA, 47 bp, synthetic construct
  source 1..47: mol_type = other DNA
gacaaaattgataagtcagaagcaaagcggaaccctcagagaaccgc

SEQ ID NO: 520 - DNA, 40 bp, synthetic construct
  source 1..40: mol_type = other DNA
accagagcttgccagatccccctctattcattaggccaaa

SEQ ID NO: 521 - DNA, 47 bp, synthetic construct
  source 1..47: mol_type = other DNA
atcattttgcggaacaaagaaacgcgaggcgctttatttagaattga

SEQ ID NO: 522 - DNA, 55 bp, synthetic construct
  source 1..55: mol_type = other DNA
acgtggcatagaagaatcgttagcgtccgtgaaggaaggtggactccaacgtcaa

SEQ ID NO: 523 - DNA, 39 bp, synthetic construct
  source 1..39: mol_type = other DNA
gctgcgcaactgttgggaagggcgagagccacgctgaga

SEQ ID NO: 524 - DNA, 47 bp, synthetic construct
  source 1..47: mol_type = other DNA
aaacaatcaaaatagcaccttttttaatggatgaaacaaattaagtt

SEQ ID NO: 525 - DNA, 39 bp, synthetic construct
  source 1..39: mol_type = other DNA
gaaagaggccccagcgagatttgtatgcgattaggaatt

SEQ ID NO: 526 - DNA, 61 bp, synthetic construct
  source 1..61: mol_type = other DNA
acattttgacgctcaatcgttttgagtaatactgaacaaaatcggatcacccaaatcaagt

SEQ ID NO: 527 - DNA, 47 bp, synthetic construct
  source 1..47: mol_type = other DNA
agccagcaaaatcactggcatgattaagactcaacatgaatagtagt

SEQ ID NO: 528 - DNA, 47 bp, synthetic construct
  source 1..47: mol_type = other DNA
cagaacaccgagtaggcggtttgcgtatgcacaggcggcctttagtg

SEQ ID NO: 529 - DNA, 39 bp, synthetic construct
  source 1..39: mol_type = other DNA
gattgtttggattatacttctgaatatcccaaatttcat

SEQ ID NO: 530 - DNA, 39 bp, synthetic construct
  source 1..39: mol_type = other DNA
agcgatacgaactgacatatttaacaacgccgtcagatg

SEQ ID NO: 531 - DNA, 64 bp, synthetic construct
  source 1..64: mol_type = other DNA
atagtaaaacgaggcatgtatgggattttttttggccatcttttttttcataattattctgaaa

SEQ ID NO: 532 - DNA, 39 bp, synthetic construct
  source 1..39: mol_type = other DNA
caaatggtagtttgagtaacattcgttattaattttaaa

SEQ ID NO: 533 - DNA, 48 bp, synthetic construct
  source 1..48: mol_type = other DNA
atcggcctcaggaagaggaattgaatggtttggcgcctgtgtaagaat

SEQ ID NO: 534 - DNA, 39 bp, synthetic construct
  source 1..39: mol_type = other DNA
tcagatgatggcaattcatcaataccaatcagcgagaaa

SEQ ID NO: 535 - DNA, 39 bp, synthetic construct
  source 1..39: mol_type = other DNA
gctctcaccccggaatttgtaaaacgacggctttcccag

SEQ ID NO: 536 - DNA, 47 bp, synthetic construct
  source 1..47: mol_type = other DNA
gaaaggatagcgtttctaaacaatttatcagacaagaacaagctgct

SEQ ID NO: 537 - DNA, 38 bp, synthetic construct
  source 1..38: mol_type = other DNA
ttttgttatgagagtctgagaagaaacaggagtaataa

SEQ ID NO: 538 - DNA, 38 bp, synthetic construct
  source 1..38: mol_type = other DNA
cattaaatcagccagctttccggcaccgctttctggtc

SEQ ID NO: 539 - DNA, 38 bp, synthetic construct
  source 1..38: mol_type = other DNA
gttcttcgaatcagagtgcgcgttatcaggtcattgcc

SEQ ID NO: 540 - DNA, 38 bp, synthetic construct
  source 1..38: mol_type = other DNA
cgtcaatagcatctgaataattcgcgtctgtctagctg

SEQ ID NO: 541 - DNA, 32 bp, synthetic construct
  source 1..32: mol_type = other DNA
atcaatataaagattcgatgcaaaatttacat

SEQ ID NO: 542 - DNA, 38 bp, synthetic construct
  source 1..38: mol_type = other DNA
gaacaatgccaacagtttttgatagaacccaatatcca

SEQ ID NO: 543 - DNA, 30 bp, synthetic construct
  source 1..30: mol_type = other DNA
aacaggtcaggattataagaggaagcccga

SEQ ID NO: 544 - DNA, 27 bp, synthetic construct
  source 1..27: mol_type = other DNA
aacagtacagctttcacgtacagcgcc

SEQ ID NO: 545 - DNA, 38 bp, synthetic construct
  source 1..38: mol_type = other DNA
gctggcgagggtaacgccagggtcagtgccaataaatc

SEQ ID NO: 546 - DNA, 38 bp, synthetic construct
  source 1..38: mol_type = other DNA
gctgaaaagaacgagtagatttagtttgaccattagat

SEQ ID NO: 547 - DNA, 38 bp, synthetic construct
  source 1..38: mol_type = other DNA
atgaacggatgtttagactggatagcgtttattaaagg

SEQ ID NO: 548 - DNA, 38 bp, synthetic construct
  source 1..38: mol_type = other DNA
attatagaggtcatttttgcggatggcttagaaaatac

SEQ ID NO: 549 - DNA, 38 bp, synthetic construct
  source 1..38: mol_type = other DNA
attcgagcatcggtggttgataatcagaaaaatcatat

SEQ ID NO: 550 - DNA, 38 bp, synthetic construct
  source 1..38: mol_type = other DNA
ccgaggaaaaggccggaaacgtcaaccatcgcccacgc

SEQ ID NO: 551 - DNA, 38 bp, synthetic construct
  source 1..38: mol_type = other DNA
ctatatgtataaattgacagtcaaatcacccccgattt

SEQ ID NO: 552 - DNA, 38 bp, synthetic construct
  source 1..38: mol_type = other DNA
caccggagaattttctagtaagacgccaaattaagaac

SEQ ID NO: 553 - DNA, 31 bp, synthetic construct
  source 1..31: mol_type = other DNA
aaattcggataaataagttggcattgcgtag

SEQ ID NO: 554 - DNA, 31 bp, synthetic construct
  source 1..31: mol_type = other DNA
gtggtcgggaaacctcggcaaactaacggca

SEQ ID NO: 555 - DNA, 37 bp, synthetic construct
  source 1..37: mol_type = other DNA
attcgtaagccgggcgcgtactaggttgggccagtaa

SEQ ID NO: 556 - DNA, 32 bp, synthetic construct
  source 1..32: mol_type = other DNA
caatcatagccgacaatgacaacaccaatgaa

SEQ ID NO: 557 - DNA, 37 bp, synthetic construct
  source 1..37: mol_type = other DNA
gaaatagcccttatacaactaatgtatcggctttcaa

SEQ ID NO: 558 - DNA, 37 bp, synthetic construct
  source 1..37: mol_type = other DNA
atgaccataacagtgccgccagcattgacaggaggtt

SEQ ID NO: 559 - DNA, 37 bp, synthetic construct
  source 1..37: mol_type = other DNA
cggggttcgtactcaggaggtttagtagagcgaataa

SEQ ID NO: 560 - DNA, 30 bp, synthetic construct
  source 1..30: mol_type = other DNA
tgaccttcatcaaaggcttgccctgacgag

SEQ ID NO: 561 - DNA, 37 bp, synthetic construct
  source 1..37: mol_type = other DNA
gttacaaaatcgcgcttcaaagcgaaccagcgtttta

SEQ ID NO: 562 - DNA, 36 bp, synthetic construct
  source 1..36: mol_type = other DNA
atgttacagcccttctgctcaaggtagagaactaac

SEQ ID NO: 563 - DNA, 29 bp, synthetic construct
  source 1..29: mol_type = other DNA
taaccaataggaacgaaatatactaatag

SEQ ID NO: 564 - DNA, 23 bp, synthetic construct
  source 1..23: mol_type = other DNA
agtttccgtcaggaccattcaac

SEQ ID NO: 565 - DNA, 36 bp, synthetic construct
  source 1..36: mol_type = other DNA
caccgggttttgtttaccagagaataccagttggga

SEQ ID NO: 566 - DNA, 35 bp, synthetic construct
  source 1..35: mol_type = other DNA
atcaaaaacaaaggcaaccaccacacccgccgcgc

SEQ ID NO: 567 - DNA, 35 bp, synthetic construct
  source 1..35: mol_type = other DNA
ctcagagcccctgcctattttcattgaagggggta

SEQ ID NO: 568 - DNA, 35 bp, synthetic construct
  source 1..35: mol_type = other DNA
gaggcaggtcagacgattggccttgatacaataac

SEQ ID NO: 569 - DNA, 35 bp, synthetic construct
  source 1..35: mol_type = other DNA
caataacggattcgcctgattgctttgtcttacca

SEQ ID NO: 570 - DNA, 35 bp, synthetic construct
  source 1..35: mol_type = other DNA
acagaatattagcaaaattttttaagcaataaagc

SEQ ID NO: 571 - DNA, 35 bp, synthetic construct
  source 1..35: mol_type = other DNA
tttcattccatataacagttgattcccgaaggaaa

SEQ ID NO: 572 - DNA, 34 bp, synthetic construct
  source 1..34: mol_type = other DNA
ccatttgggaattaggcagggaggcgtcagactg

SEQ ID NO: 573 - DNA, 34 bp, synthetic construct
  source 1..34: mol_type = other DNA
agcagaagtgaggctcctgagacggccagaatgc

SEQ ID NO: 574 - DNA, 34 bp, synthetic construct
  source 1..34: mol_type = other DNA
gagaatataaagtgacaagtgaatttgaccgtgt

SEQ ID NO: 575 - DNA, 24 bp, synthetic construct
  source 1..24: mol_type = other DNA
aagcgcatccgggtcagtcagcag

SEQ ID NO: 576 - DNA, 33 bp, synthetic construct
  source 1..33: mol_type = other DNA
caatagctatcttacctaaatcggggggtcagt

SEQ ID NO: 577 - DNA, 25 bp, synthetic construct
  source 1..25: mol_type = other DNA
atcgtaaccgtgataataaaagaac

SEQ ID NO: 578 - DNA, 33 bp, synthetic construct
  source 1..33: mol_type = other DNA
tatcataaaagaagttcaccaccggaacaacct

SEQ ID NO: 579 - DNA, 25 bp, synthetic construct
  source 1..25: mol_type = other DNA
aaatctaaagtttttatcaccttgc

SEQ ID NO: 580 - DNA, 31 bp, synthetic construct
  source 1..31: mol_type = other DNA
cataaagtgtaaagccgctggcaatccctta

SEQ ID NO: 581 - DNA, 29 bp, synthetic construct
  source 1..29: mol_type = other DNA
ggcgggccgttttcctgcagcttaaacga

SEQ ID NO: 582 - DNA, 32 bp, synthetic construct
  source 1..32: mol_type = other DNA
ttctgacctaaatttaggaaggttttatttgc

SEQ ID NO: 583 - DNA, 32 bp, synthetic construct
  source 1..32: mol_type = other DNA
tcatttcatcacgacgcgggcctcttcgctat

SEQ ID NO: 584 - DNA, 32 bp, synthetic construct
  source 1..32: mol_type = other DNA
attacctgaaatatcgaccggaagcaaactcc

SEQ ID NO: 585 - DNA, 32 bp, synthetic construct
  source 1..32: mol_type = other DNA
gcgaaaggggtcacgccgggagctagtcaata

SEQ ID NO: 586 - DNA, 29 bp, synthetic construct
  source 1..29: mol_type = other DNA
gagcacgtataacgctgagagatgaaagc

SEQ ID NO: 587 - DNA, 32 bp, synthetic construct
  source 1..32: mol_type = other DNA
aaaccctcaatcaatactggtgcccgttaata

SEQ ID NO: 588 - DNA, 21 bp, synthetic construct
  source 1..21: mol_type = other DNA
ttctgaccctacctttttttt

SEQ ID NO: 589 - DNA, 29 bp, synthetic construct
  source 1..29: mol_type = other DNA
aaagcactccctgaatcaatccgcaccgt

SEQ ID NO: 590 - DNA, 32 bp, synthetic construct
  source 1..32: mol_type = other DNA
ttcacaaagtgtactggtaataagtaagaggc

SEQ ID NO: 591 - DNA, 32 bp, synthetic construct
  source 1..32: mol_type = other DNA
gccttgagtaaatcatacaggcaatacgcagt

SEQ ID NO: 592 - DNA, 31 bp, synthetic construct
  source 1..31: mol_type = other DNA
agcgggcgccagcacgcaaatcgtgcggtcc

SEQ ID NO: 593 - DNA, 32 bp, synthetic construct
  source 1..32: mol_type = other DNA
tcagggatagcaagtttttccaataggaaccc

SEQ ID NO: 594 - DNA, 32 bp, synthetic construct
  source 1..32: mol_type = other DNA
taccttttttaatacatctcacgcaagtacgc

SEQ ID NO: 595 - DNA, 31 bp, synthetic construct
  source 1..31: mol_type = other DNA
accctcaaggctccaatcagcggagcttacg

SEQ ID NO: 596 - DNA, 31 bp, synthetic construct
  source 1..31: mol_type = other DNA
attgtgtcgaaatccgatttcaactttaatc

SEQ ID NO: 597 - DNA, 30 bp, synthetic construct
  source 1..30: mol_type = other DNA
tccagagcgctacaattttatccaagcctt

SEQ ID NO: 598 - DNA, 31 bp, synthetic construct
  source 1..31: mol_type = other DNA
cttgctttcgaggtgattttcggtcatagcc

SEQ ID NO: 599 - DNA, 30 bp, synthetic construct
  source 1..30: mol_type = other DNA
caataataagagcaatgtaatactacagga

SEQ ID NO: 600 - DNA, 31 bp, synthetic construct
  source 1..31: mol_type = other DNA
tacgagcatgtagaaataatcctattagagc

SEQ ID NO: 601 - DNA, 31 bp, synthetic construct
  source 1..31: mol_type = other DNA
taagacgctggagcaagctggcaagtgtagc

SEQ ID NO: 602 - DNA, 30 bp, synthetic construct
  source 1..30: mol_type = other DNA
ggtttctgctagggcgtttggaacaagagt

SEQ ID NO: 603 - DNA, 30 bp, synthetic construct
  source 1..30: mol_type = other DNA
attttcaggtttaacaacatgtaaataaga

SEQ ID NO: 604 - DNA, 31 bp, synthetic construct
  source 1..31: mol_type = other DNA
ccctcgtcgtctttcgagtttcgtcaccagt

SEQ ID NO: 605 - DNA, 30 bp, synthetic construct
  source 1..30: mol_type = other DNA
taagaaccaccagaacgacaactcgtatta

SEQ ID NO: 606 - DNA, 30 bp, synthetic construct
  source 1..30: mol_type = other DNA
tacgccaaagacttcagcaaaagaagatga

SEQ ID NO: 607 - DNA, 30 bp, synthetic construct
  source 1..30: mol_type = other DNA
tcatggtagagcttctatcagggcgatggc

SEQ ID NO: 608 - DNA, 31 bp, synthetic construct
  source 1..31: mol_type = other DNA
ggcaaagacaagtttgcctccaatactgcgg

SEQ ID NO: 609 - DNA, 31 bp, synthetic construct
  source 1..31: mol_type = other DNA
aaaacattatgacccgaaacaattgagactc

SEQ ID NO: 610 - DNA, 31 bp, synthetic construct
  source 1..31: mol_type = other DNA
gtgtaggtgatgttgatataagtatagcccg

SEQ ID NO: 611 - DNA, 30 bp, synthetic construct
  source 1..30: mol_type = other DNA
taccagtggaaaaaaatatatgtgagtgaa

SEQ ID NO: 612 - DNA, 30 bp, synthetic construct
  source 1..30: mol_type = other DNA
aacgagataaatatcggaacctatcaaaat

SEQ ID NO: 613 - DNA, 30 bp, synthetic construct
  source 1..30: mol_type = other DNA
acgtaaaacagaaattgcagaacaaatacc

SEQ ID NO: 614 - DNA, 47 bp, synthetic construct
  source 1..47: mol_type = other DNA
aatttttagaacgggtattagttgcaaatcagatatagaaggcgggt

SEQ ID NO: 615 - DNA, 69 bp, synthetic construct
  source 1..69: mol_type = other DNA
ttgattatccggaaaccaagtaccgcactcaatagcaagctactgaaatggatttccattttaaatgca

SEQ ID NO: 616 - DNA, 51 bp, synthetic construct
  source 1..51: mol_type = other DNA
ttgattaacgtcaaaaatgaagaaacgaccagttacaacggcagccgggcg

SEQ ID NO: 617 - DNA, 37 bp, synthetic construct
  source 1..37: mol_type = other DNA
ataatcgtggcagatcgtaggaagagagaataatttt

SEQ ID NO: 618 - DNA, 31 bp, synthetic construct
  source 1..31: mol_type = other DNA
tcggtcattcaccagctcatggatgagaaag

SEQ ID NO: 619 - DNA, 63 bp, synthetic construct
  source 1..63: mol_type = other DNA
tttccataaaaaatttatcccaatccaaataaaatagcagcctttacatcattaccgcgccca

SEQ ID NO: 620 - DNA, 31 bp, synthetic construct
  source 1..31: mol_type = other DNA
gctgtcttaagcaagccgtttttattttcat

SEQ ID NO: 621 - DNA, 44 bp, synthetic construct
  source 1..44: mol_type = other DNA
aattgagcgctaaccttgcacccactaatttgttttttgtggat

SEQ ID NO: 622 - DNA, 55 bp, synthetic construct
  source 1..55: mol_type = other DNA
ttaccgcatcggcaatttcttgaactgtttttccaactttgaaagataggctggc

SEQ ID NO: 623 - DNA, 50 bp, synthetic construct
  source 1..50: mol_type = other DNA
caaataaatcctcattaaagccaaatcctttggagaagcttttagcgaat

SEQ ID NO: 624 - DNA, 23 bp, synthetic construct
  source 1..23: mol_type = other DNA
tgctaacgacgcaaccggtcatt

SEQ ID NO: 625 - DNA, 35 bp, synthetic construct
  source 1..35: mol_type = other DNA
tagtaaataaagcgaagcccccgtaatcagtagcg

SEQ ID NO: 626 - DNA, 53 bp, synthetic construct
  source 1..53: mol_type = other DNA
ctatattttcatttggggcgcgactcagagcgcagatagtagcagcatttttt

SEQ ID NO: 627 - DNA, 50 bp, synthetic construct
  source 1..50: mol_type = other DNA
tgacttgcgggaggttttgtgaatctttcacgttgaaaaggttgtatcac

SEQ ID NO: 628 - DNA, 33 bp, synthetic construct
  source 1..33: mol_type = other DNA
tagcgcgttttatagttgcagggaaccaaacat

SEQ ID NO: 629 - DNA, 27 bp, synthetic construct
  source 1..27: mol_type = other DNA
ctgataaccgctttttacactaatgat

SEQ ID NO: 630 - DNA, 51 bp, synthetic construct
  source 1..51: mol_type = other DNA
tgctacactcatctttgaccaaaagaagcgggatcccgtcaccaatcgtca

SEQ ID NO: 631 - DNA, 53 bp, synthetic construct
  source 1..53: mol_type = other DNA
tccttaccagcgtggcaacatataaaagaaacacaatcaattatcagtcaccc

SEQ ID NO: 632 - DNA, 48 bp, synthetic construct
  source 1..48: mol_type = other DNA
tcagcagcacaacggattataccaagcgcgaggtaaaatacgtaaact

SEQ ID NO: 633 - DNA, 39 bp, synthetic construct
  source 1..39: mol_type = other DNA
aacaaagtgaaagacaggcaccaattcatatggttccct

SEQ ID NO: 634 - DNA, 56 bp, synthetic construct
  source 1..56: mol_type = other DNA
taccatgccactacgaagcatcggcggaaataaattaacaatttcagatcgcctcc

SEQ ID NO: 635 - DNA, 42 bp, synthetic construct
  source 1..42: mol_type = other DNA
taagaataagtttattttgtcgcaaagaaaacgtagagctta

SEQ ID NO: 636 - DNA, 28 bp, synthetic construct
  source 1..28: mol_type = other DNA
gacttgagatgttagccaccacggaaat

SEQ ID NO: 637 - DNA, 57 bp, synthetic construct
  source 1..57: mol_type = other DNA
tgtattaacactacaaataataccaagccagcagcaaatgaaccattcaggattgta

SEQ ID NO: 638 - DNA, 43 bp, synthetic construct
  source 1..43: mol_type = other DNA
tctaaaaaacaaacttaaattcatagtttgtagcattccacag

SEQ ID NO: 639 - DNA, 49 bp, synthetic construct
  source 1..49: mol_type = other DNA
aagggggatgtgctggagccaccttgcatcgaaaacaattcaaccgatt

SEQ ID NO: 640 - DNA, 30 bp, synthetic construct
  source 1..30: mol_type = other DNA
cgacgatagactttttgctacagaggcttt

SEQ ID NO: 641 - DNA, 34 bp, synthetic construct
  source 1..34: mol_type = other DNA
tagggaaggtaaatattgaaacgagggtttgcaa

SEQ ID NO: 642 - DNA, 51 bp, synthetic construct
  source 1..51: mol_type = other DNA
tgggcgcctggtgctgagtgttttgacggctcaaatcgtcagaggtgaggt

SEQ ID NO: 643 - DNA, 49 bp, synthetic construct
  source 1..49: mol_type = other DNA
ttcaccagtgatataatcagataaaacgctattatgcgttaaacaggaa

SEQ ID NO: 644 - DNA, 21 bp, synthetic construct
  source 1..21: mol_type = other DNA
gcctgagcaaccgacaacatt

SEQ ID NO: 645 - DNA, 46 bp, synthetic construct
  source 1..46: mol_type = other DNA
aggcgttcaataaacaatggctaagtaataagctcactgcccgtat

SEQ ID NO: 646 - DNA, 49 bp, synthetic construct
  source 1..49: mol_type = other DNA
tatttttgaacatgtttctgtccagacgacgaaatttaggcagagtttt

SEQ ID NO: 647 - DNA, 54 bp, synthetic construct
  source 1..54: mol_type = other DNA
taacaaggtaaagtaatcagctaaaaagaaaaatcaacagttgaaatcgcactc

SEQ ID NO: 648 - DNA, 37 bp, synthetic construct
  source 1..37: mol_type = other DNA
tctcgctttccaagctaatgacatcactttgcgttgc

SEQ ID NO: 649 - DNA, 39 bp, synthetic construct
  source 1..39: mol_type = other DNA
tggggcattttcgagccttagtcttttgattaggccgat

SEQ ID NO: 650 - DNA, 48 bp, synthetic construct
  source 1..48: mol_type = other DNA
aatttttagaacgggtattagttgcaaatcagatatagaaggcttttt

SEQ ID NO: 651 - DNA, 70 bp, synthetic construct
  source 1..70: mol_type = other DNA
tttttttatccggaaaccaagtaccgcactcaatagcaagctactgaaatggatttccattttaaatgca

SEQ ID NO: 652 - DNA, 52 bp, synthetic construct
  source 1..52: mol_type = other DNA
tttttttaacgtcaaaaatgaagaaacgaccagttacaacggcagccgggcg

SEQ ID NO: 653 - DNA, 38 bp, synthetic construct
  source 1..38: mol_type = other DNA
ataatcgtggcagatcgtaggaagagagaataattttt

SEQ ID NO: 654 - DNA, 32 bp, synthetic construct
  source 1..32: mol_type = other DNA
ttttttcattcaccagctcatggatgagaaag

SEQ ID NO: 655 - DNA, 64 bp, synthetic construct
  source 1..64: mol_type = other DNA
tttttcataaaaaatttatcccaatccaaataaaatagcagcctttacatcattaccgcgccca

SEQ ID NO: 656 - DNA, 32 bp, synthetic construct
  source 1..32: mol_type = other DNA
gctgtcttaagcaagccgtttttatttttttt

SEQ ID NO: 657 - DNA, 45 bp, synthetic construct
  source 1..45: mol_type = other DNA
aattgagcgctaaccttgcacccactaatttgttttttgtttttt

SEQ ID NO: 658 - DNA, 28 bp, synthetic construct
  source 1..28: mol_type = other DNA
ctgataaccgctttttacactaattttt

SEQ ID NO: 659 - DNA, 52 bp, synthetic construct
  source 1..52: mol_type = other DNA
tttttacactcatctttgaccaaaagaagcgggatcccgtcaccaatcgtca

SEQ ID NO: 660 - DNA, 54 bp, synthetic construct
  source 1..54: mol_type = other DNA
ttttttaccagcgtggcaacatataaaagaaacacaatcaattatcagtcaccc

SEQ ID NO: 661 - DNA, 49 bp, synthetic construct
  source 1..49: mol_type = other DNA
tcagcagcacaacggattataccaagcgcgaggtaaaatacgtattttt

SEQ ID NO: 662 - DNA, 40 bp, synthetic construct
  source 1..40: mol_type = other DNA
aacaaagtgaaagacaggcaccaattcatatggttttttt

SEQ ID NO: 663 - DNA, 57 bp, synthetic construct
  source 1..57: mol_type = other DNA
tttttatgccactacgaagcatcggcggaaataaattaacaatttcagatcgcctcc

SEQ ID NO: 664 - DNA, 43 bp, synthetic construct
  source 1..43: mol_type = other DNA
tttttaataagtttattttgtcgcaaagaaaacgtagagctta

SEQ ID NO: 665 - DNA, 29 bp, synthetic construct
  source 1..29: mol_type = other DNA
gacttgagatgttagccaccacggttttt

SEQ ID NO: 666 - DNA, 30 bp, synthetic construct
  source 1..30: mol_type = other DNA
ggaacaactgtgtacatcgacatcgcagtg

SEQ ID NO: 667 - DNA, 23 bp, synthetic construct
  source 1..23: mol_type = other DNA
tgtacagaccaggcgcaggacag

SEQ ID NO: 668 - DNA, 31 bp, synthetic construct
  source 1..31: mol_type = other DNA
caaccgcaagaatgccaaaataagagaatta

SEQ ID NO: 669 - DNA, 30 bp, synthetic construct
  source 1..30: mol_type = other DNA
cattaccctcatttcagtttcagcgccgcc

SEQ ID NO: 670 - DNA, 16 bp, synthetic construct
  source 1..16: mol_type = other DNA
cggtggtgccatccca

SEQ ID NO: 671 - DNA, 23 bp, synthetic construct
  source 1..23: mol_type = other DNA
attgtgaattaccttatcatcgc

SEQ ID NO: 672 - DNA, 31 bp, synthetic construct
  source 1..31: mol_type = other DNA
gcaggcgcgtccagaaccgccaccctcagag

SEQ ID NO: 673 - DNA, 31 bp, synthetic construct
  source 1..31: mol_type = other DNA
tgggcggtcaaaatcctttgatggtggttcc

SEQ ID NO: 674 - DNA, 22 bp, synthetic construct
  source 1..22: mol_type = other DNA
ctgcggcaaaccgtgacgggga

SEQ ID NO: 675 - DNA, 29 bp, synthetic construct
  source 1..29: mol_type = other DNA
taatgcataacactcagacgttagtaaat

SEQ ID NO: 676 - DNA, 31 bp, synthetic construct
  source 1..31: mol_type = other DNA
agaaaaatctacgttaataaaacaagattca

SEQ ID NO: 677 - DNA, 24 bp, synthetic construct
  source 1..24: mol_type = other DNA
cattcagtgaatagagtaatcttg

SEQ ID NO: 678 - DNA, 15 bp, synthetic construct
  source 1..15: mol_type = other DNA
cccgtaaaaaaagcc

SEQ ID NO: 679 - DNA, 23 bp, synthetic construct
  source 1..23: mol_type = other DNA
tggctcattataccaattaaacg

SEQ ID NO: 680 - DNA, 24 bp, synthetic construct
  source 1..24: mol_type = other DNA
aaacaccagaacgagtcagacggt

SEQ ID NO: 681 - DNA, 31 bp, synthetic construct
  source 1..31: mol_type = other DNA
cggttgcggtgaaccaaccctaaagggagcc

SEQ ID NO: 682 - DNA, 31 bp, synthetic construct
  source 1..31: mol_type = other DNA
ccaccacctgtccagcaaaggagcctttaat

SEQ ID NO: 683 - DNA, 32 bp, synthetic construct
  source 1..32: mol_type = other DNA
tcagttgaacaacgccagcgtaacgatctaaa

SEQ ID NO: 684 - DNA, 23 bp, synthetic construct
  source 1..23: mol_type = other DNA
tgctgattgccgttcgtcgtgcc

SEQ ID NO: 685 - DNA, 39 bp, synthetic construct
  source 1..39: mol_type = other DNA
ccactatttgttcagcgtgcctcctaactcacattaact

SEQ ID NO: 686 - DNA, 34 bp, synthetic construct
  source 1..34: mol_type = other DNA
aatagcccgcatcagagcactctgagggtggttt

SEQ ID NO: 687 - DNA, 31 bp, synthetic construct
  source 1..31: mol_type = other DNA
gaaatcggattattactttgccgccagcagt

SEQ ID NO: 688 - DNA, 14 bp, synthetic construct
  source 1..14: mol_type = other DNA
acaaactgatttag

SEQ ID NO: 689 - DNA, 24 bp, synthetic construct
  source 1..24: mol_type = other DNA
atgtaccggatacataagcaacac

SEQ ID NO: 690 - DNA, 34 bp, synthetic construct
  source 1..34: mol_type = other DNA
tcagatgttgttccaagagttgcagcaagcggtc

SEQ ID NO: 691 - DNA, 24 bp, synthetic construct
  source 1..24: mol_type = other DNA
tttttgggtttcgcaccaaagtca

SEQ ID NO: 692 - DNA, 29 bp, synthetic construct
  source 1..29: mol_type = other DNA
ccactacgtatgagtagacggacagccat

SEQ ID NO: 693 - DNA, 23 bp, synthetic construct
  source 1..23: mol_type = other DNA
cactggtgaaagaacggaagaaa

SEQ ID NO: 694 - DNA, 25 bp, synthetic construct
  source 1..25: mol_type = other DNA
ccagaggaagatcggccttgctggt

SEQ ID NO: 695 - DNA, 31 bp, synthetic construct
  source 1..31: mol_type = other DNA
gctggaggcaaatcaacgtaacacggatatt

SEQ ID NO: 696 - DNA, 23 bp, synthetic construct
  source 1..23: mol_type = other DNA
gttttttcgtctcgtctggggtg

SEQ ID NO: 697 - DNA, 23 bp, synthetic construct
  source 1..23: mol_type = other DNA
aacgtcagcgtggtggtttcctg

SEQ ID NO: 698 - DNA, 76 bp, synthetic construct
  source 1..76: mol_type = other DNA
gcagtagagtaggtagagattaggcattttttttttttttttttttggaacaactgtgtacatcgacatcgcagtg

SEQ ID NO: 699 - DNA, 69 bp, synthetic construct
  source 1..69: mol_type = other DNA
gcagtagagtaggtagagattaggcatttttttttttttttttttttgtacagaccaggcgcaggacag

SEQ ID NO: 700 - DNA, 77 bp, synthetic construct
  source 1..77: mol_type = other DNA
gcagtagagtaggtagagattaggcattttttttttttttttttttcaaccgcaagaatgccaaaataagagaatta

SEQ ID NO: 701 - DNA, 76 bp, synthetic construct
  source 1..76: mol_type = other DNA
gcagtagagtaggtagagattaggcattttttttttttttttttttcattaccctcatttcagtttcagcgccgcc

SEQ ID NO: 702 - DNA, 62 bp, synthetic construct
  source 1..62: mol_type = other DNA
gcagtagagtaggtagagattaggcattttttttttttttttttttcggtggtgccatccca

SEQ ID NO: 703 - DNA, 69 bp, synthetic construct
  source 1..69: mol_type = other DNA
gcagtagagtaggtagagattaggcattttttttttttttttttttattgtgaattaccttatcatcgc

SEQ ID NO: 704 - DNA, 77 bp, synthetic construct
  source 1..77: mol_type = other DNA
gcagtagagtaggtagagattaggcattttttttttttttttttttgcaggcgcgtccagaaccgccaccctcagag

SEQ ID NO: 705 - DNA, 77 bp, synthetic construct
  source 1..77: mol_type = other DNA
gcagtagagtaggtagagattaggcatttttttttttttttttttttgggcggtcaaaatcctttgatggtggttcc

SEQ ID NO: 706 - DNA, 68 bp, synthetic construct
  source 1..68: mol_type = other DNA
gcagtagagtaggtagagattaggcattttttttttttttttttttctgcggcaaaccgtgacgggga

SEQ ID NO: 707 - DNA, 75 bp, synthetic construct
  source 1..75: mol_type = other DNA
gcagtagagtaggtagagattaggcatttttttttttttttttttttaatgcataacactcagacgttagtaaat

SEQ ID NO: 708 - DNA, 77 bp, synthetic construct
  source 1..77: mol_type = other DNA
gcagtagagtaggtagagattaggcattttttttttttttttttttagaaaaatctacgttaataaaacaagattca

SEQ ID NO: 709 - DNA, 70 bp, synthetic construct
  source 1..70: mol_type = other DNA
gcagtagagtaggtagagattaggcattttttttttttttttttttcattcagtgaatagagtaatcttg

SEQ ID NO: 710 - DNA, 61 bp, synthetic construct
  source 1..61: mol_type = other DNA
gcagtagagtaggtagagattaggcattttttttttttttttttttcccgtaaaaaaagcc

SEQ ID NO: 711 - DNA, 69 bp, synthetic construct
  source 1..69: mol_type = other DNA
gcagtagagtaggtagagattaggcatttttttttttttttttttttggctcattataccaattaaacg

SEQ ID NO: 712 - DNA, 70 bp, synthetic construct
  source 1..70: mol_type = other DNA
gcagtagagtaggtagagattaggcattttttttttttttttttttaaacaccagaacgagtcagacggt

SEQ ID NO: 713 - DNA, 77 bp, synthetic construct
  source 1..77: mol_type = other DNA
gcagtagagtaggtagagattaggcattttttttttttttttttttcggttgcggtgaaccaaccctaaagggagcc

SEQ ID NO: 714 - DNA, 77 bp, synthetic construct
  source 1..77: mol_type = other DNA
gcagtagagtaggtagagattaggcattttttttttttttttttttccaccacctgtccagcaaaggagcctttaat

SEQ ID NO: 715 - DNA, 78 bp, synthetic construct
  source 1..78: mol_type = other DNA
gcagtagagtaggtagagattaggcatttttttttttttttttttttcagttgaacaacgccagcgtaacgatctaaa

SEQ ID NO: 716 - DNA, 69 bp, synthetic construct
  source 1..69: mol_type = other DNA
gcagtagagtaggtagagattaggcatttttttttttttttttttttgctgattgccgttcgtcgtgcc

SEQ ID NO: 717 - DNA, 85 bp, synthetic construct
  source 1..85: mol_type = other DNA
gcagtagagtaggtagagattaggcattttttttttttttttttttccactatttgttcagcgtgcctcctaactcacattaact

SEQ ID NO: 718 - DNA, 80 bp, synthetic construct
  source 1..80: mol_type = other DNA
gcagtagagtaggtagagattaggcattttttttttttttttttttaatagcccgcatcagagcactctgagggtggttt

SEQ ID NO: 719 - DNA, 77 bp, synthetic construct
  source 1..77: mol_type = other DNA
gcagtagagtaggtagagattaggcattttttttttttttttttttgaaatcggattattactttgccgccagcagt

SEQ ID NO: 720 - DNA, 60 bp, synthetic construct
  source 1..60: mol_type = other DNA
gcagtagagtaggtagagattaggcattttttttttttttttttttacaaactgatttag

SEQ ID NO: 721 - DNA, 70 bp, synthetic construct
  source 1..70: mol_type = other DNA
gcagtagagtaggtagagattaggcattttttttttttttttttttatgtaccggatacataagcaacac

SEQ ID NO: 722 - DNA, 80 bp, synthetic construct
  source 1..80: mol_type = other DNA
gcagtagagtaggtagagattaggcatttttttttttttttttttttcagatgttgttccaagagttgcagcaagcggtc

SEQ ID NO: 723 - DNA, 70 bp, synthetic construct
  source 1..70: mol_type = other DNA
gcagtagagtaggtagagattaggcatttttttttttttttttttttttttgggtttcgcaccaaagtca

SEQ ID NO: 724 - DNA, 75 bp, synthetic construct
  source 1..75: mol_type = other DNA
gcagtagagtaggtagagattaggcattttttttttttttttttttccactacgtatgagtagacggacagccat

SEQ ID NO: 725 - DNA, 69 bp, synthetic construct
  source 1..69: mol_type = other DNA
gcagtagagtaggtagagattaggcattttttttttttttttttttcactggtgaaagaacggaagaaa

SEQ ID NO: 726 - DNA, 71 bp, synthetic construct
  source 1..71: mol_type = other DNA
gcagtagagtaggtagagattaggcattttttttttttttttttttccagaggaagatcggccttgctggt

SEQ ID NO: 727 - DNA, 77 bp, synthetic construct
  source 1..77: mol_type = other DNA
gcagtagagtaggtagagattaggcattttttttttttttttttttgctggaggcaaatcaacgtaacacggatatt

SEQ ID NO: 728 - DNA, 69 bp, synthetic construct
  source 1..69: mol_type = other DNA
gcagtagagtaggtagagattaggcattttttttttttttttttttgttttttcgtctcgtctggggtg

SEQ ID NO: 729 - DNA, 69 bp, synthetic construct
  source 1..69: mol_type = other DNA
gcagtagagtaggtagagattaggcattttttttttttttttttttaacgtcagcgtggtggtttcctg

SEQ ID NO: 730 - DNA, 44 bp, synthetic construct
  source 1..44: mol_type = other DNA
tgaccgtatttgaggggacgacggcagaagttttgtttaacgtg

SEQ ID NO: 731 - DNA, 49 bp, synthetic construct
  source 1..49: mol_type = other DNA
tcagttgaggagcaaaataaccgaccgaccgtaataattttttcacgtt

SEQ ID NO: 732 - DNA, 57 bp, synthetic construct
  source 1..57: mol_type = other DNA
gatttaggatctacgttaataaaaagagccaggcccacgctttacattggcagattt

SEQ ID NO: 733 - DNA, 39 bp, synthetic construct
  source 1..39: mol_type = other DNA
aagcaataaagcctcagagcataaatgcagaacaaaggc

SEQ ID NO: 734 - DNA, 52 bp, synthetic construct
  source 1..52: mol_type = other DNA
gtaagcaagctggcggttatccggtattcgctgagactcctttgccccagca

SEQ ID NO: 735 - DNA, 59 bp, synthetic construct
  source 1..59: mol_type = other DNA
agagcacatccaacaatcgcggaaaaagcgctaggtttttcgctgggtggtgaagggat

SEQ ID NO: 736 - DNA, 48 bp, synthetic construct
  source 1..48: mol_type = other DNA
catgttacattccatagcgaaccagaccggaagcctgatttcgcaaga

SEQ ID NO: 737 - DNA, 63 bp, synthetic construct
  source 1..63: mol_type = other DNA
ttagccggtcgcctgaaacggctaatatattttagttaatttttttcatcttctgacctaaat

SEQ ID NO: 738 - DNA, 50 bp, synthetic construct
  source 1..50: mol_type = other DNA
aacgaggccattcagtgaataagggaattgagtcttaaacagcttgatat

SEQ ID NO: 739 - DNA, 61 bp, synthetic construct
  source 1..61: mol_type = other DNA
gtaagagctgaattaccttatgcgaatctttttaatagtaaattgggcttgaggagggggt

SEQ ID NO: 740 - DNA, 54 bp, synthetic construct
  source 1..54: mol_type = other DNA
cagcctttacagagagaataacgaggaaacagcccaataataagagtttgctca

SEQ ID NO: 741 - DNA, 23 bp, synthetic construct
  source 1..23: mol_type = other DNA
taactttaatcattgaacactat

SEQ ID NO: 742 - DNA, 55 bp, synthetic construct
  source 1..55: mol_type = other DNA
tcattacccaaatcaaggagcactatcggtttagtaataaacaattcgaggaaat

SEQ ID NO: 743 - DNA, 47 bp, synthetic construct
  source 1..47: mol_type = other DNA
taatcttgcaatagatttaaaagttcctttgctttctgtatgggatt

SEQ ID NO: 744 - DNA, 59 bp, synthetic construct
  source 1..59: mol_type = other DNA
gaatcttaccaacgcttggccttccgcactcgccatcttaatatcccaacatgttcagc

SEQ ID NO: 745 - DNA, 56 bp, synthetic construct
  source 1..56: mol_type = other DNA
gctgcgcaaggagcggagaacgtctcctgtttgagttgcagcaagcggtccacgct

SEQ ID NO: 746 - DNA, 48 bp, synthetic construct
  source 1..48: mol_type = other DNA
tcatacaaccaggcaaagctttttccattcgccattcagttttcacgg

SEQ ID NO: 747 - DNA, 40 bp, synthetic construct
  source 1..40: mol_type = other DNA
gaggtgaggcggtcagaccgtgcaggttgatatagcatgt

SEQ ID NO: 748 - DNA, 56 bp, synthetic construct
  source 1..56: mol_type = other DNA
tattaacaacgttaatattttgacggagtgagtagtatcatatgcgttctagaaaa

SEQ ID NO: 749 - DNA, 52 bp, synthetic construct
  source 1..52: mol_type = other DNA
ccgcctgcctgatagccctaaaacctgagaagatacctacgagctaaattga

SEQ ID NO: 750 - DNA, 36 bp, synthetic construct
  source 1..36: mol_type = other DNA
ccagacgacgataaaatttttccaaaatagcgagag

SEQ ID NO: 751 - DNA, 39 bp, synthetic construct
  source 1..39: mol_type = other DNA
agagccgtacaagaacaacgggtatgacccccaagggtt

SEQ ID NO: 752 - DNA, 32 bp, synthetic construct
  source 1..32: mol_type = other DNA
aatagtaagaacgagttcaaatatcgcgtttt

SEQ ID NO: 753 - DNA, 48 bp, synthetic construct
  source 1..48: mol_type = other DNA
aagccagaaaatcaagattagttgttttttattttgcacccatcatta

SEQ ID NO: 754 - DNA, 56 bp, synthetic construct
  source 1..56: mol_type = other DNA
aggcgcataacgcaaagacaccttagagcttaattgctgaggacagatctcatctt

SEQ ID NO: 755 - DNA, 47 bp, synthetic construct
  source 1..47: mol_type = other DNA
caatattgaaaaatctaaagcatgacgttggggcttgcatatcaggt

SEQ ID NO: 756 - DNA, 32 bp, synthetic construct
  source 1..32: mol_type = other DNA
ctttacaaaagggacaaaccgttgtagcaata

SEQ ID NO: 757 - DNA, 30 bp, synthetic construct
  source 1..30: mol_type = other DNA
cctcagagccaccacactcgtcaccaatga

SEQ ID NO: 758 - DNA, 55 bp, synthetic construct
  source 1..55: mol_type = other DNA
ccaccggaaccgcctacaaacaaataaatccgctacaatattttcatactgaaca

SEQ ID NO: 759 - DNA, 51 bp, synthetic construct
  source 1..51: mol_type = other DNA
tccagcgcagcagatttttgccgggttacctgcagccagccattcaggaaa

SEQ ID NO: 760 - DNA, 47 bp, synthetic construct
  source 1..47: mol_type = other DNA
tgcggtattcataaacatcccttaaaaccgggtcgaggtgccgtaaa

SEQ ID NO: 761 - DNA, 32 bp, synthetic construct
  source 1..32: mol_type = other DNA
gtccatcacgcaaattttctggccacacgacc

SEQ ID NO: 762 - DNA, 47 bp, synthetic construct
  source 1..47: mol_type = other DNA
atcaacagttgaaagcttgccctagggtagctaaattgtaaatccaa

SEQ ID NO: 763 - DNA, 50 bp, synthetic construct
  source 1..50: mol_type = other DNA
aacttaaatttccgttttttcgtctcgttttttgctggcagcctccggcc

SEQ ID NO: 764 - DNA, 48 bp, synthetic construct
  source 1..48: mol_type = other DNA
aaaaatcccgcgtctggtcggattgaagatcgcactccagttcgcgtc

SEQ ID NO: 765 - DNA, 56 bp, synthetic construct
  source 1..56: mol_type = other DNA
ctccgtgggaacaaacctcgcacaggcggcctttagtgatgaagggtaaacaaccc

SEQ ID NO: 766 - DNA, 28 bp, synthetic construct
  source 1..28: mol_type = other DNA
ggcggattaggaacaaatcagagtgagc

SEQ ID NO: 767 - DNA, 46 bp, synthetic construct
  source 1..46: mol_type = other DNA
ggtgagatacttttgcgggagaagcctttatggtgtagcgtaaaac

SEQ ID NO: 768 - DNA, 48 bp, synthetic construct
  source 1..48: mol_type = other DNA
aaggccggagacagtccagtacattcaatagtgaatttatttgaaata

SEQ ID NO: 769 - DNA, 52 bp, synthetic construct
  source 1..52: mol_type = other DNA
tgactattatagtcatttttaagcaaagcggattgcaaatggtcaataacct

SEQ ID NO: 770 - DNA, 46 bp, synthetic construct
  source 1..46: mol_type = other DNA
gaaagactagatttagtttgaccattagatacaaaagaagattgat

SEQ ID NO: 771 - DNA, 55 bp, synthetic construct
  source 1..55: mol_type = other DNA
gcttcaaataacagttgattcccaattctgcaatgtttagactggcgaacaccag

SEQ ID NO: 772 - DNA, 38 bp, synthetic construct
  source 1..38: mol_type = other DNA
gctccttctgtagctcaacatgttttaaattaagggaa

SEQ ID NO: 773 - DNA, 32 bp, synthetic construct
  source 1..32: mol_type = other DNA
ttcatatggtttaccataggtgtaaaccgcca

SEQ ID NO: 774 - DNA, 39 bp, synthetic construct
  source 1..39: mol_type = other DNA
tagcaatagctatcttggtcaagagaaggattaggatta

SEQ ID NO: 775 - DNA, 32 bp, synthetic construct
  source 1..32: mol_type = other DNA
accgaagcgctggtctgaaagggggaagggcg

SEQ ID NO: 776 - DNA, 52 bp, synthetic construct
  source 1..52: mol_type = other DNA
tttctcccaagtgtaccgagattttttgggttgagtgttgttccggcgaaaa

SEQ ID NO: 777 - DNA, 32 bp, synthetic construct
  source 1..32: mol_type = other DNA
gccttcctcacacccgtcactgccttccacac

SEQ ID NO: 778 - DNA, 39 bp, synthetic construct
  source 1..39: mol_type = other DNA
tatccccctcaaatgaaatcaaatcatacaggcaaggca

SEQ ID NO: 779 - DNA, 47 bp, synthetic construct
  source 1..47: mol_type = other DNA
cgcgaaacttttcatgatggcaataaaaggctccaaaaggcaacttt

SEQ ID NO: 780 - DNA, 48 bp, synthetic construct
  source 1..48: mol_type = other DNA
agaatacatggcacggaataagtttattttgtaccgaaataaagaaat

SEQ ID NO: 781 - DNA, 45 bp, synthetic construct
  source 1..45: mol_type = other DNA
cgattgaaataaacagagcctaatttgcccattgacagtcaccga

SEQ ID NO: 782 - DNA, 48 bp, synthetic construct
  source 1..48: mol_type = other DNA
agcgtggtccttgccggacttgtgagacgcaccctgagagatggtggt

SEQ ID NO: 783 - DNA, 45 bp, synthetic construct
  source 1..45: mol_type = other DNA
agctctcagcgaaacgtacagtttttgccatgtttaccagtcccg

SEQ ID NO: 784 - DNA, 46 bp, synthetic construct
  source 1..46: mol_type = other DNA
gatattcccctcagagccgccaaccgtaatttttcggtcatagccc

SEQ ID NO: 785 - DNA, 54 bp, synthetic construct
  source 1..54: mol_type = other DNA
ctcattttcattaaatataaaacaccgaacgaaccaccagctttttagacagga

SEQ ID NO: 786 - DNA, 48 bp, synthetic construct
  source 1..48: mol_type = other DNA
atcagaaagaataaacaatagaaaggaacaacgtttcagcgctcaatc

SEQ ID NO: 787 - DNA, 47 bp, synthetic construct
  source 1..47: mol_type = other DNA
aagattgtcaagagaagagaagagaaatcaatatatgtgagtgaata

SEQ ID NO: 788 - DNA, 43 bp, synthetic construct
  source 1..43: mol_type = other DNA
cggtgcccacgcgtgcctgttcccagctttaaccacgtagcca

SEQ ID NO: 789 - DNA, 47 bp, synthetic construct
  source 1..47: mol_type = other DNA
gggagttaaaggccgcttttgcgaactggctcgccaaaaggaattac

SEQ ID NO: 790 - DNA, 30 bp, synthetic construct
  source 1..30: mol_type = other DNA
gacagcatcggaacggacgagaacctgctc

SEQ ID NO: 791 - DNA, 32 bp, synthetic construct
  source 1..32: mol_type = other DNA
cagaggctggtgaattgaaggttaatagaacc

SEQ ID NO: 792 - DNA, 38 bp, synthetic construct
  source 1..38: mol_type = other DNA
aaatacgtgaattatcaaagaaaccaccagaaatgaat

SEQ ID NO: 793 - DNA, 53 bp, synthetic construct
  source 1..53: mol_type = other DNA
ataacatcaatattaccgccagccattgcaacacaactctattagaatagatt

SEQ ID NO: 794 - DNA, 31 bp, synthetic construct
  source 1..31: mol_type = other DNA
aagctaaagatattcaaccgttctatttcat

SEQ ID NO: 795 - DNA, 40 bp, synthetic construct
  source 1..40: mol_type = other DNA
aatattgagtctttccagccatattatttatctacataca

SEQ ID NO: 796 - DNA, 55 bp, synthetic construct
  source 1..55: mol_type = other DNA
cggaaattaagggcgaaccctcagtcaccgtactcaggaggtttagtcacaatca

SEQ ID NO: 797 - DNA, 56 bp, synthetic construct
  source 1..56: mol_type = other DNA
ccgttttttttatcctaacgattttttgtttatattacgcagcgctaatatcagag

SEQ ID NO: 798 - DNA, 45 bp, synthetic construct
  source 1..45: mol_type = other DNA
aatcattacacagggaagcgcatttttttgacggaatgaaaatag

SEQ ID NO: 799 - DNA, 47 bp, synthetic construct
  source 1..47: mol_type = other DNA
ttcaaaagacggtaatatgattacaggtagaaagattcaccctgtaa

SEQ ID NO: 800 - DNA, 39 bp, synthetic construct
  source 1..39: mol_type = other DNA
gccggcgagctgcgcgtccggcactaaatgtgagcgagt

SEQ ID NO: 801 - DNA, 46 bp, synthetic construct
  source 1..46: mol_type = other DNA
tatccgcagctgttttttttggtctatcagggcgatggcccactac

SEQ ID NO: 802 - DNA, 47 bp, synthetic construct
  source 1..47: mol_type = other DNA
agtatgttagcaaacgtagaaaaccaatccaaaagacaaatcgagaa

SEQ ID NO: 803 - DNA, 54 bp, synthetic construct
  source 1..54: mol_type = other DNA
cgtaaaaaaagccatatattttaatggggagaggcggtttgcgtattgggcgcc

SEQ ID NO: 804 - DNA, 61 bp, synthetic construct
  source 1..61: mol_type = other DNA
gcaaaattgccggagaaaacgagacatcaagatgggtttttttataggcgaattattcatt

SEQ ID NO: 805 - DNA, 44 bp, synthetic construct
  source 1..44: mol_type = other DNA
ttgagtaacattacttccattacggatataccaactttgaaaga

SEQ ID NO: 806 - DNA, 34 bp, synthetic construct
  source 1..34: mol_type = other DNA
gtctttccgcagtctctgcccgtaaccagagcca

SEQ ID NO: 807 - DNA, 55 bp, synthetic construct
  source 1..55: mol_type = other DNA
gcaccgtcggtggcctatttgctcaaatcagttccgggaggttttgaacgttgta

SEQ ID NO: 808 - DNA, 48 bp, synthetic construct
  source 1..48: mol_type = other DNA
aagggaagaagagctttttcccgatttagagcttgacggcaacgcttt

SEQ ID NO: 809 - DNA, 47 bp, synthetic construct
  source 1..47: mol_type = other DNA
gctacagggccatcaatcggccaacgcgctttcccttaggcctaatg

SEQ ID NO: 810 - DNA, 55 bp, synthetic construct
  source 1..55: mol_type = other DNA
tcaccagtcaacagagtctaaaatatctttacgtaacaataaagactaaagtaca

SEQ ID NO: 811 - DNA, 32 bp, synthetic construct
  source 1..32: mol_type = other DNA
ccgaacgttccagaacacttgcctgagtagaa

SEQ ID NO: 812 - DNA, 51 bp, synthetic construct
  source 1..51: mol_type = other DNA
tcaccagtgagccagtgtaatttaggcagaggacgacgacctattagcaag

SEQ ID NO: 813 - DNA, 47 bp, synthetic construct
  source 1..47: mol_type = other DNA
agcaccattacctataccgccacgaggcaggtcagacgataacgagc

SEQ ID NO: 814 - DNA, 32 bp, synthetic construct
  source 1..32: mol_type = other DNA
tagcgtttattcattacaaaaggtaacgcctg

SEQ ID NO: 815 - DNA, 48 bp, synthetic construct
  source 1..48: mol_type = other DNA
tcccacccattatcaacaatagataagtctcgcggaaccttattaaag

SEQ ID NO: 816 - DNA, 48 bp, synthetic construct
  source 1..48: mol_type = other DNA
aaggtttcctttgacgtaccgagctcctcacagttgaggaacagtatc

SEQ ID NO: 817 - DNA, 41 bp, synthetic construct
  source 1..41: mol_type = other DNA
cgattaaagggaggtaatgggtagtgaaagccaacgctcaa

SEQ ID NO: 818 - DNA, 23 bp, synthetic construct
  source 1..23: mol_type = other DNA
tcatggaatgtttttataatcag

SEQ ID NO: 819 - DNA, 43 bp, synthetic construct
  source 1..43: mol_type = other DNA
taacgcgtctgaagcgcgaaaacagtgccacgctgcgaactaa

SEQ ID NO: 820 - DNA, 34 bp, synthetic construct
  source 1..34: mol_type = other DNA
gccggaaaatcggccttgcgccaacattttccag

SEQ ID NO: 821 - DNA, 51 bp, synthetic construct
  source 1..51: mol_type = other DNA
gcgttttttttcatcggcacagtagcgacagaatctttttagtttgccttt

SEQ ID NO: 822 - DNA, 44 bp, synthetic construct
  source 1..44: mol_type = other DNA
ttaattacatttaacaagctgatgagatcttacataacattata

SEQ ID NO: 823 - DNA, 39 bp, synthetic construct
  source 1..39: mol_type = other DNA
tcaaaagaagatgatgaaacaaaatgaccatctttaaac

SEQ ID NO: 824 - DNA, 45 bp, synthetic construct
  source 1..45: mol_type = other DNA
tcaattacggaagcccaaatattcttttgccaatggtttaatttc

SEQ ID NO: 825 - DNA, 39 bp, synthetic construct
  source 1..39: mol_type = other DNA
tcgcgcagataactatatgtaaatgctgatgcgtcgaaa

SEQ ID NO: 826 - DNA, 54 bp, synthetic construct
  source 1..54: mol_type = other DNA
gctttgaataccaagttacaaaaaattcgatccgatagcgtccaatcagcgaaa

SEQ ID NO: 827 - DNA, 47 bp, synthetic construct
  source 1..47: mol_type = other DNA
cagatgaatatacagtaacagtactttaattataccaagccgaactg

SEQ ID NO: 828 - DNA, 48 bp, synthetic construct
  source 1..48: mol_type = other DNA
tgcgtagattttcaggtttgcggactaaaacagaacggtgatcaagag

SEQ ID NO: 829 - DNA, 42 bp, synthetic construct
  source 1..42: mol_type = other DNA
tcgagaggttcaggtttttatagcaaggtaccaggcggataa

SEQ ID NO: 830 - DNA, 54 bp, synthetic construct
  source 1..54: mol_type = other DNA
tgccgagataacccatttttaagaattgagttagcaataataacggaataccca

SEQ ID NO: 831 - DNA, 47 bp, synthetic construct
  source 1..47: mol_type = other DNA
gcggggtcaagaaacgaggcgttttaaaacgcgcccaatgtaccgta

SEQ ID NO: 832 - DNA, 45 bp, synthetic construct
  source 1..45: mol_type = other DNA
tccgaatgattgcccttctttttccgcctgggaaacagcggatca

SEQ ID NO: 833 - DNA, 44 bp, synthetic construct
  source 1..44: mol_type = other DNA
ccttataaatcggctttcatggcggttgacgatgctgattgccg

SEQ ID NO: 834 - DNA, 47 bp, synthetic construct
  source 1..47: mol_type = other DNA
ggaaaccttttcttttcaccagtgagacggagcagttgcaaatagac

SEQ ID NO: 835 - DNA, 48 bp, synthetic construct
  source 1..48: mol_type = other DNA
aaaacatatctgtaaatcgtcgctattaattaaatgcaatttaaccaa

SEQ ID NO: 836 - DNA, 40 bp, synthetic construct
  source 1..40: mol_type = other DNA
gcgatagcttagattattaaataaagccccaaaattgtaa

SEQ ID NO: 837 - DNA, 48 bp, synthetic construct
  source 1..48: mol_type = other DNA
taggtctgttgaattaccttttttaatggaaaaaatcaccgagagtct

SEQ ID NO: 838 - DNA, 39 bp, synthetic construct
  source 1..39: mol_type = other DNA
agagactacctttttaacctccgatttttgaaaattaat

SEQ ID NO: 839 - DNA, 55 bp, synthetic construct
  source 1..55: mol_type = other DNA
caaagaacgcgagatcacggagatcaacaggtaaagtacggtgtctggaagtttc

SEQ ID NO: 840 - DNA, 48 bp, synthetic construct
  source 1..48: mol_type = other DNA
ctgattgtatcgggagaaacaataacggattcgcaaactcttgtatca

SEQ ID NO: 841 - DNA, 30 bp, synthetic construct
  source 1..30: mol_type = other DNA
ccctcagaccagtacaagcaagaccaagta

SEQ ID NO: 842 - DNA, 46 bp, synthetic construct
  source 1..46: mol_type = other DNA
ccctcattgttgatataagtatagcccggaagccagagggtaattg

SEQ ID NO: 843 - DNA, 55 bp, synthetic construct
  source 1..55: mol_type = other DNA
cccaataggaaaaagtattaagagtaagaacgcaatgaaaaacaaagttaccaga

SEQ ID NO: 844 - DNA, 55 bp, synthetic construct
  source 1..55: mol_type = other DNA
ccagtcaaaagaatagcgcggtcacacgtggcgtctgccagccctgcatcagacg

SEQ ID NO: 845 - DNA, 48 bp, synthetic construct
  source 1..48: mol_type = other DNA
cgttgcgcccgcgcttgaaattgtcgtgagcctcgaattcgtaatcat

SEQ ID NO: 846 - DNA, 55 bp, synthetic construct
  source 1..55: mol_type = other DNA
cataattaaatccttgcaatcatagcctgagtcaaggataaaaatttttagaacc

SEQ ID NO: 847 - DNA, 56 bp, synthetic construct
  source 1..56: mol_type = other DNA
ttaatggtcaaaatcacattgcctatcaatattcggttgtaccaaaaacattatga

SEQ ID NO: 848 - DNA, 32 bp, synthetic construct
  source 1..32: mol_type = other DNA
atcagatgaggaagttttaattgtaacaacta

SEQ ID NO: 849 - DNA, 29 bp, synthetic construct
  source 1..29: mol_type = other DNA
cacagacgaaggtattatcaccggaggtt

SEQ ID NO: 850 - DNA, 47 bp, synthetic construct
  source 1..47: mol_type = other DNA
tagcattcgaaccgcccattcaacatagaaaataaaggtggcaacat

SEQ ID NO: 851 - DNA, 53 bp, synthetic construct
  source 1..53: mol_type = other DNA
caaactacatcctaatttacgagcatgtagatttttaccaatcaataactgaa

SEQ ID NO: 852 - DNA, 46 bp, synthetic construct
  source 1..46: mol_type = other DNA
acactgagtttcgtcagccaccaccctgaacaaagtgccaataaga

SEQ ID NO: 853 - DNA, 32 bp, synthetic construct
  source 1..32: mol_type = other DNA
tatattcgcaaccatccagcaaattttgaatg

SEQ ID NO: 854 - DNA, 48 bp, synthetic construct
  source 1..48: mol_type = other DNA
aacatacgaaatcaagtcctgtgttttgctcggagccgggtcactgtt

SEQ ID NO: 855 - DNA, 40 bp, synthetic construct
  source 1..40: mol_type = other DNA
gcataaagtaactcacattaattgttaatgaaaaataatt

SEQ ID NO: 856 - DNA, 31 bp, synthetic construct
  source 1..31: mol_type = other DNA
tgtaaagcctggggtatacaaatctttcctc

SEQ ID NO: 857 - DNA, 37 bp, synthetic construct
  source 1..37: mol_type = other DNA
ttgcgaatgtgataaataaggcgagacgcttcgatga

SEQ ID NO: 858 - DNA, 46 bp, synthetic construct
  source 1..46: mol_type = other DNA
ttgaaaatgctttcgattgaggacagctgctgcagacggtcaatca

SEQ ID NO: 859 - DNA, 32 bp, synthetic construct
  source 1..32: mol_type = other DNA
ttgtcgtctagttagcgtaacgataaattatt

SEQ ID NO: 860 - DNA, 32 bp, synthetic construct
  source 1..32: mol_type = other DNA
tttcaacgaatgtgtaggtaaagaaccttgct

SEQ ID NO: 861 - DNA, 55 bp, synthetic construct
  source 1..55: mol_type = other DNA
tcttaccagtataaccatcacccagccggaatatggttgaatgcgccggcctcag

SEQ ID NO: 862 - DNA, 52 bp, synthetic construct
  source 1..52: mol_type = other DNA
ttcaacataaaggaaatatttaaaacaggcggaacaacattggcgcatcgta

SEQ ID NO: 863 - DNA, 55 bp, synthetic construct
  source 1..55: mol_type = other DNA
ttgctaaaagctcattttgcggaacatcatattcctgattaataatggagcgatt

SEQ ID NO: 864 - DNA, 48 bp, synthetic construct
  source 1..48: mol_type = other DNA
acgttagtctaaagttatttgggatacgaaggtgaccttctacagacc

SEQ ID NO: 865 - DNA, 43 bp, synthetic construct
  source 1..43: mol_type = other DNA
caagaaaaatttcataatcaaaatcatttttcggataaacagt

SEQ ID NO: 866 - DNA, 53 bp, synthetic construct
  source 1..53: mol_type = other DNA
gcactaaatcactgcatcgttaacggcattgtcactgagaatgcggcgggccg

SEQ ID NO: 867 - DNA, 39 bp, synthetic construct
  source 1..39: mol_type = other DNA
gcgaacactggtgtttttgttcagcaaaaccggggtcat

SEQ ID NO: 868 - DNA, 37 bp, synthetic construct
  source 1..37: mol_type = other DNA
cagtagggcttaacaggaggcgttagaattaaaaata

SEQ ID NO: 869 - DNA, 60 bp, synthetic construct
  source 1..60: mol_type = other DNA
gaatcgccatatttaacaactggtaataattagagcccgccaccagaacacatttgagga

SEQ ID NO: 870 - DNA, 40 bp, synthetic construct
  source 1..40: mol_type = other DNA
caataaacaaagtaattctgtccaagtaccgaaaggtgaa

SEQ ID NO: 871 - DNA, 46 bp, synthetic construct
  source 1..46: mol_type = other DNA
aagttaatgtacatcgacataaagggtggttgtcgtgccagctgca

SEQ ID NO: 872 - DNA, 55 bp, synthetic construct
  source 1..55: mol_type = other DNA
ttccggcagaatttgtgagagcatcgcttctggtgccggacgggggttagaaagg

SEQ ID NO: 873 - DNA, 47 bp, synthetic construct
  source 1..47: mol_type = other DNA
caccaacctaaaaaggagccctcaaataagagaatataagcattttc

SEQ ID NO: 874 - DNA, 48 bp, synthetic construct
  source 1..48: mol_type = other DNA
aatgccactattaattaatcaccaccagagccgccgccagaggctggc

SEQ ID NO: 875 - DNA, 40 bp, synthetic construct
  source 1..40: mol_type = other DNA
atcagcttctccaaaatcatcaatataaaaactttttcaa

SEQ ID NO: 876 - DNA, 55 bp, synthetic construct
  source 1..55: mol_type = other DNA
cagaggtggagctgggattaacctcaccggatcataacggaacgttttaagaaaa

SEQ ID NO: 877 - DNA, 29 bp, synthetic construct
  source 1..29: mol_type = other DNA
gcttttgatgcactccatcagcagcttac

SEQ ID NO: 878 - DNA, 40 bp, synthetic construct
  source 1..40: mol_type = other DNA
aaacgacggccagtgcttgggtaaattacgccgatagccg

SEQ ID NO: 879 - DNA, 53 bp, synthetic construct
  source 1..53: mol_type = other DNA
aagaattagaggcataagttcagaacccttttttcagactgcggaatcgtcat

SEQ ID NO: 880 - DNA, 56 bp, synthetic construct
  source 1..56: mol_type = other DNA
atataatgttgataagaggtcatttttaacgtagaacctaccatatcaaaggagcg

SEQ ID NO: 881 - DNA, 48 bp, synthetic construct
  source 1..48: mol_type = other DNA
aaagaactggcatgattaagactcctacgtcaaagagaattacgtagg

SEQ ID NO: 882 - DNA, 55 bp, synthetic construct
  source 1..55: mol_type = other DNA
ataaaagaagttacaagggcgaaagaggcaaatgcacgtaaaacagccaccctca

SEQ ID NO: 883 - DNA, 40 bp, synthetic construct
  source 1..40: mol_type = other DNA
aaaattcgtgtacccctctgccagatgggataggtcacgt

SEQ ID NO: 884 - DNA, 48 bp, synthetic construct
  source 1..48: mol_type = other DNA
atgcaactcaggattagagagtacccttttacttggattatacttctg

SEQ ID NO: 885 - DNA, 58 bp, synthetic construct
  source 1..58: mol_type = other DNA
tccgatagttgcgccgacaatgacaagtcgctgagaagaaaaaataccacattcaact

SEQ ID NO: 886 - DNA, 54 bp, synthetic construct
  source 1..54: mol_type = other DNA
atatagaaggctcagacaagagtccacattattctgaaacatgcccatagcaag

SEQ ID NO: 887 - DNA, 44 bp, synthetic construct
  source 1..44: mol_type = other DNA
aacgcggtctgctcatttgccgccgcaacagcatcggcaaaatc

SEQ ID NO: 888 - DNA, 32 bp, synthetic construct
  source 1..32: mol_type = other DNA
cagagcggattttgttagcctgttaccggaat

SEQ ID NO: 889 - DNA, 51 bp, synthetic construct
  source 1..51: mol_type = other DNA
taatgcaactgtagctaatgcccagtaacagtgaatttacggggtcaatgt

SEQ ID NO: 890 - DNA, 40 bp, synthetic construct
  source 1..40: mol_type = other DNA
agtttggacagcaaccgcaaggaaactgtcgccacgaggt

SEQ ID NO: 891 - DNA, 64 bp, synthetic construct
  source 1..64: mol_type = other DNA
aacgtggggaaccctaaaggagcaatgccaacggcactgcggcccgcgcctgtgatacagtaat

SEQ ID NO: 892 - DNA, 51 bp, synthetic construct
  source 1..51: mol_type = other DNA
taaaacgagccttatggaaagcttatcattcctttttagaacgggtattaa

SEQ ID NO: 893 - DNA, 54 bp, synthetic construct
  source 1..54: mol_type = other DNA
aggaaaccatagcgaacctcccgacttggctcgccagggttttcccagtcacat

SEQ ID NO: 894 - DNA, 32 bp, synthetic construct
  source 1..32: mol_type = other DNA
taacgagtgtactggtttatcggtgtgtggtg

SEQ ID NO: 895 - DNA, 44 bp, synthetic construct
  source 1..44: mol_type = other DNA
ttaagtgccttgcatacatgagttttaaccgttccacgggcctc

SEQ ID NO: 896 - DNA, 41 bp, synthetic construct
  source 1..41: mol_type = other DNA
tgatggaacggagtgctgcaaggcgattaagcaagctaata

SEQ ID NO: 897 - DNA, 33 bp, synthetic construct
  source 1..33: mol_type = other DNA
agcgtcaggtgtccaggtaagcgtcctgtgcca

SEQ ID NO: 898 - DNA, 30 bp, synthetic construct
  source 1..30: mol_type = other DNA
ggctggaggaacgcgcctgtacgtcaaagg

SEQ ID NO: 899 - DNA, 32 bp, synthetic construct
  source 1..32: mol_type = other DNA
cataacccccaataaaaatcaggtctttaccc

SEQ ID NO: 900 - DNA, 48 bp, synthetic construct
  source 1..48: mol_type = other DNA
aaaccctcattttaagggatcgtcgggtagctgcttaggtaaacaaaa

SEQ ID NO: 901 - DNA, 32 bp, synthetic construct
  source 1..32: mol_type = other DNA
gaactcaacctcagagcagcaaaacttgagcc

SEQ ID NO: 902 - DNA, 32 bp, synthetic construct
  source 1..32: mol_type = other DNA
tgaggccatctttaatatggattaataagcaa

SEQ ID NO: 903 - DNA, 32 bp, synthetic construct
  source 1..32: mol_type = other DNA
gccctgcgtccccgggagcacgtagcgcgtac

SEQ ID NO: 904 - DNA, 40 bp, synthetic construct
  source 1..40: mol_type = other DNA
gtttagctatattttcatttggggctttttcgagctgaaa

SEQ ID NO: 905 - DNA, 46 bp, synthetic construct
  source 1..46: mol_type = other DNA
aggtggcatcaattctactaatagtagtagcattaacattcgttta

SEQ ID NO: 906 - DNA, 24 bp, synthetic construct
  source 1..24: mol_type = other DNA
acggtacgccagaatcatcgccat

SEQ ID NO: 907 - DNA, 24 bp, synthetic construct
  source 1..24: mol_type = other DNA
aaccatcgatagcagcccctcaga

SEQ ID NO: 908 - DNA, 31 bp, synthetic construct
  source 1..31: mol_type = other DNA
cttctttgattagtatttagaaggtattaaa

SEQ ID NO: 909 - DNA, 40 bp, synthetic construct
  source 1..40: mol_type = other DNA
cttctgacctgaaagcgttttttagaatacgtggcacaga

SEQ ID NO: 910 - DNA, 31 bp, synthetic construct
  source 1..31: mol_type = other DNA
ccagtcagcaccttgctgaacctcaaatatc

SEQ ID NO: 911 - DNA, 37 bp, synthetic construct
  source 1..37: mol_type = other DNA
gcttttgcatttcgcatcaaaaagattaagactgagt

SEQ ID NO: 912 - DNA, 48 bp, synthetic construct
  source 1..48: mol_type = other DNA
tgcaggcgcttttttttgcactcaatccgccgggcgcggtggtggtgc

SEQ ID NO: 913 - DNA, 24 bp, synthetic construct
  source 1..24: mol_type = other DNA
gctattagccgagtaaaagagtct

SEQ ID NO: 914 - DNA, 24 bp, synthetic construct
  source 1..24: mol_type = other DNA
aacgagtatctggtcagttggcaa

SEQ ID NO: 915 - DNA, 87 bp, synthetic construct
  source 1..87: mol_type = other DNA
gcagtagagtaggtagagattaggcattgcagtagagtaggtagagattaggcaagcgtcaggtgtccaggtaagcgtcctgtgcca

SEQ ID NO: 916 - DNA, 84 bp, synthetic construct
  source 1..84: mol_type = other DNA
gcagtagagtaggtagagattaggcattgcagtagagtaggtagagattaggcaggctggaggaacgcgcctgtacgtcaaagg

SEQ ID NO: 917 - DNA, 86 bp, synthetic construct
  source 1..86: mol_type = other DNA
gcagtagagtaggtagagattaggcattgcagtagagtaggtagagattaggcacataacccccaataaaaatcaggtctttaccc

SEQ ID NO: 918 - DNA, 102 bp, synthetic construct
  source 1..102: mol_type = other DNA
gcagtagagtaggtagagattaggcattgcagtagagtaggtagagattaggcaaaaccctcattttaagggatcgtcgggtagctgcttaggtaaacaaaa

SEQ ID NO: 919 - DNA, 86 bp, synthetic construct
  source 1..86: mol_type = other DNA
gcagtagagtaggtagagattaggcattgcagtagagtaggtagagattaggcagaactcaacctcagagcagcaaaacttgagcc

SEQ ID NO: 920 - DNA, 86 bp, synthetic construct
  source 1..86: mol_type = other DNA
gcagtagagtaggtagagattaggcattgcagtagagtaggtagagattaggcatgaggccatctttaatatggattaataagcaa

SEQ ID NO: 921 - DNA, 86 bp, synthetic construct
  source 1..86: mol_type = other DNA
gcagtagagtaggtagagattaggcattgcagtagagtaggtagagattaggcagccctgcgtccccgggagcacgtagcgcgtac

SEQ ID NO: 922 - DNA, 94 bp, synthetic construct
  source 1..94: mol_type = other DNA
gcagtagagtaggtagagattaggcattgcagtagagtaggtagagattaggcagtttagctatattttcatttggggctttttcgagctgaaa

SEQ ID NO: 923 - DNA, 100 bp, synthetic construct
  source 1..100: mol_type = other DNA
gcagtagagtaggtagagattaggcattgcagtagagtaggtagagattaggcaaggtggcatcaattctactaatagtagtagcattaacattcgttta

SEQ ID NO: 924 - DNA, 78 bp, synthetic construct
  source 1..78: mol_type = other DNA
gcagtagagtaggtagagattaggcattgcagtagagtaggtagagattaggcaacggtacgccagaatcatcgccat

SEQ ID NO: 925 - DNA, 78 bp, synthetic construct
  source 1..78: mol_type = other DNA
gcagtagagtaggtagagattaggcattgcagtagagtaggtagagattaggcaaaccatcgatagcagcccctcaga

SEQ ID NO: 926 - DNA, 85 bp, synthetic construct
  source 1..85: mol_type = other DNA
gcagtagagtaggtagagattaggcattgcagtagagtaggtagagattaggcacttctttgattagtatttagaaggtattaaa

SEQ ID NO: 927 - DNA, 94 bp, synthetic construct
  source 1..94: mol_type = other DNA
gcagtagagtaggtagagattaggcattgcagtagagtaggtagagattaggcacttctgacctgaaagcgttttttagaatacgtggcacaga

SEQ ID NO: 928 - DNA, 85 bp, synthetic construct
  source 1..85: mol_type = other DNA
gcagtagagtaggtagagattaggcattgcagtagagtaggtagagattaggcaccagtcagcaccttgctgaacctcaaatatc

SEQ ID NO: 929 - DNA, 91 bp, synthetic construct
  source 1..91: mol_type = other DNA
gcagtagagtaggtagagattaggcattgcagtagagtaggtagagattaggcagcttttgcatttcgcatcaaaaagattaagactgagt

SEQ ID NO: 930 - DNA, 102 bp, synthetic construct
  source 1..102: mol_type = other DNA
gcagtagagtaggtagagattaggcattgcagtagagtaggtagagattaggcatgcaggcgcttttttttgcactcaatccgccgggcgcggtggtggtgc

SEQ ID NO: 931 - DNA, 78 bp, synthetic construct
  source 1..78: mol_type = other DNA
gcagtagagtaggtagagattaggcattgcagtagagtaggtagagattaggcagctattagccgagtaaaagagtct

SEQ ID NO: 932 - DNA, 78 bp, synthetic construct
  source 1..78: mol_type = other DNA
gcagtagagtaggtagagattaggcattgcagtagagtaggtagagattaggcaaacgagtatctggtcagttggcaa

SEQ ID NO: 933 - DNA, 42 bp, synthetic construct
  source 1..42: mol_type = other DNA
tagttgcaaggaattgcgaataccctcgttccaatactgcgg

SEQ ID NO: 934 - DNA, 45 bp, synthetic construct
  source 1..45: mol_type = other DNA
agtaacagttaattttccagcttacggctgggtgatgaaatttac

SEQ ID NO: 935 - DNA, 32 bp, synthetic construct
  source 1..32: mol_type = other DNA
atcctgaggcgggccagcaaaacagataaatc

SEQ ID NO: 936 - DNA, 24 bp, synthetic construct
  source 1..24: mol_type = other DNA
tgatggcaatcatcatattcctga

SEQ ID NO: 937 - DNA, 32 bp, synthetic construct
  source 1..32: mol_type = other DNA
ttgtttggatcattttgcggaacaaagtttga

SEQ ID NO: 938 - DNA, 45 bp, synthetic construct
  source 1..45: mol_type = other DNA
cagaaggagcacgcgtgcctgtttttttttcgcgtccgtggtttt

SEQ ID NO: 939 - DNA, 32 bp, synthetic construct
  source 1..32: mol_type = other DNA
atttgcactaaacagttaatagtacagcgaaa

SEQ ID NO: 940 - DNA, 32 bp, synthetic construct
  source 1..32: mol_type = other DNA
ttatcagagcatcagaagccagcggctaaaca

SEQ ID NO: 941 - DNA, 42 bp, synthetic construct
  source 1..42: mol_type = other DNA
tattaattttaaaagaatttttccacgcccgaacaaatcgcg

SEQ ID NO: 942 - DNA, 55 bp, synthetic construct
  source 1..55: mol_type = other DNA
caaacaatttacctgatctttaggttgctttgcacacccgaagaaagcgaaagga

SEQ ID NO: 943 - DNA, 38 bp, synthetic construct
  source 1..38: mol_type = other DNA
tcaaaattatggaaggcagaaccagacataaagaaatt

SEQ ID NO: 944 - DNA, 48 bp, synthetic construct
  source 1..48: mol_type = other DNA
tttcaggtttaacgtcagatgaatattttaacaccgcctggagtgagc

SEQ ID NO: 945 - DNA, 39 bp, synthetic construct
  source 1..39: mol_type = other DNA
gtaccttttatagataatacatttgagattagcagaggc

SEQ ID NO: 946 - DNA, 40 bp, synthetic construct
  source 1..40: mol_type = other DNA
atttcggagatacagggagccaccagtaccgccaccctca

SEQ ID NO: 947 - DNA, 28 bp, synthetic construct
  source 1..28: mol_type = other DNA
gtttcctaattcgtgctattattattta

SEQ ID NO: 948 - DNA, 55 bp, synthetic construct
  source 1..55: mol_type = other DNA
tagaatcatactatggagcactaaacagttgaaaggaattgaggaaggtaaccac

SEQ ID NO: 949 - DNA, 55 bp, synthetic construct
  source 1..55: mol_type = other DNA
gtgtgaaacattaatgttcaccgcctggcccttgacggggcttataaatcaaaag

SEQ ID NO: 950 - DNA, 56 bp, synthetic construct
  source 1..56: mol_type = other DNA
tcttttcacgccaccctcagaaccttgccgccgcacaggcggcctttaaggtgtcc

SEQ ID NO: 951 - DNA, 55 bp, synthetic construct
  source 1..55: mol_type = other DNA
gataccgaagacgttagtaaagtgcaccagtacaaactacgacaagaatttgata

SEQ ID NO: 952 - DNA, 54 bp, synthetic construct
  source 1..54: mol_type = other DNA
gaggtgagaaaatctccaaaaaacgaggcaattcatcagttgagatacgaacta

SEQ ID NO: 953 - DNA, 60 bp, synthetic construct
  source 1..60: mol_type = other DNA
tcagtagcaggcaggttaagcccatttgtgaggccttttttccgaccggaaacaatcggc

SEQ ID NO: 954 - DNA, 31 bp, synthetic construct
  source 1..31: mol_type = other DNA
cgtcagaccagagccgccgccagcggatagc

SEQ ID NO: 955 - DNA, 39 bp, synthetic construct
  source 1..39: mol_type = other DNA
gacagaatcaagtttgcctttagcagagcctcaaacaaa

SEQ ID NO: 956 - DNA, 47 bp, synthetic construct
  source 1..47: mol_type = other DNA
acttgagccatttgggaattagagctttttagcaaaatcaccagtag

SEQ ID NO: 957 - DNA, 52 bp, synthetic construct
  source 1..52: mol_type = other DNA
caccgtaaagccataaattgagtcttcttttttgacaacaaagtcagagggt

SEQ ID NO: 958 - DNA, 38 bp, synthetic construct
  source 1..38: mol_type = other DNA
caccattaccattagcaaggccggaaacgtcaacgatt

SEQ ID NO: 959 - DNA, 32 bp, synthetic construct
  source 1..32: mol_type = other DNA
gcgggcgcggtggtttcgtaagccaacgctca

SEQ ID NO: 960 - DNA, 55 bp, synthetic construct
  source 1..55: mol_type = other DNA
gaaaattcattacgcagtatgttatcacgacgttcgccattcaggctggcaccgc

SEQ ID NO: 961 - DNA, 56 bp, synthetic construct
  source 1..56: mol_type = other DNA
acggaataacatacataaaggtggattaagttagggcgatcggtgcggctcaggaa

SEQ ID NO: 962 - DNA, 51 bp, synthetic construct
  source 1..51: mol_type = other DNA
atcagggcgatgaaagaacgtggactcctttgctgagagccagcagcaaat

SEQ ID NO: 963 - DNA, 31 bp, synthetic construct
  source 1..31: mol_type = other DNA
aaagccggcgaacgtggcgagaaaacagctg

SEQ ID NO: 964 - DNA, 45 bp, synthetic construct
  source 1..45: mol_type = other DNA
taaagggagcccccgatttagagctgagagaggaaaatccgctca

SEQ ID NO: 965 - DNA, 28 bp, synthetic construct
  source 1..28: mol_type = other DNA
gtactcagactcctacatgaagaagaaa

SEQ ID NO: 966 - DNA, 51 bp, synthetic construct
  source 1..51: mol_type = other DNA
cgatatattcggtcgcgccgatcgtctttcagcgcaaaatagagactggat

SEQ ID NO: 967 - DNA, 56 bp, synthetic construct
  source 1..56: mol_type = other DNA
gaataggtgtatcaccgcctaattgtatcggtttatcagcttgctttcatataagt

SEQ ID NO: 968 - DNA, 57 bp, synthetic construct
  source 1..57: mol_type = other DNA
taccaacgttttgaagccttaaatcaaaaacagctgatgcgcaatgcctgagtaatt

SEQ ID NO: 969 - DNA, 44 bp, synthetic construct
  source 1..44: mol_type = other DNA
ggaggtttaccctcattttcaggaacggtattctgaaccattag

SEQ ID NO: 970 - DNA, 49 bp, synthetic construct
  source 1..49: mol_type = other DNA
ctgaatctgaaagcgcaaatccaaccacgcaacccttagaatccttgat

SEQ ID NO: 971 - DNA, 37 bp, synthetic construct
  source 1..37: mol_type = other DNA
ctttacagaaggtgaagtaagcagatagccgcagcgt

SEQ ID NO: 972 - DNA, 60 bp, synthetic construct
  source 1..60: mol_type = other DNA
ttatttataaggcttatccggtacgcatttttttaataggaatcattaccgcacagggaa

SEQ ID NO: 973 - DNA, 36 bp, synthetic construct
  source 1..36: mol_type = other DNA
ttttgtttaacgtcaatttttaatgaaaatagcagc

SEQ ID NO: 974 - DNA, 31 bp, synthetic construct
  source 1..31: mol_type = other DNA
tccagtctttttggaaacctgtcgtctgata

SEQ ID NO: 975 - DNA, 46 bp, synthetic construct
  source 1..46: mol_type = other DNA
atcttaacgcctgtagctttttttccacagacagccctcaagagta

SEQ ID NO: 976 - DNA, 40 bp, synthetic construct
  source 1..40: mol_type = other DNA
actagcatgtcaatcacaccctcaagtgtactatacatgg

SEQ ID NO: 977 - DNA, 36 bp, synthetic construct
  source 1..36: mol_type = other DNA
tcattacccaaatcaaacactgtacggtgtcagtac

SEQ ID NO: 978 - DNA, 40 bp, synthetic construct
  source 1..40: mol_type = other DNA
cattcagtgaataagggatagcaaagttgattcaagagaa

SEQ ID NO: 979 - DNA, 48 bp, synthetic construct
  source 1..48: mol_type = other DNA
ttaaaattttctaagaaatttcatcagacgatcgctggcaagatagac

SEQ ID NO: 980 - DNA, 47 bp, synthetic construct
  source 1..47: mol_type = other DNA
ctcattttttaaccagagaacaacgaccgtgatataaagacgtgccg

SEQ ID NO: 981 - DNA, 55 bp, synthetic construct
  source 1..55: mol_type = other DNA
tatgtaccttaatgccaaaatttttaaccttcgccgggcgcggttgcgctttcgc

SEQ ID NO: 982 - DNA, 32 bp, synthetic construct
  source 1..32: mol_type = other DNA
tgcgcgaagccagctgttgttatcctgtttga

SEQ ID NO: 983 - DNA, 60 bp, synthetic construct
  source 1..60: mol_type = other DNA
gccctaaaacccgcttaattccacgcgttgcgtgccccatttttcaggcttgcagcaagc

SEQ ID NO: 984 - DNA, 32 bp, synthetic construct
  source 1..32: mol_type = other DNA
aaatatttcccgacttgcgagaaataaatcct

SEQ ID NO: 985 - DNA, 59 bp, synthetic construct
  source 1..59: mol_type = other DNA
aatcggccaacgcgctttgaatgaatcatggggaaaaacccgggggttcactgcgcgcc

SEQ ID NO: 986 - DNA, 39 bp, synthetic construct
  source 1..39: mol_type = other DNA
tcaacattataatcgggcctgtttgccatattgttggga

SEQ ID NO: 987 - DNA, 50 bp, synthetic construct
  source 1..50: mol_type = other DNA
aggaacaactagccgacaatgacaacaatttttcatcgcccacgcataac

SEQ ID NO: 988 - DNA, 48 bp, synthetic construct
  source 1..48: mol_type = other DNA
aaaggctcattagcggatagcccgggaacccatgtaccgtacgtaaca

SEQ ID NO: 989 - DNA, 54 bp, synthetic construct
  source 1..54: mol_type = other DNA
caaaaggagccttaccaccggaagaaatgcagatacataacgccaaaaggaatt

SEQ ID NO: 990 - DNA, 47 bp, synthetic construct
  source 1..47: mol_type = other DNA
gccaccctcgttccagacccacccagctacaattttatctcggtcat

SEQ ID NO: 991 - DNA, 46 bp, synthetic construct
  source 1..46: mol_type = other DNA
cagagccagcccccttattagcgtttgccagaaccgcctaagcgtc

SEQ ID NO: 992 - DNA, 47 bp, synthetic construct
  source 1..47: mol_type = other DNA
accaccctcagagccgaacagcgggttaaacgatgctgagtgccatc

SEQ ID NO: 993 - DNA, 33 bp, synthetic construct
  source 1..33: mol_type = other DNA
tctagctgataaattaatgccggaggtataagc

SEQ ID NO: 994 - DNA, 46 bp, synthetic construct
  source 1..46: mol_type = other DNA
taccagaaaatattgacggaaattattcattaagagaatagctaat

SEQ ID NO: 995 - DNA, 29 bp, synthetic construct
  source 1..29: mol_type = other DNA
ggtccacgctggttctcactgggtgccgt

SEQ ID NO: 996 - DNA, 54 bp, synthetic construct
  source 1..54: mol_type = other DNA
tccttatcattccaagggccttccgtgaatttaaaagggtcggcggatagaagc

SEQ ID NO: 997 - DNA, 47 bp, synthetic construct
  source 1..47: mol_type = other DNA
accaatcaaaatgtgacatcgtaaaggtcacgttttttaatggaaac

SEQ ID NO: 998 - DNA, 56 bp, synthetic construct
  source 1..56: mol_type = other DNA
ccgcgcttaatgcgccgcaaatcacaactaataggatttatcgtattaaatccttt

SEQ ID NO: 999 - DNA, 43 bp, synthetic construct
  source 1..43: mol_type = other DNA
ttgttccagtttggaactaaagcattcgcctgatacagtaaca

SEQ ID NO: 1000 - DNA, 52 bp, synthetic construct
  source 1..52: mol_type = other DNA
atagctatcttaccgtttttagccctttttaagaaaattatcaccgtcaccg

SEQ ID NO: 1001 - DNA, 27 bp, synthetic construct
  source 1..27: mol_type = other DNA
aggtgcgggctttgcgaaaaaccgtct

SEQ ID NO: 1002 - DNA, 39 bp, synthetic construct
  source 1..39: mol_type = other DNA
ttctgtacatgttttatccccctttttcaaatgcttgta

SEQ ID NO: 1003 - DNA, 32 bp, synthetic construct
  source 1..32: mol_type = other DNA
ggttttgctctggaagacggaacatgaattac

SEQ ID NO: 1004 - DNA, 38 bp, synthetic construct
  source 1..38: mol_type = other DNA
ttatcagagagataagagcaagaaaccatcgatagcag

SEQ ID NO: 1005 - DNA, 47 bp, synthetic construct
  source 1..47: mol_type = other DNA
gtaatttacggaatcattctggtgttagattaagacgctgaatcaat

SEQ ID NO: 1006 - DNA, 37 bp, synthetic construct
  source 1..37: mol_type = other DNA
acgagcattcttttcgtattgggcgccagaagcgtaa

SEQ ID NO: 1007 - DNA, 53 bp, synthetic construct
  source 1..53: mol_type = other DNA
agggcgcgtcatagctgaattattttctgccagcggaattattcatcaatata

SEQ ID NO: 1008 - DNA, 53 bp, synthetic construct
  source 1..53: mol_type = other DNA
accaggtcatagttttcaacagaatgctgttttttgctcaatgggattttgct

SEQ ID NO: 1009 - DNA, 44 bp, synthetic construct
  source 1..44: mol_type = other DNA
aaacaactagcgtaacgatcttttttaagttttgtcgtctttcc

SEQ ID NO: 1010 - DNA, 32 bp, synthetic construct
  source 1..32: mol_type = other DNA
aaatatgcttgctcctccggatatacggtgta

SEQ ID NO: 1011 - DNA, 31 bp, synthetic construct
  source 1..31: mol_type = other DNA
aactaaagagtttcgtccgtcgagagggttg

SEQ ID NO: 1012 - DNA, 32 bp, synthetic construct
  source 1..32: mol_type = other DNA
tttcattctaatttcaaagctgctacaccaga

SEQ ID NO: 1013 - DNA, 32 bp, synthetic construct
  source 1..32: mol_type = other DNA
agtattaaacctattaatcgtaaaacaagaga

SEQ ID NO: 1014 - DNA, 32 bp, synthetic construct
  source 1..32: mol_type = other DNA
ggtaataaagcatcaggcttctgtaaatcgtc

SEQ ID NO: 1015 - DNA, 32 bp, synthetic construct
  source 1..32: mol_type = other DNA
gttttaacataaacagccggttgactatcagg

SEQ ID NO: 1016 - DNA, 47 bp, synthetic construct
  source 1..47: mol_type = other DNA
gtgccttgagtctctgagggtaaaatcaaacttaaatttctgctcat

SEQ ID NO: 1017 - DNA, 31 bp, synthetic construct
  source 1..31: mol_type = other DNA
tcgcaagatgtaaatggaagattagggtagc

SEQ ID NO: 1018 - DNA, 54 bp, synthetic construct
  source 1..54: mol_type = other DNA
tcaaatcagatatagcccaatccaaataagaaccaatgaaacaatgaaatagca

SEQ ID NO: 1019 - DNA, 29 bp, synthetic construct
  source 1..29: mol_type = other DNA
aatggtttgaaatacgcaagcctaaagta

SEQ ID NO: 1020 - DNA, 31 bp, synthetic construct
  source 1..31: mol_type = other DNA
gcgaacctaaattgtaaacgttaatattttg

SEQ ID NO: 1021 - DNA, 32 bp, synthetic construct
  source 1..32: mol_type = other DNA
taattactagtcaatatgtagccaggaacaaa

SEQ ID NO: 1022 - DNA, 30 bp, synthetic construct
  source 1..30: mol_type = other DNA
agtatcatgatcgcagacgacagtatcggc

SEQ ID NO: 1023 - DNA, 47 bp, synthetic construct
  source 1..47: mol_type = other DNA
atgcgttagatgggcggcgatagaacccttctgacctgatcctaatt

SEQ ID NO: 1024 - DNA, 47 bp, synthetic construct
  source 1..47: mol_type = other DNA
tcttaccagtataataaagatgattgccgggttacctgccgttaacg

SEQ ID NO: 1025 - DNA, 30 bp, synthetic construct
  source 1..30: mol_type = other DNA
cggtcacgctgcgcgttctggcgaaagggg

SEQ ID NO: 1026 - DNA, 40 bp, synthetic construct
  source 1..40: mol_type = other DNA
ttcctcgtgcggtttgcaccagtgagacgggcaggaaggg

SEQ ID NO: 1027 - DNA, 31 bp, synthetic construct
  source 1..31: mol_type = other DNA
aacggaatcgacattcaaccgattgagggag

SEQ ID NO: 1028 - DNA, 38 bp, synthetic construct
  source 1..38: mol_type = other DNA
agaaaatagtttattttgtcacaatcaataaatgcaga

SEQ ID NO: 1029 - DNA, 47 bp, synthetic construct
  source 1..47: mol_type = other DNA
actttttcaaatatattttagttacgcgaggcagttacaaaataaac

SEQ ID NO: 1030 - DNA, 29 bp, synthetic construct
  source 1..29: mol_type = other DNA
acaacatattgaatacctttttagttaca

SEQ ID NO: 1031 - DNA, 38 bp, synthetic construct
  source 1..38: mol_type = other DNA
agtgtaaagcctaaccgagaatgaccaggtgtaacatt

SEQ ID NO: 1032 - DNA, 38 bp, synthetic construct
  source 1..38: mol_type = other DNA
actttaagggcttgttcaaataagcaaagcggattgca

SEQ ID NO: 1033 - DNA, 48 bp, synthetic construct
  source 1..48: mol_type = other DNA
cccaattcaactggctaatctacgttaataaattaggaatggattagg

SEQ ID NO: 1034 - DNA, 55 bp, synthetic construct
  source 1..55: mol_type = other DNA
agaacgtcagcgtgcacattttcggactccttatatggtttaccagcgccaaaga

SEQ ID NO: 1035 - DNA, 48 bp, synthetic construct
  source 1..48: mol_type = other DNA
aagcgccattgtaaaacgacggccagtgccaaatgattaaagccagta

SEQ ID NO: 1036 - DNA, 58 bp, synthetic construct
  source 1..58: mol_type = other DNA
tgcttaggttgggttatataactatacaaagaacgcgggaggctaacgagcgtctttc

SEQ ID NO: 1037 - DNA, 40 bp, synthetic construct
  source 1..40: mol_type = other DNA
atcaaaatgcgatagcccggaaaccagggtgctggtctgg

SEQ ID NO: 1038 - DNA, 44 bp, synthetic construct
  source 1..44: mol_type = other DNA
ccgtgcatctgccagagaaaaactgtcttgtttatcaacaatag

SEQ ID NO: 1039 - DNA, 56 bp, synthetic construct
  source 1..56: mol_type = other DNA
gattttagaattacatgaaacaaacaaagaaagcctcttcacagtagaaagtgtag

SEQ ID NO: 1040 - DNA, 39 bp, synthetic construct
  source 1..39: mol_type = other DNA
attaaaggctgagaaggcatcagagtgtgttcagcaaat

SEQ ID NO: 1041 - DNA, 35 bp, synthetic construct
  source 1..35: mol_type = other DNA
agaggtcattgcaatttttctccaacaggtcagga

SEQ ID NO: 1042 - DNA, 31 bp, synthetic construct
  source 1..31: mol_type = other DNA
ccaactttgaaaatttcaagagtacctttaa

SEQ ID NO: 1043 - DNA, 32 bp, synthetic construct
  source 1..32: mol_type = other DNA
gtcaataaccctgcctactcaatccggggtca

SEQ ID NO: 1044 - DNA, 55 bp, synthetic construct
  source 1..55: mol_type = other DNA
tgtgtaggtagtcaaaaaataattcgcgtctaacgggtaataaacacggcagagg

SEQ ID NO: 1045 - DNA, 47 bp, synthetic construct
  source 1..47: mol_type = other DNA
tgtttttataatccaagtcacactggcacagacaatattggggagag

SEQ ID NO: 1046 - DNA, 56 bp, synthetic construct
  source 1..56: mol_type = other DNA
ttgcaacagagcgggagtgccggtcatttcaatcgacaacgaagtattagacttta

SEQ ID NO: 1047 - DNA, 44 bp, synthetic construct
  source 1..44: mol_type = other DNA
tttctccgtggtgaagattgacaatattcaaatttgccgtttta

SEQ ID NO: 1048 - DNA, 37 bp, synthetic construct
  source 1..37: mol_type = other DNA
tccatgtttaccagtcccggaaataataacccacaag

SEQ ID NO: 1049 - DNA, 45 bp, synthetic construct
  source 1..45: mol_type = other DNA
gaaacgtaaacaaagtaattgagcacataaaagcccaatagcaag

SEQ ID NO: 1050 - DNA, 39 bp, synthetic construct
  source 1..39: mol_type = other DNA
ataacctcgccagagcacatcctcataacggataccgac

SEQ ID NO: 1051 - DNA, 54 bp, synthetic construct
  source 1..54: mol_type = other DNA
aggtggagccgccacgggaacggcaataataaaactgaacaccctgctaaattt

SEQ ID NO: 1052 - DNA, 47 bp, synthetic construct
  source 1..47: mol_type = other DNA
gggtaacgccagggttttcccaggcaaacgtgccaacatacgcgcct

SEQ ID NO: 1053 - DNA, 48 bp, synthetic construct
  source 1..48: mol_type = other DNA
gatgtgctgcaaggcgcaacatatggcttaattgaacaagatgtagaa

SEQ ID NO: 1054 - DNA, 43 bp, synthetic construct
  source 1..43: mol_type = other DNA
atcaaaccagaaactttttataacggatcaccttgctgaacct

SEQ ID NO: 1055 - DNA, 53 bp, synthetic construct
  source 1..53: mol_type = other DNA
caaataatagcccgatttttatagggttgagtgaaagcactaaatcggaaccc

SEQ ID NO: 1056 - DNA, 29 bp, synthetic construct
  source 1..29: mol_type = other DNA
gaaaaatcaagagttaactcacattaatt

SEQ ID NO: 1057 - DNA, 39 bp, synthetic construct
  source 1..39: mol_type = other DNA
aaagaagtaacgtcaagacagcatcggggggtgcctaat

SEQ ID NO: 1058 - DNA, 43 bp, synthetic construct
  source 1..43: mol_type = other DNA
agcgttaccagacgactttttataaaaacgagtgagaatagaa

SEQ ID NO: 1059 - DNA, 44 bp, synthetic construct
  source 1..44: mol_type = other DNA
aatcgtcataggcaggcggattcacgttatttcttaaacagctt

SEQ ID NO: 1060 - DNA, 47 bp, synthetic construct
  source 1..47: mol_type = other DNA
tagaaagtagtaagagcaacactatcataaataatttttaatgaatt

SEQ ID NO: 1061 - DNA, 31 bp, synthetic construct
  source 1..31: mol_type = other DNA
accacattcaactcataaaaaaagacgttgg

SEQ ID NO: 1062 - DNA, 48 bp, synthetic construct
  source 1..48: mol_type = other DNA
aaaaagccagcagttgggcggttgtgtacatcccgcctccgaggctga

SEQ ID NO: 1063 - DNA, 47 bp, synthetic construct
  source 1..47: mol_type = other DNA
ccggcaaatctcacggaaaaagagacgcagaccaccagaccagaatg

SEQ ID NO: 1064 - DNA, 39 bp, synthetic construct
  source 1..39: mol_type = other DNA
cgcggtccgttttttcgtctcgttggccttgggaggttg

SEQ ID NO: 1065 - DNA, 32 bp, synthetic construct
  source 1..32: mol_type = other DNA
tgagtgaatagaaccctttcaacggggcgcga

SEQ ID NO: 1066 - DNA, 55 bp, synthetic construct
  source 1..55: mol_type = other DNA
ccgggtcactgtaagaggaagccattttaagtgcgaacgcatataacgcccaata

SEQ ID NO: 1067 - DNA, 30 bp, synthetic construct
  source 1..30: mol_type = other DNA
gctattacgccagatctaaaatagcaaaag

SEQ ID NO: 1068 - DNA, 31 bp, synthetic construct
  source 1..31: mol_type = other DNA
agccgtcatggtggttccgaatacattgccc

SEQ ID NO: 1069 - DNA, 46 bp, synthetic construct
  source 1..46: mol_type = other DNA
acatcgggctcaatcaatatctggtcagttggcatcggcaaaatcc

SEQ ID NO: 1070 - DNA, 31 bp, synthetic construct
  source 1..31: mol_type = other DNA
caacagtgccacgccgcgtagatctaccata

SEQ ID NO: 1071 - DNA, 40 bp, synthetic construct
  source 1..40: mol_type = other DNA
agagggggtcagaaaagagtagagcttaattgctgaatat

SEQ ID NO: 1072 - DNA, 55 bp, synthetic construct
  source 1..55: mol_type = other DNA
ttgcaggctcccgtaacttttgatctcagagctaatcaaaatcaccggaaccaga

SEQ ID NO: 1073 - DNA, 55 bp, synthetic construct
  source 1..55: mol_type = other DNA
gtcggtgttgccgttcattaaagaccaccactgtagcgcgttttcatcggcattt

SEQ ID NO: 1074 - DNA, 29 bp, synthetic construct
  source 1..29: mol_type = other DNA
acgtgctggaggccggaatacggaccagt

SEQ ID NO: 1075 - DNA, 30 bp, synthetic construct
  source 1..30: mol_type = other DNA
ttagttgacattattacaaatattcattga

SEQ ID NO: 1076 - DNA, 40 bp, synthetic construct
  source 1..40: mol_type = other DNA
cttatgcgcgaaagacagatggtttccgcgacctcatctt

SEQ ID NO: 1077 - DNA, 47 bp, synthetic construct
  source 1..47: mol_type = other DNA
cattataccagtcagggtatgagatacatttttgtatcacgcgaaac

SEQ ID NO: 1078 - DNA, 46 bp, synthetic construct
  source 1..46: mol_type = other DNA
taaacatacataggtcaaataagattaaaccacaacatgttcagct

SEQ ID NO: 1079 - DNA, 30 bp, synthetic construct
  source 1..30: mol_type = other DNA
aaaaatgttagaacattatacttctgaata

SEQ ID NO: 1080 - DNA, 47 bp, synthetic construct
  source 1..47: mol_type = other DNA
attctgtccaaaagggacccaaaagaactggcgctttcaggacttgt

SEQ ID NO: 1081 - DNA, 48 bp, synthetic construct
  source 1..48: mol_type = other DNA
gacgggagaattaagggtttttattttcatcgatttttgttaaatcag

SEQ ID NO: 1082 - DNA, 52 bp, synthetic construct
  source 1..52: mol_type = other DNA
ttatatggctattaatgcccgtggggtcagttgctattttgctcagaaccgc

SEQ ID NO: 1083 - DNA, 55 bp, synthetic construct
  source 1..55: mol_type = other DNA
agtacataagaagtttgaggggacctccagccagctttccgcgcaacttaacaac

SEQ ID NO: 1084 - DNA, 32 bp, synthetic construct
  source 1..32: mol_type = other DNA
gaattaccttggtgtaaaacattaaagcaata

SEQ ID NO: 1085 - DNA, 44 bp, synthetic construct
  source 1..44: mol_type = other DNA
tttcatttacaaaattacaggaatacaaattacgagcaaaaata

SEQ ID NO: 1086 - DNA, 59 bp, synthetic construct
  source 1..59: mol_type = other DNA
cacggtcatagctcatggaaataccttttttcattataccgaatgctgtttaataacat

SEQ ID NO: 1087 - DNA, 63 bp, synthetic construct
  source 1..63: mol_type = other DNA
cagacgacataagagatgataaattcagcagcaaccgcaatttttaatgccaacggcagcacc

SEQ ID NO: 1088 - DNA, 32 bp, synthetic construct
  source 1..32: mol_type = other DNA
tagtagcattaacatcccggagacaaagattc

SEQ ID NO: 1089 - DNA, 43 bp, synthetic construct
  source 1..43: mol_type = other DNA
tctttatcatataacaaaggtaatcagaaaagccccaagatta

SEQ ID NO: 1090 - DNA, 26 bp, synthetic construct
  source 1..26: mol_type = other DNA
tttttgaggcgcagataacgggtaaa

SEQ ID NO: 1091 - DNA, 53 bp, synthetic construct
  source 1..53: mol_type = other DNA
tgtgcacatcggcctcgaaccaccagagaggctttgagtttcggccagaatgc

SEQ ID NO: 1092 - DNA, 55 bp, synthetic construct
  source 1..55: mol_type = other DNA
atatcccatagggcgctggcaaagaaacgcaaagacaccataagtcctgagaatc

SEQ ID NO: 1093 - DNA, 50 bp, synthetic construct
  source 1..50: mol_type = other DNA
gacaataaaagtaccgcactcatcataggaactacctttttaacctccgt

SEQ ID NO: 1094 - DNA, 32 bp, synthetic construct
  source 1..32: mol_type = other DNA
gcgcattaggaaggtaggaaaccgaggaaacg

SEQ ID NO: 1095 - DNA, 32 bp, synthetic construct
  source 1..32: mol_type = other DNA
aaggcgtttgagagacgccatcaatcaccatc

SEQ ID NO: 1096 - DNA, 48 bp, synthetic construct
  source 1..48: mol_type = other DNA
taacacttaaattgacgctcattttttcgtctgaaatggagtctttaa

SEQ ID NO: 1097 - DNA, 43 bp, synthetic construct
  source 1..43: mol_type = other DNA
gaggcggtcagtagctcgagccggctcacagtttagcctgagt

SEQ ID NO: 1098 - DNA, 48 bp, synthetic construct
  source 1..48: mol_type = other DNA
ttgccaggatggctggtagcaacggctaccagaagatctcaagcataa

SEQ ID NO: 1099 - DNA, 40 bp, synthetic construct
  source 1..40: mol_type = other DNA
ccactattgcccactacgtgaaccatgtcgattgggcact

SEQ ID NO: 1100 - DNA, 46 bp, synthetic construct
  source 1..46: mol_type = other DNA
tcaagttttcatcgccatgaggatccctttttgggtaccgagctcg

SEQ ID NO: 1101 - DNA, 40 bp, synthetic construct
  source 1..40: mol_type = other DNA
aaacagaacggaatttgcctgctgaccttcacttgcaggt

SEQ ID NO: 1102 - DNA, 40 bp, synthetic construct
  source 1..40: mol_type = other DNA
cttcaaagcggtcgaccggtcaatcataactgacgaaatt

SEQ ID NO: 1103 - DNA, 34 bp, synthetic construct
  source 1..34: mol_type = other DNA
taagtgctcagaaatgtttcgagaggcttttgca

SEQ ID NO: 1104 - DNA, 31 bp, synthetic construct; identical to SEQ ID NO: 405
  source 1..31: mol_type = other DNA

SEQ ID NO: 1105 - DNA, 41 bp, synthetic construct; identical to SEQ ID NO: 406
  source 1..41: mol_type = other DNA

SEQ ID NO: 1106 - DNA, 35 bp, synthetic construct; identical to SEQ ID NO: 407
  source 1..35: mol_type = other DNA

SEQ ID NO: 1107 - DNA, 29 bp, synthetic construct
  source 1..29: mol_type = other DNA
cccctcagtgtcgatgcaatgcctgagta

SEQ ID NO: 1108 - DNA, 16 bp, synthetic construct
  source 1..16: mol_type = other DNA
tacatggccttagccg

SEQ ID NO: 1109 - DNA, 24 bp, synthetic construct
  source 1..24: mol_type = other DNA
gtctctgaataagggagaacggtg

SEQ ID NO: 1110 - DNA, 32 bp, synthetic construct; identical to SEQ ID NO: 411
  source 1..32: mol_type = other DNA

SEQ ID NO: 1111 - DNA, 24 bp, synthetic construct
  source 1..24: mol_type = other DNA
tttttgggaagacaaatcatcgag

SEQ ID NO: 1112 - DNA, 28 bp, synthetic construct
  source 1..28: mol_type = other DNA
ccactactatatttccaagaagcgcctg

SEQ ID NO: 1113 - DNA, 31 bp, synthetic construct; identical to SEQ ID NO: 414
  source 1..31: mol_type = other DNA

SEQ ID NO: 1114 - DNA, 33 bp, synthetic construct; identical to SEQ ID NO: 415
  source 1..33: mol_type = other DNA

SEQ ID NO: 1115 - DNA, 31 bp, synthetic construct; identical to SEQ ID NO: 416
  source 1..31: mol_type = other DNA

SEQ ID NO: 1116 - DNA, 29 bp, synthetic construct
  source 1..29: mol_type = other DNA
catgttattttgatggggtcagtgccttg

SEQ ID NO: 1117 - DNA, 29 bp, synthetic construct
  source 1..29: mol_type = other DNA
gtcaatcatttacctaaacagttaatgcc

SEQ ID NO: 1118 - DNA, 23 bp, synthetic construct
  source 1..23: mol_type = other DNA
ggcttgcagggagttatattcgg

SEQ ID NO: 1119 - DNA, 33 bp, synthetic construct; identical to SEQ ID NO: 420
  source 1..33: mol_type = other DNA

SEQ ID NO: 1120 - DNA, 23 bp, synthetic construct
  source 1..23: mol_type = other DNA
cttagattaagacgcataaataa

SEQ ID NO: 1121 - DNA, 31 bp, synthetic construct; identical to SEQ ID NO: 422
  source 1..31: mol_type = other DNA

SEQ ID NO: 1122 - DNA, 24 bp, synthetic construct
  source 1..24: mol_type = other DNA
ctgacctaaaaaccgtcggggaaa

SEQ ID NO: 1123 - DNA, 25 bp, synthetic construct
  source 1..25: mol_type = other DNA
aattaagcctccagtataaagccaa

SEQ ID NO: 1124 - DNA, 37 bp, synthetic construct; identical to SEQ ID NO: 425
  source 1..37: mol_type = other DNA

SEQ ID NO: 1125 - DNA, 22 bp, synthetic construct
  source 1..22: mol_type = other DNA
ttaatgccggagagggaattac

SEQ ID NO: 1126 - DNA, 15 bp, synthetic construct
  source 1..15: mol_type = other DNA
ggccggagacagtca

SEQ ID NO: 1127 - DNA, 31 bp, synthetic construct; identical to SEQ ID NO: 428
  source 1..31: mol_type = other DNA

SEQ ID NO: 1128 - DNA, 31 bp, synthetic construct; identical to SEQ ID NO: 429
  source 1..31: mol_type = other DNA

SEQ ID NO: 1129 - DNA, 22 bp, synthetic construct
  source 1..22: mol_type = other DNA
tgacccccagcgattgaatttt

SEQ ID NO: 1130 - DNA, 22 bp, synthetic construct
  source 1..22: mol_type = other DNA
aggcaaaagaatacactttaat

SEQ ID NO: 1131 - DNA, 24 bp, synthetic construct
  source 1..24: mol_type = other DNA
cattaaacgggtaaaattgcgccg

SEQ ID NO: 1132 - DNA, 26 bp, synthetic construct
  source 1..26: mol_type = other DNA
aggactaaagactcaccctcagcagc

SEQ ID NO: 1133 - DNA, 30 bp, synthetic construct; identical to SEQ ID NO: 434
  source 1..30: mol_type = other DNA

SEQ ID NO: 1134 - DNA, 31 bp, synthetic construct; identical to SEQ ID NO: 435
  source 1..31: mol_type = other DNA

SEQ ID NO: 1135 - DNA, 16 bp, synthetic construct
  source 1..16: mol_type = other DNA
ctacctttttaacctc

SEQ ID NO: 1136 - DNA, 57 bp, synthetic construct; identical to SEQ ID NO: 437
  source 1..57: mol_type = other DNA

SEQ ID NO: 1137 - DNA, 67 bp, synthetic construct; identical to SEQ ID NO: 438
  source 1..67: mol_type = other DNA

SEQ ID NO: 1138 - DNA, 61 bp, synthetic construct; identical to SEQ ID NO: 439
  source 1..61: mol_type = other DNA

SEQ ID NO: 1139 - DNA, 55 bp, synthetic construct; identical to SEQ ID NO: 440
  source 1..55: mol_type = other DNA

SEQ ID NO: 1140 - DNA, 42 bp, synthetic construct; identical to SEQ ID NO: 441
  source 1..42: mol_type = other DNA

SEQ ID NO: 1141 - DNA, 50 bp, synthetic construct; identical to SEQ ID NO: 442
  source 1..50: mol_type = other DNA

SEQ ID NO: 1142 - DNA, 58 bp, synthetic construct; identical to SEQ ID NO: 443
  source 1..58: mol_type = other DNA

SEQ ID NO: 1143 - DNA, 50 bp, synthetic construct; identical to SEQ ID NO: 444
  source 1..50: mol_type = other DNA

SEQ ID NO: 1144 - DNA, 54 bp, synthetic construct; identical to SEQ ID NO: 445
  source 1..54: mol_type = other DNA

SEQ ID NO: 1145 - DNA, 57 bp, synthetic construct; identical to SEQ ID NO: 446
  source 1..57: mol_type = other DNA

SEQ ID NO: 1146 - DNA, 59 bp, synthetic construct; identical to SEQ ID NO: 447
  source 1..59: mol_type = other DNA

SEQ ID NO: 1147 - DNA, 57 bp, synthetic construct; identical to SEQ ID NO: 448
  source 1..57: mol_type = other DNA

SEQ ID NO: 1148 - DNA, 55 bp, synthetic construct; identical to SEQ ID NO: 449
  source 1..55: mol_type = other DNA

SEQ ID NO: 1149 - DNA, 55 bp, synthetic construct; identical to SEQ ID NO: 450
  source 1..55: mol_type = other DNA

SEQ ID NO: 1150 - DNA, 49 bp, synthetic construct; identical to SEQ ID NO: 451
  source 1..49: mol_type = other DNA

SEQ ID NO: 1151 - DNA, 59 bp, synthetic construct; identical to SEQ ID NO: 452
  source 1..59: mol_type = other DNA

SEQ ID NO: 1152 - DNA, 49 bp, synthetic construct; identical to SEQ ID NO: 453
  source 1..49: mol_type = other DNA

SEQ ID NO: 1153 - DNA, 57 bp, synthetic construct; identical to SEQ ID NO: 454
  source 1..57: mol_type = other DNA

SEQ ID NO: 1154 - DNA, 50 bp, synthetic construct; identical to SEQ ID NO: 455
  source 1..50: mol_type = other DNA

SEQ ID NO: 1155 - DNA, 51 bp, synthetic construct; identical to SEQ ID NO: 456
  source 1..51: mol_type = other DNA

SEQ ID NO: 1156 - DNA, 63 bp, synthetic construct; identical to SEQ ID NO: 457
  source 1..63: mol_type = other DNA

SEQ ID NO: 1157 - DNA, 48 bp, synthetic construct; identical to SEQ ID NO: 458
  source 1..48: mol_type = other DNA

SEQ ID NO: 1158 - DNA, 41 bp, synthetic construct; identical to SEQ ID NO: 459
  source 1..41: mol_type = other DNA

SEQ ID NO: 1159 - DNA, 57 bp, synthetic construct; identical to SEQ ID NO: 460
  source 1..57: mol_type = other DNA

SEQ ID NO: 1160 - DNA, 57 bp, synthetic construct; identical to SEQ ID NO: 461
  source 1..57: mol_type = other DNA

SEQ ID NO: 1161 - DNA, 48 bp, synthetic construct; identical to SEQ ID NO: 462
  source 1..48: mol_type = other DNA

SEQ ID NO: 1162 - DNA, 48 bp, synthetic construct; identical to SEQ ID NO: 463
  source 1..48: mol_type = other DNA

SEQ ID NO: 1163 - DNA, 50 bp, synthetic construct; identical to SEQ ID NO: 464
  source 1..50: mol_type = other DNA

SEQ ID NO: 1164 - DNA, 52 bp, synthetic construct; identical to SEQ ID NO: 465
  source 1..52: mol_type = other DNA

SEQ ID NO: 1165 - DNA, 56 bp, synthetic construct; identical to SEQ ID NO: 466
  source 1..56: mol_type = other DNA

SEQ ID NO: 1166 - DNA, 57 bp, synthetic construct; identical to SEQ ID NO: 467
  source 1..57: mol_type = other DNA

SEQ ID NO: 1167 - DNA, 42 bp, synthetic construct; identical to SEQ ID NO: 468
  source 1..42: mol_type = other DNA

SEQ ID NO: 1168 - DNA, 54 bp, synthetic construct
  source 1..54: mol_type = other DNA
ccgtttttttagcgttagagccagcaaaatagggagccgctttccagtcgggaa

SEQ ID NO: 1169 - DNA, 54 bp, synthetic construct
  source 1..54: mol_type = other DNA
gaataacctgtcgtgtttttcagctgcattaatgttttttggggtcgaggtgcc

SEQ ID NO: 1170 - DNA, 54 bp, synthetic construct
  source 1..54: mol_type = other DNA
agtcagaatcttgacattacgagaccaacgccacgttggtgtagatccgtaatg

SEQ ID NO: 1171 - DNA, 54 bp, synthetic construct
  source 1..54: mol_type = other DNA
aaaatcctgtttaacgggtaaaaacatgctttgaataccaagttacaaaatcgc

SEQ ID NO: 1172 - DNA, 54 bp, synthetic construct
  source 1..54: mol_type = other DNA
ttctttgctcgtcatttagatggtggttccgaaatcggcaaaatccggtaatgg

SEQ ID NO: 1173 - DNA, 53 bp, synthetic construct
  source 1..53: mol_type = other DNA
gtgagaactcatagttagcgtattgcgggaagtagtagcattaacgggcgcga

SEQ ID NO: 1174 - DNA, 54 bp, synthetic construct
  source 1..54: mol_type = other DNA
cggattgagaacggtctgaccaatgccactaactttttcatgaggaagtttcca

SEQ ID NO: 1175 - DNA, 53 bp, synthetic construct
  source 1..53: mol_type = other DNA
cttataaaacgctggtttgccccgcagaggcttacatcgggagaaagtttaac

SEQ ID NO: 1176 - DNA, 53 bp, synthetic construct
  source 1..53: mol_type = other DNA
tttatcccagagcctcttcatcgaccaggcttaccgttccagtcatcagatgc

SEQ ID NO: 1177 - DNA, 53 bp, synthetic construct
  source 1..53: mol_type = other DNA
tttcatcacccaaaaagacctgctttgaccgcagcgaaagacagcatcggaac

SEQ ID NO: 1178 - DNA, 53 bp, synthetic construct
  source 1..53: mol_type = other DNA
tcattgacaggaggttgccccctgcctatttaaccgatcgccaccctcagaac

SEQ ID NO: 1179 - DNA, 53 bp, synthetic construct
  source 1..53: mol_type = other DNA
acccagctaacttgaccattagactatattttcatttgatccaataagcaaac

SEQ ID NO: 1180 - DNA, 53 bp, synthetic construct
  source 1..53: mol_type = other DNA
gagataggtgaggatccccgcactctgtggtgctgcggaaagtttgcagttgg

SEQ ID NO: 1181 - DNA, 52 bp, synthetic construct
  source 1..52: mol_type = other DNA
cgccaccctcagagctttttaccaccctcattttcacggtttatcagcttgc

SEQ ID NO: 1182 - DNA, 54 bp, synthetic construct
  source 1..54: mol_type = other DNA
ttttttaaccaaattggcaacatagacagcctagaaaggaacaactaaaggaat

SEQ ID NO: 1183 - DNA, 53 bp, synthetic construct
  source 1..53: mol_type = other DNA
cgtaaccgtgcacacgacgttgtcctaccactgctcataaccagctcagacga

SEQ ID NO: 1184 - DNA, 51 bp, synthetic construct
  source 1..51: mol_type = other DNA
cccttcaccgcgttgttccagtttggaatttttaagagtccactattaaag

SEQ ID NO: 1185 - DNA, 51 bp, synthetic construct
  source 1..51: mol_type = other DNA
ataacggaataccactacgcgaggcaggcaaatatattttgtttgaaatac

SEQ ID NO: 1186 - DNA, 50 bp, synthetic construct
  source 1..50: mol_type = other DNA
ccgccgcgtccggcagatatagtgtttaccaacgctcgcgttaaataaga

SEQ ID NO: 1187 - DNA, 50 bp, synthetic construct
  source 1..50: mol_type = other DNA
ttcatatgcaccctcagaaccgcacctttaacagagagaataacataaat

SEQ ID NO: 1188 - DNA, 50 bp, synthetic construct
  source 1..50: mol_type = other DNA
ggcttgcacgtataacaggaggtaagtttttcccacgtcgagagggttga

SEQ ID NO: 1189 - DNA, 55 bp, synthetic construct
  source 1..55: mol_type = other DNA
tgacgacgaaaagaagtttttgataagaggtcactccctcttaagaaataaaggt

SEQ ID NO: 1190 - DNA, 48 bp, synthetic construct
  source 1..48: mol_type = other DNA
gatgatacacttagcctgaacaaatgtgagccagttacaaaataaact

SEQ ID NO: 1191 - DNA, 48 bp, synthetic construct
  source 1..48: mol_type = other DNA
agcgaaccgattggccaattgagtgatttgtatgtaaacgatcaccat

SEQ ID NO: 1192 - DNA, 48 bp, synthetic construct
  source 1..48: mol_type = other DNA
tagccatataaatagcaagccctttagagccgcgtttaccagcgccaa

SEQ ID NO: 1193 - DNA, 57 bp, synthetic construct
  source 1..57: mol_type = other DNA
aacgtggactacgcgtggctgtttctctggtcatttttcagcaagaattcgtaatca

SEQ ID NO: 1194 - DNA, 56 bp, synthetic construct
  source 1..56: mol_type = other DNA
ttgctaaagtcgtctttccagacggtaccaaacaggcaaggcaaagaatcaataac

SEQ ID NO: 1195 - DNA, 53 bp, synthetic construct
  source 1..53: mol_type = other DNA
tttttagaaccctcatatattttcgtcaccgtggcggataagtgcataataag

SEQ ID NO: 1196 - DNA, 54 bp, synthetic construct
  source 1..54: mol_type = other DNA
cgctgcgcgtaaccaaattttccgtcaatactacctttaacagtagggcttaat

SEQ ID NO: 1197 - DNA, 56 bp, synthetic construct
  source 1..56: mol_type = other DNA
gagctaaatgcgggagcgttttagcgaacctttagcaaatgattaaatggagcggg

SEQ ID NO: 1198 - DNA, 56 bp, synthetic construct
  source 1..56: mol_type = other DNA
aggagtgtaatggaaagcgcagtcattaagagaacaccctaaccctcgtttaccat

SEQ ID NO: 1199 - DNA, 47 bp, synthetic construct
  source 1..47: mol_type = other DNA
attttaccagaatgcggcgtttttgccgttttcacggtacgttatta

SEQ ID NO: 1200 - DNA, 56 bp, synthetic construct
  source 1..56: mol_type = other DNA
cagactgtagcgcgtttaaagcattgccgtaatatttagcgccatcttcgctatta

SEQ ID NO: 1201 - DNA, 54 bp, synthetic construct
  source 1..54: mol_type = other DNA
agcacgtacttaatgtgagaagacttagaatccttgaaaacatagcgcaagtgt

SEQ ID NO: 1202 - DNA, 52 bp, synthetic construct
  source 1..52: mol_type = other DNA
actaaagcgaaggcaccaacctatcaggtaactagcatgtcaatggattctc

SEQ ID NO: 1203 - DNA, 51 bp, synthetic construct
  source 1..51: mol_type = other DNA
ccaccaccggaaccgctttttgctaacgtcaaaatagtacggtgttaatgc

SEQ ID NO: 1204 - DNA, 55 bp, synthetic construct
  source 1..55: mol_type = other DNA
aattattcaagaaagcgaaagaattttctgtatgggattgagggaagtattacgc

SEQ ID NO: 1205 - DNA, 47 bp, synthetic construct
  source 1..47: mol_type = other DNA
gtaaagcactaaatcggaaccctaacaccagtaagtgagcttcgctg

SEQ ID NO: 1206 - DNA, 47 bp, synthetic construct
  source 1..47: mol_type = other DNA
ctactaatgaagcctttatttcaacgcaaggcattccacataaaaga

SEQ ID NO: 1207 - DNA, 47 bp, synthetic construct
  source 1..47: mol_type = other DNA
tcaggattagagagtcaccctcaaatagcaaagacaccattgttaaa

SEQ ID NO: 1208 - DNA, 47 bp, synthetic construct
  source 1..47: mol_type = other DNA
cgaggaaacccaattctgcaacaccgcctgcaacagtgcattagcgt

SEQ ID NO: 1209 - DNA, 47 bp, synthetic construct
  source 1..47: mol_type = other DNA
ttttttgtaaatgctgatgcaaatcctttcaaagcgccattctggtg

SEQ ID NO: 1210 - DNA, 46 bp, synthetic construct
  source 1..46: mol_type = other DNA
tataaagccagtcccggaatttgttttttagagatagacttatcgg

SEQ ID NO: 1211 - DNA, 46 bp, synthetic construct
  source 1..46: mol_type = other DNA
agaggcaacaacggctacagaggctttgaggtcgttaaccataagg

SEQ ID NO: 1212 - DNA, 46 bp, synthetic construct
  source 1..46: mol_type = other DNA
tttcgaggtgaatttcttaaacagtttttttgataccgatagttgc

SEQ ID NO: 1213 - DNA, 46 bp, synthetic construct
  source 1..46: mol_type = other DNA
tggctgacaatttgcgagtaacaacccgtccatatgtaggcgcaga

SEQ ID NO: 1214 - DNA, 46 bp, synthetic construct
  source 1..46: mol_type = other DNA
acaatcaatgcgaatactacaacgcctgtagataaaaatcagctca

SEQ ID NO: 1215 - DNA, 46 bp, synthetic construct
  source 1..46: mol_type = other DNA
ttgctcagtaccattaccctcagagccgccagcaaactccaacagg

SEQ ID NO: 1216 - DNA, 46 bp, synthetic construct
  source 1..46: mol_type = other DNA
aaaacgaacggtcaatggaagcgtcatacatggcttttgagggtag

SEQ ID NO: 1217 - DNA, 46 bp, synthetic construct
  source 1..46: mol_type = other DNA
tcagctaatctgtaaatcgtcgctattaattccacgctcactgccc

SEQ ID NO: 1218 - DNA, 46 bp, synthetic construct
  source 1..46: mol_type = other DNA
gaacggtacgccgtgaaccaccacagcaaatgaaaaatcttcatcg

SEQ ID NO: 1219 - DNA, 46 bp, synthetic construct
  source 1..46: mol_type = other DNA
gcataggcggaacgaccccggtttttttgagagatctacaaaggct

SEQ ID NO: 1220 - DNA, 46 bp, synthetic construct
  source 1..46: mol_type = other DNA
taggtaaaataggaactataagtagaaggattagagccgccgccag

SEQ ID NO: 1221 - DNA, 46 bp, synthetic construct
  source 1..46: mol_type = other DNA
caggaaggctgataaattaatgccggagagcactcatctccatgtt

SEQ ID NO: 1222 - DNA, 45 bp, synthetic construct
  source 1..45: mol_type = other DNA
ccatgtaaaaggctccaaaaggagcctttacctcaagatagccct

SEQ ID NO: 1223 - DNA, 45 bp, synthetic construct
  source 1..45: mol_type = other DNA
ttatttgcacgtaaagggcgcatcgagaaacctacattaggaaaa

SEQ ID NO: 1224 - DNA, 45 bp, synthetic construct
  source 1..45: mol_type = other DNA
tagatacagagcaacttgaatcggattgcatcaaaaagtctgaat

SEQ ID NO: 1225 - DNA, 45 bp, synthetic construct
  source 1..45: mol_type = other DNA
aagaatacactaaaaggtagctagataatcagaaaagcacattaa

SEQ ID NO: 1226 - DNA, 45 bp, synthetic construct
  source 1..45: mol_type = other DNA
agtagatttagtgatgaacaaagaccagagttcaaccgattgagg

SEQ ID NO: 1227 - DNA, 45 bp, synthetic construct
  source 1..45: mol_type = other DNA
cttacacagcggtgccggtgcctcaaaaatatcaacgtgccctga

SEQ ID NO: 1228 - DNA, 45 bp, synthetic construct
  source 1..45: mol_type = other DNA
tggtcatacctgttcttcgcgtttttccgtgagcctcctcacagt

SEQ ID NO: 1229 - DNA, 45 bp, synthetic construct
  source 1..45: mol_type = other DNA
aatcgtaacattgcctgagagtctggagcaatacgtaaagcgggg

SEQ ID NO: 1230 - DNA, 47 bp, synthetic construct
  source 1..47: mol_type = other DNA
gcacagactaacgtgccaggctgcaaggcttagacaaagttaacctc

SEQ ID NO: 1231 - DNA, 47 bp, synthetic construct
  source 1..47: mol_type = other DNA
aacattatgaccctgtaatacttacgatctagtagaaaagggcgaca

SEQ ID NO: 1232 - DNA, 44 bp, synthetic construct
  source 1..44: mol_type = other DNA
caatcagaagatgatgaatttttcaaacatccaacagctgattg

SEQ ID NO: 1233 - DNA, 44 bp, synthetic construct
  source 1..44: mol_type = other DNA
cgagctcccgcaagacgggtattttttaaaccaagtgtattttt

SEQ ID NO: 1234 - DNA, 44 bp, synthetic construct
  source 1..44: mol_type = other DNA
tacatacaagtaagcgctgaaaccaatccaaataagaacaattt

SEQ ID NO: 1235 - DNA, 47 bp, synthetic construct
  source 1..47: mol_type = other DNA
taacaattgggtggttatccgctcacaattcccggacttaacaagca

SEQ ID NO: 1236 - DNA, 43 bp, synthetic construct
  source 1..43: mol_type = other DNA
aaagctaaatcggttttagtaagactcctgtaaatattttcat

SEQ ID NO: 1237 - DNA, 43 bp, synthetic construct
  source 1..43: mol_type = other DNA
caatatgatattcaaagcgcgatgataaatcagtgccacaaac

SEQ ID NO: 1238 - DNA, 43 bp, synthetic construct
  source 1..43: mol_type = other DNA
accttgcttgcagatttttcgcgcctgatcaatatatgtgagt

SEQ ID NO: 1239 - DNA, 42 bp, synthetic construct
  source 1..42: mol_type = other DNA
catatttaacaattctttttttccagtgagaatcaatagcaa

SEQ ID NO: 1240 - DNA, 42 bp, synthetic construct
  source 1..42: mol_type = other DNA
ctttccttaacatgagccgagcggtcctcaaaagaatagccc

SEQ ID NO: 1241 - DNA, 47 bp, synthetic construct
  source 1..47: mol_type = other DNA
atatcagagagataacccacaagttgatattcttgagtaacagtgcc

SEQ ID NO: 1242 - DNA, 47 bp, synthetic construct
  source 1..47: mol_type = other DNA
aatcaaaagaatataatgcgaacgcatataactagttgctattttgc

SEQ ID NO: 1243 - DNA, 41 bp, synthetic construct
  source 1..41: mol_type = other DNA
tttttataagagagagacacatcgactcagcgtggtgctgg

SEQ ID NO: 1244 - DNA, 41 bp, synthetic construct
  source 1..41: mol_type = other DNA
gttgagtctggccctgagagagagcaaaaataatcggctgt

SEQ ID NO: 1245 - DNA, 40 bp, synthetic construct
  source 1..40: mol_type = other DNA
acaacatgtcataggtctgagagagtgaatttgcaaatca

SEQ ID NO: 1246 - DNA, 40 bp, synthetic construct
  source 1..40: mol_type = other DNA
taagacgccgccgctaagcggtcacgaacgtggcgagaaa

SEQ ID NO: 1247 - DNA, 40 bp, synthetic construct
  source 1..40: mol_type = other DNA
ggttagaaaaaacgaccctgattggattatttccagaaca

SEQ ID NO: 1248 - DNA, 47 bp, synthetic construct
  source 1..47: mol_type = other DNA
agcaggcgaatccgccgtaaaggttccagcgcagtgtcaccaaagaa

SEQ ID NO: 1249 - DNA, 39 bp, synthetic construct
  source 1..39: mol_type = other DNA
taccctgactattatcgggttacagaggacagaaccgaa

SEQ ID NO: 1250 - DNA, 47 bp, synthetic construct
  source 1..47: mol_type = other DNA
taacaaagtatcaaaattgcgtagattttcagcaataacgcgcactc

SEQ ID NO: 1251 - DNA, 47 bp, synthetic construct
  source 1..47: mol_type = other DNA
agtaccttgaattattcatttcaattacctgttgcagcaggtggtac

SEQ ID NO: 1252 - DNA, 41 bp, synthetic construct
  source 1..41: mol_type = other DNA
gaatcggccaacgcgtacataatttatcaagacgacaataa

SEQ ID NO: 1253 - DNA, 39 bp, synthetic construct
  source 1..39: mol_type = other DNA
cagagcggttagacagccggaaacctttccggcaccgct

SEQ ID NO: 1254 - DNA, 39 bp, synthetic construct
  source 1..39: mol_type = other DNA
attgtataagcaaatatttaaattcatcgccaacaaagt

SEQ ID NO: 1255 - DNA, 57 bp, synthetic construct
  source 1..57: mol_type = other DNA
gggagttacaacggatttagtacagtcaattaattttttatttgcctgagtaatgtg

SEQ ID NO: 1256 - DNA, 39 bp, synthetic construct
  source 1..39: mol_type = other DNA
taaagggtgagaaaggccggagaccgccaccaccgtact

SEQ ID NO: 1257 - DNA, 39 bp, synthetic construct
  source 1..39: mol_type = other DNA
aaaaatgatatttatcaggtggcatcataggaacgccat

SEQ ID NO: 1258 - DNA, 39 bp, synthetic construct
  source 1..39: mol_type = other DNA
gccgacaatgacaacaaccatcgcccacgcatcggaacc

SEQ ID NO: 1259 - DNA, 39 bp, synthetic construct
  source 1..39: mol_type = other DNA
aaaggccgcttttgcgggatcgttaacggggttgtgtcg

SEQ ID NO: 1260 - DNA, 57 bp, synthetic construct
  source 1..57: mol_type = other DNA
taacagggaagcgcattagacgggagaggtaattgaagccagactggtaataagttt

SEQ ID NO: 1261 - DNA, 39 bp, synthetic construct
  source 1..39: mol_type = other DNA
cagggcgcgtgaattaggtcatagcccccttcacgctga

SEQ ID NO: 1262 - DNA, 31 bp, synthetic construct
  source 1..31: mol_type = other DNA
gatgaacgcgtgggaataacgagcgtctttc

SEQ ID NO: 1263 - DNA, 39 bp, synthetic construct
  source 1..39: mol_type = other DNA
gatattcactttgaactgcagcctggtgtgttcagcaaa

SEQ ID NO: 1264 - DNA, 39 bp, synthetic construct
  source 1..39: mol_type = other DNA
aaatgcaattgttaaaattcgcattaaatttcggaataa

SEQ ID NO: 1265 - DNA, 31 bp, synthetic construct
  source 1..31: mol_type = other DNA
aatcagttttttgcgacagaatcaaaaaccc

SEQ ID NO: 1266 - DNA, 38 bp, synthetic construct
  source 1..38: mol_type = other DNA
ctttgacggcattttctcaccgtcaccgactaagccgg

SEQ ID NO: 1267 - DNA, 38 bp, synthetic construct
  source 1..38: mol_type = other DNA
aaataagaacgcgagaaaacttaatcgcaatccggtat

SEQ ID NO: 1268 - DNA, 38 bp, synthetic construct
  source 1..38: mol_type = other DNA
tatacttcaatataatggccagtttgggtaacgccagg

SEQ ID NO: 1269 - DNA, 38 bp, synthetic construct
  source 1..38: mol_type = other DNA
ataaacacggaaacatctccgtgccgcacatcataacg

SEQ ID NO: 1270 - DNA, 38 bp, synthetic construct
  source 1..38: mol_type = other DNA
aaggagcggaattatccctgcattacggctggcgcttt

SEQ ID NO: 1271 - DNA, 57 bp, synthetic construct
  source 1..57: mol_type = other DNA
ttttttgacctaagccttaacccgactcaggaggcgcaatattgccatcttgacgga

SEQ ID NO: 1272 - DNA, 38 bp, synthetic construct
  source 1..38: mol_type = other DNA
tggaataggtgtatcctcagaacatattcggtcgctga

SEQ ID NO: 1273 - DNA, 38 bp, synthetic construct
  source 1..38: mol_type = other DNA
gatagctctagtatcacataattactagaaaatttttt

SEQ ID NO: 1274 - DNA, 38 bp, synthetic construct
  source 1..38: mol_type = other DNA
aagttttcaactttcaacagtttcagcggaagacaaaa

SEQ ID NO: 1275 - DNA, 31 bp, synthetic construct
  source 1..31: mol_type = other DNA
tcgagccaaccgcactcctaatttaattgtt

SEQ ID NO: 1276 - DNA, 38 bp, synthetic construct
  source 1..38: mol_type = other DNA
ttatcattttgcggaatgcgcgcccgtcggtttgcggt

SEQ ID NO: 1277 - DNA, 37 bp, synthetic construct
  source 1..37: mol_type = other DNA
aattgcttcaccggattaccagagtatgttaatcata

SEQ ID NO: 1278 - DNA, 37 bp, synthetic construct
  source 1..37: mol_type = other DNA
gagccgccacggggagcactagtaataaacatcactt

SEQ ID NO: 1279 - DNA, 31 bp, synthetic construct
  source 1..31: mol_type = other DNA
cggcagcactgtgcactgttgccctgcggct

SEQ ID NO: 1280 - DNA, 31 bp, synthetic construct
  source 1..31: mol_type = other DNA
gggcgcggggtgccagtcagatgaatggaag

SEQ ID NO: 1281 - DNA, 36 bp, synthetic construct
  source 1..36: mol_type = other DNA
cgaccgtgtgattgggaaggtcgccatttttcctcg

SEQ ID NO: 1282 - DNA, 36 bp, synthetic construct
  source 1..36: mol_type = other DNA
ggcatttagcctgtttcacggaaaaaatataaagta

SEQ ID NO: 1283 - DNA, 31 bp, synthetic construct
  source 1..31: mol_type = other DNA
aaataaattcgcgttttaattcgagcttcaa

SEQ ID NO: 1284 - DNA, 36 bp, synthetic construct
  source 1..36: mol_type = other DNA
tattattctgaaacattttttaaagtattaagaggc

SEQ ID NO: 1285 - DNA, 36 bp, synthetic construct
  source 1..36: mol_type = other DNA
tgagactattgtatgggatagcaagcccagattcat

SEQ ID NO: 1286 - DNA, 35 bp, synthetic construct
  source 1..35: mol_type = other DNA
gcggttgtgtgcagtttttaacagcggatcaaact

SEQ ID NO: 1287 - DNA, 35 bp, synthetic construct
  source 1..35: mol_type = other DNA
taatggaaacagcggggagacggaagcataaagtg

SEQ ID NO: 1288 - DNA, 34 bp, synthetic construct
  source 1..34: mol_type = other DNA
tgtttagactggatagcgtccaatacgaaagact

SEQ ID NO: 1289 - DNA, 34 bp, synthetic construct
  source 1..34: mol_type = other DNA
aattaagcaataatagttatataactatattttt

SEQ ID NO: 1290 - DNA, 34 bp, synthetic construct
  source 1..34: mol_type = other DNA
gaacgtgcacacaacgaacaagaattaccttttt

SEQ ID NO: 1291 - DNA, 28 bp, synthetic construct
  source 1..28: mol_type = other DNA
cgctagggcgctggaagcctcagagcat

SEQ ID NO: 1292 - DNA, 32 bp, synthetic construct
  source 1..32: mol_type = other DNA
gcggggtttccaaaaaccgtaacactgagttt

SEQ ID NO: 1293 - DNA, 32 bp, synthetic construct
  source 1..32: mol_type = other DNA
ccagcatcttacccaacaggtcttgagaatga

SEQ ID NO: 1294 - DNA, 28 bp, synthetic construct
  source 1..28: mol_type = other DNA
attagcaaggccggaaacgtcaaatcaa

SEQ ID NO: 1295 - DNA, 58 bp, synthetic construct
  source 1..58: mol_type = other DNA
acagttaatgaggcaggtcagacagactttttcggaaccagaaccaccaccaggatta

SEQ ID NO: 1296 - DNA, 26 bp, synthetic construct
  source 1..26: mol_type = other DNA
tttctttaaagggcgaaaaaccgtct

SEQ ID NO: 1297 - DNA, 32 bp, synthetic construct
  source 1..32: mol_type = other DNA
agatagcctttttgttggatggctaactaaag

SEQ ID NO: 1298 - DNA, 28 bp, synthetic construct
  source 1..28: mol_type = other DNA
atggttgttagaatgagccaggcagaag

SEQ ID NO: 1299 - DNA, 28 bp, synthetic construct
  source 1..28: mol_type = other DNA
ttaccgagcctttattgctcctttgcca

SEQ ID NO: 1300 - DNA, 32 bp, synthetic construct
  source 1..32: mol_type = other DNA
tgaatcttgcatagtaataacgcctttcaact

SEQ ID NO: 1301 - DNA, 32 bp, synthetic construct
  source 1..32: mol_type = other DNA
atcaagatagttgatttcagttgaaaaacgaa

SEQ ID NO: 1302 - DNA, 29 bp, synthetic construct
  source 1..29: mol_type = other DNA
ggccagaggaatcattatttttcgcgccc

SEQ ID NO: 1303 - DNA, 32 bp, synthetic construct
  source 1..32: mol_type = other DNA
aagaactggctcattacttttgcataaaaacc

SEQ ID NO: 1304 - DNA, 32 bp, synthetic construct
  source 1..32: mol_type = other DNA
caccctcacccagcgattataccaccgttcta

SEQ ID NO: 1305 - DNA, 32 bp, synthetic construct
  source 1..32: mol_type = other DNA
aacgcggtgatgctgatcaccttgatcgccat

SEQ ID NO: 1306 - DNA, 32 bp, synthetic construct
  source 1..32: mol_type = other DNA
aaggaaacctgtttagtacatttcgcaaatgg

SEQ ID NO: 1307 - DNA, 32 bp, synthetic construct
  source 1..32: mol_type = other DNA
caaatatcgtttgccttcgtctcgaactcaca

SEQ ID NO: 1308 - DNA, 31 bp, synthetic construct
  source 1..31: mol_type = other DNA
atcaaaatttaattgcgttgcacttgggaat

SEQ ID NO: 1309 - DNA, 28 bp, synthetic construct
  source 1..28: mol_type = other DNA
aatcccgtatctttagaacggatagggg

SEQ ID NO: 1310 - DNA, 31 bp, synthetic construct
  source 1..31: mol_type = other DNA
agtacaaaataattttttcacgttgaaaatc

SEQ ID NO: 1311 - DNA, 28 bp, synthetic construct
  source 1..28: mol_type = other DNA
cggcttaggttgggcttagattctaaga

SEQ ID NO: 1312 - DNA, 58 bp, synthetic construct
  source 1..58: mol_type = other DNA
atcagggcgatggcgtattgggcgccatcatttgaaaaataaccgacaaataggcaga

SEQ ID NO: 1313 - DNA, 31 bp, synthetic construct
  source 1..31: mol_type = other DNA
aaaaaaaggtgaaggagccgtttatgtaatt

SEQ ID NO: 1314 - DNA, 31 bp, synthetic construct
  source 1..31: mol_type = other DNA
agcaagaaacaatgagagccaccattttgtc

SEQ ID NO: 1315 - DNA, 29 bp, synthetic construct
  source 1..29: mol_type = other DNA
atttaatgagttaatttcatcttcttttt

SEQ ID NO: 1316 - DNA, 31 bp, synthetic construct
  source 1..31: mol_type = other DNA
gattcgcctgatagagaatcgatacagaaat

SEQ ID NO: 1317 - DNA, 29 bp, synthetic construct
  source 1..29: mol_type = other DNA
tcaatagataaatctgattctgctcattt

SEQ ID NO: 1318 - DNA, 29 bp, synthetic construct
  source 1..29: mol_type = other DNA
ccggaattatgcgttatacaaacgccaac

SEQ ID NO: 1319 - DNA, 30 bp, synthetic construct
  source 1..30: mol_type = other DNA
atgccaagccgccagagtaacatttagaag

SEQ ID NO: 1320 - DNA, 59 bp, synthetic construct
  source 1..59: mol_type = other DNA
cgaaacgtacttgaatggctattagtttttttttaaattgaggtagaatttgttgtagc

SEQ ID NO: 1321 - DNA, 30 bp, synthetic construct
  source 1..30: mol_type = other DNA
tcaaatacctcattaagcgctaaaatccgc

SEQ ID NO: 1322 - DNA, 31 bp, synthetic construct
  source 1..31: mol_type = other DNA
aaacatcctcattgcaggaggtgtaaagaaa

SEQ ID NO: 1323 - DNA, 23 bp, synthetic construct
  source 1..23: mol_type = other DNA
ttatttttaagtcctatacgagc

SEQ ID NO: 1324 - DNA, 31 bp, synthetic construct
  source 1..31: mol_type = other DNA
cccgatttagagcttgacggggatgagccat

SEQ ID NO: 1325 - DNA, 30 bp, synthetic construct
  source 1..30: mol_type = other DNA
tcccacgctttggataccaccagatcagat

SEQ ID NO: 1326 - DNA, 30 bp, synthetic construct
  source 1..30: mol_type = other DNA
cgagtagtagaaccgggataggtcaaacgg

SEQ ID NO: 1327 - DNA, 30 bp, synthetic construct
  source 1..30: mol_type = other DNA
cgccagcaacagagaaaggttatctaaaat

SEQ ID NO: 1328 - DNA, 30 bp, synthetic construct
  source 1..30: mol_type = other DNA
ctgtgtgaacgagcaaaaattaattacatt

SEQ ID NO: 1329 - DNA, 30 bp, synthetic construct
  source 1..30: mol_type = other DNA
taaatataatatacagtatcattccaagaa

SEQ ID NO: 1330 - DNA, 29 bp, synthetic construct
  source 1..29: mol_type = other DNA
ggaagggattaaaggtcaaaagaactggc

SEQ ID NO: 1331 - DNA, 30 bp, synthetic construct
  source 1..30: mol_type = other DNA
agtcagagattaactggaagccctgcggaa

SEQ ID NO: 1332 - DNA, 30 bp, synthetic construct
  source 1..30: mol_type = other DNA
gtgtacaaagagtaagcaaagccccctcaa

SEQ ID NO: 1333 - DNA, 30 bp, synthetic construct
  source 1..30: mol_type = other DNA
tatcccatcatcgaggtagaacgataaaaa

SEQ ID NO: 1334 - DNA, 30 bp, synthetic construct
  source 1..30: mol_type = other DNA
aggtaaagtaattctgtccagaccaataga

SEQ ID NO: 1335 - DNA, 60 bp, synthetic construct
  source 1..60: mol_type = other DNA
tcaatcaatgcaccgtcagcctcctaaagcctggggtgcttttttaatggcaccattacc

SEQ ID NO: 1336 - DNA, 62 bp, synthetic construct
  source 1..62: mol_type = other DNA
tagaaaaaacgcaatagctatccaaaaataattcgcgtctttttggccttcctgtagccagc

SEQ ID NO: 1337 - DNA, 50 bp, synthetic construct
  source 1..50: mol_type = other DNA
tctgccagcccaacgtctcaccagtgagacgggaagaaaactgtagaaac

SEQ ID NO: 1338 - DNA, 27 bp, synthetic construct
  source 1..27: mol_type = other DNA
tttaaatcctttgcccgacataccggt

SEQ ID NO: 1339 - DNA, 45 bp, synthetic construct
  source 1..45: mol_type = other DNA
tcgatagcaatctggtttagtgatgatttttgggtaaagttaaac

SEQ ID NO: 1340 - DNA, 28 bp, synthetic construct
  source 1..28: mol_type = other DNA
tttttgaccaacaactaaattcgacaat

SEQ ID NO: 1341 - DNA, 40 bp, synthetic construct
  source 1..40: mol_type = other DNA
catcgtagcacatccggcggcctcagttggcaaatcaatt

SEQ ID NO: 1342 - DNA, 40 bp, synthetic construct
  source 1..40: mol_type = other DNA
ggcggtttgcccactacgtgaaccatcacccaccaatgat

SEQ ID NO: 1343 - DNA, 48 bp, synthetic construct
  source 1..48: mol_type = other DNA
tagttgaaaggatgcgcgaactttttgatagccctaaaacctgaacct

SEQ ID NO: 1344 - DNA, 28 bp, synthetic construct
  source 1..28: mol_type = other DNA
aaattgggatcgtttttcctcaggaaga

SEQ ID NO: 1345 - DNA, 39 bp, synthetic construct
  source 1..39: mol_type = other DNA
aataccacattcaacctggaagtaaatatgctagagctt

SEQ ID NO: 1346 - DNA, 34 bp, synthetic construct
  source 1..34: mol_type = other DNA
cagattcacgattaaggccaagctttcagaggtg

SEQ ID NO: 1347 - DNA, 24 bp, synthetic construct
  source 1..24: mol_type = other DNA
aggtagaaccagccaggttttgaa

SEQ ID NO: 1348 - DNA, 30 bp, synthetic construct
  source 1..30: mol_type = other DNA
caatcgtagttctgccagtttgaggggacg

SEQ ID NO: 1349 - DNA, 24 bp, synthetic construct
  source 1..24: mol_type = other DNA
cgtaagaatgcgggccgcaactgt

SEQ ID NO: 1350 - DNA, 31 bp, synthetic construct
  source 1..31: mol_type = other DNA
gttttcccctgaaatgtcagtgatcctgatt

SEQ ID NO: 1351 - DNA, 29 bp, synthetic construct
  source 1..29: mol_type = other DNA
tgggaagaaaaatctatgttttttcattc

SEQ ID NO: 1352 - DNA, 36 bp, synthetic construct
  source 1..36: mol_type = other DNA
tcgcactagattcaccgattagcggtcagtatttgt

SEQ ID NO: 1353 - DNA, 36 bp, synthetic construct
  source 1..36: mol_type = other DNA
acgcaaatttttttaccttccttctgactgtccatc

SEQ ID NO: 1354 - DNA, 30 bp, synthetic construct
  source 1..30: mol_type = other DNA
actatcggccttgctttctacatttgagga

SEQ ID NO: 1355 - DNA, 31 bp, synthetic construct
  source 1..31: mol_type = other DNA
acgacagtggaaatacaccagaacagaaaac

SEQ ID NO: 1356 - DNA, 30 bp, synthetic construct
  source 1..30: mol_type = other DNA
ttaatcattgtgaataatattcaactatca

SEQ ID NO: 1357 - DNA, 23 bp, synthetic construct
  source 1..23: mol_type = other DNA
ccataaaattgcaacttgacgct

SEQ ID NO: 1358 - DNA, 24 bp, synthetic construct
  source 1..24: mol_type = other DNA
agctcaacacgttaatgatttagg

SEQ ID NO: 1359 - DNA, 31 bp, synthetic construct
  source 1..31: mol_type = other DNA
gcgatcggtacagaatcctgagaagtgttac

SEQ ID NO: 1360 - DNA, 23 bp, synthetic construct
  source 1..23: mol_type = other DNA
gatggcaaggtaatatacattgg

SEQ ID NO: 1361 - DNA, 22 bp, synthetic construct
  source 1..22: mol_type = other DNA
cgagaggtaccagtcaggacgt

SEQ ID NO: 1362 - DNA, 23 bp, synthetic construct
  source 1..23: mol_type = other DNA
ataaaacatatttttataatcag

SEQ ID NO: 1363 - DNA, 23 bp, synthetic construct
  source 1..23: mol_type = other DNA
atgctttaggtttaaaaaaggaa

SEQ ID NO: 1364 - DNA, 31 bp, synthetic construct
  source 1..31: mol_type = other DNA
aatacttctttgattagtaataaagggacat

SEQ ID NO: 1365 - DNA, 24 bp, synthetic construct
  source 1..24: mol_type = other DNA
tcgtcatataccttatgcgatttt

SEQ ID NO: 1366 - DNA, 18 bp, synthetic construct
  source 1..18: mol_type = other DNA
gagggggtaatagtaaaa

SEQ ID NO: 1367 - DNA, 39 bp, synthetic construct
  source 1..39: mol_type = other DNA
tattagattttttttacaaacatagtttttttagagccg

SEQ ID NO: 1368 - DNA, 24 bp, synthetic construct
  source 1..24: mol_type = other DNA
gcctgagtagatttttgaactcaa

SEQ ID NO: 1369 - DNA, 24 bp, synthetic construct
  source 1..24: mol_type = other DNA
tgaggccaccgagtaaaccgaacg

SEQ ID NO: 1370 - DNA, 21 bp, synthetic construct
  source 1..21: mol_type = other DNA
taaaaataagagtcctgaaag

SEQ ID NO: 1371 - DNA, 23 bp, synthetic construct
  source 1..23: mol_type = other DNA
acgctcatcttgagataacagtt

SEQ ID NO: 1372 - DNA, 31 bp, synthetic construct
  source 1..31: mol_type = other DNA
ctaacggaacaacatgaggtgagaagggatt

SEQ ID NO: 1373 - DNA, 31 bp, synthetic construct
  source 1..31: mol_type = other DNA
atattaccgccagcccatcatatataaggct

SEQ ID NO: 1374 - DNA, 28 bp, synthetic construct
  source 1..28: mol_type = other DNA
gatgtgctgctttttaaggccagtcaca

SEQ ID NO: 1375 - DNA, 22 bp, synthetic construct
  source 1..22: mol_type = other DNA
tctggcctggcgaaaacctcac

SEQ ID NO: 1376 - DNA, 74 bp, synthetic construct
  source 1..74: mol_type = other DNA
gcagtagagtaggtagagattaggcattttttttttttttttttttaaattgggatcgtttttcctcaggaaga

SEQ ID NO: 1377 - DNA, 85 bp, synthetic construct
  source 1..85: mol_type = other DNA
gcagtagagtaggtagagattaggcattttttttttttttttttttaataccacattcaacctggaagtaaatatgctagagctt

SEQ ID NO: 1378 - DNA, 80 bp, synthetic construct
  source 1..80: mol_type = other DNA
gcagtagagtaggtagagattaggcattttttttttttttttttttcagattcacgattaaggccaagctttcagaggtg

SEQ ID NO: 1379 - DNA, 70 bp, synthetic construct
  source 1..70: mol_type = other DNA
gcagtagagtaggtagagattaggcattttttttttttttttttttaggtagaaccagccaggttttgaa

SEQ ID NO: 1380 - DNA, 76 bp, synthetic construct
  source 1..76: mol_type = other DNA
gcagtagagtaggtagagattaggcattttttttttttttttttttcaatcgtagttctgccagtttgaggggacg

SEQ ID NO: 1381 - DNA, 70 bp, synthetic construct
  source 1..70: mol_type = other DNA
gcagtagagtaggtagagattaggcattttttttttttttttttttcgtaagaatgcgggccgcaactgt

SEQ ID NO: 1382 - DNA, 77 bp, synthetic construct
  source 1..77: mol_type = other DNA
gcagtagagtaggtagagattaggcattttttttttttttttttttgttttcccctgaaatgtcagtgatcctgatt

SEQ ID NO: 1383 - DNA, 75 bp, synthetic construct
  source 1..75: mol_type = other DNA
gcagtagagtaggtagagattaggcatttttttttttttttttttttgggaagaaaaatctatgttttttcattc

SEQ ID NO: 1384 - DNA, 82 bp, synthetic construct
  source 1..82: mol_type = other DNA
gcagtagagtaggtagagattaggcatttttttttttttttttttttcgcactagattcaccgattagcggtcagtatttgt

SEQ ID NO: 1385 - DNA, 82 bp, synthetic construct
  source 1..82: mol_type = other DNA
gcagtagagtaggtagagattaggcattttttttttttttttttttacgcaaatttttttaccttccttctgactgtccatc

SEQ ID NO: 1386 - DNA, 76 bp, synthetic construct
  source 1..76: mol_type = other DNA
gcagtagagtaggtagagattaggcattttttttttttttttttttactatcggccttgctttctacatttgagga

SEQ ID NO: 1387 - DNA, 77 bp, synthetic construct
  source 1..77: mol_type = other DNA
gcagtagagtaggtagagattaggcattttttttttttttttttttacgacagtggaaatacaccagaacagaaaac

SEQ ID NO: 1388 - DNA, 76 bp, synthetic construct
  source 1..76: mol_type = other DNA
gcagtagagtaggtagagattaggcattttttttttttttttttttttaatcattgtgaataatattcaactatca

SEQ ID NO: 1389 - DNA, 69 bp, synthetic construct
  source 1..69: mol_type = other DNA
gcagtagagtaggtagagattaggcattttttttttttttttttttccataaaattgcaacttgacgct

SEQ ID NO: 1390 - DNA, 70 bp, synthetic construct
  source 1..70: mol_type = other DNA
gcagtagagtaggtagagattaggcattttttttttttttttttttagctcaacacgttaatgatttagg

SEQ ID NO: 1391 - DNA, 77 bp, synthetic construct
  source 1..77: mol_type = other DNA
gcagtagagtaggtagagattaggcattttttttttttttttttttgcgatcggtacagaatcctgagaagtgttac

SEQ ID NO: 1392 - DNA, 69 bp, synthetic construct
  source 1..69: mol_type = other DNA
gcagtagagtaggtagagattaggcattttttttttttttttttttgatggcaaggtaatatacattgg

SEQ ID NO: 1393 - DNA, 68 bp, synthetic construct
  source 1..68: mol_type = other DNA
gcagtagagtaggtagagattaggcattttttttttttttttttttcgagaggtaccagtcaggacgt

SEQ ID NO: 1394 - DNA, 69 bp, synthetic construct
  source 1..69: mol_type = other DNA
gcagtagagtaggtagagattaggcattttttttttttttttttttataaaacatatttttataatcag

SEQ ID NO: 1395 - DNA, 69 bp, synthetic construct
  source 1..69: mol_type = other DNA
gcagtagagtaggtagagattaggcattttttttttttttttttttatgctttaggtttaaaaaaggaa

SEQ ID NO: 1396 - DNA, 77 bp, synthetic construct
  source 1..77: mol_type = other DNA
gcagtagagtaggtagagattaggcattttttttttttttttttttaatacttctttgattagtaataaagggacat

SEQ ID NO: 1397 - DNA, 70 bp, synthetic construct
  source 1..70: mol_type = other DNA
gcagtagagtaggtagagattaggcatttttttttttttttttttttcgtcatataccttatgcgatttt

SEQ ID NO: 1398 - DNA, 64 bp, synthetic construct
  source 1..64: mol_type = other DNA
gcagtagagtaggtagagattaggcattttttttttttttttttttgagggggtaatagtaaaa

SEQ ID NO: 1399 - DNA, 85 bp, synthetic construct
  source 1..85: mol_type = other DNA
gcagtagagtaggtagagattaggcatttttttttttttttttttttattagattttttttacaaacatagtttttttagagccg

SEQ ID NO: 1400 - DNA, 70 bp, synthetic construct
  source 1..70: mol_type = other DNA
gcagtagagtaggtagagattaggcattttttttttttttttttttgcctgagtagatttttgaactcaa

SEQ ID NO: 1401 - DNA, 70 bp, synthetic construct
  source 1..70: mol_type = other DNA
gcagtagagtaggtagagattaggcatttttttttttttttttttttgaggccaccgagtaaaccgaacg

SEQ ID NO: 1402 - DNA, 67 bp, synthetic construct
  source 1..67: mol_type = other DNA
gcagtagagtaggtagagattaggcatttttttttttttttttttttaaaaataagagtcctgaaag

SEQ ID NO: 1403 - DNA, 69 bp, synthetic construct
  source 1..69: mol_type = other DNA
gcagtagagtaggtagagattaggcattttttttttttttttttttacgctcatcttgagataacagtt

SEQ ID NO: 1404 - DNA, 77 bp, synthetic construct
  source 1..77: mol_type = other DNA
gcagtagagtaggtagagattaggcattttttttttttttttttttctaacggaacaacatgaggtgagaagggatt

SEQ ID NO: 1405 - DNA, 77 bp, synthetic construct
  source 1..77: mol_type = other DNA
gcagtagagtaggtagagattaggcattttttttttttttttttttatattaccgccagcccatcatatataaggct

SEQ ID NO: 1406 - DNA, 74 bp, synthetic construct
  source 1..74: mol_type = other DNA
gcagtagagtaggtagagattaggcattttttttttttttttttttgatgtgctgctttttaaggccagtcaca

SEQ ID NO: 1407 - DNA, 68 bp, synthetic construct
  source 1..68: mol_type = other DNA
gcagtagagtaggtagagattaggcatttttttttttttttttttttctggcctggcgaaaacctcac

SEQ ID NO: 1408 - DNA, 26 bp, synthetic construct
  source 1..26: mol_type = other DNA
tgcctaatctctacctactctactgc